(19) United States
(12) Patent Application Publication
(10) Pub. No.: US 2003/0022277 A1
(43) Pub. Date: Jan. 30, 2003

(54) HUMAN NEUROPEPTIDE RECEPTOR

(76) Inventors: DANIEL R. SOPPET, CENTREVILLE, VA (US); YI LI, SUNNYVALE, CA (US); CRAIG A. ROSEN, LAYTONSVILLE, MD (US)

Correspondence Address: HUMAN GENOME SCIENCES INC, 9410 KEY WEST AVENUE, ROCKVILLE, MD, 20850

(21) Appl. No.: 09/393,696

(22) Filed: Sep. 10, 1999

Related U.S. Application Data
(63) Continuation-in-part of application No. 08/462,509, filed on Jun. 5, 1995.
Application No. 08/462,509 is a continuation-in-part of application No. PCT/US95/05616, filed on May 5, 1995.

(51) Int. Cl. C12Q 1/68; C12P 21/06; G01N 33/53; G01N 33/567; C07H 21/04; C12N 15/00; C07K 14/00; C07K 17/00; C07K 1/00; C12N 15/09; C12N 15/63; C12N 5/00; C12N 5/02; C12N 15/74; C12N 15/70

(52) U.S. Cl. 435/69.1; 435/6; 435/7.1; 435/320.1; 435/325; 530/350; 536/23.5

(57) ABSTRACT

The present invention relates to a novel human protein called human neuropeptide receptor, and isolated polynucleotides encoding this protein. Also provided are vectors, host cells, antibodies, and recombinant methods for producing this human protein. The invention further relates to diagnostic and therapeutic methods useful for diagnosing and treating disorders related to this novel human protein.

This application is a continuation-in-part of Ser. No. 08/462,509, filed Jun. 5, 1995, which is a continuation-in-part of application Ser. No. PCT/US95/05616, filed May 5, 1995, both of which are hereby incorporated by reference in their entirety.

### FIELD OF THE INVENTION

The present invention relates to a novel human gene encoding a polypeptide which is a member of the seven-transmembrane, G-protein coupled cell surface receptor (GPCR) family. More specifically, the present invention relates to a polynucleotide encoding a novel human polypeptide named human neuropeptide receptor, or neuropeptide receptor. This invention also relates to neuropeptide receptor polypeptides, as well as vectors, host cells, antibodies directed to neuropeptide receptor polypeptides, and the recombinant methods for producing the same. Also provided are diagnostic methods for detecting disorders related to the central nervous and peripheral nervous system, and therapeutic methods for treating such disorders. The invention further relates to screening methods for identifying agonists and antagonists of receptor neuropeptide polypeptides.

### BACKGROUND OF THE INVENTION

This invention relates to newly identified polynucleotides, polypeptides encoded by such polynucleotides, the use of such polynucleotides and polypeptides, as well as the production of such polynucleotides and polypeptides. The polypeptides of the present invention are human 7-transmembrane G-protein coupled receptors. More particularly, the polypeptides of the present invention are neuropeptide receptor polypeptides, sometimes hereinafter referred to as neuropeptide receptor polypeptides. The invention also relates to inhibiting the action of such polypeptides.

Obesity is the most common nutritional disorder in Western societies. More than three in ten adult Americans weigh at least 20% in excess of their ideal body weight (Burroa, M., The New York Times, Jul. 17, 1994). Increased body weight is an important public health problem because it is associated with Type II diabetes, hypertension, hyperlipidemia and certain cancers (Grundy, S. M., and Barnett, J. P., Disease-a-Month, 36:645-696 (1990)).

Five single-gene mutations in the mouse obesity gene (ob) which result in an obese phenotype have been described (Friedman, J. M. & Leibel, R. L., Cell, 66:217-220 (1990)). The cloning and sequencing of the mouse ob gene and its human homologue have been reported (Zhang, Y., et al., Nature, 372:425-431 (1994)). The ob gene encodes a 4.5-kb adipose tissue mRNA with a highly conserved 167-amino-acid open reading frame. The predicted amino-acid sequence is 84% identical between human and mouse and has features of a secreted protein. The ob gene product may function as part of a signalling pathway from adipose tissue that acts to regulate the size of the body fat depot (id. 425).

Of the brain regions implicated in the regulation of feeding behavior, the ventromedial nucleus of the hypothalamus (VMH) is considered to be the most important satiety center in the central nervous system (CNS). The energy balance in mammals is therefore postulated to be controlled by a feedback loop in which the amount of stored energy is sensed by the hypothalamus, which adjusts food intake and energy expenditure to maintain a constant body weight (Ombeck, J. R., Yale J. Biol. Med., 20:545-552 (1948) and Kennedy, G. C., Proc. R. Soc.148:578-592 (1953)). In the lipostasis theory, the size of the body fat depot is regulated by the CNS, with a product of body fat metabolism affecting energy balance by interacting with the hypothalamus (Kennedy, G. C., Proc. R. Soc.148:578-592 (1953)).

The inability to identify the putative signal from fat has hindered the validation of the lipostasis theory. The possibility that at least one component of the signalling system circulates in the bloodstream was first suggested by Hervey (Dietrich, W., et al., Genetics, 131:423-447 (1992)), who showed that the transfer of blood from an animal with a VMH lesion across a vascular graft to an untreated animal (a parabiosis experiment) resulted in a reduction of food intake in the intact animal. It is now significant that there is evidence that the ob gene product is secreted, suggesting that ob may encode this circulating factor.

The ob signal may act directly or indirectly on the CNS to inhibit food intake and/or regulate energy expenditure as part of a homeostatic mechanism to maintain constancy of the adipose mass (Zhang, Y., et al., Nature, 372:425-431, 431 (1994)). The ob gene apparently encodes a protein secreted by fat, and mutations apparently prevent translation or expression of the gene (Rink, T., Nature, 372:406-407 (1994)).

Parabiosis experiments suggest that the ob receptor is encoded by the mouse db (diabetes) gene (Coleman, D. L., Diabetologia, 14:141-148 (1978)). Mice having a mutation in the db gene are also obese, with the defect possibly being a receptor defect. (Id. at 406).

Neuropeptide Y is similar to the ob gene product in that it mediates the feeding response. Neuropeptide Y acts on at least four types of neuropeptide Y receptors called Y₁, Y₂, Y₃ and an atypical Y₁ receptor, which mediates the feeding response stimulated by neuropeptide Y.

Neuropeptide Y has a wide range of biological functions. Neuropeptide Y is found to be widely distributed in the central nervous system (CNS) and the peripheral nervous system (PNS). In the PNS, neuropeptide Y is found in the noradrenergic sympathetic innervation of blood vessels and other smooth muscle tissues and in neurons within the enteric nervous system. Neuropeptide Y immunoreactive fibers also occur in the non-vascular smooth muscle, surrounding exocrine glands and surface epithelia. Neuropeptide Y also occurs in subpopulations of neurons and is generally co-localized with other neurotransmitters, particular noradrenaline.

In the CNS, neuropeptide Y is contained in GABAergic interneurons in higher centers and in predominantly catecholaminergic cells that project further caudally. For example, neuropeptide Y is contained in interneurons in the cortex, hippocampus, amygdala, basal forebrain and striatum, whereas in the brain stem, neuropeptide Y is contained in noradrenergic neurons of the A₁ and A₂ groups in the medulla, and the locus coeruleus (LC). In the hypothalamus, neuropeptide Y is found predominantly in the arcuate nucleus and lateral hypothalamus.

Within the peripheral nervous system, neuropeptide Y is present in postganglionic sympathetic nerves, and is co-localized as stated above with other neurotransmitters, including catecholamines. When used pharmacologically, neuropeptide Y has been shown to have a potent vasoconstrictor activity as well as dramatically potentiating the vasoconstriction caused by many other pressor agents. Particularly high concentrations of neuropeptide Y are found in the sympathetic nerves supplying the coronary, cerebral and renal vasculature and when infused into these vascular beds, neuropeptide Y causes prolonged vasoconstriction that is not reversed by adrenergic blocking agents. These observations have lead to the proposal that neuropeptide Y is the candidate transmitter for pathological vasospasm, a major cause of morbidity and mortality when involving the coronary and cerebral vessels.

Neuropeptide Y also appears to be involved in interaction with the renin angiotensin system. Neuropeptide Y containing sympathetic nerve terminals are found on the juxta-glomerular apparatus of the renal cortex and neuropeptide Y influences renin release. These data, together with the demonstration of all durations in neuropeptide Y concentrations in hypertensive animal models and the pressor response to infusion of the peptide, have resulted in implications of this peptide in hypertension.

Within the central nervous system neuropeptide Y is located predominantly within intemeurons where it appears to have a regulatory role. It therefore has widespread and diverse effects including effects on memory and a possible role in Alzheimer's disease. Neuropeptide Y is the most potent known substance to cause an increase in feeding and may play a role in the genetic basis of Type II Diabetes Mellitus. Neuropeptide Y may also play a role as a regulatory agent and pituitary function as well as potential neuromodulatory function in stress responses and in reproductive function.

### SUMMARY OF THE INVENTION

In accordance with one aspect of the present invention, there are provided novel mature receptor polypeptides as well as biologically active and diagnostically or therapeutically useful fragments, analogs and derivatives thereof. The receptor polypeptides of the present invention are of human origin.

In accordance with another aspect of the present invention, there are provided isolated nucleic acid molecules encoding the receptor polypeptides of the present invention, including mRNAs, DNAs, cDNAs, genomic DNA as well as antisense analogs thereof and biologically active and diagnostically or therapeutically useful fragments thereof.

In accordance with a further aspect of the present invention, there are provided processes for producing such receptor polypeptides by recombinant techniques comprising culturing recombinant prokaryotic and/or eukaryotic host cells, containing nucleic acid sequences encoding the receptor polypeptides of the present invention, under conditions promoting expression of said polypeptides and subsequent recovery of said polypeptides.

In accordance with yet a further aspect of the present invention, there are provided antibodies against such receptor polypeptides.

In accordance with another aspect of the present invention there are provided methods of screening for compounds which bind to and activate or inhibit activation of the receptor polypeptides of the present invention.

In accordance with still another embodiment of the present invention there are provided processes of administering compounds to a host which bind to and activate the receptor polypeptide of the present invention which are useful in the prevention and/or treatment of obesity, hyperlipidemia, certain cancers, to stimulate neuronal growth, to regulate neurotransmission, to enhance activity levels and utilization of ingested foods.

In accordance with another aspect of the present invention there is provided a method of administering the receptor polypeptides of the present invention via gene therapy to treat conditions related to underexpression of the polypeptides or underexpression of a ligand to the receptor polypeptide.

In accordance with still another embodiment of the present invention there are provided processes of administering compounds to a host which bind to and inhibit activation of the receptor polypeptides of the present invention which are useful in the prevention and/or treatment of Alzheimer's disease, Type II Diabetes Mellitus, epilepsy, stress, anxiety, hypertension, cardiovascular disease, psychotic conditions and obesity caused by neuropeptide Y.

In accordance with yet another aspect of the present invention, there are provided nucleic acid probes comprising nucleic acid molecules of sufficient length to specifically hybridize to the polynucleotide sequences of the present invention.

In accordance with still another aspect of the present invention, there are provided diagnostic assays for detecting diseases related to mutations in the nucleic acid sequences encoding such polypeptides and for detecting an altered level of the soluble form of the receptor polypeptides.

In accordance with yet a further aspect of the present invention, there are provided processes for utilizing such receptor polypeptides, or polynucleotides encoding such polypeptides, for in vitro purposes related to scientific research, synthesis of DNA and manufacture of DNA vectors.

These and other aspects of the present invention should be apparent to those skilled in the art from the teachings herein.

### BRIEF DESCRIPTION OF THE DRAWINGS

The following drawings are illustrative of embodiments of the invention and are not meant to limit the scope of the invention as encompassed by the claims.

FIGS. 1A-F show the cDNA sequence (SEQ ID NO:1) and the corresponding deduced amino acid sequence (SEQ ID NO:2) of the neuropeptide receptor polypeptide of the present invention. The standard one-letter abbreviation for amino acids is used. Sequencing was performed using a 373 Automated DNA sequencer (Applied Biosystems, Inc.).

FIGS. 2A-E show the cDNA sequence (SEQ ID NO:3) and the corresponding deduced amino acid sequence (SEQ ID NO:4) of the neuropeptide receptor splice variant 1 polypeptide of the present invention. The standard one-letter abbreviation for amino acids is used.

FIGS. 3A-F show the cDNA sequence (SEQ ID NO:5) and the corresponding deduced amino acid sequence (SEQ ID NO:6) of the neuropeptide receptor splice variant 2 polypeptide of the present invention. The standard one-letter abbreviation for amino acids is used.

FIG. 4 illustrates the amino acid sequence and seven transmembrane regions of the neuropeptide receptor (SEQ ID NO:2). The transmembrane regions are underlined and denoted with a TM.

FIG. 5 illustrates the amino acid sequence and seven transmembrane regions of the neuropeptide receptor splice variant 1 (SEQ ID NO:4). The transmembrane regions are underlined and denoted with a TM.

FIG. 6 illustrates the amino acid sequence and seven transmembrane regions of the neuropeptide receptor splice variant 2 (SEQ ID NO:6). The transmembrane regions are underlined and denoted with a TM.

FIGS. 7A and 7B show the regions of identity between the amino acid sequence of the neuropeptide receptor protein (SEQ ID NO:2) and the translation product of human neuropeptide Y receptor protein (SEQ ID NO:23), determined by BLAST analysis. By examining the regions of conservation, the skilled artisan can readily identify conserved domains between the two polypeptides. These conserved domains are preferred embodiments of the present invention.

FIG. 8 shows an analysis of the neuropeptide receptor amino acid sequence (SEQ ID NO: 2). Alpha, beta, turn and coil regions; hydrophilicity and hydrophobicity; amphipathic regions; flexible regions; antigenic index and surface probability are shown, and all were generated using the default settings. In the “Antigenic Index or Jameson-Wolf” graph, the positive peaks indicate locations of the highly antigenic regions of the neuropeptide receptor protein, i.e., regions from which epitope-bearing peptides of the invention can be obtained. The domains defined by these graphs are contemplated by the present invention.

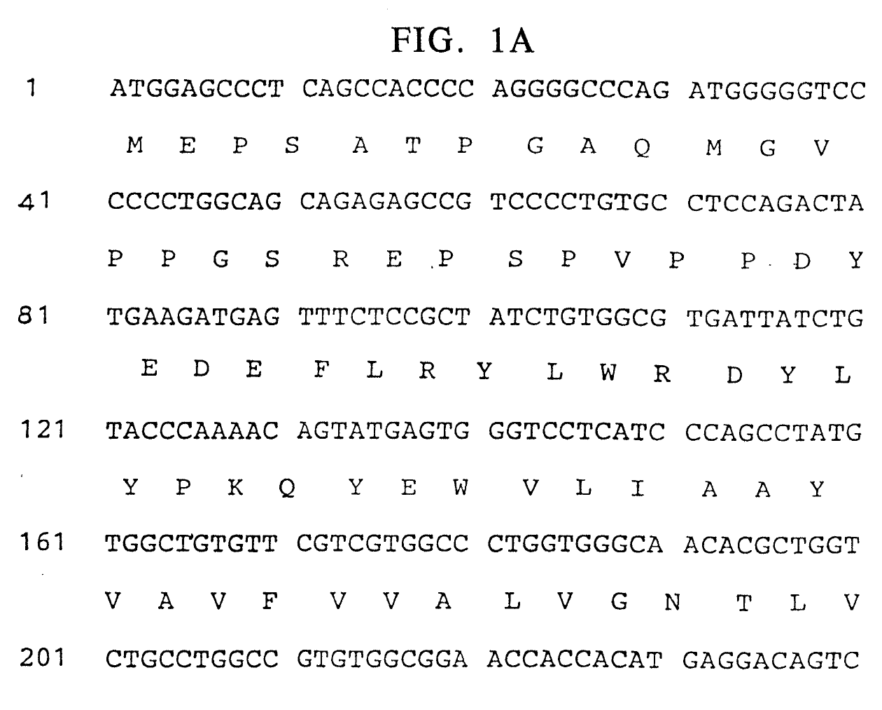

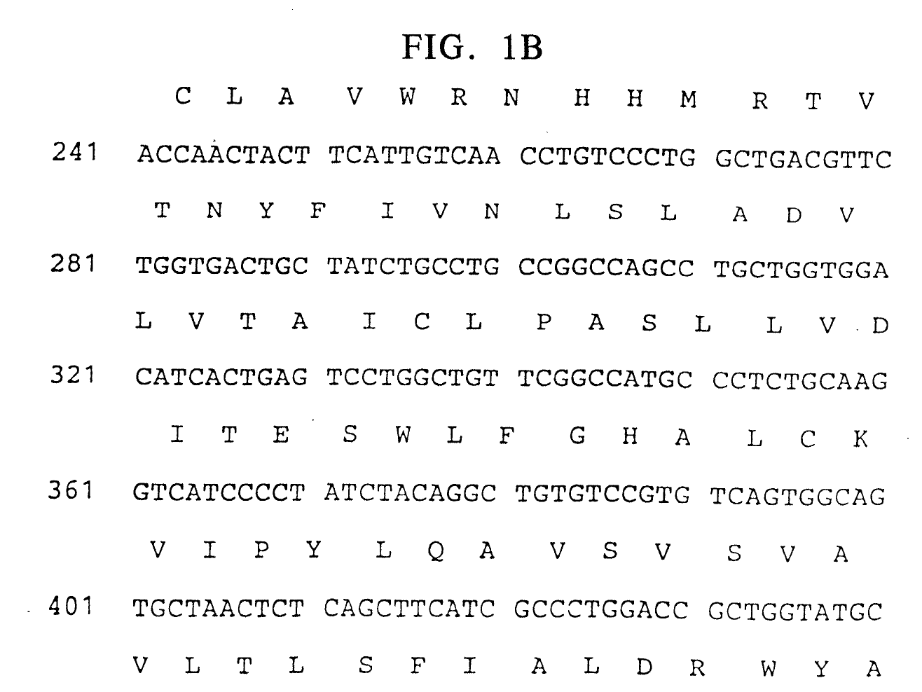

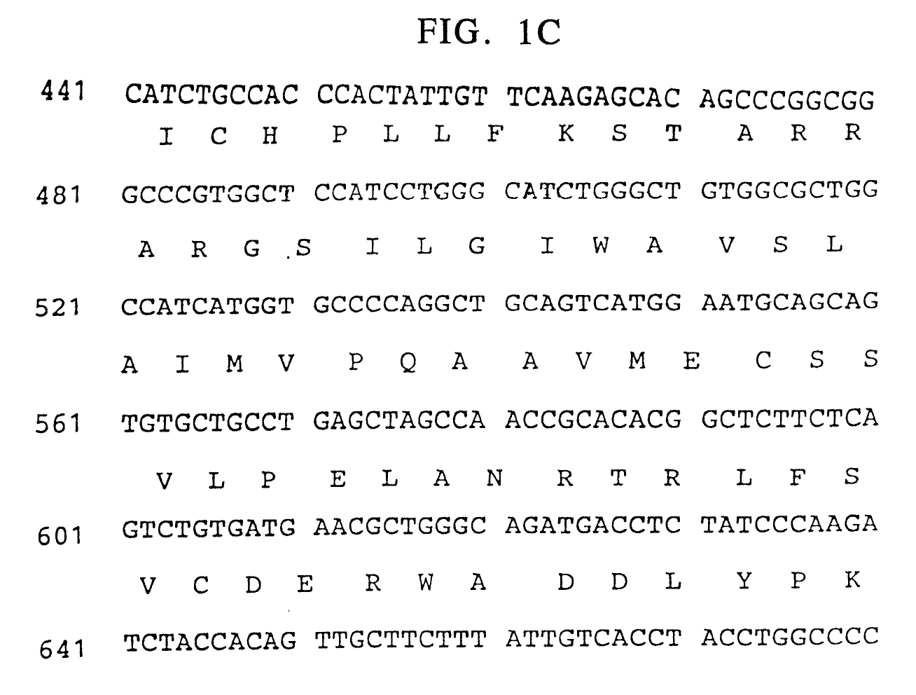

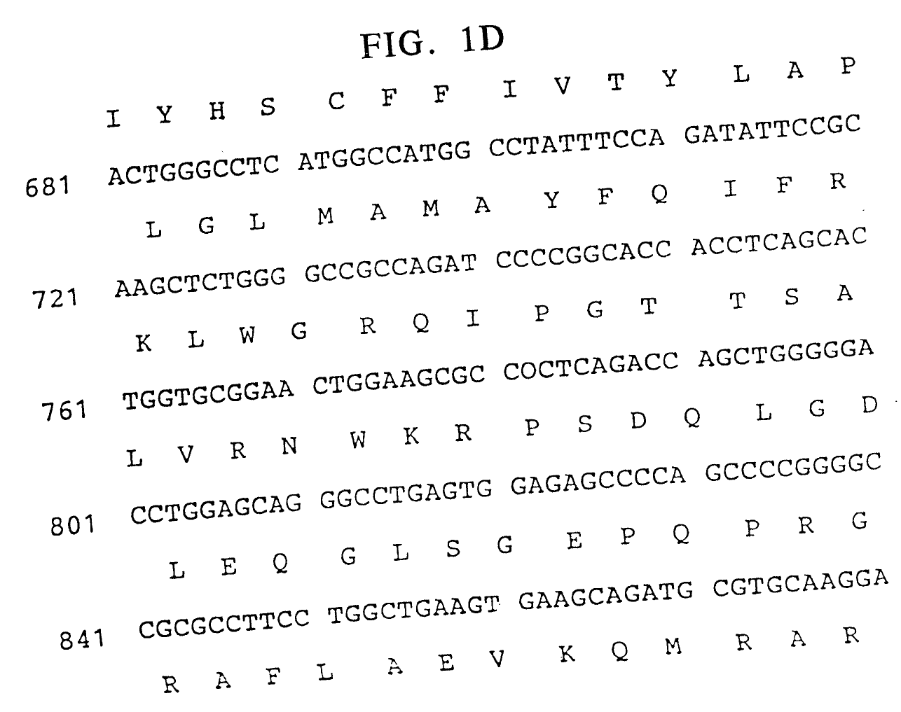

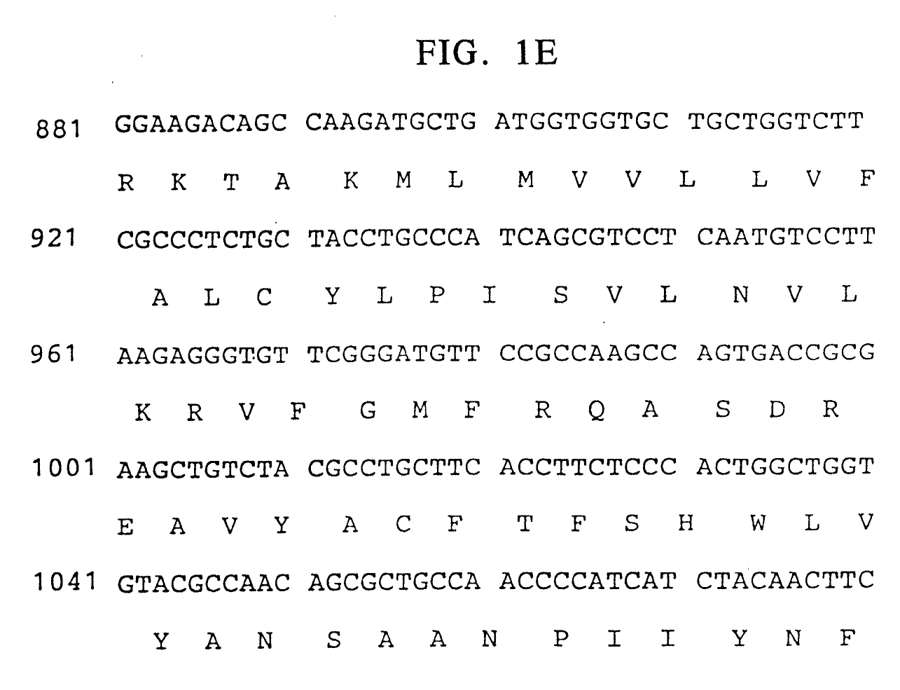

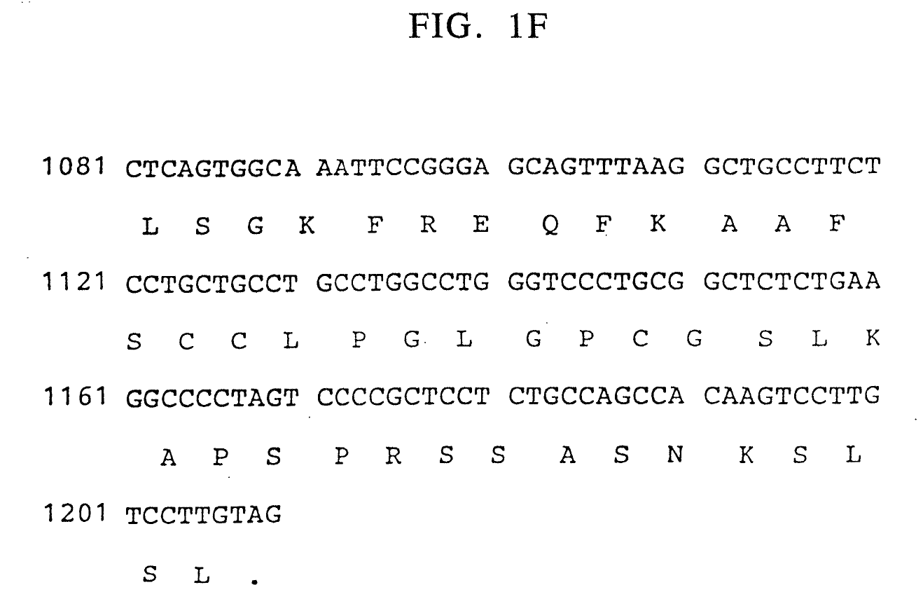

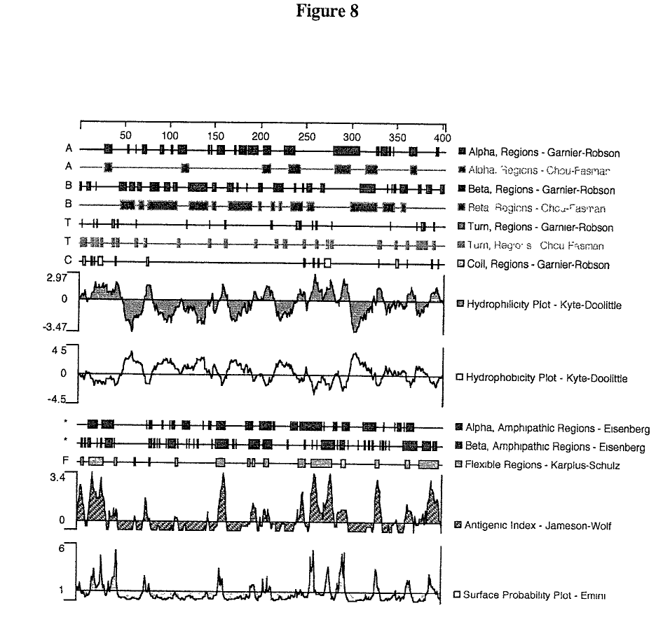

The data presented in FIG. 8 are also represented in tabular form in Table I. The columns are labeled with the headings “Res”, “Position”, and Roman Numerals I-XIV. The column headings refer to the following features of the amino acid sequence presented in FIG. 8, and Table I: “Res”: amino acid residue of SEQ ID NO:2 and FIGS. 1A-F; “Position”: position of the corresponding residue within SEQ ID NO:2 and FIGS. 1A-F; I: Alpha, Regions—Garnier-Robson; II: Alpha, Regions—Chou-Fasman; III: Beta, Regions—Garnier-Robson; IV: Beta, Regions—Chou-Fasman; V: Turn, Regions—Garnier-Robson; VI: Turn, Regions—Chou-Fasman; VII: Coil, Regions—Garnier-Robson; VIII: Hydrophilicity Plot—Kyte-Doolittle; IX: Hydrophobicity Plot—Hopp-Woods; X: Alpha, Amphipathic Regions—Eisenberg; XI: Beta, Amphipathic Regions—Eisenberg; XII: Flexible Regions—Karplus-Schulz; XIII: Antigenic Index—Jameson-Wolf; and XIV: Surface Probability Plot—Emini.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” neuropeptide receptor protein refers to a protein capable of being directed to the ER, secretory vesicles, or the extracellular space as a result of a signal sequence, as well as a neuropeptide receptor protein released into the extracellular space without necessarily containing a signal sequence. If the neuropeptide receptor secreted protein is released into the extracellular space, the neuropeptide receptor secreted protein can undergo extracellular processing to produce a “mature” neuropeptide receptor protein. Release into the extracellular space can occur by many mechanisms, including exocytosis and proteolytic cleavage.

As used herein, a neuropeptide receptor “polynucleotide” refers to a molecule having a nucleic acid sequence contained in SEQ ID NO:1, 3, or 5, or the cDNA contained within the clone deposited with the ATCC. For example, the neuropeptide receptor polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a neuropeptide receptor “polypeptide” refers to a molecule having the translated amino acid sequence generated from the polynucleotide as broadly defined (SEQ ID NO:2, 4, or 6).

The receptor polypeptides of the present invention are receptors for ligands, both known and unknown, which modulate the activity of cells in both the central nervous system and peripheral tissues regulated by the central nervous system. Examples of such ligands are neuropeptide Y, substance P, the human ob gene product and neurokinin B. Accordingly, modulation of the activity of receptor polypeptides of the present invention will have a broad range of therapeutic and diagnostic applications, particularly with respect to the treatment of obesity.

The present inventors have isolated a full-length cDNA clone encoding a human neuropeptide receptor polypeptide. The present full-length cDNA has been mapped to a location on human chromosome 1 position p31-34 which corresponds to a location on the mouse chromosome 4 where the db gene is found. The mouse db gene is thought to encode the receptor for the obesity gene product.

In the present invention, the full length neuropeptide receptor sequence identified as SEQ ID NO:1 was generated by overlapping sequences of the deposited clone (contig analysis). A representative clone containing all or most of the sequence for SEQ ID NO:1 was deposited with the American Type Culture Collection (“ATCC”) on Apr. 28, 1995, and was given the ATCC Deposit Number 97128. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

In accordance with an aspect of the present invention, there are provided isolated nucleic acids (polynucleotides) which encode for the mature polypeptide having the deduced amino acid sequence of FIGS. 1A-F (SEQ ID NO:2) or for the mature polypeptide encoded by the cDNA of the clone(s) deposited as ATCC Deposit No. 97128 on Apr. 28, 1995.

The polynucleotide of this invention was discovered in a cDNA library derived from human adult hypothalamus. It is structurally related to the G protein-coupled receptor family. The neuropeptide receptor polypeptide contains an open reading frame encoding a protein of 402 amino acid residues. The neuropeptide receptor protein exhibits the highest degree of homology to human neuropeptide Y receptor protein with 52% similarity and 26% identity over the entire amino acid sequence.

The polynucleotides of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequences which encode the mature polypeptide may be identical to the coding sequence shown in FIGS. 1A-F (SEQ ID NO:1) or that of the deposited clone(s) or may be a different coding sequence which coding sequence, as a result of the redundancy or degeneracy of the genetic code, encodes the same mature polypeptide as the DNA of FIGS. 1A-F (SEQ ID NO:1) or the deposited cDNA(s). Additionally, the neuropeptide receptor polynucleotide can be composed of any polyribonucleotide or polydeoxribonucleotide, which may be unmodified RNA or DNA or modified RNA or DNA. For example, neuropeptide receptor polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the neuropeptide receptor polynucleotides can be composed of triple-stranded regions comprising RNA or DNA or both RNA and DNA. Neuropeptide receptor polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of neuropeptide receptor coding sequence, but do not comprise all or a portion of any neuropeptide receptor intron. In another embodiment, the nucleic acid comprising neuropeptide receptor coding sequence does not contain coding sequences of a genomic flanking gene (i.e., 5′ or 3′ to the neuropeptide receptor gene in the genome).

The polynucleotides which encode for the mature polypeptide of FIGS. 1A-F, 2A-E, or 3A-F (SEQ ID NO:2, 4, or 6) or for the mature polypeptide encoded by the deposited cDNA(s) may include: only the coding sequence for the mature polypeptide; the coding sequence for the mature polypeptide (and optionally additional coding sequence) and non-coding sequence, such as introns or non-coding sequence 5′ and/or 3′ of the coding sequence for the mature polypeptide. Thus, the term “polynucleotide encoding a polypeptide” encompasses a polynucleotide which includes only coding sequence for the polypeptide as well as a polynucleotide which includes additional coding and/or non-coding sequence. A neuropeptide receptor “polynucleotide” also includes those polynucleotides capable of hybridizing, under stringent hybridization conditions, to sequences contained in SEQ ID NO:1, 3, or 5, the complement thereof, or the cDNA within the deposited clone. “Stringent hybridization conditions” refers to an overnight incubation at 42 degree C. in a solution comprising 50% formamide, 5×SSC (750 mnM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65 degree C.

Also contemplated are nucleic acid molecules that hybridize to the neuropeptide receptor polynucleotides at moderatetly high stringency hybridization conditions. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH₂PO₄; 0.02M EDTA, pH 7.4), 0.5% SDS, 30% formamide, 100 ug/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The present invention further relates to variants of the hereinabove described polynucleotides which encode for fragments, analogs and derivatives of the polypeptides having the deduced amino acid sequence of FIGS. 1A-F, 2A-E, or 3A-F (SEQ ID NO:2, 4, or 6) or the polypeptide encoded by the cDNA of the deposited clone(s). The variants of the polynucleotide may be naturally occuring allelic variants of the polynucleotides or non-naturally occurring variants of the polynucleotides.

Thus, the present invention includes polynucleotides encoding the same mature polypeptide as shown in FIGS. 1A-F, 2A-E, or 3A-F (SEQ ID NO:2, 4, or 6) or the same mature polypeptide encoded by the cDNA of the deposited clone(s) as well as variants of such polynucleotide which variants encode for a fragment, derivative or analog of the polypeptides of FIGS. 1A-F, 2A-E, or 3A-F (SEQ ID NO:2, 4, or 6) or the polypeptide encoded by the cDNA of the deposited clone(s). Such nucleotide variants include deletion variants, substitution variants and addition or insertion variants. Specific examples of such variants include the polynucleotide sequences as set forth in SEQ ID NOS: 3 and 5 which encode for splice variant 1 and 2, respectively, of the polypeptide of the present invention.

As hereinabove indicated, the polynucleotides may have a coding sequence which is a naturally occurring allelic variant of the coding sequence shown in FIGS. 1A-F, 2A-E, or 3A-F (SEQ ID NO:1, 3, or 5) or of the coding sequence of the deposited clone(s). As known in the art, an allelic variant is an alternate form of polynucleotide sequences which may have a substitution, deletion or addition of one or more nucleotides, which does not substantially alter the function of the encoded polypeptides.

The polynucleotides may also encode for a soluble form of the neuropeptide receptor polypeptide which is the extracellular portion of the polypeptide which has been cleaved from the TM and intracellular domain of the full-length polypeptide of the present invention.

The polynucleotides of the present invention may also have the coding sequence fused in frame to a marker sequence which allows for purification of the polypeptide of the present invention. The marker sequence may be a hexa-histidine tag supplied by a pQE-9 vector to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

The present invention further relates to polynucleotides which hybridize to the hereinabove-described sequences if there is at least 70%, preferably at least 90%, and more preferably at least 95% identity between the sequences. The present invention particularly relates to polynucleotides which hybridize under stringent conditions to the hereinabove-described polynucleotides. As herein used, the term “stringent conditions” means hybridization will occur only if there is at least 95% and preferably at least 97% identity between the sequences. The polynucleotides which hybridize to the hereinabove described polynucleotides in a preferred embodiment encode polypeptides which either retain substantially the same biological function or activity as the mature polypeptide encoded by the cDNAs of FIGS. 1A-F, 2A-E, or 3A-F (SEQ ID NO: 1, 3, or 5) or the deposited cDNA(s), i.e. function as a soluble neuropeptide receptor by retaining the ability to bind the ligands for the receptor even though the polypeptide does not function as a membrane bound neuropeptide receptor, for example, by eliciting a second messenger response.

Alternatively, the polynucleotides may be polynucleotides which have at least 20 bases, preferably 30 bases and more preferably at least 50 bases which hybridize to a polynucleotide of the present invention and which have an identity thereto, as hereinabove described, and which does not retain activity. Such polynucleotides may be employed as probes for the polynucleotide of SEQ ID NO: 1, 3, or 5, or for variants thereof, for example, for recovery of the polynucleotide or as a diagnostic probe or as a PCR primer.

The deposit(s) referred to herein will be maintained under the terms of the Budapest Treaty on the International Recognition of the Deposit of Micro-organisms for purposes of Patent Procedure. These deposits are provided merely as convenience to those of skill in the art and are not an admission that a deposit is required under 35 U.S.C. §112. The sequence of the polynucleotides contained in the deposited materials, as well as the amino acid sequence of the polypeptides encoded thereby, are incorporated herein by reference and are controlling in the event of any conflict with any description of sequences herein. A license may be required to make, use or sell the deposited materials, and no such license is hereby granted.

The present invention further relates to a polypeptide which has the deduced amino acid sequence of FIGS. 1A-F, 2A-E, or 3A-F (SEQ ID NO:2, 4, or 6) or which has the amino acid sequence encoded by the deposited cDNA(s), as well as fragments, analogs and derivatives of such polypeptide.

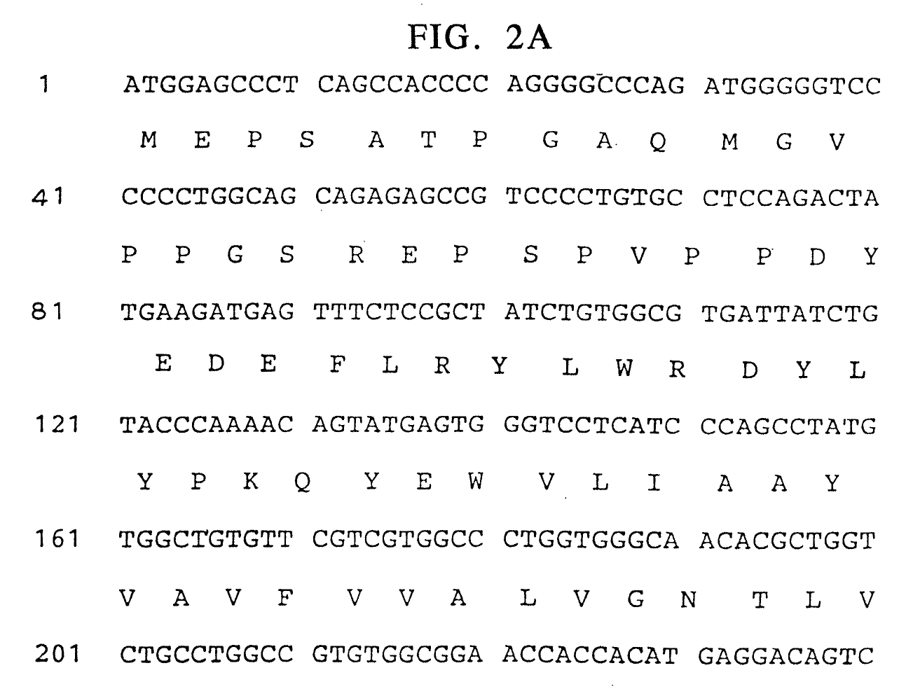

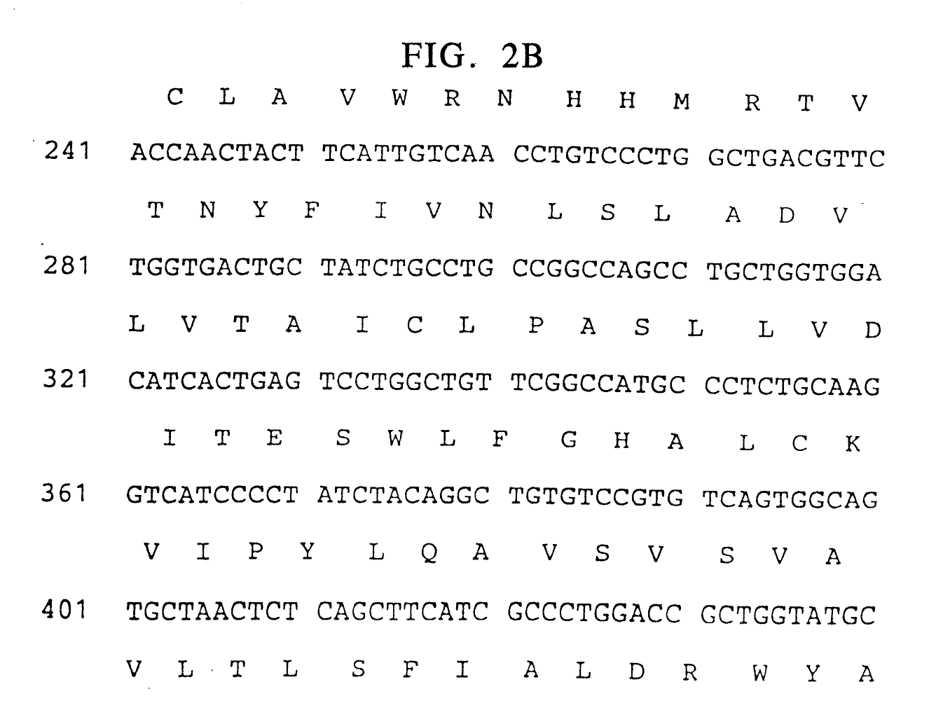

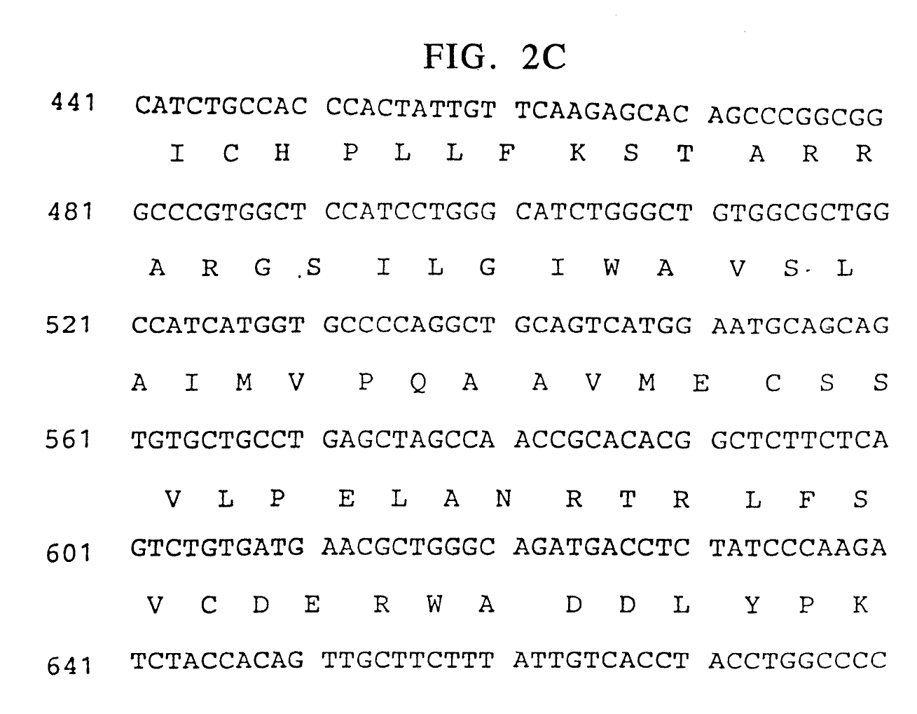

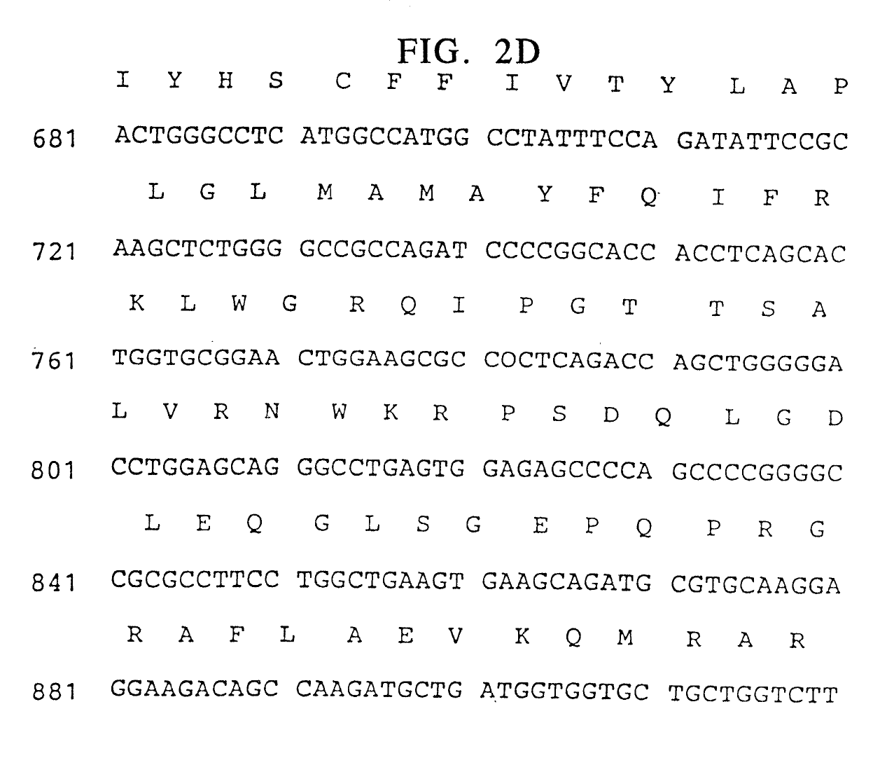

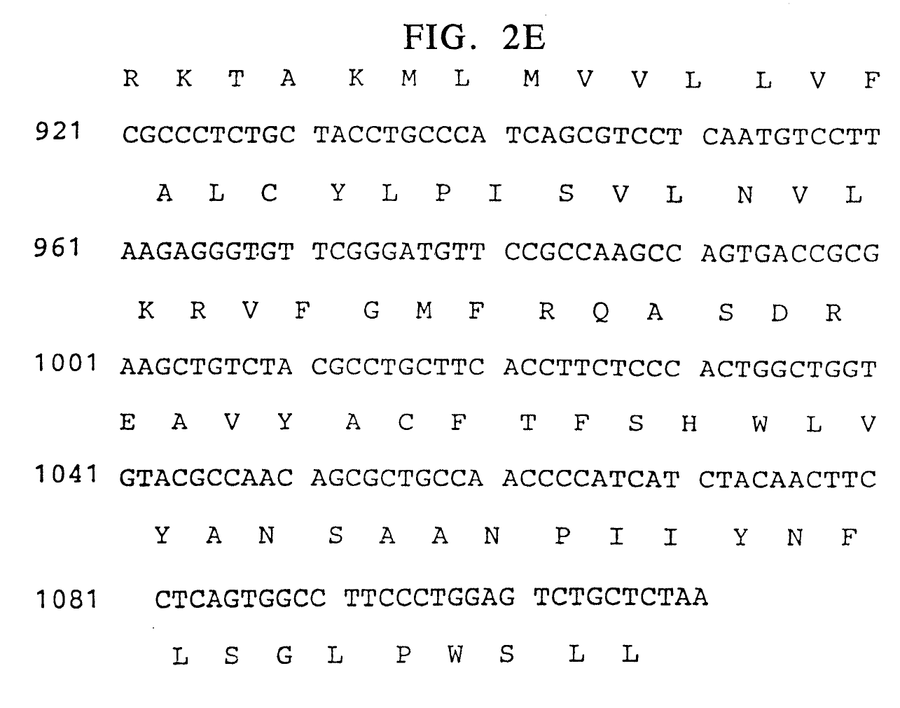

The terms “fragment,” “derivative” and “analog” when referring to the polypeptide of FIGS. 1A-F, 2A-E, or 3A-F (SEQ ID NO:2, 4, or 6) or that encoded by the deposited cDNA(s), means polypeptides which either retain substantially the same biological function or activity as such polypeptides, i.e., function as a soluble neuropeptide receptor by retaining the ability to bind the ligands of the receptors even though the polypeptides do not function as membrane bound neuropeptide receptors. An analog includes a proprotein which can be activated by cleavage of the proprotein portion to produce an active mature polypeptide. Specific examples are splice variant 1 and 2 of FIGS. 2A-E and 3A-F (SEQ ID NO:4 and 6), respectively.

The polypeptides of the present invention may be recombinant polypeptides, natural polypeptides or synthetic polypeptides, preferably recombinant polypeptides.

A fragment, derivative or analog of the polypeptides of FIGS. 1A-F, 2A-E, or 3A-F (SEQ ID NO:2, 4, or 6) or that encoded by the deposited cDNA(s) may be (i) one in which one or more of the amino acid residues are substituted with a conserved or non-conserved amino acid residue (preferably a conserved amino acid residue) and such substituted amino acid residue may or may not be one encoded by the genetic code, (ii) one in which one or more of the amino acid residues includes a substituent group, (iii) one in which the mature polypeptide is fused with another compound, such as a compound to increase the half-life of the polypeptide (for example, polyethylene glycol), (iv) one in which the additional amino acids are fused to the mature polypeptide, such as sequence which is employed for purification of the mature polypeptide sequence or (iv) splice variants of the mature polypeptide which may have one or more amino acids deleted from the mature polypeptide yet still retain activity corresponding to the mature polypeptide. Such fragments, derivatives and analogs are deemed to be within the scope of those skilled in the art from the teachings herein.

The polypeptides and polynucleotides of the present invention are preferably provided in an isolated form, and preferably are purified to homogeneity.

The term “gene” means the segment of DNA involved in producing a polypeptide chain; it includes regions preceding and following the coding region “leader and trailer” as well as intervening sequences (introns) between individual coding segments (exons).

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

Neuropeptide receptor polypeptides can be composed of amino acids joined to each other by peptide bonds or modified peptide bonds, i.e., peptide isosteres, and may contain amino acids other than the 20 gene-encoded amino acids. The neuropeptide receptor polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the neuropeptide receptor polypeptide, including the peptide backbone, the amino acid side-chains and the amino or carboxyl termini. It will be appreciated that the same type of modification may be present in the same or varying degrees at several sites in a given neuropeptide receptor polypeptide. Also, a given neuropeptide receptor polypeptide may contain many types of modifications. neuropeptide receptor polypeptides may be branched 5 for example, as a result of ubiquitination, and they may be cyclic, with or without branching. Cyclic, branched, and branched cyclic neuropeptide receptor polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:1” refers to a neuropeptide receptor polynucleotide sequence while “SEQ ID NO:2” refers to a neuropeptide receptor polypeptide sequence.

A neuropeptide receptor polypeptide fragment “having biological activity” refers to polypeptides exhibiting activity similar, but not necessarily identical to, an activity of a neuropeptide receptor polypeptide, including mature forms, as measured in a particular biological assay, with or without dose dependency. In the case where dose dependency does exist, it need not be identical to that of the neuropeptide receptor polypeptide, but rather substantially similar to the dose-dependence in a given activity as compared to the neuropeptide receptor polypeptide (i.e., the candidate polypeptide will exhibit greater activity or not more than about 25-fold less and, preferably, not more than about tenfold less activity, and most preferably, not more than about three-fold less activity relative to the neuropeptide receptor polypeptide.)

Neuropeptide Receptor Polynucleotides and Polypeptides

SEQ ID NOS:1-6 are sufficiently accurate and otherwise suitable for a variety of uses well known in the art and described further below. For instance, SEQ ID NO:1, 3, or 5 are useful for designing nucleic acid hybridization probes that will detect nucleic acid sequences contained in SEQ ID NO:1, 3, or 5 or the cDNA contained in the deposited clone. These probes will also hybridize to nucleic acid molecules in biological samples, thereby enabling a variety of forensic and diagnostic methods of the invention. Similarly, polypeptides identified from SEQ ID NO: 2, 4, or 6 may be used to generate antibodies which bind specifically to neuropeptide receptor.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO: 1, 3, and 5 and the predicted translated amino acid sequence identified as SEQ ID NO: 2, 4, and 6 but also a sample of plasmid DNA containing a human cDNA of neuropeptide receptor deposited with the ATCC. The nucleotide sequence of the deposited neuropeptide receptor clone can readily be determined by sequencing the deposited clone in accordance with known methods. The predicted neuropeptide receptor amino acid sequence can then be verified from such deposits. Moreover, the amino acid sequence of the protein encoded by the deposited clone can also be directly determined by peptide sequencing or by expressing the protein in a suitable host cell containing the deposited human neuropeptide receptor cDNA, collecting the protein, and determining its sequence.

The present invention also relates to the neuropeptide receptor gene corresponding to SEQ ID NO:1, SEQ ID NO:2, or the deposited clone. The neuropeptide receptor gene can be isolated in accordance with known methods using the sequence information disclosed herein. Such methods include preparing probes or primers from the disclosed sequence and identifying or amplifying the neuropeptide receptor gene from appropriate sources of genomic material.

Also provided in the present invention are species homologs of neuropeptide receptor. Species homologs may be isolated and identified by making suitable probes or primers from the sequences provided herein and screening a suitable nucleic acid source for the desired homologue.

The neuropeptide receptor polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The neuropeptide receptor polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

Neuropeptide receptor polypeptides are preferably provided in an isolated form, and preferably are substantially purified. A recombinantly produced version of a neuropeptide receptor polypeptide, including the secreted polypeptide, can be substantially purified by the one-step method described in Smith and Johnson, Gene 67:31-40 (1988). Neuropeptide receptor polypeptides also can be purified from natural or recombinant sources using antibodies of the invention raised against the neuropeptide receptor protein in methods which are well known in the art.

Polynucleotide and Polypeptide Variants

“Variant” refers to a polynucleotide or polypeptide differing from the neuropeptide receptor polynucleotide or polypeptide, but retaining essential properties thereof. Generally, variants are overall closely similar, and, in many regions, identical to the neuropeptide receptor polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the neuropeptide receptor polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO:1, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identiy are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:2 or to the amino acid sequence encoded by deposited DNA clone can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The neuropeptide receptor variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Neuropeptide receptor polynucleotide variants can be produced for a variety of reasons, e.g., to optimize codon expression for a particular host (change codons in the human mRNA to those preferred by a bacterial host such as E. coli).

Naturally occurring neuropeptide receptor variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the neuropeptide receptor polypeptides. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes neuropeptide receptor polypeptide variants which show substantial biological activity. Such variants include deletions, insertions, inversions, repeats, and substitutions selected according to general rules known in the art so as have little effect on activity.

The present application is directed to nucleic acid molecules at least 90%, 95%, 96%, 97%, 98% or 99% identical to the nucleic acid sequences disclosed herein, (e.g., encoding a polypeptide having the amino acid sequence of an N and/or C terminal deletion disclosed below as m-n of SEQ ID NO:2, 4, or 6), irrespective of whether they encode a polypeptide having neuropeptide receptor functional activity. This is because even where a particular nucleic acid molecule does not encode a polypeptide having neuropeptide receptor functional activity, one of skill in the art would still know how to use the nucleic acid molecule, for instance, as a hybridization probe or a polymerase chain reaction (PCR) primer. Uses of the nucleic acid molecules of the present invention that do not encode a polypeptide having neuropeptide receptor functional activity include, inter alia, (1) isolating a neuropeptide receptor gene or allelic or splice variants thereof in a cDNA library; (2) in situ hybridization (e.g., “FISH”) to metaphase chromosomal spreads to provide precise chromosomal location of the neuropeptide receptor gene, as described in Verma et al., Human Chromosomes: A Manual of Basic Techniques, Pergamon Press, New York (1988); and (3) Northern Blot analysis for detecting neuropeptide receptor mRNA expression in specific tissues.

Preferred, however, are nucleic acid molecules having sequences at least 90%, 95%, 96%, 97%, 98% or 99% identical to the nucleic acid sequences disclosed herein, which do, in fact, encode a polypeptide having neuropeptide receptor functional activity. By “a polypeptide having neuropeptide receptor functional activity” is intended polypeptides exhibiting activity similar, but not necessarily identical, to a functional activity of the neuropeptide receptor polypeptides of the present invention (e.g., complete (full-length) neuropeptide receptor, mature neuropeptide receptor and soluble neuropeptide receptor (e.g., having sequences contained in the extracellular domain of neuropeptide receptor) as measured, for example, in a particular immunoassay or biological assay. For example, a neuropeptide receptor functional activity can routinely be measured by determining the ability of a neuropeptide receptor polypeptide to bind a neuropeptide receptor ligand. Neuropeptide receptor functional activity may also be measured by determining the ability of a polypeptide, such as cognate ligand which is free or expressed on a cell surface, to induce cells expressing the polypeptide.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA, the nucleic acid sequence shown in FIGS. 1A-F, 2A-E, or 3A-F (SEQ ID NO:1, 3, or 5), or fragments thereof, will encode polypeptides “having neuropeptide receptor functional activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having neuropeptide receptor functional activity. This is because the skilled artisan is fully aware of amino acid substitutions that are either less likely or not likely to significantly effect protein function (e.g., replacing one aliphatic amino acid with a second aliphatic amino acid), as further described below.

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

For example, site directed changes at the amino acid level of neuropeptide receptor can be made by replacing a particular amino acid with a conservative amino acid. Preferred conservative mutations include: MI replaced with A, G, I, L, S, T, or V; E2 replaced with D; S4 replaced with A, G, I, L, T, M, or V; A5 replaced with G, I, L, S, T, M, or V; T6 replaced with A, G, I, L, S, M, or V; G8 replaced with A, I, L, S, T, M, or V; A9 replaced with G, I, L, S, T, M, or V; Q10 replaced with N; M11 replaced with A, G, I, L, S, T, or V; G12 replaced with A, I, L, S, T, M, or V; V13 replaced with A, G, I, L, S, T, or M; G16 replaced with A, I, L, S, T, M, or V; S17 replaced with A, G, I, L, T, M, or V; R18 replaced with H, or K; E19 replaced with D; S21 replaced with A, G, I, L, T, M, or V; V23 replaced with A, G, I, L, S, T, or M; D26 replaced with E; Y27 replaced with F, or W; E28 replaced with D; D29 replaced with E; E30 replaced with D; F31 replaced with W, or Y; L32 replaced with A, G, I, S, T, M, or V; R33 replaced with H, or K; Y34 replaced with F, or W; L35 replaced with A, G, I, S, T, M, or V; W36 replaced with F, or Y; R37 replaced with H, or K; D38 replaced with E; Y39 replaced with F, or W; L40 replaced with A, G, I, S, T, M, or V; Y41 replaced with F, or W; K43 replaced with H, or R; Q44 replaced with N; Y45 replaced with F, or W; E46 replaced with D; W47 replaced with F, or Y; V48 replaced with A, G, I, L, S, T, or M; L49 replaced with A, G, I, S, T, M, or V; I50 replaced with A, G, L, S, T, M, or V; A51 replaced with G, I, L, S, T, M, or V; A52 replaced with G, I, L, S, T, M, or V; Y53 replaced with F, or W; V54 replaced with A, G, I, L, S, T, or M; A55 replaced with G, I, L, S, T, M, or V; V56 replaced with A, G, I, L, S, T, or M; F57 replaced with W, or Y; V58 replaced with A, G, I, L, S, T, or M; V59 replaced with A, G, I, L, S, T, or M; A60 replaced with G, I, L, S, T, M, or V; L61 replaced with A, G, I, S, T, M, or V; V62 replaced with A, G, I, L, S, T, or M; G63 replaced with A, I, L, S, T, M, or V; N64 replaced with Q; T65 replaced with A, G, I, L, S, M, or V; L66 replaced with A, G, I, S, T, M, or V; V67 replaced with A, G, I, L, S, T, or M, L69 replaced with A, G, I, S, T, M, or V; A70 replaced with G, I, L, S, T, M, or V; V71 replaced with A, G, I, L, S, T, or M; W72 replaced with F, or Y; R73 replaced with H, or K; N74 replaced with Q; H75 replaced with K, or R; H76 replaced with K, or R; M77 replaced with A, G, I, L, S, T, or V; R78 replaced with H, or K; T79 replaced with A, G, I, L, S, M, or V; V80 replaced with A, G, I, L, S, T, or M; T81 replaced with A, G, I, L, S, M, or V; N82 replaced with Q; Y83 replaced with F, or W; F84 replaced with W, or Y; I85 replaced with A, G, L, S, T, M, or V; V86 replaced with A, G, I, L, S, T, or M; N87 replaced with Q; L88 replaced with A, G, I, S, T, M, or V; S89 replaced with A, G, I, L, T, M, or V; L90 replaced with A, G, I, S, T, M, or V; A91 replaced with G, I, L, S, T, M, or V; D92 replaced with E; V93 replaced with A, G, I, L, S, T, or M; L94 replaced with A, G, I, S, T, M, or V; V95 replaced with A, G, I, L, S, T, or M; T9 replaced with A, G, I, L, S, M, or V; A97 replaced with G, I, L, S, T, M, or V; I98 replaced with A, G, L, S, T, M, or V; L100 replaced with A, G, I, S, T, M, or V; A102 replaced with G, I, L, S, T, M, or V; S103 replaced with A, G, I, L, T, M, or V; L104 replaced with A, G, I, S, T, M, or V; L105 replaced with A, G, I, S, T, M, or V; V 106 replaced with A, G, I, L, S, T, or M; D 107 replaced with E; I108 replaced with A, G, L, S, T, M, or V; T109 replaced with A, G, I, L, S, M, or V; E110 replaced with D; S111 replaced with A, G, I, L, T, M, or V; W112 replaced with F, or Y; L113 replaced with A, G, I, S, T, M, or V; F114 replaced with W, or Y; G115 replaced with A, I, L, S, T, M, or V; H116 replaced with K, or R; A117 replaced with G, I, L, S, T, M, or V; L118 replaced with A, G, I, S, T, M, or V; K120 replaced with H, or R; V121 replaced with A, G, I, L, S, T, or M; I122 replaced with A, G, L, S, T, M, or V; Y124 replaced with F, or W; L125 replaced with A, G, I, S, T, M, or V; Q 126 replaced with N; A127 replaced with G, I, L, S, T, M, or V; V 128 replaced with A, G, I, L, S, T, or M; S129 replaced with A, G, I, L, T, M, or V; V130 replaced with A, G, I, L, S, T, or M; S131 replaced with A, G, I, L, T, M, or V; V132 replaced with A, G, I, L, S, T, or M; A133 replaced with G, G, L, S, T, M, or V; V134 replaced with A, G, I, L, S, T, or M; L135 replaced with A, G, L, S, T, M, or V; T136 replaced with A, G, I, L, S, M, or V; L137 replaced with A, G, I, S, T, M, or V; S138 replaced with A, G, I, L, T, M, or V; F139 replaced with W, or Y; I140 replaced with A, G, L, S, T, M, or V; A141 replaced with G, I, L, S, T, M, or V; L142 replaced with A, G, I, S, T, M, or V; D143 replaced with E; R144 replaced with H, or K; W145 replaced with F, or Y; Y146 replaced with F, or W; A147 replaced with G, I, L, S, T, M, or V; I148 replaced with A, G, L, S, T, M, or V; H150 replaced with K, or R; L152 replaced with A, G, I, S, T, M, or V; L153 replaced with A, G, I, S, T, M, or V; F154 replaced with W, or Y; K155 replaced with H, or R; S156 replaced with A, G, I, L, T, M, or V; T157 replaced with A, G, I, L, S, M, or V; A158 replaced with G, I, L, S, T, M, or V; R1 59 replaced with H, or K; R160 replaced with H, or K; A161 replaced with G, I, L, S, T, M, or V; R162 replaced with H, or K; G163 replaced with A, I, L, S, T, M, or V; S164 replaced with A, G, I, L, T, M, or V; I165 replaced with A, G, L, S, T, M, or V; L166 replaced with A, G, I, S, T, M, or V; G167 replaced with A, I, L, S, T, M, or V; I168 replaced with A, G, L, S, T, M, or V; W169 replaced with F, or Y; A170 replaced with G, I, L, S, T, M, or V; V171 replaced with A, G, I, L, S, T, or M; S172 replaced with A, G, I, L, T, M, or V; L173 replaced with A, G, I, S, T, M, or V; A174 replaced with G, I, L, S, T, M, or V; I175 replaced with A, G, L, S, T, M, or V; M176 replaced with A, G, I, L, S, T, or V; V177 replaced with A, G, I, L, S, T, or M; Q179 replaced with N; A180 replaced with G, I, L, S, T, M, or V; A181 replaced with G, I, L, S, T, M, or V; V182 replaced with A, G, I, L, S, T, or M; M183 replaced with A, G, I, L, S, T, or V; E184 replaced with D; S186 replaced with A, G, I, L, T, M, or V; S187 replaced with A, G, I, L, T, M, or V; V188 replaced with A, G, I, L, S, T, or M; L189 replaced with A, G, I, S, T, M, or V; E191 replaced with D; L192 replaced with A, G, I, S, T, M, or V; A193 replaced with G, I, L, S, T, M, or V; N194 replaced with Q; R195 replaced with H, or K; T196 replaced with A, G, I, L, S, M, or V; R197 replaced with H, or K; L198 replaced with A, G, I, S, T, M, or V; F199 replaced with W, or Y; S200 replaced with A, G, I, L, T, M, or V; V201 replaced with A, G, I, L, S, T, or M; D203 replaced with E; E204 replaced with D; R205 replaced with H, or K; W206 replaced with F, or Y; A207 replaced with G, I, L, S, T, M, or V; D208 replaced with E; D209 replaced with E; L210 replaced with A, G, I, S, T, M, or V; Y211 replaced with F, or W; K213 replaced with H, or R; I214 replaced with A, G, L, S, T, M, or V; Y215 replaced with F, or W; H216 replaced with K, or R; S217 replaced with A, G, I, L, T, M, or V; F219 replaced with W, or Y; F220 replaced with W, or Y; I221 replaced with A, G, L, S, T, M, or V; V222 replaced with A, G, I, L, S, T, or M; T223 replaced with A, G, I, L, S, M, or V; Y224 replaced with F, or W; L225 replaced with A, G, I, S, T, M, or V; A226 replaced with G, I, L, S, T, M, or V; L228 replaced with A, G, I, S, T, M, or V; G229 replaced with A, I, L, S, T, M, or V; L230 replaced with A, G, I, S, T, M, or V; M231 replaced with A, G, I, L, S, T, or V; A232 replaced with G, I, L, S, T, M, or V; M233 replaced with A, G, I, L, S, T, or V; A234 replaced with G, I, L, S, T, M, or V; Y235 replaced with F, or W; F236 replaced with W, or Y; Q237 replaced with N; I238 replaced with A, G, L, S, T, M, or V; F239 replaced with W, or Y; R240 replaced with H, or K; K241 replaced with H, or R; L242 replaced with A, G, I, S, T, M, or V; W243 replaced with F, or Y; G244 replaced with A, I, L, S, T, M, or V; R245 replaced with H, or K; Q246 replaced with N; I247 replaced with A, G, L, S, T, M, or V; G249 replaced with A, I, L, S, T, M, or V; T250 replaced with A, G, I, L, S, M, or V; T251 replaced with A, G, I, L, S, M, or V; S252 replaced with A, G, I, L, T, M, or V; A253 replaced with G, I, L, S, T, M, or V; L254 replaced with A, G, I, S, T, M, or V; V255 replaced with A, G, I, L, S, T, or M; R256 replaced with H, or K; N257 replaced with Q; W258 replaced with F, or Y; K259 replaced with H, or R; R260 replaced with H, or K; S262 replaced with A, G, I, L, T, M, or V; D263 replaced with E; Q264 replaced with N; L265 replaced with A, G, I, S, T, M, or V; G266 replaced with A, I, L, S, T, M, or V; D267 replaced with E; L268 replaced with A, G, I, S, T, M, or V; E269 replaced with D; Q270 replaced with N; G271 replaced with A, I, L, S, T, M, or V; L272 replaced with A, G, I, S, T, M, or V; S273 replaced with A, G, I, L, T, M, or V; G274 replaced with A, I, L, S, T, M, or V; E275 replaced with D; Q277 replaced with N; R279 replaced with H, or K; G280 replaced with A, I, L, S, T, M, or V; R281 replaced with H, or K; A282 replaced with G, I, L, S, T, M, or V; F283 replaced with W, or Y; L284 replaced with A, G, I, S, T, M, or V; A285 replaced with G, I, L, S, T, M, or V; E286 replaced with D; V287 replaced with A, G, I, L, S, T, or M; K288 replaced with H, or R; Q289 replaced with N; M290 replaced with A, G, I, L, S, T, or V; R291 replaced with H, or K; A292 replaced with G, I, L, S, T, M, or V; R293 replaced with H, or K; R294 replaced with H, or K; K295 replaced with H, or R; T296 replaced with A, G, I, L, S, M, or V; A297 replaced with G, I, L, S, T, M, or V; K298 replaced with H, or R; M299 replaced with A, G, I, L, S, T, or V; L300 replaced with A, G, I, S, T, M, or V; M301 replaced with A, G, I, L, S, T, or V; V302 replaced with A, G, I, L, S, T, or M; V303 replaced with A, G, I, L, S, T, or M; L304 replaced with A, G, I, S, T, M, or V; L305 replaced with A, G, I, S, T, M, or V; V306 replaced with A, G, I, L, S, T, or M; F307 replaced with W, or Y; A308 replaced with G, I, L, S, T, M, or V; L309 replaced with A, G, I, S, T, M, or V; Y311 replaced with F, or W; L312 replaced with A, G, I, S, T, M, or V; 1314 replaced with A, G, L, S, T, M, or V; S315 replaced with A, G, I, L, T, M, or V; V316 replaced with A, G, I, L, S, T, or M; L317 replaced with A, G, I, S, T, M, or V; N318 replaced with Q; V319 replaced with A, G, I, L, S, T, or M; L320 replaced with A, G, I, S, T, M, or V; K321 replaced with H, or R; R322 replaced with H, or K; V323 replaced with A, G, I, L, S, T, or M; F324 replaced with W. or Y; G325 replaced with A, I, L, S, T, M, or V; M326 replaced with A, G, I, L, S, T, or V; F327 replaced with W, or Y; R328 replaced with H, or K; Q329 replaced with N; A330 replaced with G, I, L, S, T, M, or V; S331 replaced with A, G, I, L, T, M, or V; D332 replaced with E; R333 replaced with H, or K; E334 replaced with D; A335 replaced with G, I, L, S, T, M, or V; V336 replaced with A, G, I, L, S, T, or M; Y337 replaced with F, or W; A338 replaced with G, I, L, S, T, M, or V; F340 replaced with W, or Y; T341 replaced with A, G. I, L, S, M, or V; F342 replaced with W, or Y; S343 replaced with A, G, I, L, T, M, or V; H344 replaced with K, or R; W345 replaced with F, or Y; L346 replaced with A, G, I, S, T, M, or V; V347 replaced with A, G, I, L, S, T, or M; Y348 replaced with F, or W; A349 replaced with G, I, L, S, T, M, or V; N350 replaced with Q; S351 replaced with A, G, I, L, T, M, or V; A352 replaced with G, I, L, S, T, M, or V; A353 replaced with G, I, L, S, T, M, or V; N354 replaced with Q; I356 replaced with A, G, L, S, T, M, or V; I357 replaced with A, G, L, S, T, M, or V; Y358 replaced with F, or W; N359 replaced with Q; F360 replaced with W, or Y; L361 replaced with A, G, I, S, T, M, or V; S362 replaced with A, G, I, L, T, M, or V; or G363 replaced with A, I, L, S, T, M, or V of SEQ ID NO:2, 4, and 6.

Additional preferred conservative mutations include: K364 replaced with H, or R; F365 replaced with W, or Y; R366 replaced with H, or K; E367 replaced with D; Q368 replaced with N; F369 replaced with W, or Y; K370 replaced with H, or R; A371 replaced with G, I, L, S, T, M, or V; A372 replaced with G, I, L, S, T, M, or V; F373 replaced with W, or Y; S374 replaced with A, G, I, L, T, M, or V; L377 replaced with A, G, I, S, T, M, or V; G379 replaced with A, I, L, S, T, M, or V; L380 replaced with A, G, I, S, T, M, or V; G381 replaced with A, I, L, S, T, M, or V; G384 replaced with A, I, L, S, T, M, or V; S385 replaced with A, G, I, L, T, M, or V; L386 replaced with A, G, I, S, T, M, or V; K387 replaced with H, or R; A388 replaced with G, I, L, S, T, M, or V; S390 replaced with A, G, I, L, T, M, or V; R392 replaced with H, or K; S393 replaced with A, G, I, L, T, M, or V; S394 replaced with A, G, I, L, T, M, or V; A395 replaced with G, I, L, S, T, M, or V; S396 replaced with A, G, I, L, T, M, or V; H397 replaced with K, or R; K398 replaced with H, or R; S399 replaced with A, G, I, L, T, M, or V; L400 replaced with A, G, I, S, T, M, or V; S401 replaced with A, G, I, L, T, M, or V; or L402 replaced with A, G, I, S, T, M, or V of SEQ NO:2.

Additional preferred conservative mutations also include: L364 replaced with A, G, I, S, T, M, or V; W366 replaced with F, or Y; S367 replaced with A, G, I, L, T, M, or V; L368 replaced with A, G, I, S, T, M, or V; or L369 replaced with A, G, I, S, T, M, or V of SEQ ID NO:4.

Additional preferred conservative mutations also include: K365 replaced with H, or R; E366 replaced with D; K367 replaced with H, or R; S368 replaced with A, G, I, L, T, M, or V; L369 replaced with A, G, I, S, T, M, or V; V370 replaced with A, G, I, L, S, T, or M; L371 replaced with A, G, I, S, T, M, or V; or S372 replaced with A, G, I, L, T, M, or V of SEQ ID NO:6.

The resulting constructs can be routinely screened for activities or functions described throughout the specification and known in the art. Preferably, the resulting constructs have an increased neuropeptide receptor activity or function, while the remaining neuropeptide receptor activities or functions are maintained. More preferably, the resulting constructs have more than one increased neuropeptide receptor activity or function, while the remaining neuropeptide receptor activities or functions are maintained.

Besides conservative amino acid substitution, variants of neuropeptide receptor include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, neuropeptide receptor polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

For example, preferred non-conservative substitutions of neuropeptide receptor include: M1 replaced with D, E, H, K, R, N, Q, F, W, Y, P, or C; E2 replaced with H, K, R, A, G, I, L, S, T, M, V, N, Q, F, W, Y, P, or C; P3 replaced with D, E, H, K, R, A, G, I, L, S, T, M, V, N, Q, F, W, Y, or C; S4 replaced with D, E, H, K, R, N, Q, F, W, Y, P, or C; A5 replaced with D, E, H, K, R, N, Q, F, W, Y, P, or C; T6 replaced with D, E, H, K, R, N, Q, F, W, Y, P, or C; P7 replaced with D, E, H, K, R, A, G, I, L, S, T, M, V, N, Q, F, W, Y, or C; G8 replaced with D, E, H, K, R, N, Q, F, W, Y, P, or C; A9 replaced with D, E, H, K, R, N, Q, F, W, Y, P, or C; Q10 replaced with D, E, H, K, R, A, G, I, L, S, T, M, V, F, W, Y, P, or C; M1 replaced with D, E, H, K, R, N, Q, F, W, Y, P, or C; G12 replaced with D, E, H, K, R, N, Q, F, W, Y, P, or C; V13 replaced with D, E, H, K, R, N, Q, F, W, Y, P, or C; P14 replaced with D, E, H, K, R, A, G, I, L, S, T, M, V, N, Q, F, W, Y, or C; P15 replaced with D, E, H, K, R, A, G, I, L, S, T, M, V, N, Q, F, W, Y, or C; G16 replaced with D, E, H, K, R, N, Q, F, W, Y, P, or C; S17 replaced with D, E, H, K, R, N, Q, F, W, Y, P, or C; R18 replaced with D, E, A, G, I, L, S, T, M, V, N, Q, F, W, Y, P, or C; E19 replaced with H, K, R, A, G, I, L, S, T, M, V, N, Q, F, W, Y, P, or C; P20 replaced with D, E, H, K, R, A, G, I, L, S, T, M, V, N, Q, F, W, Y, or C; S21 replaced with D, E, H, K, R, N, Q, F, W, Y, P, or C; P22 replaced with D, E, H, K, R, A, G, I, L, S, T, M, V, N, Q, F, W, Y, or C; V23 replaced with D, E, H, K, R, N, Q, F, W, Y, P, or C; P24 replaced with D, E, H, K, R, A, G, I, L, S, T, M, V, N, Q, F, W, Y, or C; P25 replaced with D, E, H, K, R, A, G, I, L, S, T, M, V, N, Q, F, W, Y, or C; D26 replaced with H, K, R, A, G, I, L, S, T, M, V, N, Q, F, W, Y, P, or C; Y27 replaced with D, E, H, K, R, N, Q, A, G, I, L, S, T, M, V, P, or C; E28 replaced with H, K, R, A, G, I, L, S, T, M, V, N, Q, F, W, Y, P, or C; D29 replaced with H, K, R, A, G, I, L, S, T, M, V, N, Q, F, W, Y, P, or C; E30 replaced with H, K, R, A, G, I, L, S, T, M, V, N, Q, F, W, Y, P, or C; F31 replaced with D, E, H, K, R, N, Q, A, G, I, L, S, T, M, V, P, or C; L32 replaced with D, E, H, K, R, N, Q, F, W, Y, P, or C; R33 replaced with D, E, A, G, I, L, S, T, M, V, N, Q, F, W, Y, P, or C; Y34 replaced with D, E, H, K, R, N, Q, A, G, I, L, S, T, M, V, P, or C; L35 replaced with D, E, H, K, R, N, Q, F, W, Y, P, or C; W36 replaced with D, E, H, K, R, N, Q, A, G, I, L, S, T, M, V, P, or C; R37 replaced with D, E, A, G, I, L, S, T, M, V, N, Q, F, W, Y, P, or C; D38 replaced with H, K, R, A, G, I, L, S, T, M, V, N, Q, F, W, Y, P, or C; Y39 replaced with D, E, H, K, R, N, Q, A, G, I, L, S, T, M, V, P, or C; L40 replaced with D, E, H, K, R, N, Q, F, W, Y, P, or C; Y41 replaced with D, E, H, K, R, N, Q, A, G, I, L, S, T, M, V, P, or C; P42 replaced with D, E, H, K, R, A, G, I, L, S, T, M, V, N, Q, F, W, Y, or C; K43 replaced with D, E, A, G, I, L, S, T, M, V, N, Q, F, W, Y, P, or C; Q44 replaced with D, E, H, K, R, A, G, I, L, S, T, M, V, F, W, Y, P, or C; Y45 replaced with D, E, H, K, R, N, Q, A, G, I, L, S, T, M, V, P, or C; E46 replaced with H, K, R, A, G, I, L, S, T, M, V, N, Q, F, W, Y, P, or C; W47 replaced with D, E, H, K, R, N, Q, A, G, I, L, S, T, M, V, P, or C; V48 replaced with D, E, H, K, R, N, Q, F, W, Y, P, or C; L49 replaced with D, E, H, K, R, N, Q, F, W, Y, P, or C; I50 replaced with D, E, H, K, R, N, Q, F, W, Y, P, or C; A51 replaced with D, E, H, K, R, N, Q, F, W, Y, P, or C; A52 replaced with D, E, H, K, R, N, Q, F, W, Y, P, or C; Y53 replaced with D, E, H, K, R, N, Q, A, G, I, L, S, T, M, V, P, or C; V54 replaced with D, E, H, K, R, N, Q, F, W, Y, P, or C; A55 replaced with D, E, H, K, R, N, Q, F, W, Y, P, or C; V56 replaced with D, E, H, K, R, N, Q, F, W, Y, P, or C; F57 replaced with D, E, H, K, R, N, Q, A, G, I, L, S, T, M, V, P, or C; V58 replaced with D, E, H, K, R, N, Q, F, W, Y, P, or C; V59 replaced with D, E, H, K, R, N, Q, F, W, Y, P, or C; A60 replaced with D, E, H. K, R, N, Q, F, W, Y, P, or C; L61 replaced with D, E, H, K, R, N, Q, F, W, Y, P, or C; V62 replaced with D, E, H, K, R, N, Q, F, W, Y, P, or C; G63 replaced with D, E, H, K, R, N, Q, F, W, Y, P, or C; N64 replaced with D, E, H, K, R, A, G, I, L, S, T, M, V, F, W, Y, P, or C; T65 replaced with D, E, H, K, R, N, Q, F, W, Y, P, or C; L66 replaced with D, E, H, K, R, N, Q, F, W, Y, P, or C; V67 replaced with D, E, H, K, R, N, Q, F, W, Y, P, or C; C68 replaced with D, E, H, K, R, A, G, I, L, S, T, M, V, N, Q, F, W, Y, or P; L69 replaced with D, E, H, K, R, N, Q, F, W, Y, P, or C; A70 replaced with D, E, H, K, R, N, Q, F, W, Y, P, or C; V71 replaced with D, E, H, K, R, N, Q, F, W, Y, P, or C; W72 replaced with D, E, H, K, R, N, Q, A, G, I, L, S, T, M, V, P, or C; R73 replaced with D, E, A, G, I, L, S, T, M, V, N, Q, F, W, Y, P, or C; N74 replaced with D, E, H, K, R, A, G, I, L, S, T, M, V, F, W, Y, P, or C; H75 replaced with D, E, A, G, I, L, S, T, M, V, N, Q, F, W, Y, P, or C; H76 replaced with D, E, A, G, I, L, S, T, M, V, N, Q, F, W, Y, P, or C; M77 replaced with D, E, H, K, R, N, Q, F, W, Y, P, or C; R78 replaced with D, E, A, G, I, L, S, T, M, V, N, Q, F, W, Y, P, or C; T79 replaced with D, E, H, K, R, N, Q, F, W, Y, P, or C; V80 replaced with D, E, H, K, R, N, Q, F, W, Y, P, or C; T81 replaced with D, E, H, K, R, N, Q, F, W, Y, P, or C; N82 replaced with D, E, H, K, R, A, G, I, L, S, T, M, V, F, W, Y, P, or C; Y83 replaced with D, E, H, K, R, N, Q, A, G, I, L, S, T, M, V, P, or C; F84 replaced with D, E, H, K, R, N, Q, A, G, I, L, S, T, M, V, P, or C; I85 replaced with D, E, H, K, R, N, Q, F, W, Y, P, or C; V86 replaced with D, E, H, K, R, N, Q, F, W, Y, P, or C; N87 replaced with D, E, H, K, R, A, G, I, L, S, T, M, V, F, W, Y, P, or C; L88 replaced with D, E, H, K, R, N, Q, F, W, Y, P, or C; S89 replaced with D, E, H, K, R, N, Q, F, W, Y, P, or C; L90 replaced with D, E, H, K, R, N, Q, F, W, Y, P, or C; A91 replaced with D, E, H, K, R, N, Q, F, W, Y, P, or C; D92 replaced with H, K, R, A, G, I, L, S, T, M, V, N, Q, F, W, Y, P, or C; V93 replaced with D, E, H, K, R, N, Q, F, W, Y, P, or C; L94 replaced with D, E, H, K, R, N, Q, F, W, Y, P, or C; V95 replaced with D, E, H, K, R, N, Q, F, W, Y, P, or C; T96 replaced with D, E, H, K, R, N, Q, F, W, Y, P, or C; A97 replaced with D, E, H, K, R, N, Q, F, W, Y, P, or C; I98 replaced with D, E, H, K, R, N, Q, F, W, Y, P, or C; C99 replaced with D, E, H, K, R, A, G, I, L, S, T, M, V, N, Q, F, W, Y, or P; L100 replaced with D, E, H, K, R, N, Q, F, W, Y, P, or C; P101 replaced with D, E, H, K, R, A, G, I, L, S, T, M, V, N, Q, F, W, Y, or C; A102 replaced with D, E, H, K, R, N, Q, F, W, Y, P, or C; S103 replaced with D, E, H, K, R, N, Q, F, W, Y, P, or C; L104 replaced with D, E, H, K, R, N, Q, F, W, Y, P, or C; L105 replaced with D, E, H, K, R, N, Q, F, W, Y, P, or C; V106 replaced with D, E, H, K, R, N, Q, F, W, Y, P, or C; D107 replaced with H, K, R, A, G, I, L, S, T, M, V, N, Q, F, W, Y, P, or C; I108 replaced with D, E, H, K, R, N, Q, F, W, Y, P, or C; T109 replaced with D, E, H, K, R, N, Q, F, W, Y, P, or C; E110 replaced with H, K, R, A, G, I, L, S, T, M, V, N, Q, F, W, Y, P, or C; S111 replaced with D, E, H, K, R, N, Q, F, W, Y, P, or C; W112 replaced with D, E, H, K, R, N, Q, A, G, I, L, S, T, M, V, P, or C; L113 replaced with D, E, H, K, R, N, Q, F, W, Y, P, or C; F114 replaced with D, E, H, K, R, N, Q, A, G, I, L, S, T, M, V, P, or C; G115 replaced with D, E, H, K, R, N, Q, F, W, Y, P, or C; H116 replaced with D, E, A, G, I, L, S, T, M, V, N, Q, F, W, Y, P, or C; A117 replaced with D, E, H, K, R, N, Q, F, W, Y, P, or C; L118 replaced with D, E, H, K, R, N, Q, F, W, Y, P, or C; C119 replaced with D, E, H, K, R, A, G, I, L, S, T, M, V, N, Q, F, W, Y, or P; K120 replaced with D, E, A, G, I, L, S, T, M, V, N, Q, F, W, Y, P, or C; V121 replaced with D, E, H, K, R, N, Q, F, W, Y, P, or C; I122 replaced with D, E, H, K, R, N, Q, F, W, Y, P, or C; P123 replaced with D, E, H, K, R, A, G, I, L, S, T, M, V, N, Q, F, W, Y, or C; Y124 replaced with D, E, H, K, R, N, Q, A, G, I, L, S, T, M, V, P, or C; L125 replaced with D, E, H, K, R, N, Q, F, W, Y, P, or C; Q126 replaced with D, E, H, K, R, A, G, I, L, S, T, M, V, F, W, Y, P, or C; A127 replaced with D, E, H, K, R, N, Q, F, W, Y, P, or C; V128 replaced with D, E, H, K, R, N, Q, F, W, Y, P, or C; S129 replaced with D, E, H, K, R, N, Q, F, W, Y, P, or C; V130 replaced with D, E, H, K, R, N, Q, F, W, Y, P, or C; S131 replaced with D, E, H, K, R, N, Q, F, W, Y, P, or C; V132 replaced with D, E, H, K, R, N, Q, F, W, Y, P, or C; A133 replaced with D, E, H, K, R, N, Q, F, W, Y, P, or C; V134 replaced with D, E, H, K, R, N, Q, F, W, Y, P, or C; L135 replaced with D, E, H, K, R, N, Q, F, W, Y, P, or C; T136 replaced with D, E, H, K, R, N, Q, F, W, Y, P, or C; L137 replaced with D, E, H, K, R, N, Q, F, W, Y, P, or C; S138 replaced with D, E, H, K, R, N, Q, F, W, Y, P, or C; F139 replaced with D, E, H, K, R, N, Q, A, G, I, L, S, T, M, V, P, or C; I140 replaced with D, E, H, K, R, N, Q, F, W, Y, P, or C; A141 replaced with D, E, H, K, R, N, Q, F, W, Y, P, or C; L142 replaced with D, E, H, K, R, N, Q, F, W, Y, P, or C; D143 replaced with H, K, R, A, G, I, L, S, T, M, V, N, Q, F, W, Y, P, or C; R144 replaced with D, E, A, G, I, L, S, T, M, V, N, Q, F, W, Y, P, or C; W145 replaced with D, E, H, K, R, N, Q, A, G, I, L, S, T, M, V, P, or C; Y146 replaced with D, E, H, K, R, N, Q, A, G, I, L, S, T, M, V, P, or C; A147 replaced with D, E, H, K, R, N, Q, F, W, Y, P, or C; I148 replaced with D, E, H, K, R, N, Q, F, W, Y, P, or C; C149 replaced with D, E, H, K, R, A, G, I, L, S, T, M, V, N, Q, F, W, Y, or P; H150 replaced with D, E, A, G, I, L, S, T, M, V, N, Q, F, W, Y, P, or C; P151 replaced with D, E, H, K, R, A, G, I, L, S, T, M, V, N, Q, F, W, Y, or C; L152 replaced with D, E, H, K, R, N, Q, F, W, Y, P, or C; L153 replaced with D, E, H, K, R, N, Q, F, W, Y, P, or C; F154 replaced with D, E, H, K, R, N, Q, A, G, I, L, S, T, M, V, P, or C; K155 replaced with D, E, A, G, I, L, S, T, M, V, N, Q, F, W, Y, P, or C; S156 replaced with D, E, H, K, R, N, Q, F, W, Y, P, or C; T157 replaced with D, E, H, K, R, N, Q, F, W, Y, P, or C; A158 replaced with D, E, H, K, R, N, Q, F, W, Y, P, or C; R159 replaced with D, E, A, G, I, L, S, T, M, V, N, Q, F, W, Y, P, or C; R160 replaced with D, E, A, G, I, L, S, T, M, V, N, Q, F, W, Y, P, or C; A161 replaced with D, E, H, K, R, N, Q, F, W, Y, P, or C; R162 replaced with D, E, A, G, I, L, S, T, M, V, N, Q, F, W, Y, P, or C; G163 replaced with D, E, H, K, R, N, Q, F, W, Y, P, or C; S164 replaced with D, E, H, K, R, N, Q, F, W, Y, P, or C; I165 replaced with D, E, H, K, R, N, Q, F, W, Y, P, or C; L166 replaced with D, E, H, K, R, N, Q, F, W, Y, P, or C; G167 replaced with D, E, H, K, R, N, Q, F, W, Y, P, or C; I168 replaced with D, E, H, K, R, N, Q, F, W, Y, P, or C; W169 replaced with D, E, H, K, R, N, Q, A, G, I, L, S, T, M, V, P, or C; A170 replaced with D, E, H, K, R, N, Q, F, W, Y, P, or C; V171 replaced with D, E, H, K, R, N, Q, F, W, Y, P, or C; S172 replaced with D, E, H, K, R, N, Q, F, W, Y, P, or C; L173 replaced with D, E, H, K, R, N, Q, F, W, Y, P, or C; A174 replaced with D, E, H, K, R, N, Q, F, W, Y, P, or C; I175 replaced with D, E, H, K, R, N, Q, F, W, Y, P, or C; M176 replaced with D, E, H, K, R, N, Q, F, W, Y, P, or C; V177 replaced with D, E, H, K, R, N, Q, F, W, Y, P, or C; P178 replaced with D, E, H, K, R, A, G, I, L, S, T, M, V, N, Q, F, W, Y, or C; Q179 replaced with D, E, H, K, R, A, G, I, L, S, T, M, V, F, W, Y, P, or C; A180 replaced with D, E, H, K, R, N, Q, F, W, Y, P, or C; A181 replaced with D, E, H, K, R, N, Q, F, W, Y, P, or C; V182 replaced with D, E, H, K, R, N, Q, F, W, Y, P, or C; M183 replaced with D, E, H, K, R, N, Q, F, W, Y, P, or C; E184 replaced with H, K, R, A, G, I, L, S, T, M, V, N, Q, F, W, Y, P, or C; C185 replaced with D, E, H, K, R, A, G, I, L, S, T, M, V, N, Q, F, W, Y, or P; S186 replaced with D, E, H, K, R, N, Q, F, W, Y, P, or C; S187 replaced with D, E, H, K, R, N, Q, F, W, Y, P, or C; V188 replaced with D, E, H, K, R, N, Q, F, W, Y, P, or C; L189 replaced with D, E, H, K, R, N, Q, F, W, Y, P, or C; P190 replaced with D, E, H, K, R, A, G, I, L, S, T, M, V, N, Q, F, W, Y, or C; E191 replaced with H, K, R, A, G, I, L, S, T, M, V, N, Q, F, W, Y, P, or C; L192 replaced with D, E, H, K, R, N, Q, F, W, Y, P, or C; A193 replaced with D, E, H, K, R, N, Q, F, W, Y, P, or C; N194 replaced with D, E, H, K, R, A, G, I, L, S, T, M, V, F, W, Y, P, or C; R195 replaced with D, E, A, G, I, L, S, T, M, V, N, Q, F, W, Y, P, or C; T196 replaced with D, E, H, K, R, N, Q, F, W, Y, P, or C; R197 replaced with D, E, A, G, I, L, S, T, M, V, N, Q, F, W, Y, P, or C; L198 replaced with D, E, H, K, R, N, Q, F, W, Y, P, or C; F199 replaced with D, E, H, K, R, N, Q, A, G, I, L, S, T, M, V, P, or C; S200 replaced with D, E, H, K, R, N, Q, F, W, Y, P, or C; V201replaced with D, E, H, K, R, N, Q, F, W, Y, P, or C; C202 replaced with D, E, H, K, R, A, G, I, L, S, T, M, V, N, Q, F, W, Y, or P; D203 replaced with H, K, R, A, G, I, L, S, T, M, V, N, Q, F, W, Y, P, or C; E204 replaced with H, K, R, A, G, I, L, S, T, M, V, N, Q, F, W, Y, P, or C; R205 replaced with D, E, A, G, I, L, S, T, M, V, N, Q, F, W, Y, P, or C; W206 replaced with D, E, H, K, R, N, Q, A, G, I, L, S, T, M, V, P, or C; A207 replaced with D, E, H, K, R, N, Q, F, W, Y, P, or C; D208 replaced with H, K, R, A, G, I, L, S, T, M, V, N, Q, F, W, Y, P, or C; D209 replaced with H, K, R, A, G, I, L, S, T, M, V, N, Q, F, W, Y, P, or C; L210 replaced with D, E, H, K, R, N, Q, F, W, Y, P, or C; Y211 replaced with D, E, H, K, R, N, Q, A, G, I, L, S, T, M, V, P, or C; P212 replaced with D, E, H, K, R, A, G, I, L, S, T, M, V, N, Q, F, W, Y, or C; K213 replaced with D, E, A, G, I, L, S, T, M, V, N, Q, F, W, Y, P, or C; I214 replaced with D, E, H, K, R, N, Q, F, W, Y, P, or C; Y215 replaced with D, E, H, K, R, N, Q, A, G, I, L, S, T, M, V, P, or C; H216 replaced with D, E, A, G, I, L, S, T, M, V, N, Q, F, W, Y, P, or C; S217 replaced with D, E, H, K, R, N, Q, F, W, Y, P, or C; C218 replaced with D, E, H, K, R, A, G, I, L, S, T, M, V, N, Q, F, W, Y, or P; F219 replaced with D, E, H, K, R, N, Q, A, G, I, L, S, T, M, V, P, or C; F220 replaced with D, E, H, K, R, N, Q, A, G, I, L, S, T, M, V, P, or C; I221 replaced with D, E, H, K, R, N, Q, F, W, Y, P, or C; V222 replaced with D, E, H, K, R, N, Q, F, W, Y, P, or C; T223 replaced with D, E, H, K, R, N, Q, F, W, Y, P, or C; Y224 replaced with D, E, H, K, R, N, Q, A, G, I, L, S, T, M, V, P, or C; L225 replaced with D, E, H, K, R, N, Q, F, W, Y, P, or C; A226 replaced with D, E, H, K, R, N, Q, F, W, Y, P, or C; P227 replaced with D, E, H, K, R, A, G, I, L, S, T, M, V, N, Q, F, W, Y, or C; L228 replaced with D, E, H, K, R, N, Q, F, W, Y, P, or C; G229 replaced with D, E, H, K, R, N, Q, F, W, Y, P, or C; L230 replaced with D, E, H, K, R, N, Q, F, W, Y, P, or C; M231 replaced with D, E, H, K, R, N, Q, F, W, Y, P, or C; A232 replaced with D, E, H, K, R, N, Q, F, W, Y, P, or C; M233 replaced with D, E, H, K, R, N, Q, F, W, Y, P, or C; A234 replaced with D, E, H, K, R, N, Q, F, W, Y, P, or C; Y235 replaced with D, E, H, K, R, N, Q, A, G, I, L, S, T, M, V, P, or C; F236 replaced with D, E, H, K, R, N, Q, A, G, I, L, S, T, M, V, P, or C; Q237 replaced with D, E, H, K, R, A, G, I, L, S, T, M, V, F, W, Y, P, or C; I238 replaced with D, E, H, K, R, N, Q, F, W, Y, P, or C; F239 replaced with D, E, H, K, R, N, Q, A, G, I, L, S, T, M, V, P, or C; R240 replaced with D, E, A, G, I, L, S, T, M, V, N, Q, F, W, Y, P, or C; K241 replaced with D, E, A, G, I, L, S, T, M, V, N, Q, F, W, Y, P, or C; L242 replaced with D, E, H, K, R, N, Q, F, W, Y, P, or C; W243 replaced with D, E, H, K, R, N, Q, A, G, I, L, S, T, M, V, P, or C; G244 replaced with D, E, H, K, R, N, Q, F, W, Y, P, or C; R245 replaced with D, E, A, G, I, L, S, T, M, V, N, Q, F, W, Y, P, or C; Q246 replaced with D, E, H, K, R, A, G, I, L, S, T, M, V, F, W, Y, P, or C; I247 replaced with D, E, H, K, R, N, Q, F, W, Y, P, or C; P248 replaced with D, E, H, K, R, A, G, I, L, S, T, M, V, N, Q, F, W, Y, or C; G249 replaced with D, E, H, K, R, N, Q, F, W, Y, P, or C; T250 replaced with D, E, H, K, R, N, Q, F, W, Y, P, or C; T251 replaced with D, E, H, K, R, N, Q, F, W, Y, P, or C; S252 replaced with D, E, H, K, R, N, Q, F, W, Y, P, or C; A253 replaced with D, E, H, K, R, N, Q, F, W, Y, P, or C; L254 replaced with D, E, H, K, R, N, Q, F, W, Y, P, or C; V255 replaced with D, E, H, K, R, N, Q, F, W, Y, P, or C; R256 replaced with D, E, A, G, I, L, S, T, M, V, N, Q, F, W, Y, P, or C; N257 replaced with D, E, H, K, R, A, G, I, L, S, T, M, V, F, W, Y, P, or C; W258 replaced with D, E, H, K, R, N, Q, A, G, I, L, S, T, M, V, P, or C; K259 replaced with D, E, A, G, I, L, S, T, M, V, N, Q, F, W, Y, P, or C; R260 replaced with D, E, A, G, I, L, S, T, M, V, N, Q, F, W, Y, P, or C; P261 replaced with D, E, H, K, R, A, G, I, L, S, T, M, V, N, Q, F, W, Y, or C; S262 replaced with D, E, H, K, R, N, Q, F, W, Y, P, or C; D263 replaced with H, K, R, A, G, I, L, S, T, M, V, N, Q, F, W, Y, P, or C; Q264 replaced with D, E, H, K, R, A, G, I, L, S, T, M, V, F, W, Y, P, or C; L265 replaced with D, E, H, K, R, N, Q, F, W, Y, P, or C; G266 replaced with D, E, H, K, R, N, Q, F, W, Y, P, or C; D267 replaced with H, K, R, A, G, I, L, S, T, M, V, N, Q, F, W, Y, P, or C; L268 replaced with D, E, H, K, R, N, Q, F, W, Y, P, or C; E269 replaced with H, K, R, A, G, I, L, S, T, M, V, N, Q, F, W, Y, P, or C; Q270 replaced with D, E, H, K, R, A, G, I, L, S, T, M, V, F, W, Y, P, or C; G271 replaced with D, E, H, K, R, N, Q, F, W, Y, P, or C; L272 replaced with D, E, H, K, R, N, Q, F, W, Y, P, or C; S273 replaced with D, E, H, K, R, N, Q, F, W, Y, P, or C; G274 replaced with D, E, H, K, R, N, Q, F, W, Y, P, or C; E275 replaced with H, K, R, A, G, , L, S, T, M, V, N, Q, F, W, Y, P, or C; P276 replaced with D, E, H, K, R, A, G, I, L, S, T, M, V, N, Q, F, W, Y, or C; Q277 replaced with D, E, H, K, R, A, G, I, L, S, T, M, V, F, W, Y, P, or C; P278 replaced with D, E, H, K, R, A, G, I, L, S, T, M, V, N, Q, F, W, Y, or C; R279 replaced with D, E, A, G, I, L, S, T, M, V, N, Q, F, W, Y, P, or C; G280 replaced with D, E, H, K, R, N, Q, F, W, Y, P, or C; R281 replaced with D, E, A, G, I, L, S, T, M, V, N, Q, F, W, Y, P, or C; A282 replaced with D, E, H, K, R, N, Q, F, W, Y, P, or C; F283 replaced with D, E, H, K, R, N, Q, A, G, I, L, S, T, M, V, P, or C; L284 replaced with D, E, H, K, R, N, Q, F, W, Y, P, or C; A285 replaced with D, E, H, K, R, N, Q, F, W, Y, P, or C; E286 replaced with H, K, R, A, G, I, L, S, T, M, V, N, Q, F, W, Y, P, or C; V287 replaced with D, E, H, K, R, N, Q, F, W, Y, P, or C; K288 replaced with D, E, A, G, I, L, S, T, M, V, N, Q, F, W, Y, P, or C; Q289 replaced with D, E, H, K, R, A, G, I, L, S, T, M, V, F, W, Y, P, or C; M290 replaced with D, E, H, K, R, N, Q, F, W, Y, P, or C; R291 replaced with D, E, A, G, I, L, S, T, M, V, N, Q, F, W, Y, P, or C; A292 replaced with D, E, H, K, R, N, Q, F, W, Y, P, or C; R293 replaced with D, E, A, G, I, L, S, T, M, V, N, Q, F, W, Y, P, or C; R294 replaced with D, E, A, G, I, L, S, T, M, V, N, Q, F, W, Y, P, or C; K295 replaced with D, E, A, G, I, L, S, T, M, V, N, Q, F, W, Y, P, or C; T296 replaced with D, E, H, K, R, N, Q, F, W, Y, P, or C; A297 replaced with D, E, H, K, R, N, Q, F, W, Y, P, or C; K298 replaced with D, E, A, G, I, L, S, T, M, V, N, Q, F, W, Y, P, or C; M299 replaced with D, E, H, K, R, N, Q, F, W, Y, P, or C; L300 replaced with D, E, H, K, R, N, Q, F, W, Y, P, or C; M301 replaced with D, E, H, K, R, N, Q, F, W, Y, P, or C; V302 replaced with D, E, H, K, R, N, Q, F, W, Y, P, or C; V303 replaced with D, E, H, K, R, N, Q, F, W, Y, P, or C; L304 replaced with D, E, H, K, R, N, Q, F, W, Y, P, or C; L305 replaced with D, E, H, K, R, N, Q, F, W, Y, P, or C; V306 replaced with D, E, H, K, R, N, Q, F, W, Y, P, or C; F307 replaced with D, E, H, K, R, N, Q, A, G, I, L, S, T, M, V, P, or C; A308 replaced with D, E, H, K, R, N, Q, F, W, Y, P, or C; L309 replaced with D, E, H, K, R, N, Q, F, W, Y, P, or C; C310 replaced with D, E, H, K, R, A, G, I, L, S, T, M, V, N, Q, F, W, Y, or P; Y311 replaced with D, E, H, K, R, N, Q, A, G, I, L, S, T, M, V, P, or C; L312 replaced with D, E, H, K, R, N, Q, F, W, Y, P, or C; P313 replaced with D, E, H, K, R, A, G, I, L, S, T, M, V, N, Q, F, W, Y, or C; I314 replaced with D, E, H, K, R, N, Q, F, W, Y, P, or C; S315 replaced with D, E, H, K, R, N, Q, F, W, Y, P, or C; V316 replaced with D, E, H, K, R, N, Q, F, W, Y, P, or C; L317 replaced with D, E, H, K, R, N, Q, F, W, Y, P, or C; N318 replaced with D, E, H, K, R, A, G, I, L, S, T, M, V, F, W, Y, P, or C; V319 replaced with D, E, H, K, R, N, Q, F, W, Y, P, or C; L320 replaced with D, E, H, K, R, N, Q, F, W, Y, P, or C; K321 replaced with D, E, A, G, I, L, S, T, M, V, N, Q, F, W, Y, P, or C; R322 replaced with D, E, A, G, I, L, S, T, M, V, N, Q, F, W, Y, P, or C; V323 replaced with D, E, H, K, R, N, Q, F, W, Y, P, or C; F324 replaced with D, E, H, K, R, N, Q, A, G, I, L, S, T, M, V, P, or C; G325 replaced with D, E, H, K, R, N, Q, F, W, Y, P, or C; M326 replaced with D, E, H, K, R, N, Q, F, W, Y, P, or C; F327 replaced with D, E, H, K, R, N, Q, A, G, I, L, S, T, M, V, P, or C; R328 replaced with D, E, A, G, I, L, S, T, M, V, N, Q, F. W, Y, P, or C; Q329 replaced with D, E, H, K, R, A, G, I, L, S, T, M, V, F, W, Y, P, or C; A330 replaced with D, E, H, K, R, N, Q, F, W, Y, P, or C; S331 replaced with D, E, H, K, R, N, Q, F, W, Y, P, or C; D332 replaced with H, K, R, A, G, I, L, S, T, M, V, N, Q, F, W, Y, P, or C; R333 replaced with D, E, A, G, I, L, S, T, M, V, N, Q, F, W, Y, P, or C; E334 replaced with H, K, R, A, G, I, L, S, T, M, V, N, Q, F, W, Y, P, or C; A335 replaced with D, E, H, K, R, N, Q, F, W, Y, P, or C; V336 replaced with D, E, H, K, R, N, Q, F, W, Y, P, or C; Y337 replaced with D, E, H, K, R, N, Q, A, G, I, L, S, T, M, V, P, or C; A338 replaced with D, E, H, K, R, N, Q, F, W, Y, P, or C; C339 replaced with D, E, H, K, R, A, G, I, L, S, T, M, V, N, Q, F, W, Y, or P; F340 replaced with D, E, H, K, R, N, Q, A, G, I, L, S, T, M, V, P, or C; T341 replaced with D, E, H, K, R, N, Q, F, W, Y, P, or C; F342 replaced with D, E, H, K, R, N, Q, A, G, I, L, S, T, M, V, P, or C; S343 replaced with D, E, H, K, R, N, Q, F, W, Y, P, or C; H344 replaced with D, E, A, G, I, L, S, T, M, V, N, Q, F, W, Y, P, or C; W345 replaced with D, E, H, K, R, N, Q, A, G, I, L, S, T, M, V, P, or C; L346 replaced with D, E, H, K, R, N, Q, F, W, Y, P, or C; V347replaced with D, E, H, K, R, N, Q, F, W, Y, P, or C; Y348 replaced with D, E, H, K, R, N, Q, A, G, I, L, S, T, M, V, P, or C; A349 replaced with D, E, H, K, R, N, Q, F, W, Y, P, or C; N350 replaced with D, E, H, K, R, A, G, I, L, S, T, M, V, F, W, Y, P, or C; S351 replaced with D, E, H, K, R, N, Q, F, W, Y, P, or C; A352 replaced with D, E, H, K, R, N, Q, F, W, Y, P, or C; A353 replaced with D, E, H, K, R, N, Q, F, W, Y, P, or C; N354 replaced with D, E, H, K, R, A, G, I, L, S, T, M, V, F, W, Y, P, or C; P355 replaced with D, E, H, K, R, A, G, I, L, S, T, M, V, N, Q, F, W, Y, or C; I356 replaced with D, E, H, K, R, N, Q, F, W, Y, P, or C; I357 replaced with D, E, H, K, R, N, Q, F, W, Y, P, or C; Y358 replaced with D, E, H, K, R, N, Q, A, G, I, L, S, T, M, V, P, or C; N359 replaced with D, E, H, K, R, A, G, I, L, S, T, M, V, F, W, Y, P, or C; F360 replaced with D, E, H, K, R, N, Q, A, G, I, L, S, T, M, V, P, or C; L361 replaced with D, E, H, K, R, N, Q, F, W, Y, P, or C; S362 replaced with D, E, H, K, R, N, Q, F, W, Y, P, or C; or G363 replaced with D, E, H, K, R, N, Q, F, W, Y, P, or C of SEQ ID NO:2, 4, or 6.

Additional preferred non-conservative mutations include: K364 replaced with D, E, A, G, I, L, S, T, M, V, N, Q, F, W, Y, P, or C; F365 replaced with D, E, H, K, R, N, Q, A, G, I, L, S, T, M, V, P, or C; R366 replaced with D, E, A, G, I, L, S, T, M, V, N, Q, F, W, Y, P, or C; E367 replaced with H, K, R, A, G, I, L, S, T, M, V, N, Q, F, W, Y, P, or C; Q368 replaced with D, E, H, K, R, A, G, I, L, S, T, M, V, F, W, Y, P, or C; F369 replaced with D, E, H, K, R, N, Q, A, G, I, L, S, T, M, V, P, or C; K370 replaced with D, E, A, G, I, L, S, T, M, V, N, Q, F, W, Y, P, or C; A371 replaced with D, E, H, K, R, N, Q, F, W, Y, P, or C; A372 replaced with D, E, H, K, R, N, Q, F, W, Y, P, or C; F373 replaced with D, E, H, K, R, N, Q, A, G, I, L, S, T, M, V, P, or C; S374 replaced with D, E, H, K, R, N, Q, F, W, Y, P, or C; C375 replaced with D, E, H, K, R, A, G, I, L, S, T, M, V, N, Q, F, W, Y, or P; C376 replaced with D, E, H, K, R, A, G, I, L, S, T, M, V, N, Q, F, W, Y, or P; L377 replaced with D, E, H, K, R, N, Q, F, W, Y, P, or C; P378 replaced with D, E, H, K, R, A, G, I, L, S, T, M, V, N, Q, F, W, Y, or C; G379 replaced with D, E, H, K, R, N, Q, F, W, Y, P, or C; L380 replaced with D, E, H, K, R, N, Q, F, W, Y, P, or C; G381 replaced with D, E, H, K, R, N, Q, F, W, Y, P, or C; P382 replaced with D, E, H, K, R, A, G, I, L, S, T, M, V, N, Q, F, W, Y, or C; C383 replaced with D, E, H, K, R, A, G, I, L, S, T, M, V, N, Q, F, W, Y, or P; G384 replaced with D, E, H, K, R, N, Q, F, W, Y, P, or C; S385 replaced with D, E, H, K, R, N, Q, F, W, Y, P, or C; L386 replaced with D, E, H, K, R, N, Q, F, W, Y, P, or C; K387 replaced with D, E, A, G, I, L, S, T, M, V, N, Q, F, W, Y, P, or C; A388 replaced with D, E, H, K, R, N, Q, F, W, Y, P, or C; P389 replaced with D, E, H, K, R, A, G, I, L, S, T, M, V, N, Q, F, W, Y, or C; S390 replaced with D, E, H, K, R, N, Q, F, W, Y, P, or C; P391 replaced with D, E, H, K, R, A, G, I, L, S, T, M, V, N, Q, F, W, Y, or C; R392 replaced with D, E, A, G, I, L, S, T, M, V, N, Q, F, W, Y, P, or C; S393 replaced with D, E, H, K, R, N, Q, F, W, Y, P, or C; S394 replaced with D, E, H, K, R, N, Q, F, W, Y, P, or C; A395 replaced with D, E, H, K, R, N, Q, F, W, Y, P, or C; S396 replaced with D, E, H, K, R, N, Q, F, W, Y, P, or C; H397 replaced with D, E, A, G, I, L, S, T, M, V, N, Q, F, W, Y, P, or C; K398 replaced with D, E, A, G, I, L, S, T, M, V, N, Q, F, W, Y, P, or C; S399 replaced with D, E, H, K, R, N, Q, F, W, Y, P, or C; L400 replaced with D, E, H, K, R, N, Q, F, W, Y, P, or C; S401 replaced with D, E, H, K, R, N, Q, F, W, Y, P, or C; or L402 replaced with D, E, H, K, R, N, Q, F, W, Y, P, or C of SEQ ID NO:2.

Additional preferred non-conservative mutations include: L364 replaced with D, E, H, K, R, N, Q, F, W, Y, P, or C; P365 replaced with D, E, H, K, R, A, G, I, L, S, T, M, V, N, Q, F, W, Y, or C; W366 replaced with D, E, H, K, R, N, Q, A, G, I, L, S, T, M, V, P, or C; S367 replaced with D, E, H, K, R, N, Q, F, W, Y, P, or C; L368 replaced with D, E, H, K, R, N, Q, F, W, Y, P, or C; or L369 replaced with D, E, H, K, R, N, Q, F, W, Y, P, or C of SEQ ID NO:4.

Additional preferred non-conservative mutations include: C364 replaced with D, E, H, K, R, A, G, I, L, S, T, M, V, N, Q, F, W, Y, or P; K365 replaced with D, E, A, G, I, L, S, T, M, V, N, Q, F, W, Y, P, or C; E366 replaced with H, K, R, A, G, I, L, S, T, M, V, N, Q, F, W, Y, P, or C; K367 replaced with D, E, A, G, I, L, S, T, M, V, N, Q, F, W, Y, P, or C; S368 replaced with D, E, H, K, R, N, Q, F, W, Y, P, or C; L369 replaced with D, E, H, K, R, N, Q, F, W, Y, P, or C; V370 replaced with D, E, H, K, R, N, Q, F, W, Y, P, or C; L371 replaced with D, E, H, K, R, N, Q, F, W, Y, P, or C; or S372 replaced with D, E, H, K, R, N, Q, F, W, Y, P, or C of SEQ ID NO:6.

The resulting constructs can be routinely screened for activities or functions described throughout the specification and known in the art. Preferably, the resulting constructs have loss of a neuropeptide receptor activity or function, while the remaining neuropeptide receptor activities or functions are maintained. More preferably, the resulting constructs have more than one loss of neuropeptide receptor activity or function, while the remaining neuropeptide receptor activities or functions are maintained.

Additionally, more than one amino acid (e.g., 2, 3, 4, 5, 6, 7, 8, 9 and 10) can be replaced with the substituted amino acids as described above (either conservative or nonconservative). The substituted amino acids can occur in the full length, mature, or proprotein form of neuropeptide receptor protein, as well as the N- and C-terminal deletion mutants, having the general formula m-n, listed below.

A further embodiment of the invention relates to a polypeptide which comprises the amino acid sequence of a neuropeptide receptor polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of a neuropeptide receptor polypeptide, which contains at least one, but not more than 10, 9, 8, 7, 6, 5, 4, 3, 2 or 1 amino acid substitutions. In specific embodiments, the number of additions, substitutions, and/or deletions in the amino acid sequences of FIGS. 1A-F, 2A-E, or 3A-F or fragments thereof (e.g., the mature form and/or other fragments described herein), is 1-5, 5-10, 5-25, 5-50, 10-50 or 50-150, conservative amino acid substitutions are preferable.

Polynucleotide and Polypeptide Fragments

The present invention is further directed to fragments of the isolated nucleic acid molecules described herein. By a fragment of an isolated nucleic acid molecule having, for example, the nucleotide sequence of the deposited cDNA (clone HFGAN72), a nucleotide sequence encoding the polypeptide sequence encoded by the deposited cDNA, a nucleotide sequence encoding the polypeptide sequence depicted in FIGS. 1A-F, 2A-E, or 3A-F (SEQ ID NO:2, 4, or 6), the nucleotide sequence shown in FIGS. 1A-F, 2A-E, or 3A-F (SEQ ID NO:1, 2, or 3), or the complementary strand thereto, is intended fragments at least 15 nt, and more preferably at least about 20 nt, still more preferably at least 30 nt, and even more preferably, at least about 40, 50, 100, 150, 200, 250, 300, 325, 350, 375, 400, 450, 500, 550, or 600 nt in length. These fragments have numerous uses that include, but are not limited to, diagnostic probes and primers as discussed herein. Of course, larger fragments, such as those of 501-1500 nt in length are also useful according to the present invention as are fragments corresponding to most, if not all, of the nucleotide sequences of the deposited cDNA (clone HFGAN72) or as shown in FIGS. 1A-F, 2A-E, or 3A-F (SEQ ID NO:1, 3, or 5). By a fragment at least 20 nt in length, for example, is intended fragments which include 20 or more contiguous bases from, for example, the nucleotide sequence of the deposited cDNA, or the nucleotide sequence as shown in FIGS. 1A-F, 2A-E, or 3A-F (SEQ ID NO:1, 3, or 5).

Moreover, representative examples of neuropeptide receptor polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, and/or 2001 to the end of SEQ ID NO:1 or the complementary strand thereto, or the cDNA contained in the deposited clone. In this context “about” includes the particularly recited ranges, larger or smaller by several (5, 4, 3, 2, or 1) nucleotides, at either terminus or at both termini.

Preferably, the polynucleotide fragments of the invention encode a polypeptide which demonstrates a neuropeptide receptor functional activity. By a polypeptide demonstrating a neuropeptide receptor “functional activity” is meant, a polypeptide capable of displaying one or more known functional activities associated with a full-length (complete) neuropeptide receptor protein. Such functional activities include, but are not limited to, biological activity, antigenicity [ability to bind (or compete with a neuropeptide receptor polypeptide for binding) to an anti-neuropeptide receptor antibody], immunogenicity (ability to generate antibody which binds to a neuropeptide receptor polypeptide), ability to form multimers with neuropeptide receptor polypeptides of the invention, and ability to bind to a receptor or ligand for a neuropeptide receptor polypeptide.

The functional activity of neuropeptide receptor polypeptides, and fragments, variants derivatives, and analogs thereof, can be assayed by various methods.

For example, in one embodiment where one is assaying for the ability to bind or compete with full-length neuropeptide receptor polypeptide for binding to anti-neuropeptide receptor antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where a neuropeptide receptor ligand is identified, or the ability of a polypeptide fragment, variant or derivative of the invention to multimerize is being evaluated, binding can be assayed, e.g., by means well-known in the art, such as, for example, reducing and non-reducing gel chromatography, protein affinity chromatography, and affinity blotting. See generally, Phizicky, E., et al., 1995, Microbiol. Rev. 59:94-123. In another embodiment, physiological correlates of neuropeptide receptor binding to its substrates (signal transduction) can be assayed.

In addition, assays described herein (see Examples) and otherwise known in the art may routinely be applied to measure the ability of neuropeptide receptor polypeptides and fragments, variants derivatives and analogs thereof to elicit neuropeptide receptor related biological activity (either in vitro or in vivo). Other methods will be known to the skilled artisan and are within the scope of the invention.

The present invention is further directed to fragments of the neuropeptide receptor polypeptide described herein. By a fragment of an isolated the neuropeptide receptor polypeptide, for example, encoded by the deposited cDNA (clone HFGAN72), the polypeptide sequence encoded by the deposited cDNA, the polypeptide sequence depicted in FIGS. 1A-F, 2A-E, or 3A-F (SEQ ID NO:2, 4, or 6), is intended to encompass polypeptide fragments contained in SEQ ID NO:2, 4, or 6 or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200, 201-220, 221-240, 241-260, 261-280, or 281 to the end of the coding region. Moreover, polypeptide fragments can be at least 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Even if deletion of one or more amino acids from the N-terminus of a protein results in modification of loss of one or more biological functions of the protein, other functional activities (e.g., biological activities, ability to multimerize, ability to bind neuropeptide receptor ligand) may still be retained. For example, the ability of shortened neuropeptide receptor muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that an neuropeptide receptor mutein with a large number of deleted N-terminal amino acid residues may retain some biological or immunogenic activities. In fact, peptides composed of as few as six neuropeptide receptor amino acid residues may often evoke an immune response.

Accordingly, polypeptide fragments include the secreted neuropeptide receptor protein as well as the mature form. Further preferred polypeptide fragments include the secreted neuropeptide receptor protein or the mature form having a continuous series of deleted residues from the amino or the carboxy terminus, or both. For example, any number of amino acids, ranging from 1-60, can be deleted from the amino terminus of either the secreted neuropeptide receptor polypeptide or the mature form. Similarly, any number of amino acids, ranging from 1-30, can be deleted from the carboxy terminus of the secreted neuropeptide receptor protein or mature form. Furthermore, any combination of the above amino and carboxy terminus deletions are preferred. Similarly, polynucleotide fragments encoding these neuropeptide receptor polypeptide fragments are also preferred.

Particularly, N-terminal deletions of the neuropeptide receptor polypeptide can be described by the general formula m-363, where m is an integer from 1 to 357 where m corresponds to the position of the amino acid residue identified in SEQ ID NO:2, 4, or 6. More in particular, the invention provides polynucleotides encoding polypeptides comprising, or alternatively consisting of the amino acid sequence of residues of: M-1 to G363; E-2 to G-363; P-3 to G-363; S-4 to G-363; A-5 toG-363; T-6 to G-363; P-7 to G-363; G-8 to G-363; A-9 to G-363; Q-10 to G-363; M-11 to G-363; G-12 to G-363; V-13 to G-363; P-14 to G-363; P-15 to G-363; G-16 to G-363; S-17 to G-363; R-18 to G-363; E-19 to G-363; P-20 to G-363; S-21 to G-363; P-22 to G-363; V-23 to G-363; P-24 to G-363; P-25 to G-363; D-26 to G-363; Y-27 to G-363; E-28 to G-363; D-29 to G-363; E-30 to G-363; F-31 to G-363; L-32 to G-363; R-33 to G-363; Y-34 to G-363; L-35 to G-363; W-36 to G-363; R-37 to G-363; D-38 to G-363; Y-39 to G-363; L-40 to G-363; Y-41 to G-363; P-42 to G-363; K-43 to G-363; Q-44 to G-363; Y-45 to G-363; E-46 to G-363; W-47 to G-363; V-48 to G-363; L-49 to G-363; I-50 to G-363; A-51 to G-363; A-52 to G-363; Y-53 to G-363; V-54 to G-363; A-55 to G-363; V-56 to G-363; F-57 to G-363; V-58 to G-363; V-59 to G-363; A-60 to G-363; L-61 to G-363; V-62 to G-363; G-63 to G-363; N-64 to G-363; T-65 to G-363; L-66 to G-363; V-67 to G-363; C-68 to G-363; L-69 to G-363; A-70 to G-363; V-71 to G-363; W-72 to G-363; R-73 to G-363; N-74 to G-363; H-75 to G-363; H-76 to G-363; M-77 to G-363; R-78 to G-363; T-79 to G-363; V-80 to G-363; T-81 to G-363; N-82 to G-363; Y-83 to G-363; F-84 to G-363; 1-85 to G-363; V-86 to G-363; N-87 to G-363; L-88 to G-363; S-89 to G-363; L-90 to G-363; A-91 to G-363; D-92 to G-363; V-93 to G-363; L-94 to G-363; V-95 to G-363; T-96 to G-363; A-97 to G-363; I-98 to G-363; C-99 to G-363; L-100 to G-363; P-101 to G-363; A-102 to G-363; S-103 to G-363; L-104 to G-363; L-105 to G-363; V-106 to G-363; D-107 to G-363; 1-108 to G-363; T-109 to G-363; E-110 to G-363; S-111 to G-363; W-112 to G-363; L-113 to G-363; F-114 to G-363; G-115 to G-363; H-116 to G-363; A-117 to G-363; L-118 to G-363; C-119 to G-363; K-120 to G-363; V-121 to G-363; I-122 to G-363; P-123 to G-363; Y-124 to G-363; L-125 to G-363; Q-126 to G-363; A-127 to G-363; V-128 to G-363; S-129 to G-363; V-130 to G-363; S-131 to G-363; V-132 to G-363; A-133 to G-363; V-134 to G-363; L-135 to G-363; T-136 to G-363; L-137 to G-363; S-138 to G-363; F-139 to G-363; 1-140 to G-363; A-141 to G-363; L-142 to G-363; D-143 to G-363; R-144 to G-363; W-145 to G-363; Y-146 to G-363; A-147 to G-363; I-148 to G-363; C-149 to G-363; H-150 to G-363; P-151 to G-363; L-152 to G-363; L-153 to G-363; F-154 to G-363; K-155 to G-363; S-156 to G-363; T-157 to G-363; A-158 to G-363; R-159 to G-363; R-160 to G-363; A-161 to G-363; R-162 to G-363; G-163 to G-363; S-164 to G-363; I-165 to G-363; L-166 to G-363; G-167 to G-363; 1-168 to G-363; W-169 to G-363; A-170 to G-363; V-171 to G-363; S-172 to G-363; L-173 to G-363; A-174 to G-363; I-175 to G-363; M-176 to G-363; V-177 to G-363; P-178 to G-363; Q-179 to G-363; A-180 to G-363; A-181 to G-363; V-182 to G-363; M-183 to G-363; E-184 to G-363; C-185 to G-363; S-186 to G-363; S-187 to G-363; V-188 to G-363; L-189 to G-363; P-190 to G-363; E-191 to G-363; L-192 to G-363; A-193 to G-363; N-194 to G-363; R-195 to G-363; T-196 to G-363; R-197 to G-363; L-198 to G-363; F-199 to G-363; S-200 to G-363; V-201 to G-363; C-202 to G-363; D-203 to G-363; E-204 to G-363; R-205 to G-363; W-206 to G-363; A-207 to G-363; D-208 to G-363; D-209 to G-363; L-210 to G-363; Y-211 to G-363; P-212 to G-363; K-213 to G-363; I-214 to G-363; Y-215 to G-363; H-216 to G-363; S-217 to G-363; C-218 to G-363; F-219 to G-363; F-220 to G-363; 1-221 to G-363; V-222 to G-363; T-223 to G-363; Y-224 to G-363; L-225 to G-363; A-226 to G-363; P-227 to G-363; L-228 to G-363; G-229 to G-363; L-230 to G-363; M-231 to G-363; A-232 to G-363; M-233 to G-363; A-234 to G-363; Y-235 to G-363; F-236 to G-363; Q-237 to G-363; 1-238 to G-363; F-239 to G-363; R-240 to G-363; K-241 to G-363; L-242 to G-363; W-243 to G-363; G-244 to G-363; R-245 to G-363; Q-246 to G-363; I-247 to G-363; P-248 to G-363; G-249 to G-363; T-250 to G-363; T-251 to G-363; S-252 to G-363; A-253 to G-363; L-254 to G-363; V-255 to G-363; R-256 to G-363; N-257 to G-363; W-258 to G-363; K-259 to G-363; R-260 to G-363; P-261 to G-363; S-262 to G-363; D-263 to G-363; Q-264 to G-363; L-265 to G-363; G-266 to G-363; D-267 to G-363; L-268 to G-363; E-269 to G-363; Q-270 to G-363; G-271 to G-363; L-272 to G-363; S-273 to G-363; G-274 to G-363; E-275 to G-363; P-276 to G-363; Q-277 to G-363; P-278 to G-363; R-279 to G-363; G-280 to G-363; R-281 to G-363; A-282 to G-363; F-283 to G-363; L-284 to G-363; A-285 to G-363; E-286 to G-363; V-287 to G-363; K-288 to G-363; Q-289 to G-363; M-290 to G-363; R-291 to G-363; A-292 to G-363; R-293 to G-363; R-294 to G-363; K-295 to G-363; T-296 to G-363; A-297 to G-363; K-298 to G-363; M-299 to G-363; L-300 to G-363; M-301 to G-363; V-302 to G-363; V-303 to G-363; L-304 to G-363; L-305 to G-363; V-306 to G-363; F-307 to G-363; A-308 to G-363; L-309 to G-363; C-310 to G-363; Y-311 to G-363; L-312 to G-363; P-313 to G-363; 1-314 to G-363; S-315 to G-363; V-316 to G-363; L-317 to G-363; N-318 to G-363; V-319 to G-363; L-320 to G-363; K-321 to G-363; R-322 to G-363; V-323 to G-363; F-324 to G-363; G-325 to G-363; M-326 to G-363; F-327 to G-363; R-328 to G-363; Q-329 to G-363; A-330 to G-363; S-331 to G-363; D-332 to G-363; R-333 to G-363; E-334 to G-363; A-335 to G-363; V-336 to G-363; Y-337 to G-363; A-338 to G-363; C-339 to G-363; F-340 to G-363; T-341 to G-363; F-342 to G-363; S-343 to G-363; H-344 to G-363; W-345 to G-363; L-346 to G-363; V-347 to G-363; Y-348 to G-363; A-349 to G-363; N-350 to G-363; S-351 to G-363; A-352 to G-363; A-353 to G-363; N-354 to G-363; P-355 to G-363; I-356 to G-363; or I-357 to G-363.

The above N-terminal deletion mutants (m-363) can also include the following amino acids linked to G-363: K-364; K-364 to F-365; K-364 to R-366; K-364 to E-367; K-364 to Q-368; K-364 to F-369; K-364 to K-370; K-364 to A-371; K-364 to A-372; K-364 to F-373; K-364 to S-374; K-364 to C-375; K-364 to C-376; K-364 to L-377; K-364 to P-378; K-364 to G-379; K-364 to L-380; K-364 to G-381; K-364 to P-382; K-364 to C-383; K-364 to G-384; K-364 to S-385; K-364 to L-386; K-364 to K-387; K-364 to A-388; K-364 to P-389; K-364 to S-390; K-364 to P-391; K-364 to R-392; K-364 to S-393; K-364 to S-394; K-364 to A-395; K-364 to S-396; K-364 to H-397; K-364 to K-398; K-364 to S-399; K-364 to L-400; K-364 to S-401; or K-364 to L-402 of SEQ ID NO:2. Polynucleotides encoding these polypeptides are also encompassed by the invention.

Additionally, the above N-terminal deletion mutants (m-363) can also include the following amino acids linked to G-363: L-364; L-364 to P-365; L-364 to W-366; L-364 to S-367; L-364 to L-368; or L-364 to L-369 of SEQ ID NO:4. Polynucleotides encoding these polypeptides are also encompassed by the invention.

Moreover, the above N-terminal deletion mutants (m-363) can also include the following amino acids linked to G-363: C-364; C-364 to K-365; C-364 to E-366; C-364 to K-367; C-364 to S-368; C-364 to L-369; C-364 to V-370; C-364 to L-371; or C-364 to S-372 of SEQ ID NO:6. Polynucleotides encoding these polypeptides are also encompassed by the invention.

Also as mentioned above, even if deletion of one or more amino acids from the C-terminus of a protein results in modification of loss of one or more biological functions of the protein, other functional activities (e.g., biological activities, ability to multimerize, ability to bind neuropeptide receptor ligand) may still be retained. For example the ability of the shortened neuropeptide receptor mutein to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that an neuropeptide receptor mutein with a large number of deleted C-terminal amino acid residues may retain some biological or immunogenic activities. In fact, peptides composed of as few as six neuropeptide receptor amino acid residues may often evoke an immune response.

Accordingly, the present invention further provides polypeptides having one or more residues deleted from the carboxy terminus of the amino acid sequence of the neuropeptide receptor polypeptide shown in FIGS. 1A-F, 2A-E, and 3A-F (SEQ ID NO:2, 4, and 6), as described by the general formula 1-n, where n is an integer from 6 to 363, where n corresponds to the position of amino acid residue identified in SEQ ID NO:2, 4, and 6. More in particular, the invention provides polynucleotides encoding polypeptides comprising, or alternatively consisting of, the amino acid sequence of residues of: M-1 to G-363; M-1 to S-362; M-1 to L-361; M-1 to F-360; M-1 to N-359; M-1 to Y-358; M-1 to I-357; M-1 to I-356; M-1 to P-355; M-1 to N-354; M-1 to A-353; M-1 to A-352; M-1 to S-351; M-1 to N-350; M-1 to A-349; M-1 to Y-348; M-1 to V-347; M-1 to L-346; M-1 to W-345; M-1 to H-344; M-1 to S-343; M-1 to F-342; M-1 to T-341; M-1 to F-340; M-1 to C-339; M-1 to A-338; M-1 Y-337; M-1 to V-336; M-1 to A-335; M-1 to E-334; M-1 to R-333; M-1 to D-332; M-1 to S-331: M-1 to A-330; M-1 to Q-329; M-1 to R-328; M-1 to F-327; M-1 to M-326; M-1 to G-325; M-1 to F-324; M-1 to V-323; M-1 to R-322; M-1 to K-321; M-1 to L-320; M-1 to V-319; M-1 to N-318; M-1 to L-317; M-1 to V-316; M-1 to S-315; M-1 to I-314; M-1 to P-313; M-1 to L-312; M-1 to Y-311; M-1 to C-310; M-1 to L-309; M-1 to A-308; M-1 to F-307; M-1 to V-306; M-1 to L-305; M-1 to L-304; M-1 to V-303; M-1 to V-302; M-1 to M-301; M-1 to L-300; to M-1 to M-299; M-1 to K-298; M-1 to A-297; M-1 to T-296; M-1 to K-295; M-1 to R-294; M-1 to R-293; M-1 to A-292; M-1 to R-291; M-1 to M-290; M-1 to Q-289; M-1 to K-288; M-1 to V-287; M-1 to E-286; M-1 to A-285; M-1 to L-284; M-1 to F-283; M-1 to A-282; M-1 M-1 to R-281; M-1 to G-280; M-1 to R-279; M-1 to P-278; M-1 to Q-277; M-1 to P-276; M-1 to E-275; M-1 to G-274; M-1 to S-273; M-1 to L-272; M-1 to G-271; M-1 to Q-270; M-1 to E-269; M-1 to L-268; M-1 to D-267; M-1 to G-266; M-1 to L-265; M-1 to Q-264; M-1 to D-263; M-1 to S-262; M-1 to P-261; M-1 to R-260; M-1 to K-259; M-1 to W-258; M-1 to N-257; M-1 to R-256; M-1 to V-255; M-1 to L-254; M-1 to A-253; M-1 to S-252; M-1 to T-251; M-1 to T-250; M-1 to G-249; M-1 to P-248; M-1 to I-247; M-1 to Q-246; M-1 to R-245; M-1 to G-244; M-1 to W-243; M-1 to L-242; M-1 to K-241; M-1 to R-240; M-1 to F-239; M-1 to I-238; M-1 to Q-237; M-1 to F-236; M-1 to Y-235; M-1 to A-234; M-1 to M-233; M-1 to A-232; M-1 to M-231; M-1 to L-230; M-1 to G-229; M-1 to L-228; M-1 to P-227; M-1 to A-226; M-1 to L-225; M-1 to Y-224; M-1 to T-223; M-1 to V-222; M-1 to I-221; M-1 to F-220; M-1 to F-219; M-1 to C-218; M-1 to S-217; M-1 to H-216; M-1 to Y-215; M-1 to I-214; M-1 to K-213; M-1 to P-212 M-1 to Y-211; M-1 to L-210; M-1 to D-209; M-1 to D-208; M-1 to A-207; M-1 to W-206; M-1 to R-205; M-1 to E-204; M-1 to D-203; M-1 to C-202; M-1 to V-201; M-1 to S-200; M-1 to F-199; M-1 to L-198; M-1 to R-197; M-1 to T-196; M-1 to R-195; M-1 to N-194; M-1 to A-193; M-1 to L-192; M-1 to E-191; M-1 to P-190; M-1 to L-189; M-1 to V-188; M-1 to M-1 to S-187; M-1 to S-186; M-1 to C-185; M-1 to E-184; M-1 to M-183; M-1 to V-182; M-1 to A-181; M-1 to A-180; M-1 to Q-179; M-1 to P-178; M-1 to V-177; M-1 to M-176; M-1 to I-175; M-1 to A-174; M-1 to L-173; M-1 to S-172; M-1 to V-171; M-1 to A-170; M-1 to W-169; M-1 to I-168; M-1 to G-167; M-1 to L-166; M-1 to I-165; M-1 to S-164; M-1 to G-163; M-1 to R-162; M-1 to A-161; M-1 to R-160; M-1 to R-159; M-1 to A-158; M-1 to T-157; M-1 to S-156; M-1 to K-155; M-1 to F-154; M-1 to L-153; M-1 to L-152; M-1 to P-151; M-1 to H-150; M-1 to C-149; M-1 to I-148; M-1 to A-147; M-1 to Y-146; M-1 to W-145; M-1 to R-144; M-1 to D-143; M-1 to L-142; M-1 to A-141; M-1 to I-140; M-1 to F-139; M-1 to S-138; M-1 to L-137; M-1 to T-136; M-1 to L-135; M-1 to V-134; M-1 to A-133; M-1 to V-132; M-1 to S-131; M-1 to V-130; M-1 to S-129; M-1 to V-128; M-1 to A-127; M-1 to Q-126; M-1 to L-125; M-1 to Y-124; M-1 to P-123; M-1 to I-122; M-1 to V-121; M-1 to K-120; M-1 to C-119; M-1 to L-118; M-1 to A-117; M-1 to H-116; M-1 to G-115; M-1 to F-114; M-1 to L-113; M-1 to W-112; M-1 to S-111; M-1 to E-110; M-1 to T-109; M-1 to I-108; M-1 to D-107; M-1 to V-106; M-1 to L-105; M-1 to L-104; M-1 to S-103; M-1 to A-102; M-1 to P-101; M-1 to L-100; M-1 to C-99; M-1 to I-98; M-1 to A-97; M-1 to T-96; M-1 to V-95; M-1 to L-94; M-1 to V-93; M-1 to D-92; M-1 to A-91; M-1 to L-90; M-1 to S-89; M-1 to L-88; M-1 to N-87; M-1 to V-86; M-1 to I-85; M-1 to F-84; M-1 to Y-83; M-1 to N-82; M-1 to T-81; M-1 to V-80; M-1 to T-79; M-1 to R-78; M-1 to M-77; M-1 to H-76; M-1 to H-75; M-1 to N-74; M-1 to R-73; M-1 to W-72; M-1 to V-71; M-1 to A-70; M-1 to L-69; M-1 to C-68; M-1 to V-67; M-1 to L-66; M-1 to T-65; M-1 to N-64; M-1 to G-63; M-1 to V-62; M-1 to L-61; M-1 to A-60; M-1 to V-59; M-1 to V-58; M-1 to F-57; M-1 to V-56; M-1 to A-55; M-1 to V-54; M-1 to Y-53; M-1 to A-52; M-1 to A-51; M-1 to I-50; M-1 to L-49; M-1 to V-48; M-1 to W-47; M-1 to E-46; M-1 to Y-45; M-1 to Q-44; M-1 to K-43; M-1 to P-42; M-1 to Y-41; M-1 to L-40; M-1 to Y-39; M-1 to D-38; M-1 to R-37; M-1 to W-36; M-1 to L-35; M-1 to Y-34; M-1 to R-33; M-1 to L-32; M-1 to F-31; M-1 to E-30; M-1 to D-29; M-1 to E-28; M-1 to Y-27; M-1 to D-26; M-1 to P-25; M-1 to P-24; M-1 to V-23; M-1 to P-22; M-1 to S-21; M-1 to P-20; M-1 to E-19; M-1 to R-18; M-1 to S-17; M-1 to G-16; M-1 to P-15; M-1 to P-14; M-1 to V-13; M-1 to G-12; M-1 to M-11; M-1 to Q-10; M-1 to A-9; M-1 to G-8; M-1 to P-7; or M-1 to T-6 of SEQ ID NO:2, 4, or 6. Polynucleotides encoding these polypeptides are also encompassed by the invention.

The above C-terminal deletion mutants (1-n) can also include the following: M-1 to S-401; M-1 to L-400; M-1 to S-399; M-1 to K-398; M-1 to H-397; M-1 to S-396; M-1 to A-395; M-1 to S-394; M-1 to S-393; M-1 to R-392; M-1 to P-391; M-1 to S-390; M-1 to P-389; M-1 to A-388; M-1 to K-387; M-1 to L-386; M-1 to S-385; M-1 to G-384; M-1 to C-383; M-1 to P-382; M-1 to G-381; M-1 to L-380; M-1 to G-379; M-1 to P-378; M-1 to L-377; M-1 to C-376; M-1 to C-375; M-1 to L-374; M-1 to F-373; M-1 to A-372; M-1 to A-371; M-1 to K-370; M-1 to F-369; M-1 to Q-368; M-1 to E-367; M-1 to R-366; M-1 to F-365; or M-1 to K-364 of SEQ ID NO:2; M-1 to L-369; M-1 to L-368; or M-1 to S-367; M-1 to W-366; M-1 to P-365; or M-1 to L-364 of SEQ ID NO:4; or M-1 to S-372; M-1 to L-371; M-1 to V-370; M-1 to L-369; M-1 to S-368; M-1 to K-367; M-1 to E-366; M-1 to K-365; or M-1 to C-364 of SEQ ID NO:6.

In addition, any of the above listed N- or C-terminal deletions can be combined to produce a N- and C-terminal deleted neuropeptide receptor polypeptide. The invention also provides polypeptides having one or more amino acids deleted from both the amino and the carboxyl termini, which may be described generally as having residues m-n of SEQ ID NO:2, where n and m are integers as described above. Polynucleotides encoding these polypeptides are also encompassed by the invention.

Also included are a nucleotide sequence encoding a polypeptide consisting of a portion of the complete neuropeptide receptor amino acid sequence encoded by the cDNA clone contained in ATCC Deposit No. 97128, where this portion excludes any integer of amino acid residues from 1 to about 392 amino acids from the amino terminus of the complete amino acid sequence encoded by the cDNA clone contained in ATCC Deposit No. 97128, or any integer of amino acid residues from 1 to about 392 amino acids from the carboxy terminus, or any combination of the above amino terminal and carboxy terminal deletions, of the complete amino acid sequence encoded by the cDNA clone contained in ATCC Deposit No. 97128. Polynucleotides encoding all of the above deletion mutant polypeptide forms also are provided.

The present application is also directed to proteins containing polypeptides at least 90%, 95%, 96%, 97%, 98% or 99% identical to the neuropeptide receptor polypeptide sequence set forth herein m-n. In preferred embodiments, the application is directed to proteins containing polypeptides at least 90%, 95%, 96%, 97%, 98% or 99% identical to polypeptides having the amino acid sequence of the specific neuropeptide receptor N- and C-terminal deletions recited herein. Polynucleotides encoding these polypeptides are also encompassed by the invention.

Among the especially preferred fragments of the invention are fragments characterized by structural or functional attributes of neuropeptide receptor. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of complete (i.e., full-length) neuropeptide receptor (SEQ ID NO:2). Certain preferred regions are those set out in FIG. 8 and include, but are not limited to, regions of the aforementioned types identified by analysis of the amino acid sequence depicted in FIGS. 1A-F (SEQ ID NO:2), such preferred regions include; Gamier-Robson predicted alpha-regions, beta-regions, turn-regions, and coil-regions; Chou-Fasman predicted alpha-regions, beta-regions, turn-regions, and coil-regions; Kyte-Doolittle predicted hydrophilic and hydrophobic regions; Eisenberg alpha and beta amphipathic regions; Emini surface-forming regions; and Jameson-Wolf high antigenic index regions, as predicted using the default parameters of these computer programs. Polynucleotides encoding these polypeptides are also encompassed by the invention.

In additional embodiments, the polynucleotides of the invention encode functional attributes of neuropeptide receptor. Preferred embodiments of the invention in this regard include fragments that comprise alpha-helix and alpha-helix forming regions (“alpha-regions”), beta-sheet and beta-sheet forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions and high antigenic index regions of neuropeptide receptor.

The data representing the structural or functional attributes of neuropeptide receptor set forth in FIGS. 1A-F and/or Table I, as described above, was generated using the various modules and algorithms of the DNA*STAR set on default parameters. In a preferred embodiment, the data presented in columns VIII, IX, XIII, and XIV of Table I can be used to determine regions of neuropeptide receptor which exhibit a high degree of potential for antigenicity. Regions of high antigenicity are determined from the data presented in columns VIII, IX, XIII, and/or IV by choosing values which represent regions of the polypeptide which are likely to be exposed on the surface of the polypeptide in an environment in which antigen recognition may occur in the process of initiation of an immune response

Certain preferred regions in these regards are set out in FIG. 8, but may, as shown in Table I, be represented or identified by using tabular representations of the data presented in FIG. 8. The DNA*STAR computer algorithm used to generate FIG. 8 (set on the original default parameters) was used to present the data in FIG. 8 in a tabular format (See Table I). The tabular format of the data in FIG. 8 may be used to easily determine specific boundaries of a preferred region.

The above-mentioned preferred regions set out in FIG. 8 and in Table I include, but are not limited to, regions of the aforementioned types identified by analysis of the amino acid sequence set out in FIGS. 1A-F. As set out in FIG. 8 and in Table I, such preferred regions include Garnier-Robson alpha-regions, beta-regions, turn-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index.

TABLE I

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | . | B | . | . | . | . | 0.24 | −0.24 | . | . | . | 1.15 | 1.33 |
| Glu | 2 | . | . | B | . | . | T | . | 0.32 | −0.32 | . | . | . | 1.60 | 1.05 |
| Pro | 3 | . | . | . | . | . | T | C | 0.50 | −0.50 | . | . | . | 2.05 | 1.19 |
| Ser | 4 | . | . | . | . | T | T | . | 0.54 | −0.54 | . | . | . | 2.50 | 1.85 |
| Ala | 5 | . | . | . | . | . | T | C | 0.34 | −0.34 | . | * | F | 2.20 | 1.06 |
| Thr | 6 | . | . | . | . | . | T | C | 0.94 | −0.94 | . | * | F | 1.20 | 0.69 |
| Pro | 7 | . | . | . | . | . | T | C | 0.34 | −0.34 | . | | F | 0.95 | 0.89 |
| Gly | 8 | . | . | . | . | T | T | . | 0.21 | −0.21 | . | * | F | 0.90 | 0.88 |
| Ala | 9 | . | . | B | . | . | T | . | −0.34 | 0.34 | . | * | . | 0.10 | 0.60 |
| Gln | 10 | . | . | B | . | . | . | . | 0.03 | −0.03 | . | * | . | −0.40 | 0.29 |
| Met | 11 | . | . | B | . | . | . | . | 0.13 | −0.13 | . | . | . | −0.40 | 0.45 |
| Gly | 12 | . | . | B | . | . | . | . | −0.00 | 0.00 | . | . | . | −0.06 | 0.69 |
| Val | 13 | . | . | B | . | . | . | . | 0.04 | −0.04 | * | * | . | 0.58 | 0.39 |
| Pro | 14 | . | . | . | . | . | T | C | 0.74 | −0.74 | * | * | F | 1.47 | 0.53 |
| Pro | 15 | . | . | . | . | . | T | C | 0.74 | −0.74 | * | * | F | 2.56 | 1.06 |
| Gly | 16 | . | . | . | . | T | T | . | 1.13 | −1.13 | * | . | F | 3.40 | 2.47 |
| Ser | 17 | . | . | . | . | T | T | . | 1.18 | −1.18 | * | . | F | 3.06 | 2.47 |
| Arg | 18 | . | . | . | . | . | . | C | 1.82 | −1.82 | * | . | F | 2.32 | 2.14 |
| Glu | 19 | . | . | B | . | . | T | . | 1.18 | −1.18 | * | . | F | 1.98 | 3.34 |
| Pro | 20 | . | . | . | . | . | T | . | 1.18 | −1.18 | * | . | F | 2.04 | 1.85 |
| Ser | 21 | . | . | . | . | . | T | C | 1.31 | −1.31 | * | . | F | 1.50 | 1.46 |
| Pro | 22 | . | . | . | . | . | T | C | 1.61 | −1.61 | * | * | F | 1.50 | 1.30 |
| Val | 23 | . | . | . | . | . | . | C | 1.26 | −1.26 | * | * | F | 1.60 | 1.41 |
| Pro | 24 | . | . | . | . | . | T | C | 1.26 | −1.26 | . | . | F | 1.50 | 1.65 |
| Pro | 25 | . | . | . | . | . | T | C | 1.47 | −1.47 | . | . | F | 2.40 | 1 84 |
| Asp | 26 | . | . | . | . | . | T | C | 1.77 | −1.77 | . | * | F | 3.00 | 4.15 |
| Tyr | 27 | A | . | . | . | . | T | . | 1.28 | −1.28 | . | * | F | 2.50 | 4.65 |
| Glu | 28 | A | A | . | . | . | . | . | 1.32 | −1.32 | * | * | F | 1.80 | 2.60 |
| Asp | 29 | A | A | . | . | . | . | . | 1.64 | −1.64 | * | . | F | 1.50 | 1.28 |
| Glu | 30 | A | A | . | . | . | . | . | 1.61 | −1.61 | * | . | . | 1.05 | 1.61 |
| Phe | 31 | A | A | . | . | . | . | . | 0.80 | −0.80 | * | . | . | 0.75 | 1.45 |
| Leu | 32 | A | A | . | . | . | . | . | 0.76 | −0.76 | * | * | . | 0.30 | 0.72 |
| Arg | 33 | A | A | . | . | . | . | . | 0.87 | −0.87 | * | * | . | −0.60 | 0.44 |
| Tyr | 34 | A | A | . | . | . | . | . | 0.87 | −0.87 | * | * | . | −0.60 | 0.99 |
| Leu | 35 | A | A | . | . | . | . | . | 0.62 | −0.62 | * | * | . | −0.15 | 2.00 |
| Trp | 36 | . | . | . | . | T | T | . | 0.51 | −0.51 | * | * | . | 0.65 | 1.60 |
| Arg | 37 | . | . | . | . | T | T | . | 1.08 | −1.08 | * | * | . | 0.20 | 0.84 |
| Asp | 38 | . | . | . | . | T | T | . | 0.76 | −0.76 | * | * | . | 0.35 | 1.60 |
| Tyr | 39 | . | . | . | . | T | T | . | 1.04 | −1.04 | * | * | . | 0.35 | 2.35 |
| Leu | 40 | . | . | . | . | . | . | C | 1.86 | −1.86 | * | . | . | 0.85 | 2.40 |
| Tyr | 41 | . | . | . | . | . | T | C | 1.90 | −1.90 | . | * | . | 0.45 | 2.49 |
| Pro | 42 | . | . | . | . | . | T | C | 1.79 | −1.79 | . | * | F | 0.30 | 2.49 |
| Lys | 43 | . | . | . | . | T | T | . | 1.50 | −1.50 | . | . | F | 0.80 | 5.22 |
| Gln | 44 | . | . | B | . | . | T | . | 0.89 | −0.89 | . | . | F | 0.40 | 3.50 |
| Tyr | 45 | . | . | B | B | . | . | . | 0.89 | −0.89 | . | . | . | −0.15 | 1.68 |
| Glu | 46 | . | . | B | B | . | . | . | 0.24 | −0.24 | . | . | . | −0.60 | 0.69 |
| Trp | 47 | . | . | B | B | . | . | . | 0.24 | −0.24 | . | . | . | −0.60 | 0.28 |
| Val | 48 | . | . | B | B | . | . | . | −0.39 | 0.39 | . | . | . | −0.60 | 0.28 |
| Leu | 49 | . | . | B | B | . | . | . | −0.63 | 0.63 | . | . | . | −0.60 | 0.16 |
| Ile | 50 | . | . | B | B | . | . | . | −1.24 | 1.24 | . | . | . | −0.60 | 0.24 |
| Pro | 51 | . | . | B | B | . | . | . | −1.83 | 1.83 | . | . | . | −0.60 | 0.24 |
| Ala | 52 | . | . | B | B | . | . | . | −2.40 | 2.40 | . | . | . | −0.60 | 0.29 |
| Tyr | 53 | A | . | . | B | . | . | . | −2.24 | 2.24 | . | . | . | −0.60 | 0.31 |
| Val | 54 | A | . | . | B | . | . | . | −2.29 | 2.29 | . | . | . | −0.60 | 0.17 |
| Ala | 55 | . | . | B | B | . | . | . | −2.26 | 2.26 | . | . | . | −0.60 | 0.13 |
| Val | 56 | . | . | B | B | . | | . | −2.63 | 2.63 | . | . | . | −0.60 | 0.06 |
| Phe | 57 | . | . | B | B | . | . | . | −2.86 | 2.86 | . | . | . | −0.60 | 0.08 |
| Val | 58 | . | . | B | B | . | . | . | −3.47 | 3.47 | . | . | . | −0.60 | 0.07 |
| Val | 59 | . | . | B | B | . | . | . | −2.96 | 2.96 | . | | . | −0.60 | 0.07 |
| Ala | 60 | . | . | B | B | . | . | . | −2.37 | 2.37 | . | . | . | −0.60 | 0.08 |
| Leu | 61 | . | . | B | B | . | . | . | −1.82 | 1.82 | . | . | . | −0.60 | 0.17 |
| Val | 62 | A | . | . | . | . | T | . | −1.93 | 1.93 | . | . | . | −0.20 | 0.33 |
| Gly | 63 | . | . | . | . | T | T | . | −1.93 | 1.93 | . | . | F | 0.35 | 0.27 |
| Asn | 64 | A | . | . | . | T | T | . | −1.74 | 1.74 | . | . | F | 0.35 | 0.24 |
| Thr | 65 | . | . | B | . | . | T | . | −1.97 | 1.97 | . | . | F | −0.05 | 0.17 |
| Leu | 66 | . | . | B | B | . | . | . | −1.74 | 1.74 | . | . | . | −0.60 | 0.14 |
| Val | 67 | . | . | B | B | . | . | . | −1.74 | 1.74 | . | . | . | −0.60 | 0.09 |
| Cys | 68 | . | . | B | B | . | . | . | −1.69 | 1.69 | . | . | . | −0.60 | 0.05 |
| Leu | 69 | . | . | B | B | . | . | . | −1.58 | 1.58 | . | . | . | −0.60 | 0.06 |
| Ala | 70 | A | . | . | B | . | . | . | −1.27 | 1.27 | . | . | . | −0.60 | 0.16 |
| Val | 71 | A | . | . | B | . | . | . | −0.49 | 0.49 | . | . | . | −0.60 | 0.47 |
| Trp | 72 | A | . | . | . | . | T | . | 0.33 | −0.33 | . | . | . | −0.07 | 0.77 |
| Arg | 73 | A | . | . | . | . | T | . | 0.40 | −0.40 | . | . | . | 0.21 | 1.04 |
| Asn | 74 | A | . | . | . | . | T | . | 1.32 | −1.32 | . | . | . | 0.34 | 1.39 |
| His | 75 | . | . | . | . | . | T | C | 1.60 | −1.60 | . | . | . | 1.57 | 2.59 |
| His | 76 | . | . | . | B | . | . | C | 1.60 | −1.60 | . | . | . | 1.30 | 1.91 |
| Met | 77 | . | . | . | B | . | . | C | 1.58 | −1.58 | * | . | . | 0.42 | 0.88 |
| Arg | 78 | . | . | B | B | . | . | . | 1.47 | −1.47 | . | . | . | 0.09 | 0.93 |
| Thr | 79 | . | . | B | B | . | . | . | 1.22 | −1.22 | * | * | . | 0.11 | 1.10 |
| Val | 80 | . | . | B | B | . | . | . | 0.56 | −0.56 | * | * | F | 0.13 | 1.75 |
| Thr | 81 | . | . | B | B | . | . | . | −0.30 | 0.30 | * | * | F | −0.45 | 0.77 |
| Asn | 82 | . | . | B | B | . | . | . | −0.56 | 0.56 | * | * | . | −0.60 | 0.38 |
| Tyr | 83 | . | . | B | B | . | . | . | −0.67 | 0.67 | * | * | . | −0.60 | 0.38 |
| Phe | 84 | . | . | B | B | . | . | . | −1.17 | 1.17 | . | . | . | −0.60 | 0.42 |
| Ile | 85 | . | . | B | B | . | . | . | −0.61 | 0.61 | . | * | . | −0.60 | 0.21 |
| Val | 86 | . | . | B | B | . | . | . | −1.11 | 1.11 | . | * | . | −0.60 | 0.18 |
| Asn | 87 | . | . | B | B | . | . | . | −1.70 | 1.70 | . | . | . | −0.60 | 0.17 |
| Leu | 88 | . | . | B | B | . | . | . | −1.46 | 1.46 | . | . | . | −0.60 | 0.25 |
| Ser | 89 | A | . | . | B | . | . | . | −1.61 | 1.61 | . | * | . | −0.30 | 0.57 |
| Leu | 90 | A | . | . | B | . | . | . | −1.53 | 1.53 | . | . | . | −0.30 | 0.26 |
| Ala | 91 | A | . | . | B | . | . | . | −1.53 | 1.53 | . | * | . | −0.60 | 0.26 |
| Asp | 92 | A | . | . | B | . | . | . | −1.84 | 1.84 | . | . | . | −0.60 | 0.14 |
| Val | 93 | . | . | B | B | . | . | . | −1.62 | 1.62 | * | . | . | −0.60 | 0.25 |
| Leu | 94 | . | . | B | B | . | . | . | −2.21 | 2.21 | * | . | . | −0.60 | 0.25 |
| Val | 95 | . | . | B | B | . | . | . | −2.07 | 2.07 | * | . | . | −0.60 | 0.11 |
| Thr | 96 | . | . | B | B | . | . | . | −2.29 | 2.29 | . | . | . | −0.60 | 0.08 |
| Ala | 97 | . | . | B | B | . | . | . | −2.50 | 2.50 | . | . | . | −0.60 | 0.08 |
| Ile | 98 | . | . | B | B | . | . | . | −2.23 | 2.23 | . | . | . | −0.60 | 0.16 |
| Cys | 99 | . | . | B | B | . | . | . | −1.72 | 1.72 | . | * | . | −0.60 | 0.11 |
| Leu | 100 | . | . | B | B | . | . | . | −1.68 | 1.68 | . | . | . | −0.60 | 0.15 |
| Pro | 101 | A | . | . | B | . | . | . | −2.18 | 2.18 | . | * | . | −0.60 | 0.17 |
| Ala | 102 | A | . | . | B | . | . | . | −2.44 | 2.44 | . | * | . | −0.60 | 0.27 |
| Ser | 103 | A | . | . | B | . | . | . | −1.56 | 1.56 | * | * | . | −0.60 | 0.24 |
| Leu | 104 | . | . | B | B | . | . | . | −1.78 | 1.78 | . | * | . | −0.30 | 0.26 |
| Leu | 105 | . | . | B | B | . | . | . | −1.28 | 1.28 | * | * | . | −0.60 | 0.18 |
| Val | 106 | . | . | B | B | . | . | . | −1.07 | 1.07 | * | * | . | −0.60 | 0.20 |
| Asp | 107 | . | . | B | B | . | . | . | −0.78 | 0.78 | * | * | . | −0.30 | 0.41 |
| Ile | 108 | . | . | B | B | . | . | . | −0.77 | 0.77 | * | * | . | 0 30 | 0.67 |
| Thr | 109 | A | . | . | . | . | T | . | −0.77 | 0.77 | . | * | F | 0.25 | 0.95 |
| Gln | 110 | A | . | . | . | . | T | . | −0.66 | 0.66 | . | . | F | 0.25 | 0.47 |
| Ser | 111 | A | . | . | . | . | T | . | −0.14 | 0.14 | . | * | F | −0.05 | 0.58 |
| Trp | 112 | A | . | . | . | . | T | . | −0.18 | 0.18 | . | . | . | −0.20 | 0.40 |
| Leu | 113 | A | A | . | . | . | . | . | 0.12 | −0.12 | . | . | . | −0.60 | 0.31 |
| Phe | 114 | A | A | . | . | . | . | . | −0.38 | 0.38 | . | . | . | −0.60 | 0.23 |
| Gly | 115 | A | A | . | . | . | . | . | −1.04 | 1.04 | . | . | . | −0.60 | 0.18 |
| His | 116 | A | A | . | . | . | . | . | −0.70 | 0.70 | * | * | . | −0.60 | 0.12 |
| Ala | 117 | A | A | . | . | . | . | | −1.27 | 1.27 | * | . | . | −0.60 | 0.28 |
| Leu | 118 | A | A | . | . | . | . | . | −1.34 | 1.34 | * | . | . | −0.30 | 0.21 |
| Cys | 119 | . | A | . | . | T | . | . | −0.86 | 0.86 | * | . | | −0.20 | 0.11 |
| Lys | 120 | . | A | B | . | . | . | . | −0.76 | 0.76 | * | . | . | −0.60 | 0.16 |
| Val | 121 | . | A | B | . | . | . | . | −1.53 | 1.53 | * | . | . | −0.60 | 0.31 |
| Ile | 122 | . | . | B | B | . | . | . | −0.94 | 0.94 | * | * | . | −0.60 | 0.48 |
| Pro | 123 | . | . | B | B | . | . | . | −0.72 | 0.72 | * | . | . | −0.60 | 0.41 |
| Tyr | 124 | . | . | B | B | . | . | . | −0.91 | 0.91 | * | * | . | −0.60 | 0.56 |
| Leu | 125 | . | . | B | B | . | . | . | −1.26 | 1.26 | . | . | . | −0.60 | 0.59 |
| Gln | 126 | . | . | B | B | . | . | . | −1.26 | 1.26 | . | . | . | −0.60 | 0.52 |
| Ala | 127 | . | . | B | B | . | . | . | −0.67 | 0.67 | . | . | . | −0.60 | 0.24 |
| Val | 128 | . | . | B | B | . | . | . | −1.31 | 1.31 | . | . | . | −0.60 | 0.40 |
| Ser | 129 | . | . | B | B | . | . | . | −1.66 | 1.66 | . | * | . | −0.60 | 0.17 |
| Val | 130 | . | . | B | B | . | . | . | −1.70 | 1.70 | . | . | | −0.60 | 0.17 |
| Ser | 131 | . | . | B | B | . | . | . | −2.51 | 2.51 | . | . | . | −0.60 | 0.17 |
| Val | 132 | . | . | B | B | . | . | . | −2.23 | 2.23 | . | . | . | −0.60 | 0.10 |
| Ala | 133 | . | . | B | B | . | . | . | −2.19 | 2.19 | . | * | . | −0.60 | 0.20 |
| Val | 134 | . | . | B | B | . | . | . | −2.19 | 2.19 | . | * | . | −0.60 | 0.13 |
| Leu | 135 | . | . | B | B | . | . | . | −2.03 | 2.03 | . | . | . | −0.60 | 0.23 |
| Thr | 136 | . | . | B | B | . | . | . | −2.62 | 2.62 | . | . | . | −0.60 | 0.19 |
| Leu | 137 | . | . | B | B | . | . | . | −2.36 | 2.36 | . | . | . | −0.60 | 0.18 |
| Ser | 138 | . | . | B | B | . | . | . | −2.58 | 2.58 | . | | . | −0.60 | 0.22 |
| Phe | 139 | . | . | B | B | . | . | . | −1.72 | 1.72 | * | . | . | −0.60 | 0.13 |
| Ile | 140 | . | . | B | B | . | . | . | −0.80 | 0.80 | * | * | . | −0.60 | 0.26 |
| Ala | 141 | . | . | B | B | . | . | . | −0.78 | 0.78 | . | . | . | −0.30 | 0.38 |
| Leu | 142 | A | . | . | B | . | . | . | −0.21 | 0.21 | . | . | . | −0.60 | 0.46 |
| Asp | 143 | A | . | . | . | . | T | . | −0.50 | 0.50 | . | . | . | −0.05 | 1.03 |
| Arg | 144 | . | . | . | . | T | T | . | −0.69 | 0.69 | . | . | . | 0.65 | 1.03 |
| Trp | 145 | . | . | . | . | T | T | . | −0.47 | 0.47 | . | . | . | 0.20 | 0.87 |
| Tyr | 146 | A | . | . | . | . | T | . | 0.09 | −0.09 | * | . | . | −0.20 | 0.28 |
| Ala | 147 | . | . | B | B | . | . | . | 0.69 | −0.69 | * | . | . | −0.60 | 0.20 |
| Ile | 148 | . | . | B | B | . | . | . | −0.12 | 0.12 | * | . | . | −0.60 | 0.29 |
| Cys | 149 | . | . | B | B | . | . | . | −1.04 | 1.04 | . | * | . | −0.60 | 0.15 |
| His | 150 | . | . | B | B | . | . | . | −1.46 | 1.46 | . | * | . | −0.60 | 0.12 |
| Pro | 151 | . | . | B | B | . | . | . | −1.17 | 1.17 | . | . | . | −0.60 | 0.15 |
| Leu | 152 | A | . | . | . | | . | . | −0.88 | 0.88 | . | . | . | −0.40 | 0.57 |
| Leu | 153 | A | . | . | . | . | . | . | −0.30 | 0.30 | . | * | . | −0.40 | 0.56 |
| Phe | 154 | A | . | . | . | . | . | . | −0.22 | 0.22 | * | * | . | −0.40 | 0.52 |
| Lys | 155 | A | . | . | . | . | | . | −0.08 | 0.08 | * | * | F | −0.25 | 0.64 |
| Ser | 156 | A | . | . | . | . | . | . | 0.24 | −0.24 | * | * | F | 0.80 | 1.52 |
| Thr | 157 | A | . | . | . | . | . | . | 0.47 | −0.47 | * | * | F | 1.44 | 3.44 |
| Ala | 158 | A | . | . | . | . | . | . | 1.39 | −1.39 | * | * | F | 1.78 | 1.74 |
| Arg | 159 | A | . | . | . | . | . | . | 1.74 | −1.74 | * | * | F | 2.12 | 2.54 |
| Arg | 160 | A | . | . | . | . | . | . | 1.40 | −1.40 | * | * | F | 2.46 | 1.74 |
| Ala | 161 | . | . | . | . | T | T | . | 0.81 | −0.81 | * | * | F | 3.40 | 2.31 |
| Arg | 162 | . | . | . | . | T | T | . | 0.31 | −0.31 | * | * | F | 2.91 | 0.83 |
| Gly | 163 | . | . | B | . | . | T | . | 0.56 | −0.56 | * | * | F | 1.87 | 0.35 |
| Ser | 164 | . | . | B | . | . | T | . | −0.44 | 0.44 | * | * | F | 0.93 | 0.34 |
| Ile | 165 | . | . | B | B | . | . | . | −0.84 | 0.84 | . | * | . | 0.04 | 0.12 |
| Leu | 166 | . | . | B | B | . | . | . | −0.84 | 0.84 | . | * | . | −0.60 | 0.13 |
| Gly | 167 | . | . | B | B | . | . | . | −1.81 | 1.81 | . | * | . | −0.60 | 0.10 |
| Ile | 168 | . | . | B | B | . | . | . | −1.77 | 1.77 | . | . | . | −0.60 | 0.10 |
| Trp | 169 | . | . | B | B | . | . | . | −2.28 | 2.28 | . | . | . | −0.60 | 0.17 |
| Ala | 170 | . | . | B | B | . | . | . | −1.98 | 1.98 | . | . | . | −0.60 | 0.14 |
| Val | 171 | A | | . | B | . | | . | −2.06 | 2.06 | . | . | . | −0.60 | 0.20 |
| Ser | 172 | A | . | . | B | . | . | . | −2.31 | 2.31 | . | * | . | −0.60 | 0.13 |
| Leu | 173 | . | . | B | B | . | . | . | −2.28 | 2.28 | . | * | . | −0.60 | 0.13 |
| Ala | 174 | . | . | B | B | . | | . | −2.20 | 2.20 | . | * | . | −0.60 | 0.13 |
| Ile | 175 | . | . | B | B | . | . | . | −1.61 | 1.61 | . | . | . | −0.60 | 0.15 |
| Met | 176 | A | . | . | B | . | . | . | −1.34 | 1.34 | . | * | . | −0.60 | 0.32 |
| Val | 177 | A | . | . | B | . | . | . | −1.63 | 1.63 | . | . | . | −0.60 | 0.32 |
| Pro | 178 | A | . | . | B | . | . | . | −1.68 | 1.68 | . | . | . | −0.60 | 0.46 |
| Gln | 179 | A | . | . | B | . | . | . | −1.69 | 1.69 | . | . | . | −0.60 | 0.35 |
| Ala | 180 | A | . | . | B | . | . | . | −0.80 | 0.80 | . | . | . | −0.60 | 0.46 |
| Ala | 181 | A | . | . | B | . | . | . | −0.87 | 0.87 | | . | . | −0.30 | 0.52 |
| Val | 182 | A | . | . | B | . | . | . | −0.31 | 0.31 | . | . | . | −0.30 | 0.16 |
| Met | 183 | A | . | . | B | . | . | . | −0.40 | 0.40 | . | . | . | −0.30 | 0 21 |
| Glu | 184 | A | . | . | B | . | . | . | −1.26 | 1.26 | . | . | . | −0.30 | 0.28 |
| Cys | 185 | . | . | B | B | . | . | . | −1.48 | 1.48 | . | . | . | −0.30 | 0.28 |
| Ser | 186 | A | . | . | B | . | . | . | −1.10 | 1.10 | * | . | . | −0.30 | 0.23 |
| Ser | 187 | A | . | . | B | . | . | . | −0.24 | 0.24 | * | . | . | −0.30 | 0.21 |
| Val | 188 | A | . | . | B | . | . | . | −0.46 | 0.46 | * | . | . | −0.30 | 0.67 |
| Leu | 189 | A | . | . | B | . | . | . | −1.04 | 1.04 | * | . | F | −0.15 | 0.42 |
| Pro | 190 | A | . | . | B | . | . | . | −0.38 | 0.38 | * | * | F | −0.15 | 0.31 |
| Glu | 191 | A | . | . | . | . | . | | 0.03 | −0.03 | * | * | F | 0.05 | 0.68 |
| Leu | 192 | A | . | . | . | . | . | . | 0.02 | −0.02 | * | * | . | 0.65 | 1.61 |
| Ala | 193 | A | . | . | . | . | . | . | 0.99 | −0.99 | * | * | . | 0.65 | 1.50 |
| Asn | 194 | A | . | . | . | . | T | . | 0.99 | −0.99 | * | * | F | 1.30 | 1.70 |
| Arg | 195 | A | . | . | . | . | T | . | 0.50 | −0.50 | . | * | F | 1.00 | 1.70 |
| Thr | 196 | A | . | . | . | | T | . | 0.20 | −0.20 | . | * | F | 1.00 | 1.46 |
| Arg | 197 | A | . | . | . | . | T | . | 0.16 | −0.16 | . | * | F | 1.00 | 1.21 |
| Leu | 198 | . | . | B | B | . | . | . | 0.08 | −0.08 | . | * | . | 0.30 | 0.46 |
| Phe | 199 | . | . | B | B | . | . | . | 0.04 | −0.04 | . | * | . | −0.60 | 0.17 |
| Ser | 200 | . | . | B | B | . | . | . | −0.07 | 0.07 | . | * | . | −0.60 | 0.12 |
| Val | 201 | . | A | B | . | . | . | . | 0.36 | −0.36 | * | * | . | −0.60 | 0.25 |
| Cys | 202 | . | A | B | . | . | . | . | −0.04 | 0.04 | * | * | . | 0.30 | 0.56 |
| His | 203 | A | A | . | . | . | . | . | 0.18 | −0.18 | * | . | . | −0.30 | 0.44 |
| Glu | 204 | A | A | . | . | . | . | . | 0.88 | −0.88 | * | . | . | −0.30 | 0.60 |
| Arg | 205 | A | A | . | . | . | . | . | 1.18 | −1.18 | * | * | . | 0.75 | 1.88 |
| Trp | 206 | A | A | . | . | . | . | . | 1.22 | −1.22 | * | . | . | 0.75 | 2.30 |
| Ala | 207 | A | A | . | . | . | . | . | 1.64 | −1.64 | * | * | F | 0.90 | 1.10 |
| Asp | 208 | A | A | . | . | . | . | . | 1.47 | −1.47 | * | * | F | 0.45 | 0.88 |
| Asp | 209 | A | A | . | . | . | . | . | 1.51 | −1.51 | * | * | F | 0.00 | 1.29 |
| Leu | 210 | A | A | . | . | . | . | . | 0.51 | −0.51 | * | * | F | 0.90 | 2.55 |
| Tyr | 211 | . | A | B | B | . | . | . | 0.56 | −0.56 | * | * | F | 0.60 | 1.07 |
| Pro | 212 | . | . | . | B | T | . | . | 1.11 | −1.11 | * | * | F | 0.40 | 1.01 |
| Lys | 213 | . | . | . | B | T | . | . | 0.81 | −0.81 | * | * | . | −0.05 | 1.66 |
| Ile | 214 | . | . | B | B | . | . | . | 0.14 | −0.14 | * | * | . | −0.45 | 1.42 |
| Tyr | 215 | . | . | B | B | . | . | . | 0.26 | −0.26 | . | * | . | −0.30 | 0.49 |
| His | 216 | . | . | B | . | . | T | . | −0.20 | 0.20 | . | . | . | −0.20 | 0.21 |
| Ser | 217 | . | . | B | . | . | T | . | −0.88 | 0.88 | * | . | . | −0.20 | 0.26 |
| Cys | 218 | . | . | B | . | . | T | . | −1.78 | 1.78 | * | . | . | −0.20 | 0.12 |
| Phe | 219 | . | . | B | . | . | T | . | −1.20 | 1.20 | . | . | . | −0.20 | 0.06 |
| Phe | 220 | . | . | B | B | . | . | . | −1.20 | 1.20 | . | . | . | −0.60 | 0.07 |
| Ile | 221 | . | . | B | B | . | . | . | −1.98 | 1.98 | . | . | . | −0.60 | 0.20 |
| Val | 222 | . | . | B | B | . | . | . | −2.27 | 2.27 | . | . | . | −0.60 | 0.19 |
| Thr | 223 | . | . | B | B | . | . | . | −1.81 | 1.81 | . | . | . | −0.60 | 0 22 |
| Tyr | 224 | . | . | B | . | . | . | . | −1.92 | 1.92 | . | . | . | −0.40 | 0.50 |
| Leu | 225 | . | . | B | . | . | . | . | −1.57 | 1.57 | . | . | . | −0.40 | 0.55 |
| Ala | 226 | A | . | . | . | . | T | . | −1.49 | 1.49 | . | . | . | −0.20 | 0.38 |
| Pro | 227 | A | . | . | . | . | T | . | −1.23 | 1.23 | . | . | . | −0.20 | 0.20 |
| Leu | 228 | A | . | . | . | . | T | . | −1.51 | 1.51 | . | . | . | −0.20 | 0.24 |
| Gly | 229 | A | . | . | . | . | T | . | −1.87 | 1.87 | . | . | . | −0.20 | 0.24 |
| Leu | 230 | A | A | . | . | . | . | . | −1.64 | 1.64 | . | . | . | −0.60 | 0.15 |
| Met | 231 | A | A | . | . | . | . | . | −1.30 | 1.30 | . | . | . | −0.60 | 0.19 |
| Ala | 232 | A | A | . | . | . | . | . | −1.79 | 1.79 | . | * | . | −0.60 | 0.30 |
| Met | 233 | A | A | . | B | . | . | . | −0.98 | 0.98 | . | * | . | −0.60 | 0.31 |
| Ala | 234 | A | A | . | B | . | . | . | −1.52 | 1.52 | . | . | . | −0.60 | 0.54 |
| Tyr | 235 | A | A | . | B | . | . | . | −1.41 | 1.41 | * | . | . | −0.60 | 0.38 |
| Phe | 236 | A | A | . | B | . | . | . | −0.70 | 0.70 | * | . | . | −0.60 | 0.33 |
| Gln | 237 | A | A | B | B | . | . | . | −0.11 | 0.11 | * | . | . | −0.60 | 0.64 |
| Ile | 238 | . | A | B | B | . | . | . | −0.32 | 0.32 | * | . | . | −0.60 | 0.66 |
| Phe | 239 | . | A | B | B | . | . | . | −0.02 | 0.02 | * | . | . | −0.60 | 0.63 |
| Arg | 240 | . | A | . | B | T | . | . | −0.12 | 0.12 | * | . | . | −0.20 | 0.38 |
| Asn | 241 | . | A | . | B | T | . | . | 0.69 | −0.69 | * | . | . | −0.20 | 0.54 |
| Leu | 242 | . | A | . | B | T | . | . | 0.69 | −0.69 | * | . | . | 0.25 | 1.21 |
| Trp | 243 | . | A | . | B | T | . | . | 0.69 | −0.69 | * | . | . | 0.85 | 1.07 |
| Gly | 244 | . | A | . | B | . | . | C | 1.18 | −1.18 | * | . | F | −0.04 | 0.47 |
| Arg | 245 | . | . | . | B | T | . | . | 0.72 | −0.72 | . | . | F | 0.37 | 0.88 |
| Gln | 246 | . | . | B | B | . | . | . | 0.41 | −0.41 | * | . | F | 0.48 | 0.83 |
| Ile | 247 | . | . | B | . | . | T | . | 0.91 | −0.91 | * | . | F | 1.84 | 1.20 |
| Pro | 248 | . | . | . | . | . | T | C | 0.90 | −0.90 | * | . | F | 2.10 | 0.89 |
| Gly | 249 | . | . | . | . | T | T | . | 0.66 | −0.66 | * | . | F | 1.49 | 0.69 |
| Thr | 250 | . | . | . | . | . | T | C | −0.27 | 0.27 | * | . | F | 0.78 | 0.99 |
| Thr | 251 | . | . | B | B | . | . | . | −1.12 | 1.12 | * | * | F | −0.03 | 0.53 |
| Ser | 252 | . | . | B | B | . | . | . | −0.12 | 0.12 | * | * | F | −0.24 | 0.40 |
| Ala | 253 | . | . | B | B | . | . | . | 0.09 | −0.09 | * | . | . | −0.30 | 0.54 |
| Leu | 254 | . | . | B | B | . | . | . | 0.14 | −0.14 | * | . | . | −0.30 | 0.60 |
| Val | 255 | . | . | B | B | . | . | . | 0.50 | −0.50 | * | . | . | −0.60 | 0.47 |
| Arg | 256 | . | . | B | B | . | . | . | 0.92 | −0.92 | * | . | . | −0.30 | 0.93 |
| Asn | 257 | . | . | . | B | T | . | . | 1.01 | −1.01 | * | . | . | 1.19 | 2.21 |
| Trp | 258 | . | . | . | B | T | . | . | 1.30 | −1.30 | * | . | F | 1.98 | 4.60 |
| Lys | 259 | . | . | . | B | . | . | C | 2.11 | −2.11 | * | . | F | 2.12 | 3.15 |
| Arg | 260 | . | . | . | . | . | . | C | 2.97 | −2.97 | * | * | F | 2.66 | 3 27 |
| Pro | 261 | . | . | . | . | T | T | . | 2.04 | −2.04 | * | . | F | 3.40 | 5.38 |
| Ser | 262 | . | . | . | . | T | T | . | 1.70 | −1.70 | * | . | F | 3.06 | 2.22 |
| Asp | 263 | . | . | . | . | T | T | . | 1.99 | −1.99 | * | . | F | 2.72 | 1.12 |
| Gln | 264 | . | . | . | . | . | T | C | 1.13 | −1.13 | * | . | F | 2.18 | 1.21 |
| Leu | 265 | . | . | . | . | . | . | C | 1.02 | −1.02 | * | * | F | 1.19 | 0.75 |
| Gly | 266 | . | . | B | . | . | . | . | 1.23 | −1.23 | * | . | F | 0.95 | 0.77 |
| Asp | 267 | . | . | B | . | . | . | . | 1.19 | −1.19 | * | . | F | 0.65 | 0.77 |
| Leu | 268 | . | . | B | . | . | . | . | 0.38 | −0.38 | * | . | F | 0.65 | 0.93 |
| Glu | 269 | . | . | B | . | . | . | . | 0.08 | −0.08 | * | . | F | 0.65 | 0.77 |
| Gln | 270 | . | . | B | . | . | . | . | 0.54 | −0.54 | * | * | F | 0.65 | 0.62 |
| Gly | 271 | . | . | . | . | . | . | C | 0.89 | −0.89 | . | * | F | 0.25 | 0.74 |
| Leu | 272 | . | . | . | . | . | T | C | 0.68 | −0.68 | * | * | F | 1.35 | 0.74 |
| Ser | 273 | . | . | . | . | . | T | C | 1.49 | −1.49 | * | * | F | 1.05 | 0.66 |
| Gly | 274 | . | . | . | . | . | T | C | 1.28 | −1.28 | . | * | F | 1.20 | 1.16 |
| Glu | 275 | . | . | . | . | . | T | C | 1.39 | −1.39 | . | * | F | 1.54 | 2.18 |
| Pro | 276 | . | . | . | . | . | . | C | 1.39 | −1.39 | . | * | F | 1.98 | 3.19 |
| Gln | 277 | . | . | . | . | . | T | C | 2.31 | −2.31 | . | * | F | 2.52 | 3.19 |
| Pro | 278 | . | . | . | . | . | T | C | 2.02 | −2.02 | . | * | F | 2.86 | 3.60 |
| Arg | 279 | . | . | . | . | T | T | . | 1.67 | −1.67 | . | * | F | 3.40 | 2.35 |
| Gly | 280 | A | . | . | . | . | T | . | 0.86 | −0.86 | . | * | F | 2.36 | 1.18 |
| Arg | 281 | A | A | . | . | . | . | . | 0.48 | −0.48 | . | * | F | 1.47 | 0.63 |
| Ala | 282 | A | A | . | . | . | . | . | 0.48 | −0.48 | . | * | . | 0.98 | 0.32 |
| Phe | 283 | A | A | . | . | . | . | . | −0.17 | 0.17 | * | * | . | 0.64 | 0.57 |
| Leu | 284 | A | A | . | . | . | . | . | −0.23 | 0.23 | * | * | . | −0.30 | 0.21 |
| Ala | 285 | A | A | . | . | . | . | . | 0.11 | −0.11 | * | . | . | −0.30 | 0.43 |
| Glu | 286 | A | A | . | . | . | . | . | −0.60 | 0.60 | * | . | . | −0.30 | 0.85 |
| Val | 287 | A | A | . | . | . | . | . | 0.10 | −0.10 | . | . | . | 0.45 | 1.02 |
| Lys | 288 | A | A | . | . | . | . | . | 0.21 | −0.21 | . | * | . | 0.75 | 1.98 |
| Gln | 289 | A | A | . | . | . | . | . | 1.13 | −1.13 | . | . | . | 0.75 | 1.15 |
| Met | 290 | A | A | . | . | . | . | . | 1.83 | −1.83 | . | * | . | 0.75 | 3.04 |
| Arg | 291 | A | A | . | . | . | . | . | 1.88 | −1.88 | . | * | . | 0.75 | 2.98 |
| Ala | 292 | A | A | . | . | . | . | . | 2.42 | −2.42 | . | * | F | 0.90 | 3.44 |
| Arg | 293 | A | A | . | . | . | . | . | 1.79 | −1.79 | * | * | F | 0.90 | 5.02 |
| Arg | 294 | A | A | . | . | . | . | . | 1.83 | −1.83 | * | * | F | 0.90 | 2.59 |
| Lys | 295 | A | A | . | . | . | . | . | 1.83 | −1.83 | * | * | F | 0.90 | 5.13 |
| Thr | 296 | A | A | . | . | . | . | . | 0.91 | −0.91 | * | * | F | 0.90 | 2.59 |
| Ala | 297 | A | A | . | . | . | . | . | 0.90 | −0.90 | * | . | F | 0.90 | 1.09 |
| Lys | 298 | A | A | . | . | . | . | . | −0.07 | 0.07 | * | . | . | 0.30 | 0.54 |
| Met | 299 | A | . | . | B | . | . | . | −1.03 | 1.03 | * | * | . | −0.60 | 0.28 |
| Leu | 300 | A | . | . | B | . | . | . | −1.89 | 1.89 | * | . | . | −0.60 | 0.20 |
| Met | 301 | A | . | . | B | . | . | . | −2.39 | 2.39 | . | . | . | −0.60 | 0.08 |
| Val | 302 | A | . | . | B | . | . | . | −2.66 | 2.66 | . | . | . | −0.60 | 0.07 |
| Val | 303 | A | . | . | B | . | . | . | −3.40 | 3.40 | . | . | . | −0.60 | 0.06 |
| Leu | 304 | A | . | . | B | . | . | . | −3.39 | 3.39 | . | . | . | −0.60 | 0.06 |
| Leu | 305 | A | . | . | B | . | . | . | −3.39 | 3.39 | . | . | . | −0.60 | 0.08 |
| Val | 306 | A | . | . | B | . | . | . | −3.46 | 3.46 | . | . | . | −0.60 | 0.08 |
| Phe | 307 | A | . | . | B | . | . | . | −2.84 | 2.84 | . | . | . | −0.60 | 0.05 |
| Ala | 308 | A | . | . | B | . | . | . | −2.80 | 2.80 | . | . | . | −0.60 | 0.10 |
| Leu | 309 | . | . | B | B | . | . | . | −2.20 | 2.20 | . | . | . | −0.60 | 0.11 |
| Cys | 310 | . | . | B | B | . | . | . | −2.28 | 2.28 | . | . | . | −0.60 | 0.20 |
| Tyr | 311 | . | . | B | B | . | . | . | −1.72 | 1.72 | . | * | . | −0 60 | 0.14 |
| Leu | 312 | . | . | B | B | . | . | . | −1.88 | 1.88 | . | . | . | −0.60 | 0.23 |
| Pro | 313 | . | . | B | B | . | . | . | −2.10 | 2.10 | . | . | . | −0.60 | 0.32 |
| Ile | 314 | . | . | B | B | . | . | . | −1.29 | 1.29 | . | . | . | −0.60 | 0.17 |
| Ser | 315 | . | . | B | B | . | . | . | −1.48 | 1.48 | . | . | . | −0.60 | 0.33 |
| Val | 316 | . | A | B | B | . | . | . | −2.04 | 2.04 | * | . | . | −0.60 | 0.16 |
| Leu | 317 | . | A | B | B | . | . | . | −1.19 | 1.19 | * | * | . | −0.60 | 0.19 |
| Asn | 318 | . | A | B | B | . | . | . | −0.87 | 0.87 | * | * | . | −0.60 | 0.28 |
| Val | 319 | . | A | B | B | . | . | . | −0.83 | 0.83 | * | . | . | −0.30 | 0.73 |
| Leu | 320 | . | A | B | B | . | . | . | −1.23 | 1.23 | * | . | . | −0.30 | 0.66 |
| Lys | 321 | . | A | B | B | . | . | . | −0.72 | 0.72 | * | . | . | −0.30 | 0.35 |
| Arg | 322 | . | A | B | B | . | . | . | −0.51 | 0.51 | * | * | . | −0.30 | 0.47 |
| Val | 323 | . | A | B | B | . | . | . | −1.21 | 1.21 | * | * | . | −0.30 | 0.57 |
| Phe | 324 | . | A | B | B | . | . | . | −0.24 | 0.24 | * | * | . | −0.30 | 0.24 |
| Gly | 325 | . | A | B | B | . | . | . | 0.57 | −0.57 | * | . | . | −0.30 | 0.24 |
| Met | 326 | . | A | B | B | . | . | . | −0.07 | 0.07 | * | * | . | −0.60 | 0.57 |
| Phe | 327 | A | A | . | B | . | . | . | −0.48 | 0.48 | * | * | . | −0.30 | 0.67 |
| Arg | 328 | A | A | . | B | . | . | . | 0.38 | −0.38 | * | . | . | 0.30 | 0.90 |
| Gln | 329 | A | A | . | B | . | . | . | 1.19 | −1.19 | * | . | F | 1.50 | 1.52 |
| Ala | 330 | A | . | . | . | . | T | . | 1.53 | −1.53 | * | . | F | 2.50 | 3.44 |
| Ser | 331 | . | . | . | . | . | T | C | 1.54 | −1.54 | . | . | F | 3.00 | 3.04 |
| Asp | 332 | A | . | . | . | . | T | . | 1.39 | −1.39 | . | . | F | 2.50 | 1.78 |
| Arg | 333 | A | . | . | . | . | T | . | 1.03 | −1.03 | . | . | F | 2.20 | 1.30 |
| Glu | 334 | A | . | . | B | . | . | . | 0.44 | −0.44 | . | * | . | 1.35 | 1.53 |
| Ala | 335 | A | . | . | B | . | . | . | 0.37 | −0.37 | . | * | . | 0.90 | 0.92 |
| Val | 336 | A | . | . | B | . | . | . | −0.03 | 0.03 | . | . | . | 0.30 | 0.25 |
| Tyr | 337 | A | . | . | B | . | . | . | −0.34 | 0.34 | . | * | . | −0.60 | 0.13 |
| Ala | 338 | A | . | . | B | . | . | . | −1.16 | 1.16 | * | * | . | −0.60 | 0.18 |
| Cys | 339 | A | . | B | B | . | . | . | −1.46 | 1.46 | . | . | . | −0.60 | 0.21 |
| Phe | 340 | . | . | B | B | . | . | . | −0.90 | 0.90 | . | . | . | −0.60 | 0.18 |
| Thr | 341 | A | . | . | B | . | . | . | −0.33 | 0.33 | . | * | . | −0.60 | 0.24 |
| Phe | 342 | A | . | . | B | . | . | . | −0.90 | 0.90 | . | . | . | −0.60 | 0.48 |
| Ser | 343 | A | . | . | B | . | . | . | −1.17 | 1.17 | . | . | . | −0.60 | 0.45 |
| His | 344 | . | . | . | B | T | . | . | −0.74 | 0.74 | . | . | . | −0.20 | 0.23 |
| Trp | 345 | . | . | B | B | . | . | . | −0.63 | 0.63 | . | * | . | −0.60 | 0.42 |
| Leu | 346 | A | . | . | B | . | . | . | −0.32 | 0.32 | . | . | . | −0.60 | 0.32 |
| Val | 347 | . | . | B | B | . | . | . | 0.08 | −0.08 | . | . | . | −0.60 | 0.38 |
| Tyr | 348 | . | . | B | . | . | T | . | −0.21 | 0.21 | . | . | . | −0.20 | 0.48 |
| Ala | 349 | . | . | B | . | . | T | . | −0.77 | 0.77 | . | . | . | −0.20 | 0.59 |
| Asn | 350 | . | . | . | . | . | T | C | −0.48 | 0.48 | . | . | . | 0.00 | 0.80 |
| Ser | 351 | . | . | . | . | . | T | C | 0.12 | −0.12 | * | . | . | 0.00 | 0.82 |
| Ala | 352 | . | . | . | . | . | . | C | 0.09 | −0.09 | . | . | . | 0.25 | 1.25 |
| Ala | 353 | . | . | . | . | . | . | C | −0.56 | 0.56 | * | . | . | 0.10 | 0.55 |
| Asn | 354 | . | . | B | . | . | . | . | −0.21 | 0.21 | * | . | . | −0.40 | 0.29 |
| Pro | 355 | . | . | B | B | . | . | . | −0.21 | 0.21 | * | . | . | −0.60 | 0.44 |
| Ile | 356 | . | . | B | B | . | . | . | −0.61 | 0.61 | * | . | . | −0.60 | 0.71 |
| Ile | 357 | . | . | B | B | . | . | . | −0.83 | 0.83 | * | . | . | −0.60 | 0.38 |
| Tyr | 358 | . | . | B | B | . | . | . | −0.54 | 0.54 | * | . | . | −0.60 | 0.20 |
| Asn | 359 | . | . | B | B | . | . | . | −0.89 | 0.89 | . | . | . | −0.60 | 0.39 |
| Phe | 360 | . | . | B | B | . | . | . | −0.63 | 0.63 | . | * | . | −0.60 | 0.55 |
| Leu | 361 | . | . | B | . | . | T | . | −0.44 | 0.44 | * | * | . | −0.20 | 0.70 |
| Ser | 362 | . | . | . | . | . | T | C | 0.56 | −0.56 | * | * | F | 0.15 | 0.38 |
| Gly | 363 | . | . | . | . | . | T | C | 0.80 | −0.80 | . | * | F | 0.45 | 0.85 |
| Lys | 364 | A | . | . | . | . | T | . | 0.80 | −0.80 | * | * | F | 1.30 | 1.79 |
| Phe | 365 | A | A | . | . | . | . | . | 0.80 | −0.80 | * | * | F | 0.90 | 2.31 |
| Arg | 366 | A | A | . | . | . | . | . | 1.66 | −1.66 | * | * | F | 0.90 | 2.02 |
| Glu | 367 | A | A | . | . | . | . | . | 1.37 | −1.37 | * | * | F | 0.90 | 2 02 |
| Gln | 368 | A | A | . | . | . | . | . | 1.12 | −1.12 | * | * | F | 0.90 | 2.36 |
| Phe | 369 | A | A | . | . | . | . | . | 0.38 | −0.38 | * | * | . | 0.75 | 1.22 |
| Lys | 370 | A | A | . | . | . | . | . | 0.78 | −0.78 | * | * | . | 0.30 | 0.61 |
| Ala | 371 | A | A | . | . | . | . | . | −0.00 | 0.00 | * | * | . | −0.30 | 0.47 |
| Ala | 372 | A | A | . | . | . | . | . | −0.67 | 0.67 | * | * | . | −0.60 | 0.29 |
| Phe | 373 | . | . | . | . | T | T | . | −1.48 | 1.48 | . | * | . | 0.50 | 0.08 |
| Ser | 374 | . | . | B | . | . | T | . | −0.99 | 0.99 | . | * | . | −0.20 | 0.06 |
| Cys | 375 | . | . | B | . | . | T | . | −1.38 | 1.38 | . | * | . | −0.20 | 0.10 |
| Cys | 376 | . | . | B | . | . | T | . | −1.60 | 1.60 | . | . | . | −0.20 | 0.11 |
| Leu | 377 | . | . | B | . | . | T | . | −1.36 | 1.36 | . | . | . | −0.20 | 0.07 |
| Pro | 378 | . | . | . | . | T | T | . | −0.87 | 0.87 | . | . | F | 0.35 | 0.13 |
| Gly | 379 | . | . | . | . | T | T | . | −1.23 | 1.23 | . | . | F | 0.35 | 0.37 |
| Leu | 380 | . | . | . | . | T | T | . | −0.91 | 0.91 | . | . | F | 0.35 | 0.24 |
| Gly | 381 | . | . | . | . | . | T | C | −0.54 | 0.54 | . | . | F | 0.15 | 0.15 |
| Pro | 382 | . | . | . | . | T | T | . | −0.54 | 0.54 | . | * | F | 0.65 | 0.21 |
| Cys | 383 | . | . | B | . | . | T | . | −0.29 | 0.29 | . | * | F | −0.05 | 0.21 |
| Gly | 384 | . | . | B | . | . | T | . | −0.53 | 0.53 | . | * | F | 0.85 | 0.42 |
| Ser | 385 | . | . | B | . | . | . | . | 0.07 | −0.07 | . | * | F | 0.05 | 0.27 |
| Leu | 386 | . | . | B | . | . | . | . | 0.11 | −0.11 | . | * | F | 0.35 | 0.78 |
| Lys | 387 | . | . | B | . | . | . | . | 0.11 | −0.11 | . | * | F | 1.40 | 1.06 |
| Ala | 388 | . | . | B | . | . | . | . | 0.89 | −0.89 | . | * | F | 1.70 | 1.22 |
| Pro | 389 | . | . | . | . | . | . | C | 0.93 | −0.93 | . | * | F | 2.50 | 2.91 |
| Ser | 390 | . | . | . | . | . | T | C | 0.93 | −0.93 | . | * | F | 3.00 | 1.95 |
| Pro | 391 | . | . | . | . | . | T | C | 1.16 | −1.16 | . | * | F | 2.40 | 2.58 |
| Arg | 392 | . | . | . | . | T | T | . | 0.81 | −0.81 | . | . | F | 2.30 | 1.69 |
| Ser | 393 | A | . | . | . | . | T | . | 1.37 | −1.37 | . | * | F | 2.07 | 1.69 |
| Ser | 394 | A | . | . | . | . | . | . | 1.62 | −1.62 | . | * | F | 1.44 | 1.49 |
| Ala | 395 | A | . | . | | . | . | . | 1.62 | −1.62 | . | * | F | 1.61 | 1.52 |
| Ser | 396 | A | . | . | . | . | . | . | 1.02 | −1.02 | . | * | F | 1.48 | 1.52 |
| His | 397 | . | . | . | . | . | . | C | 0.61 | −0.61 | . | * | F | 1.70 | 0.93 |
| Lys | 398 | . | . | B | . | . | . | . | 0.10 | −0.10 | . | . | F | 1.48 | 1.24 |
| Ser | 399 | . | . | B | . | . | . | . | 0.01 | −0.01 | . | . | F | 0.56 | 0.76 |
| Leu | 400 | . | . | B | . | . | . | . | 0.21 | −0.21 | . | . | . | 0.24 | 0.72 |
| Ser | 401 | . | . | B | . | . | . | . | 0.12 | −0.12 | . | . | . | 0.07 | 0.46 |
| Leu | 402 | . | . | B | . | . | . | . | −0.23 | 0.23 | . | . | . | −0.40 | 0.44 |

Among highly preferred fragments in this regard are those that comprise regions of neuropeptide receptor that combine several structural features, such as several of the features set out above.

Other preferred fragments are biologically active neuropeptide receptor fragments. Biologically active fragments are those exhibiting activity similar, but not necessarily identical, to an activity of the neuropeptide receptor polypeptide. The biological activity of the fragments may include an improved desired activity, or a decreased undesirable activity.

However, many polynucleotide sequences, such as EST sequences, are publicly available and accessible through sequence databases. Some of these sequences are related to SEQ ID NO:1 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a₁-b₁, where a is any integer between 1 to 1195 of SEQ ID NO:1, b₁ is an integer of 15 to 1209, where both a₁ and b₁ correspond to the positions of nucleotide residues shown in SEQ ID NO:1, and where the b, is greater than or equal to a₁+14.

Additionally, many polynucleotide sequences, such as EST sequences, are publicly available and accessible through sequence databases. Some of these sequences are related to SEQ ID NO:3 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a₂−b₂, where a is any integer between 1 to 1096 of SEQ ID NO:3, b₂ is an integer of 15 to 1110, where both a₂ and b₂ correspond to the positions of nucleotide residues shown in SEQ ID NO:3, and where the b₂ is greater than or equal to a₂+14.

Moreover, many polynucleotide sequences, such as EST sequences, are publicly available and accessible through sequence databases. Some of these sequences are related to SEQ ID NO:5 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a3−b₃, where a is any integer between 1 to 1119 of SEQ ID NO:5, b₃ is an integer of 15 to 1133, where both a₃ and b₃ correspond to the positions of nucleotide residues shown in SEQ ID NO:1 and where the b₃ is greater than or equal to a₃30 14.

Epitope-Bearing Portions

In another aspect, the invention provides peptides and polypeptides comprising epitope-bearing portions of the polypeptides of the present invention. These epitopes are immunogenic or antigenic epitopes of the polypeptides of the present invention. An “immunogenic epitope” is defined as a part of a protein that elicits an antibody response in vivo when the whole polypeptide of the present invention, or fragment thereof, is the immunogen. On the other hand, a region of a polypeptide to which an antibody can bind is defined as an “antigenic determinant” or “antigenic epitope.” The number of in vivo immunogenic epitopes of a protein generally is less than the number of antigenic epitopes. See, e.g., Geysen, et al. (1983) Proc. Natl. Acad. Sci. USA 81:3998-4002. However, antibodies can be made to any antigenic epitope, regardless of whether it is an immunogenic epitope, by using methods such as phage display. See e.g., Petersen G. et al. (1995) Mol. Gen. Genet. 249:425-431. Therefore, included in the present invention are both immunogenic epitopes and antigenic epitopes.

A list of exemplified amino acid sequences comprising immunogenic epitopes are shown in Table 1 below. It is pointed out that Table 1 only lists amino acid residues comprising epitopes predicted to have the highest degree of antigenicity using the algorithm of Jameson and Wolf, (1988) Comp. Appl. Biosci. 4:181-186 (said references incorporated by reference in their entireties). The Jameson-Wolf antigenic analysis was performed using the computer program PROTEAN, using default parameters (Version 3.11 for the Power MacIntosh, DNASTAR, Inc., 1228 South Park Street Madison, Wis.). Table 1 and portions of polypeptides not listed in Table 1 are not considered non-immunogenic. The immunogenic epitopes of Table 1 is an exemplified list, not an exhaustive list, because other immunogenic epitopes are merely not recognized as such by the particular algorithm used. Amino acid residues comprising other immunogenic epitopes may be routinely determined using algorithms similar to the Jameson-Wolf analysis or by in vivo testing for an antigenic response using methods known in the art. See, e.g., Geysen et al., supra; U.S. Pat. Nos. 4,708,781; 5,194,392; 4,433,092; and 5,480,971 (said references incorporated by reference in their entireties).

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to neuropeptide receptor -specific antibodies include: a polypeptide comprising amino acid residues in SEQ ID NO:2 from about neuropeptide receptor. These polypeptide fragments have been determined to bear antigenic epitopes of the neuropeptide receptor protein by the analysis of the Jameson-Wolf antigenic index, as shown in FIG. 8, above.

It is particularly pointed out that the amino acid sequences of Table 1 comprise immunogenic epitopes. Table 1 lists only the critical residues of immunogenic epitopes determined by the Jameson-Wolf analysis. Thus, additional flanking residues on either the N-terminal, C-terminal, or both N- and C-terminal ends may be added to the sequences of Table 1 to generate an epitope-bearing polypeptide of the present invention. Therefore, the immunogenic epitopes of Table 1 may include additional N-terminal or C-terminal amino acid residues. The additional flanking amino acid residues may be contiguous flanking N-terminal and/or C-terminal sequences from the polypeptides of the present invention, heterologous polypeptide sequences, or may include both contiguous flanking sequences from the polypeptides of the present invention and heterologous polypeptide sequences.

Polypeptides of the present invention comprising immunogenic or antigenic epitopes are at least 7 amino acids residues in length. “At least” means that a polypeptide of the present invention comprising an immunogenic or antigenic epitope may be 7 amino acid residues in length or any integer between 7 amino acids and the number of amino acid residues of the full length polypeptides of the invention. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. However, it is pointed out that each and every integer between 7 and the number of amino acid residues of the full length polypeptide are included in the present invention.

The immuno and antigenic epitope-bearing fragments may be specified by either the number of contiguous amino acid residues, as described above, or further specified by N-terminal and C-terminal positions of these fragments on the amino acid sequence of SEQ ID NO:2. Every combination of a N-terminal and C-terminal position that a fragment of, for example, at least 7 or at least 15 contiguous amino acid residues in length could occupy on the amino acid sequence of SEQ ID NO:2 is included in the invention. Again, “at least 7 contiguous amino acid residues in length” means 7 amino acid residues in length or any integer between 7 amino acids and the number of amino acid residues of the full length polypeptide of the present invention. Specifically, each and every integer between 7 and the number of amino acid residues of the full length polypeptide are included in the present invention.

Immunogenic and antigenic epitope-bearing polypeptides of the invention are useful, for example, to make antibodies which specifically bind the polypeptides of the invention, and in immunoassays to detect the polypeptides of the present invention. The antibodies are useful, for example, in affinity purification of the polypeptides of the present invention. The antibodies may also routinely be used in a variety of qualitative or quantitative immunoassays, specifically for the polypeptides of the present invention using methods known in the art. See, e.g., Harlow et al., Antibodies: A Laboratory Manual, (Cold Spring Harbor Laboratory Press; 2nd Ed. 1988).

The epitope-bearing polypeptides of the present invention may be produced by any conventional means for making polypeptides including synthetic and recombinant methods known in the art. For instance, epitope-bearing peptides may be synthesized using known methods of chemical synthesis. For instance, Houghten has described a simple method for the synthesis of large numbers of peptides, such as 10-20 mgs of 248 individual and distinct 13 residue peptides representing single amino acid variants of a segment of the HA1 polypeptide, all of which were prepared and characterized (by ELISA-type binding studies) in less than four weeks (Houghten, R. A. Proc. Natl. Acad. Sci. USA 82:5131-5135 (1985)). This “Simultaneous Multiple Peptide Synthesis (SMPS)” process is further described in U.S. Pat. No. 4,631,211 to Houghten and coworkers (1986). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously (Houghten et al. (1985) Proc. Natl. Acad. Sci. 82:5131-5135 at 5134.

Epitope-bearing polypeptides of the present invention are used to induce antibodies according to methods well known in the art including, but not limited to, in vivo immunization, in vitro immunization, and phage display methods. See, e.g., Sutcliffe, et al., supra; Wilson, et al., supra, and Bittle, et al. (1985) J. Gen. Virol. 66:2347-2354. If in vivo immunization is used, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine residues may be coupled to a carrier using a linker such as -maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μgs of peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and discussed above, the polypeptides of the present invention comprising an immunogenic or antigenic epitope can be fused to heterologous polypeptide sequences. For example, the polypeptides of the present invention may be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo. This has been shown, e.g., for chimeric proteins consisting of the first two domains of the human CD4-polypeptide and various domains of the constant regions of the heavy or light chains of mammalian immunoglobulins. See, e.g., EPA 0,394,827; Traunecker et al. (1988) Nature 331:84-86. Fusion proteins that have a disulfide-linked dimeric structure due to the IgG portion can also be more efficient in binding and neutralizing other molecules than monomeric polypeptides or fragments thereof alone. See, e.g., Fountoulakis et al. (1995) J. Biochem. 270:3958-3964. Nucleic acids encoding the above epitopes can also be recombined with a gene of interest as an epitope tag to aid in detection and purification of the expressed polypeptide.

Antibodies

The present invention further relates to antibodies and T-cell antigen receptors (TCR) which specifically bind the polypeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention include, but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes monoclonal, polyclonal, chimeric, humanized, and human monoclonal and polyclonal antibodies which specifically bind the polypeptides of the present invention. The present invention further includes antibodies which are anti-idiotypic to the antibodies of the present invention.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, A. et al. (1991) J. Immunol. 147:60-69; U.S. Pat. Nos. 5,573,920, 4,474,893, 5,601,819, 4,714,681, 4,925,648; Kostelny, S. A. et al. (1992) J. Immunol. 148:1547-1553.

Antibodies of the present invention may be described or specified in terms of the epitope(s) or portion(s) of a polypeptide of the present invention which are recognized or specifically bound by the antibody. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M , 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow et al., ANTIBODIES: A LABORATORY MANUAL, (Cold Spring Harbor Laboratory Press, 2nd ed. 1988) (incorporated by reference in the entirety).

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art and taught in Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS 563-681 (Elsevier, N.Y., 1981) (said references incorporated by reference in their entireties). Fab and F(ab′)2 fragments may be produced by proteolytic cleavage, using enzymes such as papain (to produce Fab fragments) or pepsin (to produce F(ab′)2 fragments).

Alternatively, antibodies of the present invention can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman U. et al. (1995) J. Immunol. Methods 182:41-50; Ames, R. S. et al. (1995) J. Immunol. Methods 184:177-186; Kettleborough, C. A. et al. (1994) Eur. J. Immunol. 24:952-958; Persic, L. et al. (1997) Gene 187 9-18; Burton, D. R. et al. (1994) Advances in Immunology 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)2 fragments can also be employed using methods known in the art such as those disclosed in WO 92/22324; Mullinax, R. L. et al. (1992) BioTechniques 12(6):864-869; and Sawai, H. et al. (1995) AJRI 34:26-34; and Better, M. et al. (1988) Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al. (1991) Methods in Enzymology 203:46-88; Shu, L. et al. (1993) PNAS 90:7995-7999; and Skerra, A. et al. (1988) Science 240:1038-1040. For some uses, including in vivo use of antibodies in humans and in vitro detection assays, it may be preferable to use chimeric, humanized, or human antibodies. Methods for producing chimeric antibodies are known in the art. See e.g., Morrison, Science 229:1202 (1985); Oi et al., BioTechniques 4:214 (1986); Gillies, S. D. et al. (1989) J. Immunol. Methods 125:191-202; and U.S. Pat. No. 5,807,715. Antibodies can be humanized using a variety of techniques including CDR-grafting (EP 0 239 400; WO 91/09967; U.S. Pat. No. 5,530,101; and 5,585,089), veneering or resurfacing (EP 0 592 106; EP 0 519 596; Padlan E. A., (1991) Molecular Immunology 28(4/5):489-498; Studnicka G. M. et al. (1994) Protein Engineering 7(6):805-814; Roguska M. A. et al. (1994) PNAS 91:969-973), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; and WO 98/46645, WO 98/50433, WO 98/24893, WO 98/16654, WO 96/34096, WO 96/33735, and WO 91/10741 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al. supra and WO 93/21232; EP 0 439 095; Naramura, M. et al. (1994) Immunol. Lett. 39:91-99; U.S. Pat. No. 5,474,981; Gillies, S. O. et al. (1992) PNAS 89:1428-1432; Fell, H. P. et al. (1991) J. Immunol. 146:2446-2452 (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al. (1991) PNAS 88:10535-10539; Zheng, X. X. et al. (1995) J. Immunol. 154:5590-5600; and Vil, H. et al. (1992) PNAS 89:11337-11341 (said references incorporated by reference in their entireties).

The invention further relates to antibodies that act as agonists or antagonists of the polypeptides of the present invention. Antibodies which act as agonists or antagonists of the polypeptides of the present invention include, for example, antibodies which disrupt receptor/ligand interactions with the polypeptides of the invention either partially or fully. For example, the present invention includes antibodies that disrupt the ability of the proteins of the invention to multimerize. In another example, the present invention includes antibodies which allow the proteins of the invention to multimerize, but disrupts the ability of the proteins of the invention to bind one or more neuropeptide receptor receptor(s)/ligand(s). In yet another example, the present invention includes antibodies which allow the proteins of the invention to multimerize, and bind neuropeptide receptor receptor(s)/ligand(s), but blocks biological activity associated with the neuropeptide receptor/receptor/ligand complex.

Antibodies which act as agonists or antagonists of the polypeptides of the present invention also include, both receptor-specific antibodies and ligand-specific antibodies. Included are receptor-specific antibodies that do not prevent ligand binding but prevent receptor activation. Receptor activation (i.e., signaling) may be determined by techniques described herein or otherwise known in the art. Also included are receptor-specific antibodies which both prevent ligand binding and receptor activation. Likewise, included are neutralizing antibodies which bind the ligand and prevent binding of the ligand to the receptor, as well as antibodies which bind the ligand, thereby preventing receptor activation, but do not prevent the ligand from binding the receptor. Further included are antibodies that activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al., Blood 92(6):1981-1988 (1998); Chen, Z. et al., Cancer Res. 58(16):3668-3678 (1998); Harrop, J. A. et al., J. Immunol. 161(4):1786-1794 (1998); Zhu, Z. et al., Cancer Res. 58(15):3209-3214 (1998); Yoon, D. Y. et al., J. Immunol. 160(7):3170-3179 (1998); Prat, M. et al., J. Cell. Sci. 1 1(Pt2):237-247 (1998); Pitard, V. et al., J. Immunol. Methods 205(2):177-190 (1997); Liautard, J. et al., Cytokinde 9(4):233-241 (1997); Carlson, N. G. et al., J. Biol. Chem. 272(17):11295-11301 (1997); Taryman, R. E. et al., Neuron 14(4):755-762 (1995); Muller, Y. A. et al., Structure 6(9):1153-1167 (1998); Bartunek, P. et al., Cytokine 8(1):14-20 (1996) (said references incorporated by reference in their entireties).

As discussed above, antibodies to the neuropeptide receptor proteins of the invention can, in turn, be utilized to generate anti-idiotype antibodies that “mimic” neuropeptide receptor using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to neuropeptide receptor and competitively inhibit neuropeptide receptor multimerization and/or binding to ligand can be used to generate anti-idiotypes that “mimic” the neuropeptide receptor mutimerization and/or binding domain and, as a consequence, bind to and neutralize neuropeptide receptor and/or its ligand. Such neutralizing anti-idiotypes or Fab fragments of such anti-idiotypes can be used in therapeutic regimens to neutralize neuropeptide receptor ligand. For example, such anti-idiotypic antibodies can be used to bind neuropeptide receptor, or to bind neuropeptide receptor ligands/receptors, and thereby block neuropeptide receptor biological activity.

Fusion Proteins

Any neuropeptide receptor polypeptide can be used to generate fusion proteins. For example, the neuropeptide receptor polypeptide, when fused to a second protein, can be used as an antigenic tag. Antibodies raised against the neuropeptide receptor polypeptide can be used to indirectly detect the second protein by binding to the neuropeptide receptor. Moreover, because secreted proteins target cellular locations based on trafficking signals, the neuropeptide receptor polypeptides can be used as a targeting molecule once fused to other proteins.

Examples of domains that can be fused to neuropeptide receptor polypeptides include not only heterologous signal sequences, but also other heterologous functional regions. The fusion does not necessarily need to be direct, but may occur through linker sequences.

In certain preferred embodiments, neuropeptide receptor proteins of the invention comprise fusion proteins wherein the neuropeptide receptor polypeptides are those described above as m-n. In preferred embodiments, the application is directed to nucleic acid molecules at least 90%, 95%, 96%, 97%, 98% or 99% identical to the nucleic acid sequences encoding polypeptides having the amino acid sequence of the specific N- and C-terminal deletions recited herein. Polynucleotides encoding these polypeptides are also encompassed by the invention.

Moreover, fusion proteins may also be engineered to improve characteristics of the neuropeptide receptor polypeptide. For instance, a region of additional amino acids, particularly charged amino acids, may be added to the N-terminus of the neuropeptide receptor polypeptide to improve stability and persistence during purification from the host cell or subsequent handling and storage. Also, peptide moieties may be added to the neuropeptide receptor polypeptide to facilitate purification. Such regions may be removed prior to final preparation of the neuropeptide receptor polypeptide. The addition of peptide moieties to facilitate handling of polypeptides are familiar and routine techniques in the art.

Moreover, neuropeptide receptor polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the neuropeptide receptor polypeptides can be fused to marker sequences, such as a peptide which facilitates purification of neuropeptide receptor. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the neuropeptide receptor polynucleotides or the polypeptides.

Vectors, Host Cells, and Protein Production

The present invention also relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the human neuropeptide receptor genes. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda PL promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9 (Qiagen), pbs, pD10, phagescript, psiX174, pbluescript SK, pbsks, pNH8A, pNH16a, pNH18A, pNH46A (Stratagene); pTRC99a, pKK223-3, pKK233-3, pDR540, pRIT5 (Pharmacia). Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are PKK232-8 and PCM7. Particular named bacterial promoters include lacI, lacZ, T3, T7, gpt, lambda P_{R}, P_{L}, trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology, (1986)).

The constructs in host cells can be used in a conventional manner to produce the gene product encoded by the recombinant sequence. Alternatively, the polypeptides of the invention can be synthetically produced by conventional peptide synthesizers.

Fragments of the polypeptides of the present invention may be employed for producing the corresponding full-length polypeptide by peptide synthesis, therefore, the fragments may be employed as intermediates for producing the full-length polypeptides. Fragments of the polynucleotides of the present invention may be used in a similar manner to synthesize the full-length polynucleotides of the present invention.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), Â-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The neuropeptide receptor polypeptide of the present invention can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The neuropeptide receptor polypeptide of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

The present invention also relates to vectors containing the neuropeptide receptor polynucleotide, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

Neuropeptide receptor polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The neuropeptide receptor polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that neuropeptide receptor polypeptides may in fact be expressed by a host cell lacking a recombinant vector.

Neuropeptide receptor polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Neuropeptide receptor polypeptides, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the neuropeptide receptor polypeptides may be glycosylated or may be non-glycosylated. In addition, neuropeptide receptor polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., neuropeptide receptor coding sequence), and/or to include genetic material (e.g., heterologous polynucleotide sequences) that is operably associated with neuropeptide receptor polynucleotides of the invention, and which activates, alters, and/or amplifies endogenous neuropeptide receptor polynucleotides. For example, techniques known in the art may be used to operably associate heterologous control regions (e.g., promoter and/or enhancer) and endogenous neuropeptide receptor polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

In addition, polypeptides of the invention can be chemically synthesized using techniques known in the art (eg, see Creighton, 1983, Proteins: Structures and Molecular Principles, W.H. Freeman & Co., N.Y., and Hunkapiller, M., et al., 1984, Nature 310:105-111). For example, a peptide corresponding to a fragment of the neuropeptide receptor polypeptides of the invention can be synthesized by use of a peptide synthesizer. Furthermore, if desired, nonclassical amino acids or chemical amino acid analogs can be introduced as a substitution or addition into the neuropeptide receptor polynucleotide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses neuropeptide receptor polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of neuropeptide receptor which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

The neuropeptide receptor polypeptides of the invention may be in monomers or multimers (i.e., dimers, trimers, tetramers and higher multimers). Accordingly, the present invention relates to monomers and multimers of the neuropeptide receptor polypeptides of the invention, their preparation, and compositions (preferably, pharmaceutical compositions) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only neuropeptide receptor polypeptides of the invention (including neuropeptide receptor fragments, variants, splice variants, and fusion proteins, as described herein). These homomers may contain neuropeptide receptor polypeptides having identical or different amino acid sequences. In a specific embodiment, a homomer of the invention is a multimer containing only neuropeptide receptor polypeptides having an identical amino acid sequence. In another specific embodiment, a homomer of the invention is a multimer containing neuropeptide receptor polypeptides having different amino acid sequences. In specific embodiments, the multimer of the invention is a homodimer (e.g., containing neuropeptide receptor polypeptides having identical or different amino acid sequences) or a homotrimer (e.g., containing neuropeptide receptor polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the neuropeptide receptor polypeptides of the invention. In a specific embodiment, the multimer of the invention is a heterodimer, a heterotrimer, or a heterotetramer. In additional embodiments, the homomeric multimer of the invention is at least a homodimer, at least a homotrimer, or at least a homotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the neuropeptide receptor polypeptides of the invention. Such covalent associations may involve one or more amino acid residues contained in the polypeptide sequence (e.g., that recited in SEQ ID NO:2, or contained in the polypeptide encoded by the clone HFGAN72). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a neuropeptide receptor fusion protein. In one example, covalent associations are between the heterologous sequence contained in a fusion protein of the invention (see, e.g., U.S. Pat. No. 5,478,925). In a specific example, the covalent associations are between the heterologous sequence contained in a neuropeptide receptor-Fc fusion protein of the invention (as described herein). In another specific example, covalent associations of fusion proteins of the invention are between heterologous polypeptide sequence from another neuropeptide receptor family member that is capable of forming covalently associated multimers, such as for example, oseteoprotegerin (see, e.g., International Publication No. WO 98/49305, the contents of which are herein incorporated by reference in its entirety).

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hyrophobic or signal peptide) and which can be incorporated by membrane reconstitution techniques into liposomes (see, e.g., U.S. Pat. No. 5,478,925, which is herein incorporated by reference in its entirety).

Uses of the Neuropeptide Receptor Polynucleotides

The polynucleotides of the present invention may be employed as research reagents and materials for discovery of treatments and diagnostics to human disease. The neuropeptide receptor polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available. Clone HFGAN72 was mapped to chromosome 1 position p31-34. Thus, neuropeptide receptor polynucleotides can be used in linkage analysis as a marker for chromosome 1.

Briefly, sequences can be mapped to chromosomes by preparing PCR primers (preferably 15-25 bp) from the sequences shown in SEQ ID NO:1. Primers can be selected using computer analysis so that primers do not span more than one predicted exon in the genomic DNA. These primers are then used for PCR screening of somatic cell hybrids containing individual human chromosomes. Only those hybrids containing the human neuropeptide receptor gene corresponding to the SEQ ID NO:1 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the neuropeptide receptor polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the neuropeptide receptor polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the neuropeptide receptor polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the neuropeptide receptor polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the neuropeptide receptor polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using neuropeptide receptor polynucleotides. Any of these alterations (altered expression, chromosomal rearrangement, or mutation) can be used as a diagnostic or prognostic marker.

In addition to the foregoing, a neuropeptide receptor polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Neuropeptide receptor polynucleotides are also useful in gene therapy. One goal of gene therapy is to insert a normal gene into an organism having a defective gene, in an effort to correct the genetic defect. Neuropeptide receptors offer a means of targeting such genetic defects in a highly accurate manner. Another goal is to insert a new gene that was not present in the host genome, thereby producing a new trait in the host cell.

The neuropeptide receptor polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The neuropeptide receptor polynucleotides can be used as additional DNA markers for RFLP.

The neuropeptide receptor polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, neuropeptide receptor polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from hypothalamus. Panels of such reagents can identify tissue by species and/or by organ type. In a similar fashion, these reagents can be used to screen tissue cultures for contamination.

Because neuropeptide receptor is found expressed in hypothalamus, neuropeptide receptor polynucleotides are useful as hybridization probes for differential identification of the tissue(s) or cell type(s) present in a biological sample. Similarly, polypeptides and antibodies directed to neuropeptide receptor polypeptides are useful to provide immunological probes for differential identification of the tissue(s) or cell type(s). In addition, for a number of disorders of the above tissues or cells, particularly of the central and peripheral nervous system, significantly higher or lower levels of neuropeptide receptor gene expression may be detected in certain tissues (e.g., cancerous and wounded tissues) or bodily fluids (e.g., serum, plasma, urine, synovial fluid or spinal fluid) taken from an individual having such a disorder, relative to a “standard” neuropeptide receptor gene expression level, i.e., the neuropeptide receptor expression level in healthy tissue from an individual not having the neuropeptide receptor system disorder.

Thus, the invention provides a diagnostic method of a disorder, which involves: (a) assaying neuropeptide receptor gene expression level in cells or body fluid of an individual; (b) comparing the neuropeptide receptor gene expression level with a standard neuropeptide receptor gene expression level, whereby an increase or decrease in the assayed neuropeptide receptor gene expression level compared to the standard expression level is indicative of disorder in the neuropeptide receptor system.

In the very least, the neuropeptide receptor polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of Neuropeptide Receptor Polypeptides

The polypeptides of the present invention may be employed as research reagents and materials for discovery of treatments and diagnostics to human disease. Neuropeptide receptor polypeptides can be used in numerous ways. The following description should be considered exemplary and utilizes known techniques.

Neuropeptide receptor polypeptides can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell . Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).) Thus, the invention provides a diagnostic method of a disorder, which involves (a) assaying the expression of neuropeptide receptor polypeptide in cells or body fluid of an individual; (b) comparing the level of gene expression with a standard gene expression level, whereby an increase or decrease in the assayed neuropeptide receptor polypeptide gene expression level compared to the standard expression level is indicative of a disorder.

Moreover, neuropeptide receptor polypeptides can be used to treat disease. For example, patients can be administered neuropeptide receptor polypeptides in an effort to replace absent or decreased levels of the neuropeptide receptor polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to neuropeptide receptor polypeptides can also be used to treat disease. For example, administration of an antibody directed to a neuropeptide receptor polypeptide can bind and reduce overproduction of the polypeptide. Similarly, administration of an antibody can activate the polypeptide, such as by binding to a polypeptide bound to a membrane (receptor).

The human neuropeptide receptor polypeptides of the present invention may be employed in a process for screening compounds which bind to and activate the receptor polypeptide and for compounds which bind to and inhibit activation of the receptor polypeptides of the present invention.

In general, the neuropeptide receptor is isolated, immobilized or cell bound form is contacted with a plurality of compounds and those compounds are selected which bind to and interact with the receptor. The binding or interaction can be measured directly by using radioactively labeled compounds of interest or by the second messenger effect resulting from the interaction or binding of the candidate compound. Alternatively, the candidate compounds can be subjected to competition screening assays, in which a known ligand, preferably labeled with an analytically detectable reagent, most preferably radioactivity, is introduced with the compound to be tested and the compound's capacity to inhibit or enhance the binding of the labeled ligand is measured. Compounds are screened for their increased afffinity and selectivity to the receptor polypeptide of the present invention.

One such screening procedure involves the use of melanophores which are transfected to express the neuropeptide receptor of the present invention. Such a screening technique is described in PCT WO 92/01810 published Feb. 6, 1992.

For example, to screen for compounds which inhibit activation of the receptor polypeptide of the present invention, the compound and a ligand known to bind to the receptor are both contacted with the melanophore cells. Inhibition of the signal generated by the ligand indicates that the compound inhibits activation of the receptor.

The screen may be employed for determining a compound which binds to and activates the receptor polypeptide of the present invention by contacting such cells with compounds to be screened and determining whether such compound generates a signal, i.e., activates the receptor.

Other examples include the use of cells which express a neuropeptide receptor of the present invention (for example, transfected CHO cells) in a system which measures extra-cellular pH changes caused by receptor activation, for example, as described in Science, volume 246, pages 181-296 (October 1989). For example, compounds may be contacted with a cell which expresses an neuropeptide receptor polypeptide of the present invention and a second messenger response, e.g. signal transduction or pH changes, may be measured to determine whether the potential compound is effective as an activator or inhibitor.

Another example involves introducing RNA encoding a neuropeptide receptor of the present invention into Xenopus oocytes to transiently express the receptor. The oocytes may then be contacted with the receptor ligand and a compound to be screened, followed by detection of inhibition of or an increase in intracellular calcium.

Another example involves expressing a neuropeptide receptor polypeptide of the present invention on the surface of a cell wherein the receptor is linked to a phospholipase C or D. As representative examples of such cells there may be mentioned endothelial cells, smooth muscle cells, embryonic kidney cells, etc. The screening may be accomplished as hereinabove described by detecting activation of the receptor or inhibition of activation of the receptor from the phospholipase second signal.

Another method involves determining inhibition of binding of labeled ligand to cells which have a neuropeptide receptor on the surface thereof. Such a method involves transfecting a eukaryotic cell with DNA encoding an neuropeptide receptor polypeptide of the present invention such that the cell expresses the receptor on its surface and contacting the cell with a compound in the presence of a labeled form of a known ligand. The ligand can be labeled, e.g., by radioactivity. The amount of labeled ligand bound to the receptors is measured, e.g., by measuring radioactivity of the receptors. If the compound binds to the receptor as determined by a reduction of labeled ligand which binds to the receptors, the binding of labeled ligand to the receptor is inhibited.

Another screening technique involves expressing a neuropeptide receptor polypeptide on the surface of a cell wherein the receptor is linked to a second messenger to increase cytosolic calcium levels in transfected CHO cells. An example of such a method comprises transfecting CHO cells with a nucleic acid sequence encoding a receptor of the present invention such that the receptor is expressed on the surface thereof. The transfected cell is then incubated in a reaction mixture with labeled calcium in the presence of a compound to be screened. The ability of the compound to increase calcium up-take or inhibit calcium up-take can then be determined by measuring the amount of labeled calcium transported into the cells by taking advantage of the label, e.g., radioactivity.

Compounds may also be identified by the above methods which bind to specific subregions within the CNS that are important for specific behaviors through indirect interactions with a neuropeptide receptor polypeptide of the present invention.

To measure intracellular cyclic AMP levels, cyclic AMP is assayed in whole cells treated for 15 minutes at 37° C. with 100 micromolar isobutylmethylxanthine (IBMX; Sigma). Transfected cells (1×10⁶/0.5 ml reaction) are incubated with 10 micromolar forskolin and various concentrations of known or unknown ligands to the receptor. Reactions are terminated with the addition of HCl to 0.1M, incubation at room temperature for 15 minutes, neutralization and sample dilution in 50 mM sodium acetate, pH 6.2. Cyclic AMP is quantified by using a radioimmunoassay (Dupont/NEN).

To measure levels of intracellular calcium, transfected cells are suspended in loading medium (modified RPMI 1640 medium/10 mM Hepes/1% newborn calf serum) and incubated in a spinner flask at 37° C. for 2.5 hour at 1×10⁶ cells per ml. Cells are then treated with 1 micromolar Fura-2 acetoxymethyl ester (fura-2 AM; Molecular Probes) for 30 minutes at 37° C., washed twice with loading medium, and resuspended at 5×10⁶ cells/ml. Immediately before fluorescence spectroscopy, cells are recovered by centrifugation at 1000 rpm and resuspended at 1×10 cells/ml in a modified Krebs buffer (135 mM NaCl/4.7 mM KCl/1.2 MM MgSO₄/1.2 mM KH₂PO₄/5 mM NaHCO₃/1 mM CaCl₂/2.8 mM glucose/10 mM hepes, pH 7.4) containing sulfinpyrazone. Bombesin is purchased from Sigma and Auspep. Fluorescence recordings are made on a Hitachi fluorescence spectrometer (F4010) at 340 nm (excitation) and 505 nm (emission) over 10 minutes with slit widths of 5 nm and response time of 2 seconds. Intracellular calcium is quantified by using equations described by Grynkiewicz, et al., J. Bio. Chem. 260:3440-3450, 1985.

At the very least, the neuropeptide receptor polypeptides can be used as molecular weight markers on SDS-PAGE gels or on molecular sieve gel filtration columns using methods well known to those of skill in the art. Neuropeptide receptor polypeptides can also be used to raise antibodies, which in turn are used to measure protein expression from a recombinant cell, as a way of assessing transformation of the host cell. Moreover, neuropeptide receptor polypeptides can be used to test the following biological activities.

Gene Therapy Methods

Another aspect of the present invention is to gene therapy methods for treating disorders, diseases and conditions. The gene therapy methods relate to the introduction of nucleic acid (DNA, RNA and antisense DNA or RNA) sequences into an animal to achieve expression of the neuropeptide receptor polypeptide of the present invention. This method requires a polynucleotide which codes for a neuropeptide receptor polypeptide operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a neuropeptide receptor polynucleotide ex vivo, with the engineered cells then being provided to a patient to be treated with the polypeptide. Such methods are well-known in the art. For example, see Belldegrun, A., et al., J. Natl. Cancer Inst. 85: 207-216 (1993); Ferrantini, M. et al., Cancer Research 53: 1107-1112 (1993); Ferrantini, M. et al., J. Immunology 153: 4604-4615 (1994); Kaido, T., et al., Int. J. Cancer 60: 221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the neuropeptide receptor polynucleotide constructs can be delivered by any method that delivers injectable materials to the cells of an animal, such as, injection into the interstitial space of tissues (heart, muscle, skin, lung, liver, and the like). The neuropeptide receptor polynucleotide constructs may be delivered in a phanmaceutically acceptable liquid or aqueous carrier.

In one embodiment, the neuropeptide receptor polynucleotide is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the neuropeptide receptor polynucleotides can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The neuropeptide receptor polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of neuropeptide receptor DNA. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for neuropeptide receptor.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The neuropeptide receptor polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked neuropeptide receptor DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

As is evidenced in the Examples, naked neuropeptide receptor nucleic acid sequences can be administered in vivo results in the successful expression of neuropeptide receptor polypeptide in the femoral arteries of rabbits.

The constructs may also be delivered with delivery vehicles such as viral sequences, viral particles, liposome formulations, lipofectin, precipitating agents, etc. Such methods of delivery are known in the art.

In certain embodiments, the neuropeptide receptor polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265: 10189-10192, which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/9469 (which are herein incorporated by reference) provide methods for delivering DNA-cationic lipid complexes to mammals.

In certain embodiments, cells are be engineered, ex vivo or in vivo, using a retroviral particle containing RNA which comprises a sequence encoding neuropeptide receptor. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include polynucleotide encoding neuropeptide receptor. Such retroviral vector particles then may be employed, to transduce eukaryotic cells, either in vitro or in vivo. The transduced eukaryotic cells will express neuropeptide receptor.

In certain other embodiments, cells are engineered, ex vivo or in vivo, with neuropeptide receptor polynucleotide contained in an adenovirus vector. Adenovirus can be manipulated such that it encodes and expresses neuropeptide receptor, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis.109:233-238). Finally, adenovirus mediated gene transfer has been demonstrated in a number of instances including transfer of alpha-l-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 l region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, for example, the HARP promoter of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The neuropeptide receptor polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the neuropeptide receptor polynucleotide construct. These viral particles are then used to transduce eukaryotic cells, either ex vivo or in vivo. The transduced cells will contain the neuropeptide receptor polynucleotide construct integrated into its genome, and will express neuropeptide receptor.

Another method of gene therapy involves operably associating heterologous control regions and endogenous polynucleotide sequences (e.g. encoding neuropeptide receptor) via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the neuropeptide receptor desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous neuropeptide receptor sequence is placed under the control of the promoter. The promoter then drives the expression of the endogenous neuropeptide receptor sequence.

The polynucleotides encoding neuropeptide receptor may be administered along with other polynucleotides encoding other angiongenic proteins. Angiogenic proteins include, but are not limited to, acidic and basic fibroblast growth factors, VEGF-1, epidermal growth factor alpha and beta, platelet-derived endothelial cell growth factor, platelet-derived growth factor, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding neuropeptide receptor contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Therapeutic compositions of the present invention can be administered to any animal, preferably to mammals and birds. Preferred mammals include humans, dogs, cats, mice, rats, rabbits sheep, cattle, horses and pigs, with humans being particularly preferred.

Additionally, the neuropeptide receptor polypeptides and compounds identified above which are polypeptides, may be employed in accordance with the present invention by expression of such polypeptides in vivo, which is often referred to as “gene therapy.” Thus, for example, cells from a patient may be engineered with a polynucleotide (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a polypeptide of the present invention.

Similarly, cells may be engineered in vivo for expression of a polypeptide in vivo by, for example, procedures known in the art. As known in the art, a producer cell for producing a retroviral particle containing RNA encoding the polypeptide of the present invention may be administered to a patient for engineering cells in vivo and expression of the polypeptide in vivo. These and other methods for administering a polypeptide of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and -actin promoters). Other viral promoters which may be employed include, but are not limited to, adenovirus promoters, thymidine kinase (TK) promoters, and B 19 parvovirus promoters. The selection of a suitable promoter will be apparent to those skilled in the art from the teachings contained herein.

The nucleic acid sequence encoding the polypeptide of the present invention is under the control of a suitable promoter. Suitable promoters which may be employed include, but are not limited to, adenoviral promoters, such as the adenoviral major late promoter; or hetorologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the -actin promoter; and human growth hormone promoters. The promoter also may be the native promoter which controls the genes encoding the polypeptides.

The retroviral plasmid vector is employed to transduce packaging cell lines to form producer cell lines. Examples of packaging cells which may be transfected include, but are not limited to, the PE501, PA317, -2, -AM, PA12, T19-14X, VT-19-17-H2, CRE, CRIP, GP+E-86, GP+envAm12, and DAN cell lines as described in Miller, Human Gene Therapy, Vol. 1, pgs. 5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Biological Activities of Neuropeptide Receptor

Neuropeptide receptor polynucleotides or polypeptides, or agonists or antagonists of neuropeptide receptor, can be used in assays to test for one or more biological activities. If neuropeptide receptor polynucleotides or polypeptides, or agonists or antagonists of neuropeptide receptor, do exhibit activity in a particular assay, it is likely that neuropeptide receptor may be involved in the diseases associated with the biological activity. Therefore, neuropeptide receptor could be used to treat the associated disease.

Immune Activity

Neuropeptide receptor polynucleotides or polypeptides, or agonists or antagonists of neuropeptide receptor, may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, neuropeptide receptor polynucleotides or polypeptides, or agonists or antagonists of neuropeptide receptor, can be used as a marker or detector of a particular immune system disease or disorder.

Neuropeptide receptor polynucleotides or polypeptides, or agonists or antagonists of neuropeptide receptor, may be useful in treating or detecting deficiencies or disorders of hematopoietic cells. Neuropeptide receptor polynucleotides or polypeptides, or agonists or antagonists of neuropeptide receptor, could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, neuropeptide receptor polynucleotides or polypeptides, or agonists or antagonists of neuropeptide receptor, can also be used to modulate hemostatic (the stopping of bleeding) or thrombolytic activity (clot formation). For example, by increasing hemostatic or thrombolytic activity, neuropeptide receptor polynucleotides or polypeptides, or agonists or antagonists of neuropeptide receptor, could be used to treat blood coagulation disorders (e.g., afibrinogenemia, factor deficiencies), blood platelet disorders (e.g. thrombocytopenia), or wounds resulting from trauma, surgery, or other causes. Alternatively, neuropeptide receptor polynucleotides or polypeptides, or agonists or antagonists of neuropeptide receptor, that can decrease hemostatic or thrombolytic activity could be used to inhibit or dissolve clotting, important in the treatment of heart attacks (infarction), strokes, or scarring.

Neuropeptide receptor polynucleotides or polypeptides, or agonists or antagonists of neuropeptide receptor, may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of neuropeptide receptor polynucleotides or polypeptides, or agonists or antagonists of neuropeptide receptor, that can inhibit an immune response, particularly the proliferation, differentiation, or chemotaxis of T-cells, may be an effective therapy in preventing autoimmune disorders.

Examples of autoimmune disorders that can be treated or detected include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by neuropeptide receptor polynucleotides or polypeptides, or agonists or antagonists of neuropeptide receptor. Moreover, these molecules can be used to treat anaphylaxis, hypersensitivity to an antigenic molecule, or blood group incompatibility.

Neuropeptide receptor polynucleotides or polypeptides, or agonists or antagonists of neuropeptide receptor, may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of neuropeptide receptor polynucleotides or polypeptides, or agonists or antagonists of neuropeptide receptor, that inhibits an immune response, particularly the proliferation, differentiation, or chemotaxis of T-cells, may be an effective therapy in preventing organ rejection or GVHD.

Similarly, neuropeptide receptor polynucleotides or polypeptides, or agonists or antagonists of neuropeptide receptor, may also be used to modulate inflammation. For example, neuropeptide receptor polynucleotides or polypeptides, or agonists or antagonists of neuropeptide receptor, may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

Neuropeptide receptor polynucleotides or polypeptides, or agonists or antagonists of neuropeptide receptor, can be used to treat or detect hyperproliferative disorders, including neoplasms. Neuropeptide receptor polynucleotides or polypeptides, or agonists or antagonists of neuropeptide receptor, may inhibit the proliferation of the disorder through direct or indirect interactions. Alternatively, neuropeptide receptor polynucleotides or polypeptides, or agonists or antagonists of neuropeptide receptor, may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by neuropeptide receptor polynucleotides or polypeptides, or agonists or antagonists of neuropeptide receptor, include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by neuropeptide receptor polynucleotides or polypeptides, or agonists or antagonists of neuropeptide receptor. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Cardiovascular Disorders

Neuropeptide receptor polynucleotides or polypeptides, or agonists or antagonists of neuropeptide receptor, encoding neuropeptide receptor may be used to treat cardiovascular disorders, including peripheral artery disease, such as limb ischemia.

Cardiovascular disorders include cardiovascular abnormalities, such as arterio-arterial fistula, arteriovenous fistula, cerebral arteriovenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular disorders also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve disease include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular disorders, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular disorders include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

Neuropeptide receptor polynucleotides or polypeptides, or agonists or antagonists of neuropeptide receptor, are especially effective for the treatment of critical limb ischemia and coronary disease.

Neuropeptide receptor polypeptides may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Neuropeptide receptor polypeptides may be administered as part of a pharmaceutical composition, described in more detail below. Methods of delivering neuropeptide receptor polynucleotides are described in more detail herein.

Obesity and Eating Behavior Disorders

The invention also provides a method of treating and/or preventing obesity by administering to a host a compound which binds to and activates the receptor polypeptides of the present invention. Such a compound is other than the ob gene product disclosed in Zhang, et al., Nature, 372:425-431 (1994). The receptor polypeptide of the present invention maps to a human chromosome which corresponds to the position of the mouse chromosome which encodes for the receptor of the ob gene product. The human ob gene encodes a “satiety” factor which binds to and activates the receptor polypeptide of the present invention. Accordingly, a compound which activates the receptor of the present invention will decrease appetite and prevent obesity.

The compounds described above may also be employed to enhance activity level, modify eating behavior, enhance utilization of ingested foods and regulate deposition of fat stores. Conditions related to obesity may also be treated by the compounds which bind to and activate the receptor polypeptides of the present invention including, but not limited to, hyperlidimeia, type II diabetes and certain cancers.

These compounds may also be employed to treat and/or prevent other conditions related to an underexpression of the receptor polypeptide of the present invention or ligands which bind thereto, for example, to stimulate neuronal growth.

Specific examples of compounds which inhibit activation of the receptor polypeptides of the present invention include an antibody, or in some cases an oligonucleotide, which binds to the receptor but does not elicit a second messenger response such that the activity of the receptor is prevented. Another example is proteins which are closely related to the ligands of the receptor, i.e. a fragment of the ligand, which have lost biological function and when binding to the receptor, elicit no response.

Another example includes an antisense construct prepared through the use of antisense technology. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes for the mature polypeptides of the present invention, is used to design an antisense RNA oligonucleotide of from about 10 to 40 base pairs in length. A DNA oligonucleotide is designed to be complementary to a region of the gene involved in transcription (triple helix -see Lee et al., Nucl. Acids Res., 6:3073 (1979); Cooney et al, Science, 241:456 (1988); and Dervan et al., Science, 251: 1360 (1991)), thereby preventing transcription and the production of a neuropeptide receptor polypeptide of the present invention The antisense RNA oligonucleotide hybridizes to the mRNA in vivo and blocks translation of the mRNA molecule into the receptor (antisense—Okano, J. Neurochem., 56:560 (1991); Oligodeoxynucleotides as Antisense Inhibitors of Gene Expression, CRC Press, Boca Raton, Fla. (1988)). The oligonucleotides described above can also be delivered to cells such that the antisense RNA or DNA may be expressed in vivo to inhibit production of the receptors.

Another example is a small molecule which binds to a neuropeptide receptor polypeptide of the present invention, making it inaccessible to ligands such that normal biological activity is prevented. Examples of small molecules include but are not limited to small peptides or peptide-like molecules and neuropeptide Y fragments and/or derivatives.

Soluble forms of a neuropeptide receptor polypeptide of the present invention, e.g., a fragment of the receptor, which binds to the ligand and prevents the ligand from interacting with membrane bound receptors may also inhibit activation of the receptor polypeptides of the present invention.

This invention additionally provides a method of utilizing such compounds which inhibit activation for treating abnormal conditions related to an excess of activity of a neuropeptide receptor polypeptide of the present invention for treating obesity since the neuropeptide receptor polypeptides of the present invention may bind neuropeptide Y which is the most potent known substance to cause an increase in feeding behavior and type II Diabetes Mellitus since neuropeptide Y may play a role in the genetic basis of this disease.

Nervous System Diseases

Neuropeptide receptor polynucleotides or polypeptides, or agonists or antagonists of neuropeptide receptor, may be useful in treating deficiencies or disorders of the nervous system. Moreover, neuropeptide receptor polynucleotides or polypeptides, or agonists or antagonists of neuropeptide receptor, can be used as a marker or detector of a particular nervous system disease or disorder. Nervous system diseases and disorders include, for example, central nervous system diseases, such as brain diseases (e.g., akinetic mutism, basal ganglia disease, brain abscesses, central auditory diseases (e.g., auditory perceptual disorders or central hearing loss), cerebral palsy, metabolic or chronic brain diseases, brain edemas, brain neoplasms, Canavan disease, cerebellar diseases, diffuse cerebral sclerosis, cerebrovascular diseases, dementia, encephalitis, encephalomalacia (e.g., leukomalacia), epilepsy, Hallervorden-Spatz Syndrome, hydrocephalus (e.g., Dandy-Walker Syndrome or normal pressure hydrocephalus), hypothalamic diseases (e.g., hypothalamic neoplasms), cerebral malaria, narcolepsy, cataplexy, bulbar poliomyelitis, pseudotumor cerebri, Rett Syndrome, Reye's Syndrome, thalamic diseases, cerebral toxoplasmosis, intracranial tuberculoma, or Zellweger Syndrome).

More specifically, types of basal ganglia diseases that can be treated by neuropeptide receptor polynucleotides or polypeptides, or agonists or antagonists of neuropeptide receptor include, for example, drug-induced akathisia, Alzheimer's Disease, chorea, Huntington's Disease, Creutzfeldt-Jakob Syndrome, drug-induced dyskinesia, dystonia musculorum deformans, Hallervorden-Spatz Syndrome, hepatolenticular degeneration, Meige Syndrome, Neuroleptic Malignant Syndrome, Parkinson Disease (e.g., symptomatic or postencephalitic), progressive supranuclear palsy, or Tourette Syndrome.

Moreover, types of metabolic brain diseases that can be treated by neuropeptide receptor polynucleotides or polypeptides, or agonists or antagonists of neuropeptide receptor, include for example, abetalipoproteinemia, gangliosidose (e.g., GM1 gangliosidosis, Sandhoff Disease, or Tay-Sachs Disease), Hartnup Disease, hepatic encephalopathy, hepatolenticular degeneration, homocystinuria, kernicterus, Kinky Hair Syndrome, Leigh Disease, Lesch-Nyhan Syndrome, Maple Syrup Urine Disease, mitochondrial encephalomyopathies (e.g., MELAS Syndrome or MERRF Syndrome), central pontine myelinolysis, neuronal ceroid-lipofuscinosis, Niemann-Pick Disease, phenylketonuria, pyruvate carboxylase deficiency, pyruvate dehydrogenase complex deficiency, or Wemicke's Encephalopathy.

Additionally, types of brain neoplasms that can be treated by neuropeptide receptor polynucleotides or polypeptides, or agonists or antagonists of neuropeptide receptor include, for example, cerebellar neoplasms, infratentorial neoplasms, cerebral ventricle neoplasms, choroid plexus neoplasms, hypothalamic neoplasms, or supratentorial neoplasms.

In further embodiments, types of cerebellar diseases that can be treated by neuropeptide receptor polynucleotides or polypeptides, or agonists or antagonists of neuropeptide receptor include, for example, cerebellar ataxia, spinocerebellar degeneration, ataxia telangiectasia, cerebellar dyssynergia, Friedreich's Ataxia, Machado-Joseph Disease, olivopontocerebellar atrophy, or cerebellar neoplasms (e.g., infratentorial neoplasms).

Moreover, types of diffuse cerebral sclerosis that can be treated by neuropeptide receptor polynucleotides or polypeptides, or agonists or antagonists of neuropeptide receptor include, for example, encephalitis periaxialis, globoid cell leukodystrophy, metachromatic leukodystrophy, or subacute sclerosing panencephalitis.

Additionally, types of cerebrovascular disorders that can be treated by neuropeptide receptor polynucleotides or polypeptides, or agonists or antagonists of neuropeptide receptor, include for example, carotid artery diseases (e.g., carotid artery thrombosis, carotid stenosis, or Moyamoya Disease), cerebral amyloid angiopathy, cerebral aneurysm, cerebral anoxia, cerebral arteriosclerosis, cerebral arteriovenous malformations, cerebral artery diseases, cerebral embolism and thrombosis (e.g., carotid artery thrombosis, sinus thrombosis, or Wallenberg's Syndrome), cerebral hemorrhage (e.g., epidural or subdural hematoma, or subarachnoid hemorrhage), cerebral infarction, cerebral ischemia (e.g., transient cerebral ischemia, Subclavian Steal Syndrome, or vertebrobasilar insufficiency), vascular dementia (e.g., multi-infarct), leukomalacia, periventricular, or vascular headache (e.g., cluster headache or migraines).

In further embodiments, types of dementia that can be treated by neuropeptide receptor polynucleotides or polypeptides, or agonists or antagonists of neuropeptide receptor include, for example, AIDS dementia complex, presenile dementia (e.g., Alzheimer's Disease or Creutzfeldt-Jakob Syndrome), senile dementia (e.g., Alzheimer's Disease or progressive supranuclear palsy), or vascular dementia.

Moreover, types of encephalitis that can be treated by neuropeptide receptor polynucleotides or polypeptides, or agonists or antagonists of neuropeptide receptor include, for example, periaxialis encephalitis, viral encephalitis (e.g., epidemic, Japanese, St. Louis, Tick-Borne, or West Nile Fever encephalitis), encephalomyelitis, acute disseminated meningoencephalitis (e.g., Uveomeningoencephalitic Syndrome), postencephalitic Parkinson Disease, or subacute sclerosing panencephalitis.

Additionally, types of epilepsy that can be treated by neuropeptide receptor polynucleotides or polypeptides, or agonists or antagonists of neuropeptide receptor include, for example, generalized epilepsy (e.g., absence epilepsy, myoclonic epilepsy (e.g., MERRF Syndrome), tonic-clonic epilepsy, or infantile spasms) and partial epilepsy (e.g., complex partial epilepsy, frontal lobe epilepsy, temporal lobe epilepsy, post-traumatic epilepsy, or status epilepticus (e.g., epflepsia partialis continua).

Nervous system diseases and disorders that can be treated by neuropeptide receptor polynucleotides or polypeptides, or agonists or antagonists of neuropeptide receptor also include, for example, central nervous system infections, central nervous neoplasms, demyelinating diseases, encephalomyelitis, High Pressure Nervous Syndrome, meningism, spinal cord diseases, Stiff-Man Syndrome, mental retardation, nervous system abnormalities, nervous system neoplasms, peripheral nerve neoplasms, neurological manifestations, or neuromuscular disease.

More specifically, types of central nervous system infections that can be treated by neuropeptide receptor polynucleotides or polypeptides, or agonists or antagonists of neuropeptide receptor include, for example, AIDS Dementia Complex, brain abscesses, subdural empyema, encephalitis (e.g., encephalitis periaxialis, viral encephalitis, epidemic encephalitis, Japanese encephalitis, St. Louis, Tick-Borne, or West Nile Fever encephalitis), acute disseminated encephalomyelitis, meningoencephalitis (e.g., Uveomeningoencephalitic Syndrome), postencephalitic Parkinson Disease, subacute sclerosing panencephalitis, encephalomyelitis (e.g., equine encephalomyelitis or Venezuelan equine encephalomyelitis), necrotizing hemorrhagic encephalomyelitis, visna, cerebral malaria, meningitis (e.g., arachnoiditis, aseptic meningitis, or viral meningitis (e.g., lymphocytic choriomeningitis), bacterial meningitis (e.g., Haemophilus, Listeria, Meningococcal (e.g., Waterhouse-Friderichsen Syndrome), Pneumococcal, or meningeal tuberculosis), fungal meningitis (e.g., Cryptococcal), subdural effusion, meningoencephalitis (e.g., Uveomeningoencephalitic Syndrome), myelitis (e.g., transverse myelitis), neurosyphilis (e.g., tabes dorsalis), poliomyelitis (e.g., bulbar poliomyelitis or Postpoliomyelitis Syndrome), prion diseases (e.g., Creutzfeldt-Jakob Syndrome, bovine spongiform encephalopathy, Gerstmann-Straussler Syndrome, kuru, or scrapie) or cerebral toxoplasmosis.

Additionally, types of central nervous system neoplasms that can be treated by neuropeptide receptor polynucleotides or polypeptides, or agonists or antagonists of neuropeptide receptor include, for example, brain neoplasms (e.g., cerebellar neoplasms, infratentorial neoplasms, cerebral ventricle neoplasms, choroid plexus neoplasms, hypothalamic neoplasms, supratentorial neoplasms, meningeal neoplasms, or spinal cord neoplasms (erg., epidural neoplasms).

Moreover, types of demyelinating diseases that can be treated by neuropeptide receptor polynucleotides or polypeptides, or agonists or antagonists of neuropeptide receptor include, for example, Canavan Disease, diffuse cerebral sclerosis, adrenoleukodystrophy, encephalitis periaxialis, globoid cell leukodystrophy, diffuse cerebral sclerosis, metachromatic leukodystrophy, allergic encephalomyelitis, necrotizing hemorrhagic encephalomyelitis, progressive multifocal leukoencephalopathy, multiple sclerosis, central pontine myelinolysis, transverse myelitis, neuromyelitis optica, scrapie, or swayback.

In further embodiments, types of encephalomyelitis that can be treated by neuropeptide receptor polynucleotides or polypeptides, or agonists or antagonists of neuropeptide receptor include, for example, allergic, equine, or Venezuelan equine encephalomyelitis, necrotizing hemorrhagic encephalomyelitis, visna, or Chronic Fatigue Syndrome.

Additionally, types of spinal cord diseases that can be treated by neuropeptide receptor polynucleotides or polypeptides, or agonists or antagonists of neuropeptide receptor include, for example, amyotonia congenita, amyotrophic lateral sclerosis, spinal muscular atrophy, Werdnig-Hoffinann Disease, myelitis (e.g., transverse), poliomyelitis, (e.g., bulbar and Postpollomyelitis Syndrome), spinal cord compression, spinal cord neoplasms, epidural neoplasms, syringomyelia, or tabes dorsalis.

Moreover, types of mental retardation that can be treated by neuropeptide receptor polynucleotides or polypeptides, or agonists or antagonists of neuropeptide receptor include, for example, Angelman Syndrome, Cri-du-Chat Syndrome, De Lange's Syndrome, Down Syndrome, Gangliosidoses (e.g., GM1 gangliosidosis, Sandhoff Disease, Tay-Sachs Disease, Hartnup Disease, homocystinuria, Laurence-Moon-Biedl Syndrome, Lesch-Nyhan Syndrome, Maple Syrup Urine Disease, mucolipidosis, fucosidosis, neuronal ceroid-lipofuscinosis, Oculocerebrorenal Syndrome, phenylketonuria, phenylketonuria (e.g., maternal), Prader-WilH Syndrome, Rett Syndrome, Rubinstein-Taybi Syndrome, tuberous sclerosis, or WAGR Syndrome.

In further embodiments, types of nervous system abnormalities that can be treated by neuropeptide receptor polynucleotides or polypeptides, or agonists or antagonists of neuropeptide receptor include, for example, holoprosencephaly, neural tube defects (e.g., anencephaly, hydranencephaly, amold-chiad deformity, encephalocele, meningocele, meningomyelocele, spinal dysraphism (e.g., spina bifida cystica or spina bifida occulta)), hereditary motor and sensory neuropathies (e.g., Charcot-Marie Disease, hereditary optic atrophy, Refsum's Disease, hereditary spastic paraplegia, or Werdnig-Hoffmann Disease), hereditary sensory or autonomic neuropathies (e.g., congenital analgesia or familial dysautonomia).

Additionally, types of central nervous system neoplasms that can be treated by neuropeptide receptor polynucleotides or polypeptides, or agonists or antagonists of neuropeptide receptor include, for example, brain neoplasms (e.g., cerebellar neoplasms, infratentorial neoplasms, cerebral ventricle neoplasms, choroid plexus neoplasms, hypothalamic neoplasms or supratentorial neoplasms), meningeal neoplasms, spinal cord neoplasms (e.g., epidural neoplasms), peripheral nerve neoplasms (e.g., cranial nerve neoplasms, acoustic neuroma or neurofibromatosis 2).

Moreover, types of neurologic manifestations that can be treated by neuropeptide receptor polynucleotides or polypeptides, or agonists or antagonists of neuropeptide receptor include, for example, agnosia (e.g., Gerstmann's Syndrome), amnesia (e.g., retrograde), apraxia, neurogenic bladder, cataplexy, communicative disorders (e.g., hearing disorders such as deafness, partial hearing loss, loudness recruitment, or tinnitus), language disorders, aphasia (e.g., agraphia, anomia, broca aphasia, or Wernicke Aphasia), dyslexia, acquired dyslexia, language development disorders, speech disorders (e.g., aphasia, agraphia, anomia, broca aphasia, Wernicke Aphasia, articulation disorders, dysarthria, echolia, mutism, or stuttering) or voice disorders (e.g., aphonia, hoarseness)), decerebrate state, delirium, fasciculation, hallucinations, meningism, movement disorders (e.g., Angelman Syndrome, ataxia, athetosis, chorea, dystonia, hypokinesia, muscle hypotonia, myoclonus, tic, torticollis, or tremor), muscle hypertonia, muscle rigidity, Stiff-Man Syndrome, muscle spasticity, pain (e.g., arthralgia, back pain, facial pain, headache, tension headache, neuralgia, or intractable pain), paralysis, facial paralysis, herpes zoster oticus, gasftoparesis, hemiplegia, ophthalmoplegia (e.g., diplopia, Duane's Syndrome, Horner's Syndrome, chronic progressive external ophthalmoplegia, or Kearns Syndrome), paralysis (e.g., bulbar, tropical spastic paraparesis, paraplegia, Brown-Sequard Syndrome, quadriplegia, respiratory paralysis, or vocal cord paralysis), paresis, phantom limb, abnormal reflex, seizures, convulsions, sensation disorders (e.g., anosmia, dizziness, hallucinations, hyperesthesia, hyperalgesia, hypesthesia, illusions, paresthesia, restless legs, phantom limb, taste disorders (e.g., ageusia or dysgeusia), vision disorders (e.g., amblyopia, blindness, color vision defects, diplopia, hemianopsia, scotoma, or subnormal vision), sleep disorders (e.g., hypersomnia, Kleine-Levin Syndrome, narcolepsy, insomnia, or somnambulism), spasm, trismus, unconsciousness (e.g., coma, persistent vegetative state, or syncope), or vertigo.

Additionally, types of neuromuscular diseases that can be treated by neuropeptide receptor polynucleotides or polypeptides, or agonists or antagonists of neuropeptide receptor include, for example, amyotonia congenita, amyotrophic lateral sclerosis, Lambert-Eaton Myasthenic Syndrome, motor neuron disease, muscular atrophy (e.g., Charcot-Marie Disease, spinal muscular atrophy, or Werdnig-Hoffinann Disease), Postpoliomyelitis Syndrome, muscular dystrophy, myasthenia gravis, myotonia atrophica, myotonia congenita, nemaline myopathy, familial periodic paralysis, multiplex paramyoclonus, tropical spastic paraparesis, or Stiff-Man Syndrome.

Furthermore, nervous system diseases and disorders that can be treated by neuropeptide receptor polynucleotides or polypeptides, or agonists or antagonists of neuropeptide receptor include but are not limited to peripheral nervous system diseases such as acrodynia, amyloid neuropathies, autonomic nervous system diseases, cranial nervous system diseases, facial nerve disease, ocular motility disorders, optic nerve diseases, trigeminal neuralgia, vocal cor paralysis, demyelinating diseases, diabetic neuropathies, nerve compression syndromes, neuralgia, neuritis, hereditary motor and sensory neuropathies, hereditary sensory and autonomic neuropathies, or peripheral nerve neoplasms.

In further embodiments, types of autonomic nervous system diseases that can be treated by neuropeptide receptor polynucleotides or polypeptides, or agonists or antagonists of neuropeptide receptor include, for example, Adie's Syndrome, Barre-Lieou Syndrome, familial dysautonomia, Horner's Syndrome, reflex sympathetic dystrophy, or Shy-Drager Syndrome.

Additionally, types of cranial nerve diseases that can be treated by neuropeptide receptor polynucleotides or polypeptides, or agonists or antagonists of neuropeptide receptor include, for example, acoustic nerve diseases, acousitic neuroma, Neuroribromatosis 2, cranial nerve neoplasms, acoustic neuroma, or neurofibromatosis 2.

Moreover, types of facial nerve diseases that can be treated by neuropeptide receptor polynucleotides or polypeptides, or agonists or antagonists of neuropeptide receptor include, for example, facial neuralgia, facial paralysis (e.g., herpes zoster olticus or Melkersson-Rosenthal Syndrome) or ocular motility disorders (e.g., amblyopia, nystagmus, oculomotor nerve paralysis, ophthalmoplegia (e.g., Duane's Syndrome, Horner's Syndrome, chronic progressive external ophthalmoplegiaor, or Kearns Syndrome), strabismus, esotropia, or exotropia.

More specifically, types of optic nerve diseases that can be treated by neuropeptide receptor polynucleotides or polypeptides, or agonists or antagonists of neuropeptide receptor include, for example, optic atrophy, hereditary optic atrophy, optic disk drusen, optic neuritis, neuromyelitis optica, papilledema.

In further embodiments, types of demyelinating diseases that can be treated by neuropeptide receptor polynucleotides or polypeptides, or agonists or antagonists of neuropeptide receptor include, for example, neuromyelitis optica or swayback.

More specifically, types of nerve compression syndromes that can be treated by neuropeptide receptor polynucleotides or polypeptides, or agonists or antagonists of neuropeptide receptor include, for example, Carpal Tunnel Syndrome, Tarsal Tunnel Syndrome, Thoracic Outlet Syndrome, Cervical Rib Syndrome, and Ulnar Nerve Compression Syndrome.

Additionally, types of neuralgia that can be treated by neuropeptide receptor polynucleotides or polypeptides, or agonists or antagonists of neuropeptide receptor include, for example, causalgia, cervico-brachial neuralgia, facial neuralgia, or trigeminal neuralgia.

Moreover, types of neuritis that can be treated by neuropeptide receptor polynucleotides or polypeptides, or agonists or antagonists of neuropeptide receptor include, for example, experimental allergic neuritis, optic neuritis, polyneuritis, polyradiculoneuritis, radiculitis, or polyradiculitis.

In further embodiments, types of hereditary motor and sensory neuropathies that can be treated by neuropeptide receptor polynucleotides or polypeptides, or agonists or antagonists of neuropeptide receptor include, for example, Charcot-Marie Disease, hereditary optic atrophy, refsum's disease, hereditary spastic paraplegia, or Werdnig-Hoffmann Disease.

More specifically, types of hereditary sensory and autonomic neuropathies that can be treated by neuropeptide receptor polynucleotides or polypeptides, or agonists or antagonists of neuropeptide receptor include, for example, analgesia, congenital analgesia, or familial dysautonomia.

Additionally, types of peripheral nerve neoplasms that can be treated by neuropeptide receptor polynucleotides or polypeptides, or agonists or antagonists of neuropeptide receptor include, for example, cranial nerve neoplasms (acoustic neuroma or neurofibromatosis 2), POEMS Syndrome, sciatica, gustatory sweating, or tetany.

Anti-Angiogenesis Activity

The naturally occurring balance between endogenous stimulators and inhibitors of angiogenesis is one in which inhibitory influences predominate. Rastinejad et al., Cell 56:345-355 (1989). In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye disorders, and psoriasis. See, e.g., reviews by Moses et al., Biotech. 9:630-634 (1991); Folkman et al., N. Engl. J. Med., 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases or disorders associated with neovascularization by administration of the neuropeptide receptor polynucleotides and/or polypeptides of the invention, as well as agonists or antagonists of neuropeptide receptor. Malignant and metastatic conditions which can be treated with the polynucleotides and polypeptides, or agonists or antagonists of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)):

Ocular disorders associated with neovascularization which can be treated with the neuropeptide receptor polynucleotides and polypeptides of the present invention (including neuropeptide receptor agonists and/or antagonists) include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Additionally, disorders which can be treated with the neuropeptide receptor polynucleotides and polypeptides of the present invention (including neuropeptide receptor agonist and/or antagonists) include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, disorders and/or states, which can be treated with be treated with the neuropeptide receptor polynucleotides and polypeptides of the present invention (including neuropeptide receptor agonist and/or antagonists) include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

Diseases at the Cellular Level

Diseases associated with increased cell survival or the inhibition of apoptosis that could be treated or detected by neuropeptide receptor polynucleotides or polypeptides, as well as antagonists or agonists of neuropeptide receptor, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection. In preferred embodiments, neuropeptide receptor polynucleotides, polypeptides, and/or antagonists of the invention are used to inhibit growth, progression, and/or metasis of cancers, in particular those listed above.

Additional diseases or conditions associated with increased cell survival that could be treated or detected by neuropeptide receptor polynucleotides or polypeptides, or agonists or antagonists of neuropeptide receptor, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated or detected by neuropeptide receptor polynucleotides or polypeptides, as well as agonists or antagonists of neuropeptide receptor, include AIDS; neurodegenerative disorders (such as Alzheimer's disease, Parkinson's disease, Amyotrophic lateral sclerosis, Retinitis pigmentosa, Cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

Wound Healing and Epithelial Cell Proliferation

In accordance with yet a further aspect of the present invention, there is provided a process for utilizing neuropeptide receptor polynucleotides or polypeptides, as well as agonists or antagonists of neuropeptide receptor, for therapeutic purposes, for example, to stimulate epithelial cell proliferation and basal keratinocytes for the purpose of wound healing, and to stimulate hair follicle production and healing of dermal wounds. Neuropeptide receptor polynucleotides or polypeptides, as well as agonists or antagonists of neuropeptide receptor, may be clinically useful in stimulating wound healing including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from heat exposure or chemicals, and other abnormal wound healing conditions such as uremia, malnutrition, vitamin deficiencies and complications associated with systemic treatment with steroids, radiation therapy and antineoplastic drugs and antimetabolites. Neuropeptide receptor polynucleotides or polypeptides, as well as agonists or antagonists of neuropeptide receptor, could be used to promote dermal reestablishment subsequent to dermal loss.

Neuropeptide receptor polynucleotides or polypeptides, as well as agonists or antagonists of neuropeptide receptor, could be used to increase the adherence of skin grafts to a wound bed and to stimulate re-epithelialization from the wound bed. The following are types of grafts that neuropeptide receptor polynucleotides or polypeptides, agonists or antagonists of neuropeptide receptor, could be used to increase adherence to a wound bed: autografts, artificial skin, allografts, autodermic graft, autoepdermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft. Neuropeptide receptor polynucleotides or polypeptides, as well as agonists or antagonists of neuropeptide receptor, can be used to promote skin strength and to improve the appearance of aged skin.

It is believed that neuropeptide receptor polynucleotides or polypeptides, as well as agonists or antagonists of neuropeptide receptor, will also produce changes in hepatocyte proliferation, and epithelial cell proliferation in the lung, breast, pancreas, stomach, small intesting, and large intestine. Neuropeptide receptor polynucleotides or polypeptides, as well as agonists or antagonists of neuropeptide receptor, could promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. Neuropeptide receptor polynucleotides or polypeptides, agonists or antagonists of neuropeptide receptor, may promote proliferation of endothelial cells, keratinocytes, and basal keratinocytes.

Neuropeptide receptor polynucleotides or polypeptides, as well as agonists or antagonists of neuropeptide receptor, could also be used to reduce the side effects of gut toxicity that result from radiation, chemotherapy treatments or viral infections. Neuropeptide receptor polynucleotides or polypeptides, as well as agonists or antagonists of neuropeptide receptor, may have a cytoprotective effect on the small intestine mucosa. Neuropeptide receptor polynucleotides or polypeptides, as well as agonists or antagonists of neuropeptide receptor, may also stimulate healing of mucositis (mouth ulcers) that result from chemotherapy and viral infections.

Neuropeptide receptor polynucleotides or polypeptides, as well as agonists or antagonists of neuropeptide receptor, could further be used in full regeneration of skin in full and partial thickness skin defects, including burns, (i.e., repopulation of hair follicles, sweat glands, and sebaceous glands), treatment of other skin defects such as psoriasis. Neuropeptide receptor polynucleotides or polypeptides, as well as agonists or antagonists of neuropeptide receptor, could be used to treat epidermolysis bullosa, a defect in adherence of the epidermis to the underlying dermis which results in frequent, open and painful blisters by accelerating reepithelialization of these lesions. Neuropeptide receptor polynucleotides or polypeptides, as well as agonists or antagonists of neuropeptide receptor, could also be used to treat gastric and doudenal ulcers and help heal by scar formation of the mucosal lining and regeneration of glandular mucosa and duodenal mucosal lining more rapidly. Inflamamatory bowel diseases, such as Crohn's disease and ulcerative colitis, are diseases which result in destruction of the mucosal surface of the small or large intestine, respectively. Thus, neuropeptide receptor polynucleotides or polypeptides, as well as agonists or antagonists of neuropeptide receptor, could be used to promote the resurfacing of the mucosal surface to aid more rapid healing and to prevent progression of inflammatory bowel disease. Treatment with neuropeptide receptor polynucleotides or polypeptides, agonists or antagonists of neuropeptide receptor, is expected to have a significant effect on the production of mucus throughout the gastrointestinal tract and could be used to protect the intestinal mucosa from injurious substances that are ingested or following surgery. Neuropeptide receptor polynucleotides or polypeptides, as well as agonists or antagonists of neuropeptide receptor, could be used to treat diseases associate with the under expression of neuropeptide receptor.

Moreover, neuropeptide receptor polynucleotides or polypeptides, as well as agonists or antagonists of neuropeptide receptor, could be used to prevent and heal damage to the lungs due to various pathological states. A growth factor such as neuropeptide receptor polynucleotides or polypeptides, as well as agonists or antagonists of neuropeptide receptor, which could stimulate proliferation and differentiation and promote the repair of alveoli and brochiolar epithelium to prevent or treat acute or chronic lung damage. For example, emphysema, which results in the progressive loss of aveoli, and inhalation injuries, i.e., resulting from smoke inhalation and burns, that cause necrosis of the bronchiolar epithelium and alveoli could be effectively treated using neuropeptide receptor polynucleotides or polypeptides, agonists or antagonists of neuropeptide receptor. Also, neuropeptide receptor polynucleotides or polypeptides, as well as agonists or antagonists of neuropeptide receptor, could be used to stimulate the proliferation of and differentiation of type II pneumocytes, which may help treat or prevent disease such as hyaline membrane diseases, such as infant respiratory distress syndrome and bronchopulmonary displasia, in premature infants.

Neuropeptide receptor polynucleotides or polypeptides, as well as agonists or antagonists of neuropeptide receptor, could stimulate the proliferation and differentiation of hepatocytes and, thus, could be used to alleviate or treat liver diseases and pathologies such as fulminant liver failure caused by cirrhosis, liver damage caused by viral hepatitis and toxic substances (i.e., acetaminophen, carbon tetraholoride and other hepatotoxins known in the art).

In addition, neuropeptide receptor polynucleotides or polypeptides, as well as agonists or antagonists of neuropeptide receptor, could be used treat or prevent the onset of diabetes mellitus. In patients with newly diagnosed Types I and II diabetes, where some islet cell function remains, neuropeptide receptor polynucleotides or polypeptides, as well as agonists or antagonists of neuropeptide receptor, could be used to maintain the islet function so as to alleviate, delay or prevent permanent manifestation of the disease. Also, neuropeptide receptor polynucleotides or polypeptides, as well as agonists or antagonists of neuropeptide receptor, could be used as an auxiliary in islet cell transplantation to improve or promote islet cell function.

Infectious Disease

Neuropeptide receptor polynucleotides or polypeptides, or agonists or antagonists of neuropeptide receptor, can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, neuropeptide receptor polynucleotides or polypeptides, or agonists or antagonists of neuropeptide receptor, may also directly inhibit the infectious agent, without necessarily eliciting an immune response.

Viruses are one example of an infectious agent that can cause disease or symptoms that can be treated or detected by neuropeptide receptor polynucleotides or polypeptides, or agonists or antagonists of neuropeptide receptor. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. Neuropeptide receptor polynucleotides or polypeptides, or agonists or antagonists of neuropeptide receptor, can be used to treat or detect any of these symptoms or diseases.

Similarly, bacterial or fungal agents that can cause disease or symptoms and that can be treated or detected by neuropeptide receptor polynucleotides or polypeptides, or agonists or antagonists of neuropeptide receptor, include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. Neuropeptide receptor polynucleotides or polypeptides, or agonists or antagonists of neuropeptide receptor, can be used to treat or detect any of these symptoms or diseases.

Moreover, parasitic agents causing disease or symptoms that can be treated or detected by neuropeptide receptor polynucleotides or polypeptides, or agonists or antagonists of neuropeptide receptor, include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. Neuropeptide receptor polynucleotides or polypeptides, or agonists or antagonists of neuropeptide receptor, can be used to treat or detect any of these symptoms or diseases.

Preferably, treatment using neuropeptide receptor polynucleotides or polypeptides, or agonists or antagonists of neuropeptide receptor, could either be by administering an effective amount of neuropeptide receptor polypeptide to the patient, or by removing cells from the patient, supplying the cells with neuropeptide receptor polynucleotide, and returning the engineered cells to the patient (ex vivo therapy). Moreover, the neuropeptide receptor polypeptide or polynucleotide can be used as an antigen in a vaccine to raise an immune response against infectious disease.

Regeneration

Neuropeptide receptor polynucleotides or polypeptides, or agonists or antagonists of neuropeptide receptor, can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, neuropeptide receptor polynucleotides or polypeptides, or agonists or antagonists of neuropeptide receptor, may increase regeneration of tissues difficult to heal. For example, increased tendon/ligament regeneration would quicken recovery time after damage. Neuropeptide receptor polynucleotides or polypeptides, or agonists or antagonists of neuropeptide receptor, of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using neuropeptide receptor polynucleotides or polypeptides, or agonists or antagonists of neuropeptide receptor, to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the neuropeptide receptor polynucleotides or polypeptides, or agonists or antagonists of neuropeptide receptor.

Chemotaxis

Neuropeptide receptor polynucleotides or polypeptides, or agonists or antagonists of neuropeptide receptor, may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

Neuropeptide receptor polynucleotides or polypeptides, or agonists or antagonists of neuropeptide receptor, may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. As a chemotactic molecule, neuropeptide receptor could also attract fibroblasts, which can be used to treat wounds.

It is also contemplated that neuropeptide receptor polynucleotides or polypeptides, or agonists or antagonists of neuropeptide receptor, may inhibit chemotactic activity. These molecules could also be used to treat disorders. Thus, neuropeptide receptor polynucleotides or polypeptides, or agonists or antagonists of neuropeptide receptor, could be used as an inhibitor of chemotaxis.

Binding Activity

Neuropeptide receptor polypeptides may be used to screen for molecules that bind to neuropeptide receptor or for molecules to which neuropeptide receptor binds. The binding of neuropeptide receptor and the molecule may activate (agonist), increase, inhibit (antagonist), or decrease activity of the neuropeptide receptor or the molecule bound. Examples of such molecules include antibodies, oligonucleotides, proteins (e.g., receptors),or small molecules.

Preferably, the molecule is closely related to the natural ligand of neuropeptide receptor, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which neuropeptide receptor binds, or at least, a fragment of the receptor capable of being bound by neuropeptide receptor (e.g., active site). In either case, the molecule can be rationally designed using known techniques.

Preferably, the screening for these molecules involves producing appropriate cells which express neuropeptide receptor, either as a secreted protein or on the cell membrane. Preferred cells include cells from mammals, yeast, Drosophila, or E. coli. Cells expressing neuropeptide receptor(or cell membrane containing the expressed polypeptide) are then preferably contacted with a test compound potentially containing the molecule to observe binding, stimulation, or inhibition of activity of either neuropeptide receptor or the molecule.

The assay may simply test binding of a candidate compound to neuropeptide receptor, wherein binding is detected by a label, or in an assay involving competition with a labeled competitor. Further, the assay may test whether the candidate compound results in a signal generated by binding to neuropeptide receptor.

Alternatively, the assay can be carried out using cell-free preparations, polypeptide/molecule affixed to a solid support, chemical libraries, or natural product mixtures. The assay may also simply comprise the steps of mixing a candidate compound with a solution containing neuropeptide receptor, measuring neuropeptide receptor/molecule activity or binding, and comparing the neuropeptide receptor/molecule activity or binding to a standard.

Preferably, an ELISA assay can measure neuropeptide receptor level or activity in a sample (e.g., biological sample) using a monoclonal or polyclonal antibody. The antibody can measure neuropeptide receptor level or activity by either binding, directly or indirectly, to neuropeptide receptor or by competing with neuropeptide receptor for a substrate.

Additionally, the receptor to which neuropeptide receptor binds can be identified by numerous methods known to those of skill in the art, for example, ligand panning and FACS sorting (Coligan, et al., Current Protocols in Immun., 1(2), Chapter 5, (1991)). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labelled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling, and/or codon-shuffling (collectively referred to as “DNA shuffling”) may be employed to modulate the activities of neuropeptide receptor thereby effectively generating agonists and antagonists of neuropeptide receptor. See generally, U.S. Pat. Nos. 5,605,793, 5,811,238, 5,830,721, 5,834,252, and 5,837,458, and Patten, P. A., et al., Curr. Opinion Biotechnol. 8:724-33 (1997); Harayama, S. Trends Biotechnol. 16(2):76-82 (1998); Hansson, L. O., et al., J. Mol. Biol. 287:265-76 (1999); and Lorenzo, M. M. and Blasco, R. Biotechniques 24(2):308-13 (1998) (each of these patents and publications are hereby incorporated by reference). In one embodiment, alteration of neuropeptide receptor polynucleotides and corresponding polypeptides may be achieved by DNA shuffling. DNA shuffling involves the assembly of two or more DNA segments into a desired neuropeptide receptor molecule by homologous, or site-specific, recombination. In another embodiment, neuropeptide receptor polynucleotides and corresponding polypeptides may be alterred by being subjected to random mutagenesis by error-prone PCR, random nucleotide insertion or other methods prior to recombination. In another embodiment, one or more components, motifs, sections, parts, domains, fragments, etc., of neuropeptide receptor may be recombined with one or more components, motifs, sections, parts, domains, fragments, etc. of one or more heterologous molecules. In preferred embodiments, the heterologous molecules are neuropeptide receptor family members. In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic(dpp), 60A, OP-2, dorsalin, growth differentiation factors (GDFs), nodal, MIS, inhibin-alpha, TGF-betal, TGF-beta2, TGF-beta3, TGF-beta5, and glial-derived neurotrophic factor (GDNF).

Other preferred fragments are biologically active neuropeptide receptor fragments. Biologically active fragments are those exhibiting activity similar, but not necessarily identical, to an activity of the neuropeptide receptor polypeptide. The biological activity of the fragments may include an improved desired activity, or a decreased undesirable activity.

Additionally, this invention provides a method of screening compounds to identify those which modulate the action of the polypeptide of the present invention. An example of such an assay comprises combining a mammalian fibroblast cell, a the polypeptide of the present invention, the compound to be screened and ³[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of ³[H] thymidine in each case. The amount of fibroblast cell proliferation is measured by liquid scintillation chromatography which measures the incorporation of ³[H] thymidine. Both agonist and antagonist compounds may be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the neuropeptide receptor receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat disease or to bring about a particular result in a patient (e.g., blood vessel growth) by activating or inhibiting the neuropeptide receptor/molecule. Moreover, the assays can discover agents which may inhibit or enhance the production of neuropeptide receptor from suitably manipulated cells or tissues.

Therefore, the invention includes a method of identifying compounds which bind to neuropeptide receptor comprising the steps of: (a) incubating a candidate binding compound with neuropeptide receptor; and (b) determining if binding has occurred. Moreover, the invention includes a method of identifying agonists/antagonists comprising the steps of: (a) incubating a candidate compound with neuropeptide receptor, (b) assaying a biological activity, and (b) determining if a biological activity of neuropeptide receptor has been altered.

Also, one could identify molecules bind neuropeptide receptor experimentally by using the beta-pleated sheet regions disclosed in FIG. 8 and Table 1. Accordingly, specific embodiments of the invention are directed to polynucleotides encoding polypeptides which comprise, or alternatively consist of, the amino acid sequence of each beta pleated sheet regions disclosed in FIG. 8/Table 1. Additional embodiments of the invention are directed to polynucleotides encoding neuropeptide receptor polypeptides which comprise, or alternatively consist of, any combination or all of the beta pleated sheet regions disclosed in FIG. 8/Table 1. Additional preferred embodiments of the invention are directed to polypeptides which comprise, or alternatively consist of, the neuropeptide receptor amino acid sequence of each of the beta pleated sheet regions disclosed in FIG. 8/Table 1. Additional embodiments of the invention are directed to neuropeptide receptor polypeptides which comprise, or alternatively consist of, any combination or all of the beta pleated sheet regions disclosed in FIG. 8/Table

Antisense And Ribozyme (Antagonists)

In specific embodiments, antagonists according to the present invention are nucleic acids corresponding to the sequences contained in SEQ ID NO:1, or the complementary strand thereof, and/or to nucleotide sequences contained in the deposited clone 97128. In one embodiment, antisense sequence is generated internally by the organism, in another embodiment, the antisense sequence is separately administered (see, for example, O'Connor, J., Neurochem. 56:560 (1991). Oligodeoxynucleotides as Anitsense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J., Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the neuropeptide receptor antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the neuropeptide receptor antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding neuropeptide receptor, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster, et al., Nature 296:39-42 (1982)), etc.

The antisense nucleic acids of the invention comprise a sequence complementary to at least a portion of an RNA transcript of a neuropeptide receptor gene. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded neuropeptide receptor antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a neuropeptide receptor RNA it may contain and still form a stable duplex (or triplex as the case may be). One skilled in the art can ascertain a tolerable degree of mismatch by use of standard procedures to determine the melting point of the hybridized complex.

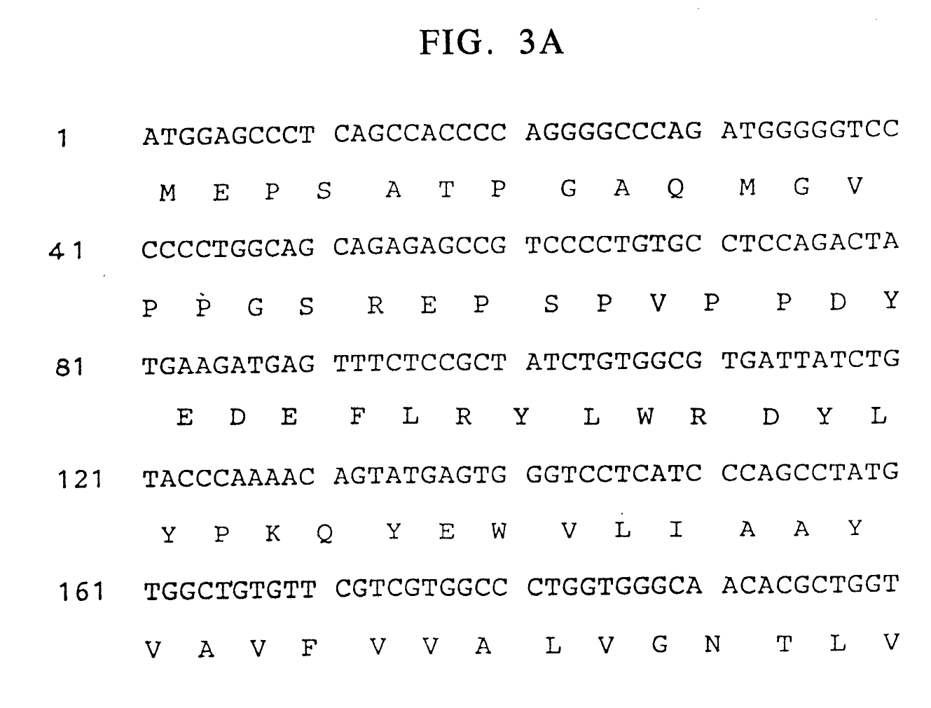

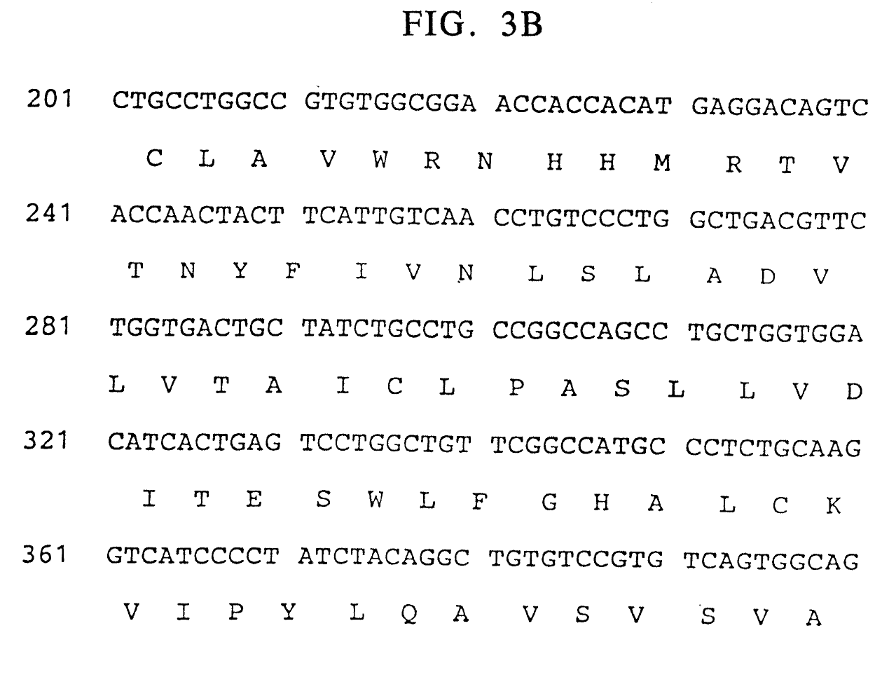

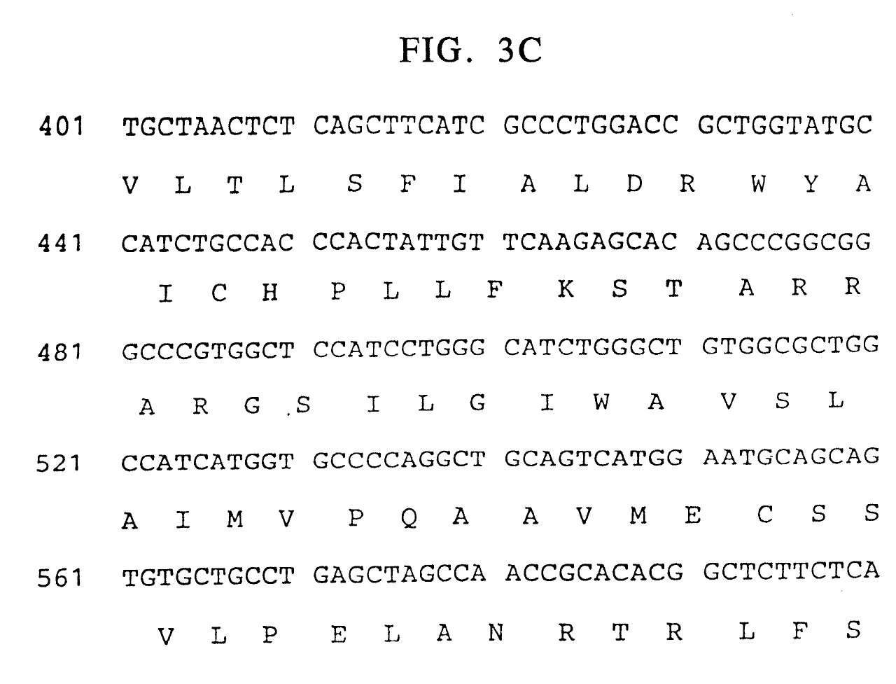

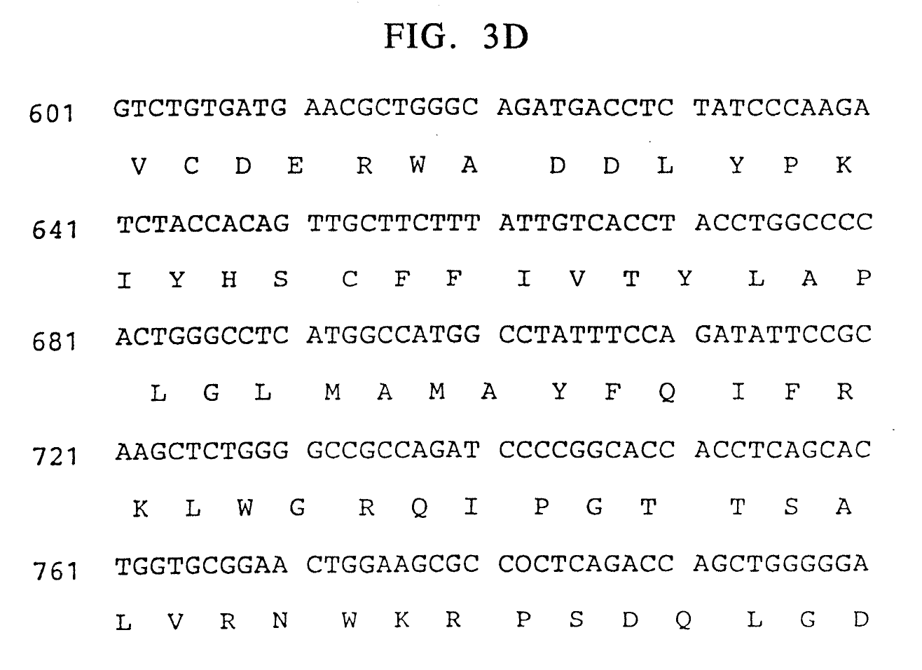

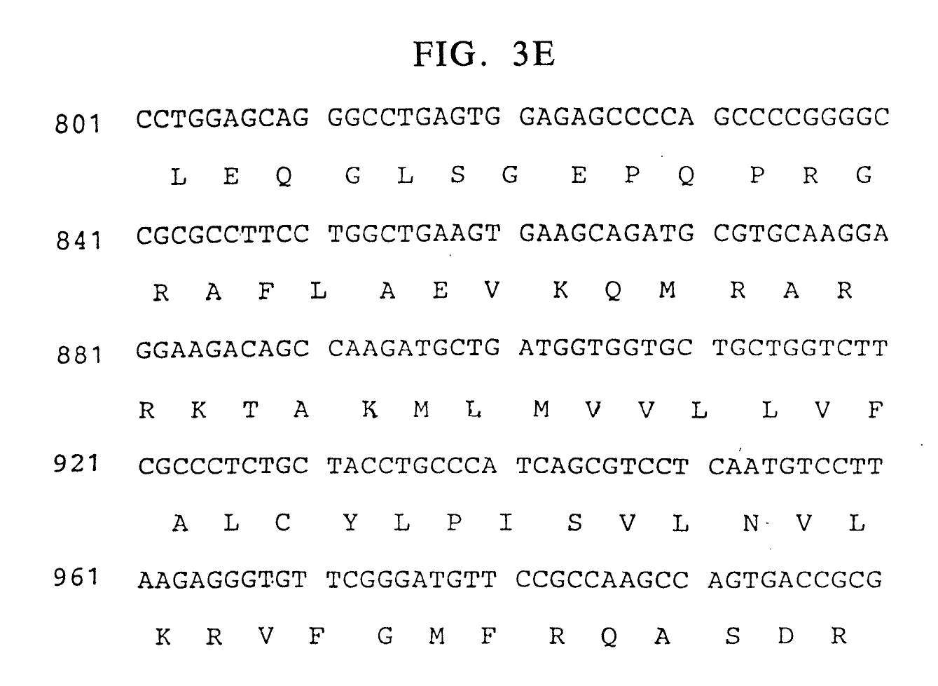

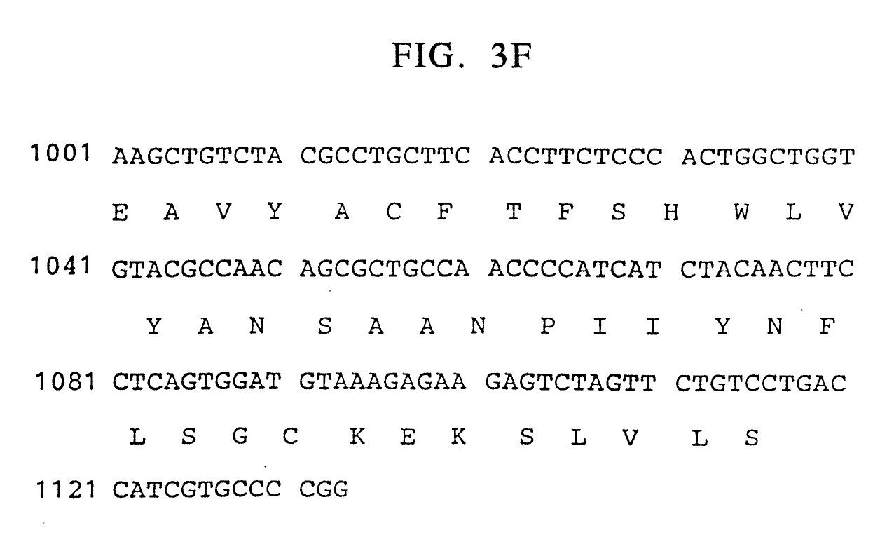

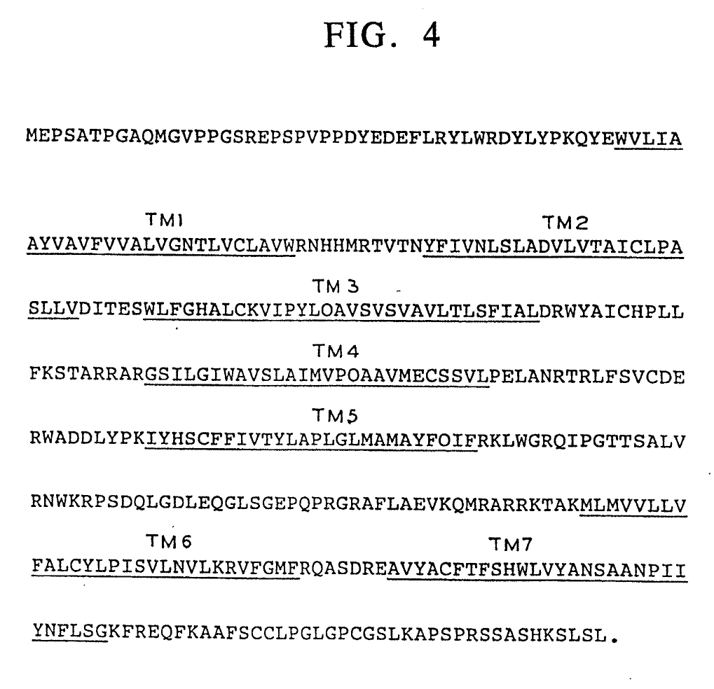

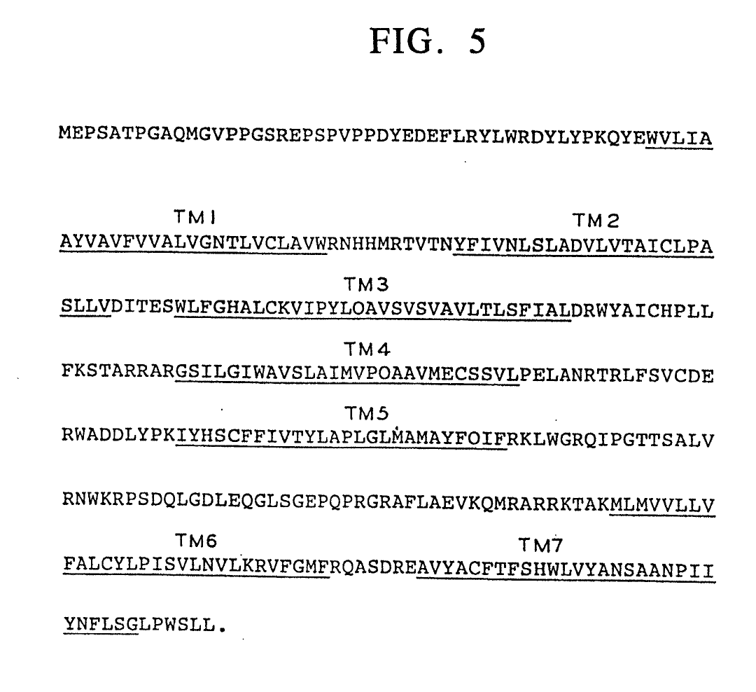

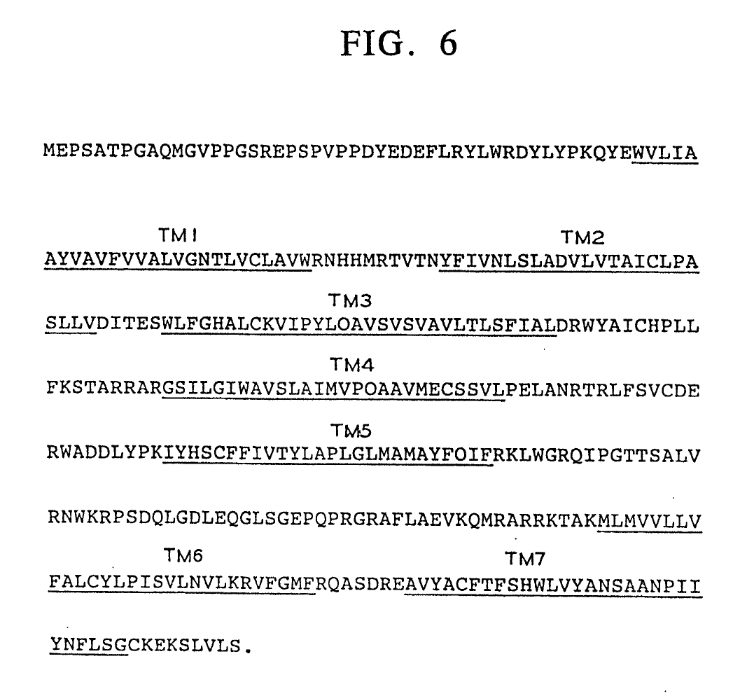

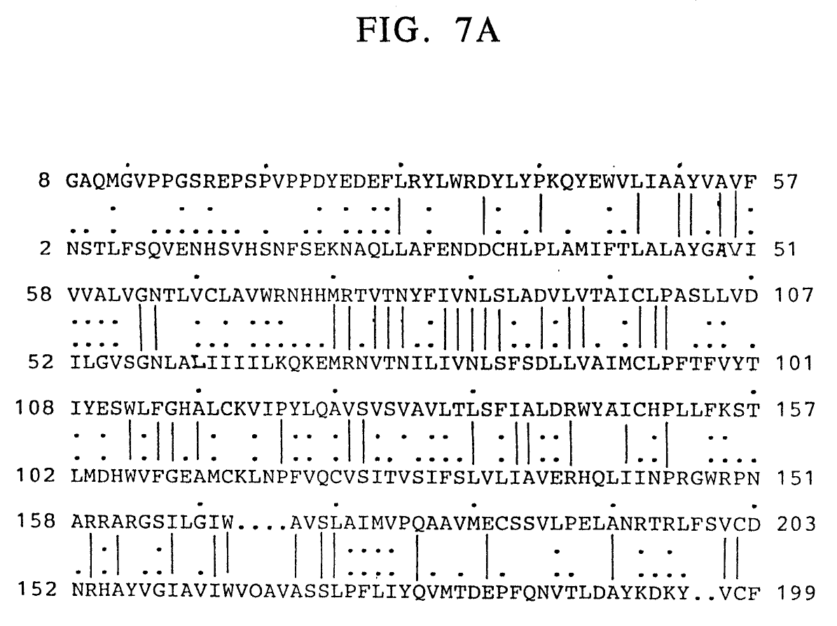

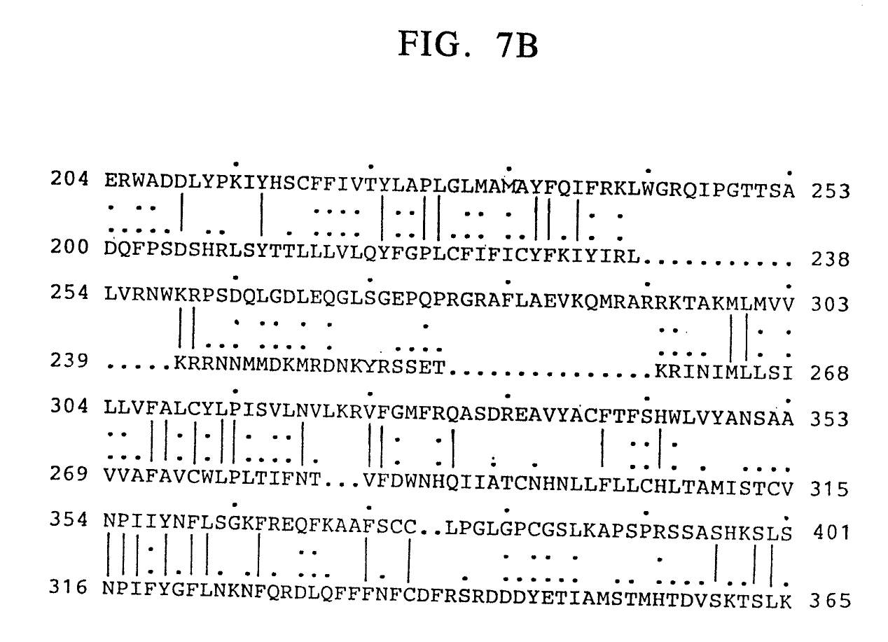

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′- non-translated, non-coding regions of neuropeptide receptor shown in FIGS. 1-3 could be used in an antisense approach to inhibit translation of endogenous neuropeptide receptor mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of neuropeptide receptor mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An a-anomeric oligonucleotide forms specific double-stranded hybrids with complementary RNA in which, contrary to the usual b-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the neuropeptide receptor coding region sequence could be used, those complementary to the transcribed untranslated region are most preferred.

Potential antagonists according to the invention also include catalytic RNA, or a ribozyme (See, e.g., PCT International Publication WO 90/11364, published Oct. 4, 1990; Sarver et al, Science 247:1222-1225 (1990). While ribozymes that cleave mRNA at site specific recognition sequences can be used to destroy neuropeptide receptor mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of neuropeptide receptor (FIGS. 1-3). Preferably, the ribozyme is engineered so that the cleavage recognition site is located near the 5′ end of the neuropeptide receptor mRNA; i.e., to increase efficiency and minimize the intracellular accumulation of non-functional mRNA transcripts.

As in the antisense approach, the ribozymes of the invention can be composed of modified oligonucleotides (e.g for improved stability, targeting, etc.) and should be delivered to cells which express neuropeptide receptor in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous neuropeptide receptor messages and inhibit translation. Since ribozymes unlike antisense molecules, are catalytic, a lower intracellular concentration is required for efficiency.

Antagonist/agonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat the diseases described herein.

Other Activities

The polypeptide of the present invention, as a result of the ability to stimulate vascular endothelial cell growth, may be employed in treatment for stimulating re-vascularization of ischemic tissues due to various disease conditions such as thrombosis, arteriosclerosis, and other cardiovascular conditions. These polypeptide may also be employed to stimulate angiogenesis and limb regeneration, as discussed above.

The polypeptide may also be employed for treating wounds due to injuries, burns, post-operative tissue repair, and ulcers since they are mitogenic to various cells of different origins, such as fibroblast cells and skeletal muscle cells, and therefore, facilitate the repair or replacement of damaged or diseased tissue.

The polypeptide of the present invention may also be employed stimulate neuronal growth and to treat and prevent neuronal damage which occurs in certain neuronal disorders or neuro-degenerative conditions such as Alzheimer's disease, Parkinson's disease, and AIDS-related complex. Neuropeptide receptor may have the ability to stimulate chondrocyte growth, therefore, they may be employed to enhance bone and periodontal regeneration and aid in tissue transplants or bone grafts.

The polypeptide of the present invention may be also be employed to prevent skin aging due to sunburn by stimulating keratinocyte growth.

The neuropeptide receptor polypeptide may also be employed for preventing hair loss, since FGF family members activate hair-forming cells and promotes melanocyte growth. Along the same lines, the polypeptides of the present invention may be employed to stimulate growth and differentiation of hematopoietic cells and bone marrow cells when used in combination with other cytokines.

The neuropeptide receptor polypeptide may also be employed to maintain organs before transplantation or for supporting cell culture of primary tissues.

The polypeptide of the present invention may also be employed for inducing tissue of mesodermal origin to differentiate in early embryos.

Neuropeptide receptor polynucleotides or polypeptides, or agonists or antagonists of neuropeptide receptor, may also increase or decrease the differentiation or proliferation of embryonic stem cells, besides, as discussed above, hematopoietic lineage.

Neuropeptide receptor polynucleotides or polypeptides, or agonists or antagonists of neuropeptide receptor, may also be used to modulate mammalian characteristics, such as body height, weight, hair color, eye color, skin, percentage of adipose tissue, pigmentation, size, and shape (e.g., cosmetic surgery). Similarly, neuropeptide receptor polynucleotides or polypeptides, or agonists or antagonists of neuropeptide receptor, may be used to modulate mammalian metabolism affecting catabolism, anabolism, processing, utilization, and storage of energy.

Neuropeptide receptor polynucleotides or polypeptides, or agonists or antagonists of neuropeptide receptor, may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

Neuropeptide receptor polynucleotides or polypeptides, or agonists or antagonists of neuropeptide receptor, may be used to treat narcolepsy and/or other sleep disorders in humans and other animals.

Neuropeptide receptor polynucleotides or polypeptides, or agonists or antagonists of neuropeptide receptor, may be used to treat osteoporesis, bulimia, acute heart failure, asthma, allergies, benign prostatic hypertrophy, osteoarthritis, nerve damage, pain, paralysis, and facial palsy.

Neuropeptide receptor polynucleotides or polypeptides, or agonists or antagonists of neuropeptide receptor, may also be used as a food additive or preservative, such as to increase or decrease storage capabilities, fat content, lipid, protein, carbohydrate, vitamins, minerals, cofactors or other nutritional components.

The compounds which inhibit activation of the neuropeptide receptor polypeptides of the present invention may be employed to treat and/or prevent hypertension since neuropeptide Y stimulates renin release and neuropeptide Y is known to have potent vasoconstrictor activity when involving the coronary and cerebral vessels.

The compounds may also be employed to treat Alzheimer's disease since neuropeptide Y receptors are prevalent in the central nervous system and are localized predominantly within intemeurons where they appear to have regulatory roles in memory and Alzheimers disease.

The compounds may also be employed to suppress excitatory transmission by neuropeptide Y in the hippocampus and therefore may be employed to treat epileptic seizure, stress and anxiety.

The prevalence of neuropeptide Y receptors in the central nervous system indicates that the compounds which inhibit the neuropeptide receptor polypeptides of the present invention may be used as an antipsychotic drug by regulating neurotransmission.

The compounds which inhibit the receptor polypeptides of the present invention may also be employed to treat pathological vasospasm involving coronary and cerebral vessels.

This invention also provides a method for determining whether a ligand not known to be capable of binding to a neuropeptide receptor of the present invention can bind thereto which comprises contacting the ligand to be identified with a cell comprising the coding sequence of a neuropeptide receptor and expressing same on its surface under conditions sufficient for binding of ligands previously identified as binding to such a receptor. In other embodiments cell membrane fractions comprising the receptor or isolated receptors free or immobilized on solid supports may be used to measure binding of the ligand to be tested. When recombinant cells are used for purposes of expression of the receptor it is preferred to use cells with little or no endogenous receptor activity so that binding, if any, is due to the presence of the expressed receptor of interest. Preferred cells include human embryonic kidney cells, monkey kidney (HEK-293 cells), fibroblast (COS) cells, Chinese hamster ovary (CHO) cells, Drosophila or murine L-cells. It is also preferred to employ as a host cell, one in which a receptor responsive second messenger system exists. Well known second messenger systems include increases or decreases in phosphoinositide hydrolysis, adenylate cyclase, guanylate cyclase, or ion channel activity in response to ligand binding to extracellular receptor domains. In a further embodiment a specifically designed indicator of receptor binding can be constructed. For example, a fusion protein can be made by fusing the receptor of this invention with a protein domain which is sensitive to receptor ligand binding. Such a domain referred to here as an indicator domain is capable, itself, or in association with accessory molecules, of generating an analytically detectable signal which is indicative or receptor ligand binding.

This invention also provides a method of detecting expression of a neuropeptide receptor polypeptide of the present invention on the surface of a cell by detecting the presence of mRNA coding for the receptor which comprises obtaining total mRNA from the cell and contacting the mRNA so obtained with a nucleic acid probe comprising a nucleic acid molecule of at least 10 nucleotides capable of specifically hybridizing with a sequence included within the sequence of a nucleic acid molecule encoding the receptor under hybridizing conditions, detecting the presence of mRNA hybridized to the probe, and thereby detecting the expression of the receptor by the cell.

The present invention also provides a method for identifying receptors related to the receptor polypeptides of the present invention. These related receptors may be identified by homology to a neuropeptide receptor polypeptide of the present invention, by low stringency cross hybridization, or by identifying receptors that interact with related natural or synthetic ligands and or elicit similar behaviors after genetic or pharmacological blockade of the neuropeptide receptor polypeptides of the present invention.

Fragments of the genes may be used as a hybridization probe for a cDNA library to isolate other genes which have a high sequence similarity to the genes of the present invention, or which have similar biological activity. Probes of this type preferably have 50 bases or more. The probe may also be used to identify a cDNA clone corresponding to a full length transcript and a genomic clone or clones that contain the complete gene of the present invention including regulatory and promoter regions, exons and introns. An example of a screen of this type comprises isolating the coding region of the gene by using the known DNA sequence to synthesize an oligonucleotide probe. Labeled oligonucleotides having a sequence complementary to that of the genes of the present invention are used to screen a library of human cDNA, genomic DNA or mRNA to determine which members of the library the probe hybridizes to.

The soluble neuropeptide receptor polypeptides and compounds which bind to and activate or inhibit activation of a receptor of the present invention may also be employed in combination with a suitable pharmaceutical carrier. Such compositions comprise a therapeutically effective amount of the soluble neuropeptide receptor polypeptide or compounds, and a pharmaceutically acceptable carrier or excipient. Such a carrier includes but is not limited to saline, buffered saline, dextrose, water, glycerol, ethanol, and combinations thereof. The formulation should suit the mode of administration.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the soluble neuropeptide receptor polypeptides or compounds of the present invention may be employed in conjunction with other therapeutic compounds.

The pharmaceutical compositions may be administered in a convenient manner such as by the topical, intravenous, intraperitoneal, intramuscular, subcutaneous, intranasal or intradermal routes. The pharmaceutical compositions are administered in an amount which is effective for treating and/or prophylaxis of the specific indication. In general, the pharmaceutical compositions will be administered in an amount of at least about 10 g/kg body weight and in most cases they will be administered in an amount not in excess of about 8 mg/Kg body weight per day. In most cases, the dosage is from about 10 g/kg to about 1 mg/kg body weight daily, taking into account the routes of administration, symptoms, etc.

The present invention also contemplates the use of the genes of the present invention as a diagnostic, for example, some diseases result from inherited defective genes. These genes can be detected by comparing the sequences of the defective gene with that of a normal one. Subsequently, one can verify that a “mutant” gene is associated with abnormal receptor activity. In addition, one can insert mutant receptor genes into a suitable vector for expression in a functional assay system (e.g., colorimetric assay, expression on MacConkey plates, complementation experiments, in a receptor deficient strain of HEK293 cells) as yet another means to verify or identify mutations. Once “mutant” genes have been identified, one can then screen population for carriers of the “mutant” receptor gene.

Individuals carrying mutations in the gene of the present invention may be detected at the DNA level by a variety of techniques. Nucleic acids used for diagnosis may be obtained from a patient's cells, including but not limited to such as from blood, urine, saliva, tissue biopsy and autopsy material. The genomic DNA may be used directly for detection or may be amplified enzymatically by using PCR (Saiki, et al., Nature, 324:163-166 1986) prior to analysis. RNA or cDNA may also be used for the same purpose. As an example, PCR primers complimentary to the nucleic acid of the instant invention can be used to identify and analyze mutations in the gene of the present invention. For example, deletions and insertions can be detected by a change in size of the amplified product in comparison to the normal genotype. Point mutations can be identified by hybridizing amplified DNA to radio labeled RNA of the invention or alternatively, radio labeled antisense DNA sequences of the invention. Perfectly matched sequences can be distinguished from mismatched duplexes by RNase A digestion or by differences in melting temperatures. Such a diagnostic would be particularly useful for prenatal or even neonatal testing.

Sequence differences between the reference gene and “mutants” may be revealed by the direct DNA sequencing method. In addition, cloned DNA segments may be used as probes to detect specific DNA segments. The sensitivity of this method is greatly enhanced when combined with PCR. For example, a sequence primer is used with double stranded PCR product or a single stranded template molecule generated by a modified PCR. The sequence determination is performed by conventional procedures with radio labeled nucleotide or by an automatic sequencing procedure with fluorescent-tags.

Genetic testing based on DNA sequence differences may be achieved by detection of alterations in the electrophoretic mobility of DNA fragments in gels with or without denaturing agents. Sequences changes at specific locations may also be revealed by nucleus protection assays, such RNase and S1 protection or the chemical cleavage method (e.g. Cotton, et al., PNAS, USA, 85:4397-4401 1985).

In addition, some diseases are a result of, or are characterized by changes in gene expression which can be detected by changes in the mRNA. Alternatively, the genes of the present invention can be used as a reference to identify individuals expressing a decrease of functions associated with receptors of this type.

The present invention also relates to a diagnostic assay for detecting altered levels of soluble forms of the neuropeptide receptor polypeptides of the present invention in various tissues. Assays used to detect levels of the soluble receptor polypeptides in a sample derived from a host are well known to those of skill in the art and include radioimmunoassays, competitive-binding assays, Western blot analysis and preferably as ELISA assay.

An ELISA assay initially comprises preparing an antibody specific to antigens of the neuropeptide receptor polypeptides, preferably a monoclonal antibody. In addition a reporter antibody is prepared against the monoclonal antibody. To the reporter antibody is attached a detectable reagent such as radioactivity, fluorescence or in this example a horseradish peroxidase enzyme. A sample is now removed from a host and incubated on a solid support, e.g. a polystyrene dish, that binds the proteins in the sample. Any free protein binding sites on the dish are then covered by incubating with a non-specific protein such as bovine serum albumin. Next, the monoclonal antibody is incubated in the dish during which time the monoclonal antibodies attach to any neuropeptide receptor proteins attached to the polystyrene dish. All unbound monoclonal antibody is washed out with buffer. The reporter antibody linked to horseradish peroxidase is now placed in the dish resulting in binding of the reporter antibody to any monoclonal antibody bound to neuropeptide receptor proteins. Unattached reporter antibody is then washed out. Peroxidase substrates are then added to the dish and the amount of color developed in a given time period is a measurement of the amount of neuropeptide receptor proteins present in a given volume of patient sample when compared against a standard curve.

The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers (preferably 15-25 bp) from the cDNA. Computer analysis of the 3′ untranslated region is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 50 or 60 bases. For a review of this technique, see Verma et al., Human Chromosomes: a Manual of Basic Techniques, Pergamon Press, New York (1988).

The above techniques were utilized to map the gene corresponding to the neuropeptide receptor of the present invention to.

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

Next, it is necessary to determine the differences in the cDNA or genomnic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

The polypeptides, their fragments or other derivatives, or analogs thereof, or cells expressing them can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

The present invention will be further described with reference to the following examples; however, it is to be understood that the present invention is not limited to such examples. All parts or amounts, unless otherwise specified, are by weight.

In order to facilitate understanding of the following examples certain frequently occurring methods and/or terms will be described.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 pg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37 C. are ordinarily used, but may vary in accordance with the supplier's instructions. After digestion the reaction is electrophoresed directly on a polyacrylamide gel to isolate the desired fragment.

Size separation of the cleaved fragments is performed using 8 percent polyacrylamide gel described by Goeddel, D. et al., Nucleic Acids Res., 8:4057 (1980).

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Ligation” refers to the process of forming phosphodiester bonds between two double stranded nucleic acid fragments (Maniatis, T., et al., Id., p. 146). Unless otherwise provided, ligation may be accomplished using known buffers and conditions with 10 units to T4 DNA ligase (“ligase”) per 0.5 μg of approximately equimolar amounts of the DNA fragments to be ligated.

Unless otherwise stated, transformation was performed as described in the method of Graham, F. and Van der Eb, A., Virology, 52:456-457 (1973).

The above-recited applications have uses in a wide variety of hosts. Such hosts include, but are not limited to, human, murine, rabbit, goat, guinea pig, camel, horse, mouse, rat, hamster, pig, micro-pig, chicken, goat, cow, sheep, dog, cat, non-human primate, and human. In specific embodiments, the host is a mouse, rabbit, goat, guinea pig, chicken, rat, hamster, pig, sheep, dog or cat. In preferred embodiments, the host is a mammal. In most preferred embodiments, the host is a human.

Having generally described the invention, the same will be more readily understood by reference to the following examples, which are provided by way of illustration and are not intended as limiting.

Having generally described the invention, the same will be more readily understood by reference to the following examples, which are provided by way of illustration and are not intended as limiting.

### EXAMPLES

### Example 1

### Isolation of the Neuropeptide Receptor cDNA Clone From the Deposited Sample

Two approaches can be used to isolate neuropeptide receptor from the deposited sample. First, the deposited clone (HFGAN72) is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. A single colony is then used to generate DNA using nucleic acid isolation techniques well known to those skilled in the art. (e.g., Sambrook et al., Molecular Cloning: A Laboratory Manual, 2nd Edit., (1989), Cold Spring Harbor Laboratory Press.)

Alternatively, two primers of 17-20 nucleotides derived from both ends of the SEQ ID NO:1 (i.e., within the region of SEQ ID NO:1 bounded by the 5′ NT and the 3′ NT of the clone) are synthesized and used to amplify the neuropeptide receptor cDNA using the deposited cDNA plasmid as a template. The polymerase chain reaction is carried out under routine conditions, for instance, in 25 ul of reaction mixture with 0.5 ug of the above cDNA template. A convenient reaction mixture is 1.5-5 mM MgCl₂, 0.01% (w/v) gelatin, 20 uM each of dATP, dCTP, dGTP, dTTP, 25 pmol of each primer and 0.25 Unit of Taq polymerase. Thirty five cycles of PCR (denaturation at 94 degree C. for 1 min; annealing at 55 degree C. for 1 min; elongation at 72 degree C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the neuropeptide receptor gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).)

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the neuropeptide receptor gene of interest is used to PCR amplify the 5′ portion of the neuropeptide receptor full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the neuropeptide receptor gene.

### Example 2

### Isolation of Neuropeptide Receptor Genomic Clones

A human genomic P1 library (Genomic Systems, Inc.) is screened by PCR using primers selected for the cDNA sequence corresponding to SEQ ID NO:1., according to the method described in Example 1. (See also, Sambrook.)

### Example 3

### Tissue Distribution of Neuropeptide Receptor Polypeptides

Tissue distribution of mRNA expression of neuropeptide receptor is determined using protocols for Northern blot analysis, described by, among others, Sambrook et al. For example, a neuropeptide receptor probe produced by the method described in Example 1 is labeled with P³² using the rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70 degree C. overnight, and the films developed according to standard procedures.

### Example 4

### Chromosomal Mapping of Neuropeptide Receptor

An oligonucleotide primer set is designed according to the sequence at the 5′ end of SEQ ID NO:1. This primer preferably spans about 100 nucleotides. This primer set is then used in a polymerase chain reaction under the following set of conditions: 30 seconds, 95 degree C.; 1 minute, 56 degree C.; 1 minute, 70 degree C. This cycle is repeated 32 times followed by one 5 minute cycle at 70 degree C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of Neuropeptide Receptor

Neuropeptide receptor polynucleotide encoding a neuropeptide receptor polypeptide invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacd repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. WPTG induces by inactivating the lacd repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified neuropeptide receptor protein is then renatured by dialyzing it against phosphate-buffered saline (PBS) or 50 mM Na-acetate, pH 6 buffer plus 200 mM NaCl. Alternatively, the neuropeptide receptor protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified neuropeptide receptor protein is stored at 4 degree C. or frozen at −80 degree C.

In addition to the above expression vector, the present invention further includes an expression vector comprising phage operator and promoter elements operatively linked to a neuropeptide receptor polynucleotide, called pHE4a. (ATCC Accession Number 209645, deposited Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (laclq). The origin of replication (oriC) is derived from pUC 19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

Alteratively, the DNA sequence encoding for neuropeptide receptor, ATCC No. 97128 is initially amplified using PCR oligonucleotide primers corresponding to the 5′ and 3′ end sequences of the processed neuropeptide receptor gene (minus the signal peptide sequence) and the vector sequences 3′ to the gene. Additional nucleotides corresponding to neuropeptide receptor nucleotide sequence are added to the 5′ and 3′ sequences respectively. The 5′ oligonucleotide primer has the sequence 5′ CACTAAAGCTTAATGGAGCCCTCAGCCACC 3′ (SEQ ID NO:7) contains a Hind III restriction enzyme site followed by 18 nucleotides of neuropeptide receptor coding sequence starting from the presumed terminal amino acid of the processed protein codon. The 3′ sequence 5′ ACAAGTCCTTGTCCTTCTAGAGGGC 3′ (SEQ ID NO:8) and contains an XbaI site. The restriction enzyme sites correspond to the restriction enzyme sites on the bacterial expression vector pQE-9 (Qiagen, Inc. Chatsworth, Calif.). pQE-9 encodes antibiotic resistance (Amp^{r}), a bacterial origin of replication (ori), an IPTG-regulatable promoter operator (P/O), a ribosome binding site (RBS), a 6-His tag and restriction enzyme sites. pQE-9 is then digested with Hind III and XbaI. The amplified sequences are ligated into pQE-9 and are inserted in frame with the sequence encoding for the histidine tag and the RBS. The ligation mixture is then used to transform E. coli strain M15/rep 4 (Qiagen, Inc.) by the procedure described in Sambrook, J. et al., Molecular Cloning: A Laboratory Manual, Cold Spring Laboratory Press, (1989). M15/rep4 contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis. Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (“Isopropyl-B-D-thiogalacto pyranoside”) is then added to a final concentration of 1 mM. IPTG induces by inactivating the lacI repressor, clearing the P/O leading to increased gene expression. Cells are grown an extra 3 to 4 hours. Cells are then harvested by centrifugation. The cell pellet is solubilized in the chaotropic agent 6 Molar Guanidine HCl. After clarification, solubilized neuropeptide receptor is purified from this solution by chromatography on a Nickel-Chelate column under conditions that allow for tight binding by proteins containing the 6-His tag (Hochuli, E. et al., J. Chromatography 411:177-184 (1984). The protein is eluted from the column in 6 molar guanidine HCl pH 5.0 and for the purpose of renaturation adjusted to 3 molar guanidine HCl, 100 mM sodium phosphate, 10 mmolar glutathione (reduced) and 2 mmolar glutathione (oxidized). After incubation in this solution for 12 hours the protein is dialyzed to 10 mmolar sodium phosphate.

### Example 6

### Purification of Neuropeptide Receptor Polypeptide From an Inclusion Body

The following alternative method can be used to purify neuropeptide receptor polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the neuropeptide receptor polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant neuropeptide receptor polypeptide should exhibit greater than 95% purity after the above refolding and purification steps. No major contaminant bands should be observed from Commassie blue stained 16% SDS-PAGE gel when 5 ug of purified protein is loaded. The purified neuropeptide receptor protein can also be tested for endotoxin/LPS contamination, and typically the LPS content is less than 0.1 ng/ml according to LAL assays.

### Example 7

### Cloning and Expression of Neuropeptide Receptor in a Baculovirus Expression System

In this example, the plasmid shuttle vector pA2 is used to insert neuropeptide receptor polynucleotide into a baculovirus to express neuropeptide receptor. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned neuropeptide receptor polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the neuropeptide receptor cDNA sequence contained in the deposited clone, including the AUG initiation codon and any naturally associated leader sequence, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transfected with 1.0 ug of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One ug of BaculoGoldT virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of ³⁵S-methionine and 5 uCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced neuropeptide receptor protein.

Alternatively, the DNA sequence encoding the full length neuropeptide receptor protein, ATCC No. 97128, is amplified using PCR oligonucleotide primers corresponding to the 5′ and 3′ sequences of the gene:

The 5′ primer has the sequence 5′CGGGATCCGCCATCATGGAGCCCTCAGCCACC 3′ (SEQ ID NO:11) and contains a BamHI restriction enzyme site (in bold) followed by 6 nucleotides resembling an efficient signal for the initiation of translation in eukaryotic cells (J. Mol. Biol. 1987, 196, 947-950, Kozak, M.). The initiation codon for translation “ATG” is underlined).

The 3′ primer has the sequence 5′ ACAAGTCCTTGTCCTTCTAGAGGGC 3′ (SEQ ID NO: 12) and contains the cleavage site for the restriction endonuclease XbaI and 5 nucleotides complementary to the 3′ non-translated sequence of the neuropeptide receptor gene. The amplified sequences are isolated from a 1% agarose gel using a commercially available kit (“Geneclean,” BIO 101 Inc., La Jolla, Calif.). The fragment is then digested with the endonucleases BamHI and XbaI and then purified as described in Example 1. This fragment is designated F2.

The vector pA2 (modification of pVL941 vector, discussed below) is used for the expression of the neuropeptide receptor protein using the baculovirus expression system (for review see: Summers, M. D. and Smith, G. E. 1987, A manual of methods for baculovirus vectors and insect cell culture procedures, Texas Agricultural Experimental Station Bulletin NO:1, 3 and 5555). This expression vector contains the strong polyhedrin promoter of the Autographa californica nuclear polyhidrosis virus (AcMNPV) followed by the recognition sites for the restriction endonucleases BamHI and XbaI. The polyadenylation site of the simian virus (SV)40 is used for efficient polyadenylation. For an easy selection of recombinant viruses the beta-galactosidase gene from E. coli is inserted in the same orientation as the polyhedrin promoter followed by the polyadenylation signal of the polyhedrin gene. The polyhedrin sequences are flanked at both sides by viral sequences for the cell-mediated homologous recombination of co-transfected wild-type viral DNA. Many other baculovirus vectors could be used in place of pRG1 such as pAc373, pVL941 and pAcIM1 (Luckow, V. A. and Summers, M. D., Virology, 170:31-39).

The plasmid is digested with the restriction enzymes BamHI and XbaI and then dephosphorylated using calf intestinal phosphatase by procedures known in the art. The DNA is then isolated from a 1% agarose gel as described in Example 1. This vector DNA is designated V2.

Fragment F2 and the dephosphorylated plasmid V2 are ligated with T4 DNA ligase. DH5 alpha are then transformed and bacteria identified that contained the plasmid (pBac neuropeptide receptor) with the neuropeptide receptor gene using the enzymes BamHI and XbaI. The sequence of the cloned fragment is confirmed by DNA sequencing.

5 μg of the plasmid pBac neuropeptide receptor are co-transfected with 1.0 μg of a commercially available linearized baculovirus (“BaculoGold baculovirus DNA”, Pharmingen, San Diego, Calif.) using the lipofection method (Felgner et al. Proc. Natl. Acad. Sci. USA, 84:7413-7417 (1987)).

1 μg of BaculoGold virus DNA and 5 μg of the plasmid pBac neuropeptide receptor are mixed in a sterile well of a microtiter plate containing 50 μl of serum free Grace's medium (Life Technologies Inc., Gaithersburg, Md.). Afterwards 10 μl Lipofectin plus 90 μl Grace's medium are added, mixed and incubated for 15 minutes at room temperature. Then the transfection mixture is added drop wise to the Sf9 insect cells (ATCC CRL 1711) seeded in a 35 mm tissue culture plate with 1 ml Grace' medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation continued at 27° C. for four days.

After four days the supernatant is collected and a plaque assay performed similar as described by Summers and Smith (supra). As a modification an agarose gel with “Blue Gal” (Life Technologies Inc., Gaithersburg) is used which allows an easy isolation of blue stained plaques. (A detailed description of a “plaque assay” can also be found in the user's guide for insect cell culture and baculovirology distributed by Life Technologies Inc., Gaithersburg, page 9-10).

Four days after the serial dilution the virus is added to the cells and blue stained plaques are picked with the tip of an Eppendorf pipette. The agar containing the recombinant viruses is then resuspended in an Eppendorf tube containing 200 μl of Grace's medium. The agar is removed by a brief centrifugation and the supernatant containing the recombinant baculoviruses is used to infect Sf9 cells seeded in 35 mm dishes. Four days later the supernatants of these culture dishes are harvested and then stored at 4° C.

Sf9 cells are grown in Grace's medium supplemented with 10% heat-inactivated FBS. The cells are infected with the recombinant baculovirus V-neuropeptide receptor at a multiplicity of infection (MOI) of 2. Six hours later the medium is removed and replaced with SF900 II medium minus methionine and cysteine (Life Technologies Inc., Gaithersburg). 42 hours later 5 μCi of ³⁵S-methionine and 5 μCi ³⁵S cysteine (Amersham) are added. The cells are further incubated for 16 hours before they are harvested by centrifugation and the labelled proteins visualized by SDS-PAGE and autoradiography.

### Example 8

### Expression of Neuropeptide Receptor in Mammalian Cells

Neuropeptide receptor polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2DHFR (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CVI, quail QCI-3 cells, mouse L cells and Chinese hamster ovary (CHO) cells.

Alternatively, neuropeptide receptor polypeptide can be expressed in stable cell lines containing the neuropeptide receptor polynucleotide integrated into a chromosome. The co-transfection with a selectable marker such as DHFR, gpt, neomycin, hygromycin allows the identification and isolation of the transfected cells.

The transfected neuropeptide receptor gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-DHFR (ATCC Accession No. 37146), the expression vectors pC4 (ATCC Accession No. 209646) and pC6 (ATCC Accession No. 209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of neuropeptide receptor. The vectors also contain the 3′ intron, the polyadenylation and termination signal of the rat preproinsulin gene, and the mouse DHFR gene under control of the SV40 early promoter.

If a naturally occurring signal sequence is used to produce a secreted protein, the vector does not need a second signal peptide. Alternatively, if a naturally occurring signal sequence is not used, the vector can be modified to include a heterologous signal sequence in an effort to secrete the protein from the cell. (See, e.g., WO 96/34891.)

The amplified fragment is then digested with the appropriate restriction enzyme and purified on a 1% agarose gel using a commercially available kit (“Geneclean,” BIO 101 Inc., La Jolla, Calif.). The isolated fragment and the dephosphorylated vector are then ligated with T4 DNA ligase. E. coli HB101 or XL-1 Blue cells are then transformed and bacteria are identified that contain the fragment inserted into plasmid pC6 or pC4 using, for instance, restriction enzyme analysis.

Chinese hamster ovary cells lacking an active DHFR gene is used for transfection. Five μg of the expression plasmid pC6 or pC4 is cotransfected with 0.5 ug of the plasmid pSVnvco using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of neuropeptide receptor is analyzed, for instance, by SDS-PAGE and Western blot or by reversed phase HPLC analysis.

Alternatively, the expression of plasmid, neuropeptide receptor HA is derived from a vector pcDNA3/Amp (Invitrogen) containing: 1) SV40 origin of replication, 2) ampicillin resistance gene, 3) E. coli replication origin, 4) CMV promoter followed by a polylinker region, a SV40 intron and polyadenylation site. A DNA fragment encoding the entire neuropeptide receptor precursor and a HA tag fused in frame to its 3′ end is cloned into the polylinker region of the vector, therefore, the recombinant protein expression is directed under the CMV promoter. The HA tag corresponds to an epitope derived from the influenza hemagglutinin protein as previously described (I. Wilson, H. Niman, R. Heighten, A Cherenson, M. Connolly, and R. Lerner, 1984, Cell 37, 767). The infusion of HA tag to the target protein allows easy detection of the recombinant protein with an antibody that recognizes the HA epitope.

The plasmid construction strategy is described as follows:

The DNA sequence encoding for neuropeptide receptor, ATCC No. 97128, is constructed by PCR using two primers: the 5′ primer 5′ CCTAGGATGCCCCTCTGCTGCAGCGG 3′ (SEQ ID NO:9) contains a BamHI site; the 3′ sequence 5′ ACAAGTCCTTGTCCTTCTAGAGGGC 3′ (SEQ ID NO:10) contains complementary sequences to an XbaI site, translation stop codon, and the last 17 nucleotides of the neuropeptide receptor coding sequence (not including the stop codon). Therefore, the PCR product contains a BaniHI site, coding sequence, a translation termination stop codon and an XbaI site. The PCR amplified DNA fragment and the vector, pcDNA3/Amp, are digested with BamHI and XbaI restriction enzymes and ligated. The ligation mixture is transformed into E. coli strain SURE (Stratagene Cloning Systems, La Jolla, Calif.) the transformed culture is plated on ampicillin media plates and resistant colonies are selected. Plasmid DNA is isolated from transformants and examined by restriction analysis for the presence of the correct fragment. For expression of the recombinant neuropeptide receptor, COS cells are transfected with the expression vector by DEAE-DEXTRAN method (J. Sambrook, E. Fritsch, T. Maniatis, Molecular Cloning: A Laboratory Manual, Cold Spring Laboratory Press, (1989)). The expression of the neuropeptide receptor HA protein is detected by radio-labelling and immunoprecipitation method (E. Harlow, D. Lane, Antibodies: A Laboratory Manual, Cold Spring Harbor Laboratory Press, (1988)). Cells are labelled for 8 hours with ³⁵S-cysteine two days post transfection. Culture media are then collected and cells are lysed with detergent (RIPA buffer (150 mM NaCl, 1% NP-40, 0.1% SDS, 1% NP-40, 0.5% DOC, 50 mM Tris, pH 7.5). (Wilson, I. et al., Id. 37:767 (1984)). Both cell lysate and culture media are precipitated with a HA specific monoclonal antibody. Proteins precipitated are analyzed on 15% SDS-PAGE gels.

### Example 9

### Construction of N-Terminal and/or C-Terminal Deletion Mutants

The following general approach may be used to clone a N-terminal or C-terminal deletion neuropeptide receptor deletion mutant. Generally, two oligonucleotide primers of about 15-25 nucleotides are derived from the desired 5′ and 3′ positions of a polynucleotide of SEQ ID NO:1. The 5′ and 3′ positions of the primers are determined based on the desired neuropeptide receptor polynucleotide fragment. An initiation and stop codon are added to the 5′ and 3′ primers respectively, if necessary, to express the neuropeptide receptor polypeptide fragment encoded by the polynucleotide fragment. Preferred neuropeptide receptor polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the neuropeptide receptor polynucleotide fragment in a desired vector may also be added to the 5′ and 3′ primer sequences. The neuropeptide receptor polynucleotide fragment is amplified from genomic DNA or from the deposited cDNA clone using the appropriate PCR oligonucleotide primers and conditions discussed herein or known in the art. The neuropeptide receptor polypeptide fragments encoded by the neuropeptide receptor polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the neuropeptide receptor polypeptide fragment S-17 to L-380 is amplified and cloned as follows: A 5′ primer is generated comprising a restriction enzyme site followed by an initiation codon in frame with the polynucleotide sequence encoding the N-terminal portion of the polypeptide fragment beginning with S-17. A complementary 3′ primer is generated comprising a restriction enzyme site followed by a stop codon in frame with the polynucleotide sequence encoding C-terminal portion of the neuropeptide receptor polypeptide fragment ending with L-380.

The amplified polynucleotide fragment and the expression vector are digested with restriction enzymes which recognize the sites in the primers. The digested polynucleotides are then ligated together. The neuropeptide receptor polynucleotide fragment is inserted into the restricted expression vector, preferably in a manner which places the neuropeptide receptor polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 10

### Protein Fusions of Neuropeptide Receptor

Neuropeptide receptor polypeptides are preferably fused to other proteins. These fusion proteins can be used for a variety of applications. For example, fusion of neuropeptide receptor polypeptides to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to neuropeptide receptor polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and neuropeptide receptor polynucleotide, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc Region

| | |
|---|---|
| GGGATCCGGAGCCCAAATCTTCTGACAAAACTCACACATGCCCACCGTGCCCAGCA | (SEQ ID NO: 13) |
| CCTGAATTCGAGGGTGCACCGTCAGTCTTCCTCTTCCCCCCAAAACCCAAGGACAC | |
| CCTCATGATCTCCCGGACTCCTGAGGTCACATGCGTGGTGGTGGACGTAAGCCACG | |
| AAGACCCTGAGGTCAAGTTCAACTGGTACGTGGACGGCGTGGAGGTGCATAATGCC | |
| AAGACAAAGCCGCGGGAGGAGCAGTACAACAGCACGTACCGTGTGGTCAGCGTCCT | |
| CACCGTCCTGCACCAGGACTGGCTGAATGGCAAGGAGTACAAGTGCAAGGTCTCCA | |
| ACAAAGCCCTCCCAACCCCCATCGAGAAAACCATCTCCAAAGCCAAAGGGCAGCCC | |
| CGAGAACCACAGGTGTACACCCTGCCCCCATCCCGGGATGAGCTGACCAAGAACCA | |
| GGTCAGCCTGACCTGCCTGGTCAAAGGCTTCTATCCAAGCGACATCGCCGTGGAGT | |
| GGGAGAGCAATGGGCAGCCGGAGAACAACTACAAGACCACGCCTCCCGTGCTGGAC | |
| TCCGACGGCTCCTTCTTCCTCTACAGCAAGCTCACCGTGGACAAGAGCAGGTGGCA | |
| GCAGGGGAACGTCTTCTCATGCTCCGTGATGCATGAGGCTCTGCACAACCACTACA | |
| CGCAGAAGAGCCTCTCCCTGTCTCCGGGTAAATGAGTGCGACGGCCGCGACTCTAG | |
| AGGAT | |

### Example 11

### Production of an Antibody

a) Hybridoma Technology

The antibodies of the present invention can be prepared by a variety of methods. (See, Current Protocols, Chapter 2.) As one example of such methods, cells expressing neuropeptide receptor is administered to an animal to induce the production of sera containing polyclonal antibodies. In a preferred method, a preparation of neuropeptide receptor protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with neuropeptide receptor polypeptide or, more preferably, with a secreted neuropeptide receptor polypeptide-expressing cell. Such cells may be cultured in any suitable tissue culture medium; however, it is preferable to culture cells in Earle's modified Eagle's medium supplemented with 10% fetal bovine serum (inactivated at about 56 degree C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the neuropeptide receptor polypeptide.

Alternatively, additional antibodies capable of binding to neuropeptide receptor polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the neuropeptide receptor protein-specific antibody can be blocked byneuropeptide receptor. Such antibodies comprise anti-idiotypic antibodies to the neuropeptide receptor protein-specific antibody and can be used to immunize an animal to induce formation of further neuropeptide receptor protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted neuropeptide receptor protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation of Antibody Fragments Directed Against Neuropeptide Receptor From a Library of scFvs

Naturally occurring V-genes isolated from human PBLs are constructed into a large library of antibody fragments which contain reactivities against neuropeptide receptor to which the donor may or may not have been exposed (see e.g., U.S. Pat. No. 5,885,793 incorporated herein in its entirety by reference).

Rescue of the Library. A library of scFvs is constructed from the RNA of human PBLs as described in WO92/01047. To rescue phage displaying antibody fragments, approximately 10⁹ E. coli harbouring the phagemid are used to inoculate 50 ml of 2×TY containing 1% glucose and 100 ug/ml of ampicillin (2×TY-AMP-GLU) and grown to an O.D. of 0.8 with shaking. Five ml of this culture is used to innoculate 50 ml of 2×TY-AMP-GLU, 2×10⁸ TU of delta gene 3 helper (M13 delta gene III, see WO92/01047) are added and the culture incubated at 37 degree C. for 45 minutes without shaking and then at 37 degree C. for 45 minutes with shaking. The culture is centrifuged at 4000 r.p.m. for 10 min. and the pellet resuspended in 2 liters of of 2×TY containing 100 ug/ml ampicillin and 50 ug/ml kanamycin and grown overnight. Phage are prepared as described in WO92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harbouring a pUC19 derivative supplying the wild type gene III protein during phage morphogenesis. The culture is incubated for 1 hour at 37 degree C. without shaking and then for a further hour at 37 degree C. with shaking. Cells are spun down (PEC-Centra 8, 4000 revs/min for 10 min), resuspended in 300 ml 2×TY broth containing 100 ug ampicillin/ml and 25 ug kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 um filter (Minisart NML; Sartorius) to give a final concentration of approximately 10¹³ transducing units/ml (ampicillin-resistant clones).

Panning of the Library. Immunotubes (Nunc) are coated overnight in PBS with 4 ml of either 100 ug/ml or 10 ug/ml of a polypeptide of the present invention. Tubes are blocked with 2% Marvel-PBS for 2 hours at 37 degree C. and then washed 3 times in PBS. Approximately 10¹³ TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.OM Tris-HCl, pH 7.4. Phage are then used to infect 10 ml of mid-log E. coli TG1 by incubating eluted phage with bacteria for 30 minutes at 37 degree C. The E. coli are then plated on TYE plates containing 1% glucose and 100 ug/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 MM bicarbonate pH 9.6. Clones positive in ELISA are further characterized by PCR fingerprinting (see e.g., WO92/01047) and then by sequencing.

### Example 12

### Production Of Neuropeptide Receptor Protein For High-Throughput Screening Assays

The following protocol produces a supernatant containing neuropeptide receptor polypeptide to be tested. This supernatant can then be used in the Screening Assays described in Examples 14-21.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5ml DMEM(Dulbecco's Modified Eagle Medium) (with 4.5 G/L glucose and L-glutamine (12-604F Biowhittaker))/10% heat inactivated FBS(14-503F Biowhittaker)/1×Penstrep(17-602E Biowhittaker). Let the cells grow overnight.

The next day, mix together in a sterile solution basin: 300 ul Lipofectamine (18324-012 Gibco/BRL) and 5ml Optimem 1 (31985070 Gibco/BRL)/96-well plate. With a small volume multi-channel pipetter, aliquot approximately 2 ug of an expression vector containing a polynucleotide insert, produced by the methods described in Examples 8-10, into an appropriately labeled 96-well round bottom plate. With a multi-channel pipetter, add 50 ul of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a12-channel pipetter with tips on every other channel, adds the 200 ul of DNA/Lipofectamine/Optimem I complex to the odd wells first, then to the even wells, to each row on the 24-well plates. Incubate at 37 degree C. for 6 hours.

While cells are incubating, prepare appropriate media, either 1%BSA in DMEM with 1×penstrep, or HGS CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄-H₂O; 71.02 mg/L of Na₂HPO4; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L- Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B ₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and Ix penstrep. (BSA (81-068-3 Bayer) 100 gm dissolved in 1L DMEM for a 10% BSA stock solution). Filter the media and collect 50 ul for endotoxin assay in 15ml polystyrene conical.

The transfection reaction is terminated, preferably by tag-teaming, at the end of the incubation period. Person A aspirates off the transfection media, while person B adds 1.5 ml appropriate media to each well. Incubate at 37 degree C. for 45 or 72 hours depending on the media used: 1% BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 14-21.

It is specifically understood that when activity is obtained in any of the assays described below using a supernatant, the activity originates from either the neuropeptide receptor polypeptide directly (e.g., as a secreted protein) or by neuropeptide receptor inducing expression of other proteins, which are then secreted into the supernatant. Thus, the invention further provides a method of identifying the protein in the supernatant characterized by an activity in a particular assay.

### Example 13

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jakl, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and L-10. The Class 1 receptors share a conserved cysteine motif (a set of four conserved cysteines and one tryptophan) and a WSXWS motif (a membrane proximal region encoding Trp-Ser-Xxx-Trp-Ser (SEQ ID NO:14)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | | JAKs | | | | | |
|---|---|---|---|---|---|---|---|
| Ligand | | tyk2 | Jak1 | Jak2 | Jak3 | STATS | GAS(elements) or ISRE |
| IFN family | | | | | | | |
| IFN-a/B | + | + | − | − | 1,2,3 | | ISRE |
| IFN-g | | | + | + | − | 1 | GAS (IRF1>Lys6>IFP) |
| I1-10 | | + | ? | ? | − | 1,3 | |
| gp130 family | | | | | | | |
| IL-6 (Pleiotrohic) | + | + | + | ? | 1,3 | | GAS (IRF1>Lys6>IFP) |
| I1-11(Pleiotrohic) | ? | + | ? | ? | 1,3 | | |
| OnM(Pleiotrohic) | ? | + | + | ? | 1,3 | | |
| LIF(Pleiotrohic) | ? | + | + | ? | 1,3 | | |
| CNTF(Pleiotrohic) | −/+ | + | + | ? | 1,3 | | |
| G-CSF(Pleiotrohic) | | ? | + | ? | ? | 1,3 | |
| IL-12(Pleiotrohic) | + | − | + | + | 1,3 | | |
| g-C family | | | | | | | |
| IL-2 (lymphocytes) | | − | + | − | + | 1,3,5 | GAS |
| IL-4 (lymph/myeloid) | | − | + | − | + | 6 | GAS (IRF1 = IFP>>Ly6)(IgH) |
| IL-7 (lymphocytes) | | − | + | − | + | 5 | GAS |
| IL-9 (lymphocytes) | | − | + | − | + | 5 | GAS |
| IL-13 (lymphocyte) | | − | + | ? | ? | 6 | GAS |
| IL-15 | | ? | + | ? | + | 5 | GAS |
| gp140 family | | | | | | | |
| IL-3 (myeloid) | | − | − | + | − | 5 | GAS (IRF1>IFP>>Ly6) |
| IL-5 (myeloid) | | − | − | + | − | 5 | GAS |
| GM-CSF (myeloid) | | − | − | + | − | 5 | GAS |
| Growth hormone family | | | | | | | |
| GH | | ? | − | + | − | 5 | |
| PRL | | ? | +/− | + | − | 1,3,5 | |
| EPO | | ? | − | + | − | 5 | GAS(B-CAS>IRF1=IFP>>Ly6) |
| Receptor Tyrosine Kinases | | | | | | | |
| EGF | | ? | + | + | − | 1,3 | GAS(IRF1) |
| PDGF | | ? | + | + | − | 1,3 | |
| CSF-1 | | ? | + | + | − | 1,3 | GAS (not IRF1) |

To construct a synthetic GAS containing promoter element, which is used in the Biological Assays described in Examples 14-15, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

| | |
|---|---|
| 5′:GCGCCTCGAGATTTCCCCGAAATCTAGATTTCCCCGAAATGATTTCCCCGAAATG | (SEQ ID NO: 15) |
| ATTTCCCCGAAATATCTGCCATCTCAATTAG:3′ | |

The downstream primer is complementary to the SV40 promoter and is flanked with a Hind III site: 5′:GCGGCAAGCTTTTTGCAAAGCCTAGGC:3′ (SEQ ID NO:16)

PCR amplification is performed using the SV40 promoter template present in the B-gal:promoter plasmid obtained from Clontech. The resulting PCR fragment is digested with XhoI/Hind III and subcloned into BLSK2-. (Stratagene.) Sequencing with forward and reverse primers confirms that the insert contains the following sequence:

| | |
|---|---|
| 5′:CTCGAGATTTCCCCGAAATCTAGATTTCCCCGAAATGATTTCCCCGAAATGATTTC | (SEQ ID NO: 17) |
| CCCGAAATATCTGCCATCTCAATTAGTCAGCAACCATAGTCCCGCCCCTAACTCCGCCC | |
| ATCCCGCCCCTAACTCCGCCCAGTTCCGCCCATTCTCCGCCCCATGGCTGACTAATTTT | |
| TTTTATTTATGCAGAGGCCGAGGCCGCCTCGGCCTCTGAGCTATTCCAGAAGTAGTGAG | |
| GAGGCTTTTTTGGAGGCCTAGGCTTTTGCAAAAAGCTT:3′ | |

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 14-15.

Other constructs can be made using the above description and replacing GAS with a different promoter sequence. For example, construction of reporter molecules containing NFK-B and EGR promoter sequences are described in Examples 16 and 17. However, many other promoters can be substituted using the protocols described in these Examples. For instance, SRE, IL-2, NFAT, or Osteocalcin promoters can be substituted, alone or in combination (e.g., GAS/NF-KB/EGR, GAS/NF-KB, I1-2/NFAT, or NF-KB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 14

### High-Throughput Screening Assay for T-cell Activity

The following protocol is used to assess T-cell activity of neuropeptide receptor by determining whether neuropeptide receptor supernatant proliferates and/or differentiates T-cells. T-cell activity is assessed using the GAS/SEAP/Neo construct produced in Example 13. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI+10% serum with 1% Pen-Strep. Combine 2.5 mis of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37 degree C. for 6 hrs. After the incubation, add 10 ml of RPMI +15% serum.

The Jurkat:GAS-SEAP stable reporter lines are maintained in RPMI +10% serum, 1 mg/ml Genticin, and 1% Pen-Strep. These cells are treated with supernatants containing neuropeptide receptor polypeptides or neuropeptide receptor induced polypeptides as produced by the protocol described in Example 12.

On the day of treatment with the supernatant, the cells should be washed and resuspended in fresh RPMI +10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100, 000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H 10, and H11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using sellophene covers) and stored at −20 degree C. until SEAP assays are performed according to Example 18. The plates containing the remaining treated cells are placed at 4 degree C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 15

### High-Throughput Screening Assay Identifying Myeloid Activity

The following protocol is used to assess myeloid activity of neuropeptide receptor by determining whether neuropeptide receptor proliferates and/or differentiates myeloid cells. Myeloid cell activity is assessed using the GAS/SEAP/Neo construct produced in Example 13. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 13, a DEAE-Dextran method (Kharbanda et. al., 1994, Cell Growth & Differentiation, 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄.7H₂O, 1 mM MgCl₂, and 675 uM CaCl₂. Incubate at 37 degree C. for 45 min.

Wash the cells with RPMI 1640 medium containing 10% FBS and then resuspend in 10 ml complete medium and incubate at 37 degree C. for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 12. Incubate at 37 degree C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 18.

### Example 16

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGRI promoter linked to reporter molecules, activation of cells can be assessed by neuropeptide receptor.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC 12 cells by neuropeptide receptor can be assessed.

The EGR/SEAP reporter construct can be assembled by the following protocol. The EGR-1 promoter sequence (−633 to +1) (Sakamoto K et al., Oncogene 6:867-871 (1991)) can be PCR amplified from human genomic DNA using the following primers:

| | |
|---|---|
| 5′GCGCTCGAGGGATGACAGCGATAGAACCCCGG-3′ | (SEQ ID NO: 18) |
| 5′GCGAAGCTTCGCGACTCCCCGGATCCGCCTC-3′ | (SEQ ID NO: 19) |

Using the GAS:SEAP/Neo vector produced in Example 13, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR 1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. #12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 12. EGR-SEAP/PC12 stable cells are obtained by growing the cells in 300 ug/ml G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 12, 37 degree C. for 48 to 72 hr. As a positive control, a growth factor known to activate PC12 cells through EGR can be used, such as 50 ng/ul of Neuronal Growth Factor (NGF). Over fifty-fold induction of SEAP is typically seen in the positive control wells. SEAP assay the supernatant according to Example 18.

### Example 17

### High-Throughput Screening Assay for T-cell Activity

NF-KB (Nuclear Factor KB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-KB regulates the expression of genes involved in immune cell activation, control of apoptosis (NF-KB appears to shield cells from apoptosis), B and T-cell development, anti-viral and antimicrobial responses, and multiple stress responses.

In non-stimulated conditions, NF-KB is retained in the cytoplasm with I-KB (Inhibitor KB). However, upon stimulation, I-KB is phosphorylated and degraded, causing NF-KB to shuttle to the nucleus, thereby activating transcription of target genes. Target genes activated by NF-KB include IL-2, IL-6, GM-CSF, ICAM-1 and class 1 MHC.

Due to its central role and ability to respond to a range of stimuli, reporter constructs utilizing the NF-KB promoter element are used to screen the supernatants produced in Example 12. Activators or inhibitors of NF-KB would be useful in treating diseases. For example, inhibitors of NF-KB could be used to treat those diseases related to the acute or chronic activation of NF-KB, such as rheumatoid arthritis.

To construct a vector containing the NF-KB promoter element, a PCR based strategy is employed. The upstream primer contains four tandem copies of the NF-KB binding site (GGGGACTTTCCC) (SEQ ID NO:20), 18 bp of sequence complementary to the 5′ end of the SV40 early promoter sequence, and is flanked with an XhoI site:

| | |
|---|---|
| 5′:GCGGCCTCGAGGGGACTTTCCCGGGGACTTTCCGGGGACTTTCCGGGACTTTCC | (SEQ ID NO: 21) |
| ATCCTGCCATCTCAATTAG:3′ | |

The downstream primer is complementary to the 3′ end of the SV40 promoter and is flanked with a Hind III site: 5′:GCGGCAAGCTTTTTGCAAAGCCTAGGC:3′ (SEQ ID NO:16)

PCR amplification is performed using the SV40 promoter template present in the pB-gal:promoter plasmid obtained from Clontech. The resulting PCR fragment is digested with XhoI and Hind III and subcloned into BLSK2-. (Stratagene) Sequencing with the T7 and T3 primers confirms the insert contains the following sequence:

| | |
|---|---|
| 5′:CTCGAGGGGACTTTCCCGGGGACTTTCCGGGGACTTTCCGGGACTTTCCATCTG | (SEQ ID NO: 22) |
| CCATCTCAATTAGTCAGCAACCATAGTCCCGCCCCTAACTCCGCCCATCCCGCCCCT | |
| AACTCCGCCCAGTTCCGCCCATTCTCCGCCCCATGGCTGACTAATTTTTTTTATTTA | |
| TGCAGAGGCCGAGGCCGCCTCGGCCTCTGAGCTATTCCAGAAGTAGTGAGGAGGCTT | |
| TTTTGGAGGCCTAGGCTTTTGCAAAAAGCTT:3′ | |

Next, replace the SV40 minimal promoter element present in the pSEAP2-promoter plasmid (Clontech) with this NF-KB/SV40 fragment using XhoI and HindIII. However, this vector does not contain a neomycin resistance gene, and therefore, is not preferred for mammalian expression systems.

In order to generate stable mammalian cell lines, the NF-KB/SV40/SEAP cassette is removed from the above NF-KB/SEAP vector using restriction enzymes SalI and NotI, and inserted into a vector containing neomycin resistance. Particularly, the NF-KB/SV40/SEAP cassette was inserted into pGFP-1 (Clontech), replacing the GFP gene, after restricting pGFP-1 with SalI and NotI.

Once NF-KB/SV40/SEAP/Neo vector is created, stable Jurkat T-cells are created and maintained according to the protocol described in Example 14. Similarly, the method for assaying supernatants with these stable Jurkat T-cells is also described in Example 14. As a positive control, exogenous TNF alpha (0.1,1, 10 ng) is added to wells H9, H10, and H11, with a 5-10 fold activation typically observed.

### Example 18

### Assay for SEAP Activity

As a reporter molecule for the assays described in Examples 14-17, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5×Dilution Buffer and dispense 15 ul of 2.5×dilution buffer into Optiplates containing 35 ul of a supernatant. Seal the plates with a plastic sealer and incubate at 65 degree C. for 30 min. Separate the Optiplates to avoid uneven heating.

Cool the samples to room temperature for 15 minutes. Empty the dispenser and prime with the Assay Buffer. Add 50 ml Assay Buffer and incubate at room temperature 5 min. Empty the dispenser and prime with the Reaction Buffer (see the table below). Add 50 ul Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 19

### High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 ul of buffer after the final wash.

A stock solution of 1 mg/ml fluo-3 is made in 10% pluronic acid DMSO. To load the cells with fluo-3, 50 ul of 12 ug/ml fluo-3 is added to each well. The plate is incubated at 37 degree C. in a CO₂ incubator for 60 min. The plate is washed four times in the Biotek washer with HBSS leaving 100 ul of buffer.

For non-adherent cells, the cells are spun down from culture media. Cells are re-suspended to 2-5×10⁶ cells/ml with HBSS in a 50-ml conical tube. 4 ul of 1 mg/ml fluo-3 solution in 10% pluronic acid DMSO is added to each ml of cell suspension. The tube is then placed in a 37 degree C. water bath for 30-60 min. The cells are washed twice with HBSS, resuspended to 1×10⁶ cells/ml, and dispensed into a microplate, 100 ul/well. The plate is centrifuged at 1000 rpm for 5 min. The plate is then washed once in Denley CellWash with 200 ul, followed by an aspiration step to 100 ul final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event caused by the a molecule, either neuropeptide receptor or a molecule induced by neuropeptide receptor, which has resulted in an increase in the intracellular Ca⁺⁺concentration.

### Example 20

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether neuropeptide receptor or a molecule induced by neuropeptide receptor is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4 degree C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200ml/well) and cultured overnight in complete medium. Cells are quiesced by incubation in serum-free basal medium for 24 hr. After 5-20 minutes treatment with EGF (60 ng/ml) or 50 ul of the supernatant produced in Example 12, the medium was removed and 100 ml of extraction buffer ((20 mM HEPES pH 7.5, 0.15 M NaCl, 1% Triton X-100, 0.1% SDS, 2 mM Na3VO4, 2 mM Na4P2O7 and a cocktail of protease inhibitors (#1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4 degree C. at 16,000×g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 ul ATP/Mg₂₊(5 mM ATP/50 mM MgCl₂), then 10 ul of 5×Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 ul of Sodium Vanadate(1 mM), and then 5 ul of water. Mix the components gently and preincubate the reaction mix at 30 degree C. for 2 min. Initial the reaction by adding 10 ul of the control enzyme or the filtered supernatant.

The tyrosine kinase assay reaction is then terminated by adding 10 ul of 120 mm EDTA and place the reactions on ice.

Tyrosine kinase activity is determined by transferring 50 ul aliquot of reaction mixture to a microtiter plate (MTP) module and incubating at 37 degree C. for 20 min. This allows the streptavadin coated 96 well plate to associate with the biotinylated peptide. Wash the MTP module with 300 ul/well of PBS four times. Next add 75 ul of anti-phospotyrosine antibody conjugated to horse radish peroxidase(anti-P-Tyr-POD(0.5 u/ml)) to each well and incubate at 37 degree C. for one hour. Wash the well as above.

Next add 100 ul of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 21

### High-Throughput Screening Assay Identifying Phosphorylation Activity

As a potential alternative and/or compliment to the assay of protein tyrosine kinase activity described in Example 20, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 ug/ml) for 2 hr at room temp, (RT). The plates are then rinsed with PBS and blocked with 3% BSA/PBS for 1 hr at RT. The protein G plates are then treated with 2 commercial monoclonal antibodies (10 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4 degree C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 ul of the supernatants obtained in Example 12 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by neuropeptide receptor or a molecule induced by neuropeptide receptor.

### Example 22

### Method of Determining Alterations in the Neuropeptide Receptor Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95 degree C. for 30 seconds; 60-120 seconds at 52-58 degree C.; and 60-120 seconds at 70 degree C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of neuropeptide receptor is also determined and genomic PCR products analyzed to confirm the results. PCR products harboring suspected mutations in neuropeptide receptor is then cloned and sequenced to validate the results of the direct sequencing.

PCR products of neuropeptide receptor are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in neuropeptide receptor not present in unaffected individuals.

Genomic rearrangements are also observed as a method of determining alterations in the neuropeptide receptor gene. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the neuropeptide receptor genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region of neuropeptide receptor (hybridized by the probe) are identified as insertions, deletions, and translocations. These neuropeptide receptor alterations are used as a diagnostic marker for an associated disease.

### Example 23

### Method of Detecting Abnormal Levels of Neuropeptide Receptor in a Biological Sample

Neuropeptide receptor polypeptides can be detected in a biological sample, and if an increased or decreased level of neuropeptide receptor is detected, this polypeptide is a marker for a particular phenotype. Methods of detection are numerous, and thus, it is understood that one skilled in the art can modify the following assay to fit their particular needs.

For example, antibody-sandwich ELISAs are used to detect neuropeptide receptor in a sample, preferably a biological sample. Wells of a microtiter plate are coated with specific antibodies to neuropeptide receptor, at a final concentration of 0.2 to 10 ug/ml. The antibodies are either monoclonal or polyclonal and are produced by the method described in Example 11. The wells are blocked so that non-specific binding of neuropeptide receptor to the well is reduced.

The coated wells are then incubated for >2 hours at RT with a sample containing neuropeptide receptor. Preferably, serial dilutions of the sample should be used to validate results. The plates are then washed three times with deionized or distilled water to remove unbounded neuropeptide receptor.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot neuropeptide receptor polypeptide concentration on the X-axis (log scale) and fluorescence or absorbance of the Y-axis (linear scale). Interpolate the concentration of the neuropeptide receptor in the sample using the standard curve.

### Example 24

### Formulation

The invention also provides methods of treatment and/or prevention of diseases or disorders (such as, for example, any one or more of the diseases or disorders disclosed herein) by administration to a subject of an effective amount of a Therapeutic. By therapeutic is meant a polynucleotides or polypeptides of the invention (including fragments and variants), agonists or antagonists thereof, and/or antibodies thereto, in combination with a pharmaceutically acceptable carrier type (e.g., a sterile carrier).

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 ug/kg/day to 10 mg/kg/day of patient body weight, although, as noted above, this win be subject to therapeutic discretion. More preferably, this dose is at least 0.01 mg/kg/day, and most preferably for humans between about 0.01 and 1 mg/kg/day for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Therapeutics can be are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrastemal, subcutaneous and intraarticular injection and infusion.

Therapeutics of the invention are also suitably administered by sustained-release systems. Suitable examples of sustained-release Therapeutics are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics include suitable polymeric materials (such as, for example, semi-permeable polymer matrices in the form of shaped articles, e.g., films, or mirocapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2- hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

Sustained-release Therapeutics also include liposomally entrapped Therapeutics of the invention (see generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing the Therapeutic are prepared by methods known per se: DE 3,218,121; Epstein et al., Proc. Natl. Acad. Sci. (USA) 82:3688-3692 (1985); Hwang et al., Proc. Natl. Acad. Sci.(USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The Therapeutic is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

The Therapeutics of the invention may be administered alone or in combination with adjuvants. Adjuvants that may be administered with the Therapeutics of the invention include, but are not limited to, alum, alum plus deoxycholate (ImmunoAg), MTP-PE (Biocine Corp.), QS21 (Genentech, Inc.), BCG, and MPL. In a specific embodiment, Therapeutics of the invention are administered in combination with alum. In another specific embodiment, Therapeutics of the invention are administered in combination with QS-21. Further adjuvants that may be administered with the Therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the Therapeutics of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The Therapeutics of the invention may be administered alone or in combination with other therapeutic agents. Therapeutic agents that may be administered in combination with the Therapeutics of the invention, include but not limited to, other members of the TNF family, chemotherapeutic agents, antibiotics, steroidal and non-steroidal anti-inflammatories, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the Therapeutics of the invention are administered in combination with members of the TNF family. TNF, TNF-related or TNF-like molecules that may be administered with the Therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892), TR10 (International Publication No. WO 98/54202), 312C2 (International Publication No. WO 98/06842), and TR12, and soluble forms CD154, CD70, and CD153.

In certain embodiments, Therapeutics of the invention are administered in combination with antiretroviral agents, nucleoside reverse transcriptase inhibitors, non-nucleoside reverse transcriptase inhibitors, and/or protease inhibitors. Nucleoside reverse transcriptase inhibitors that may be administered in combination with the Therapeutics of the invention, include, but are not limited to, RETROVIR™ (zidovudine/AZT), VIDEX™ (didanosine/ddl), HIVID™ (zalcitabine/ddC), ZERIT™ (stavudine/d4T), EPIVIR™ (lamivudine/3TC), and COMBIVIR™ (zidovudine/lamivudine). Non-nucleoside reverse transcriptase inhibitors that may be administered in combination with the Therapeutics of the invention, include, but are not limited to, VIRAMUNE™ (nevirapine), RESCRIPTOR™ (delavirdine), and SUSTIVA™ (efavirenz), Protease inhibitors that may be administered in combination with the Therapeutics of the invention, include, but are not limited to, CRIXIVAN™ (indinavir), NORVIR™ (ritonavir), INVIRASE™ (saquinavir), and VIRACEP™ (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In other embodiments, Therapeutics of the invention may be administered in combination with anti-opportunistic infection agents. Anti-opportunistic agents that may be administered in combination with the Therapeutics of the invention, include, but are not limited to, TRBMETHOPRIM-SULFAMETHOXAZOLE™, DAPSONE™, PENTAMIDINE™, ATOVAQUONE™, ISONIAZID™, RIFAMPIN™, PYRAZINAMIDE™, ETHAMBUTOL™, RIFABUTIN™, CLARITHROMYCIN™, AZITHROMYCIN™, GANCICLOVIR™, FOSCARNET™, CIDOFOVIR™, FLUCONAZOLE™, ITRACONAZOLE™, KETOCONAZOLE™, ACYCLOVIR™, FAMCICOLVIR™, PYRIMETHAMINE™, LEUCOVORIN™, NEUPOGEN™ (filgrastim/G-CSF), and LEUKINE™ (sargramostim/GM-CSF). In a specific embodiment, Therapeutics of the invention are used in any combination with TRIMETHOPRIM-SULFAMETHOXAZOLE™, DAPSONE™, PENTAMIDINE™, and/or ATOVAQUONE™ to prophylactically treat or prevent an opportunistic Pneumocystis carinii pneumonia infection. In another specific embodiment, Therapeutics of the invention are used in any combination with ISONIAZID™, RIFAMPIN™, PYRAZINAMIDE™, and/or ETHAMBUTOL™ to prophylactically treat or prevent an opportunistic Mycobacterium avium complex infection. In another specific embodiment, Therapeutics of the invention are used in any combination with RIFABUTIN™, CLARITHROMYCIN™, and/or AZITHROMYCIN™0 to prophylactically treat or prevent an opportunistic Mycobacterium tuberculosis infection. In another specific embodiment, Therapeutics of the invention are used in any combination with GANCICLOVIR™, FOSCARNET™, and/or CIDOFOVIR™ to prophylactically treat or prevent an opportunistic cytomegalovirus infection. In another specific embodiment, Therapeutics of the invention are used in any combination with FLUCONAZOLE™, ITRACONAZOLE™, and/or KETOCONAZOLE™ to prophylactically treat or prevent an opportunistic fungal infection. In another specific embodiment, Therapeutics of the invention are used in any combination with ACYCLOVIR™ and/or FAMCICOLVIR™ to prophylactically treat or prevent an opportunistic herpes simplex virus type I and/or type II infection. In another specific embodiment, Therapeutics of the invention are used in any combination with PYRIMETHAMIE™ and/or LEUCOVORIN™ to prophylactically treat or prevent an opportunistic Toxoplasma gondii infection. In another specific embodiment, Therapeutics of the invention are used in any combination with LEUCOVORIN™ and/or NEUPOGEN™ to prophylactically treat or prevent an opportunistic bacterial infection.

In a further embodiment, the Therapeutics of the invention are administered in combination with an antiviral agent. Antiviral agents that may be administered with the Therapeutics of the invention include, but are not limited to, acyclovir, ribavirin, amantadine, and remantidine.

In a further embodiment, the Therapeutics of the invention are administered in combination with an antibiotic agent. Antibiotic agents that may be administered with the Therapeutics of the invention include, but are not limited to, amoxicillin, beta-lactamases, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

Conventional nonspecific immunosuppressive agents, that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, Therapeutics of the invention are administered in combination with immunosuppressants. Immunosuppressants preparations that may be administered with the Therapeutics of the invention include, but are not limited to, ORTHOCLONE™ (OKT3), SANDIMMUNE™/NEORAL™/SANGDYA™ (cyclosporin), PROGRAF™ (tacrolimus), CELLCEPT™ (mycophenolate), Azathioprine, glucorticosteroids, and RAPAMUNE™ (sirolimus). In a specific embodiment, immunosuppressants may be used to prevent rejection of organ or bone marrow transplantation.

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR™, IVEEGAM™, SANDOGLOBULIN™, GAMMAGARD S/D™, and GAMIMUNE™. In a specific embodiment, Therapeutics of the invention are administered in combination with intravenous immune globulin preparations in transplantation therapy (e.g., bone marrow transplant).

In an additional embodiment, the Therapeutics of the invention are administered alone or in combination with an anti-inflammatory agent. Anti-inflammatory agents that may be administered with the Therapeutics of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compostions of the invention are administered in combination with a chemotherapeutic agent. Chemotherapeutic agents that may be administered with the Therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, Therapeutics of the invention are administered in combination with CHOP (cyclophosphamide, doxorubicin, vincristine, and prednisone) or any combination of the components of CHOP. In another embodiment, Therapeutics of the invention are administered in combination with Rituximab. In a further embodiment, Therapeutics of the invention are administered with Rituxmab and CHOP, or Rituxmab and any combination of the components of CHOP.

In an additional embodiment, the Therapeutics of the invention are administered in combination with cytokines. Cytokines that may be administered with the Therapeutics of the invention include, but are not limited to, IL2, IL3, IL4, IL5, IL6, IL7, IL10, IL12, IL13, IL15, anti-CD40, CD40L, IFN-gamma and TNF-alpha. In another embodiment, Therapeutics of the invention may be administered with any interleukin, including, but not limited to, IL-1 alpha, IL-1beta, IL-2, IL-3, IL-4, IL-5, IL-6, IL-7, IL-8, IL-9, IL-10, IL-11, IL-12 IL-14, IL-15, IL-16, IL-17, IL-18, IL-19, IL-20, and IL-21.

In an additional embodiment, the Therapeutics of the invention are administered in combination with angiogenic proteins. Angiogenic proteins that may be administered with the Therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-6821 10; Platelet Derived Growth Factor-B (PDGF-B), as disclosed in European Patent Number EP-282317; Placental Growth Factor (PlGF), as disclosed in International Publication Number WO 92/06194; Placental Growth Factor-2 (PlGF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the Therapeutics of the invention are administered in combination with hematopoietic growth factors. Hematopoietic growth factors that may be administered with the Therapeutics of the invention include, but are not limited to, LEUKINE™ (SARGRAMOSTIM™) and NEUPOGEN™ (FILGRASTIM™).

In an additional embodiment, the Therapeutics of the invention are administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In additional embodiments, the Therapeutics of the invention are administered in combination with other therapeutic or prophylactic regimens, such as, for example, radiation therapy.

### Example 25

### Method of Treating Decreased Levels of Neuropeptide Receptor

The present invention relates to a method for treating an individual in need of a decreased level of neuropeptide receptor activity in the body comprising, administering to such an individual a composition comprising a therapeutically effective amount of neuropeptide receptor antagonist. Preferred antagonists for use in the present invention are neuropeptide receptor-specific antibodies.

Moreover, it will be appreciated that conditions caused by a decrease in the standard or normal expression level of neuropeptide receptor in an individual can be treated by administering neuropeptide receptor, preferably in the secreted form. Thus, the invention also provides a method of treatment of an individual in need of an increased level of neuropeptide receptor polypeptide comprising administering to such an individual a pharmaceutical composition comprising an amount of neuropeptide receptor to increase the activity level of neuropeptide receptor in such an individual.

For example, a patient with decreased levels of neuropeptide receptor polypeptide receives a daily dose 0.1-100 ug/kg of the polypeptide for six consecutive days. Preferably, the polypeptide is in the secreted form. The exact details of the dosing scheme, based on administration and formulation, are provided in Example 24.

### Example 26

### Method of Treating Increased Levels of Neuropeptide Receptor

The present invention also relates to a method for treating an individual in need of an increased level of neuropeptide receptor activity in the body comprising administering to such an individual a composition comprising a therapeutically effective amount of neuropeptide receptor or an agonist thereof.

Antisense technology is used to inhibit production of neuropeptide receptor. This technology is one example of a method of decreasing levels of neuropeptide receptor polypeptide, preferably a secreted form, due to a variety of etiologies, such as cancer.

For example, a patient diagnosed with abnormally increased levels of neuropeptide receptor is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 24.

### Example 27

### Method of Treatment Using Gene Therapy—ex vivo

One method of gene therapy transplants fibroblasts, which are capable of expressing neuropeptide receptor polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding neuropeptide receptor can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted neuropeptide receptor.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the neuropeptide receptor gene is then added to the media and the packaging cells transduced with the vector. The packaging cells now produce infectious viral particles containing the neuropeptide receptor gene(the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether neuropeptide receptor protein is produced.

The engineered fibroblasts are then transplanted onto the host, either alone or after having been grown to confluence on cytodex 3 microcarrier beads.

### Example 28

### Gene Therapy Using Endogenous Neuropeptide Receptor Gene

Another method of gene therapy according to the present invention involves operably associating the endogenous neuropeptide receptor sequence with a promoter via homologous recombination as described, for example, in U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous neuropeptide receptor, flanking the promoter. The targeting sequence will be sufficiently near the 5′ end of neuropeptide receptor so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous neuropeptide receptor sequence. This results in the expression of neuropeptide receptor in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO₄, 6 mM dextrose). The cells are recentrifuged, the supernatant aspirated, and the cells resuspended in electroporation buffer containing 1 mg/nl acetylated bovine serum albumin. The final cell suspension contains approximately 3×10⁶ cells/ml. Electroporation should be performed immediately following resuspension.

Plasmid DNA is prepared according to standard techniques. For example, to construct a plasmid for targeting to the neuropeptide receptor locus, plasmid pUC 18 (MBI Fermentas, Amherst, N.Y.) is digested with HindIII. The CMV promoter is amplified by PCR with an XbaI site on the 5′ end and a BamHI site on the 3′end. Two neuropeptide receptor non-coding sequences are amplified via PCR: one neuropeptide receptor non-coding sequence (neuropeptide receptor fragment 1) is amplified with a HindIII site at the 5′ end and an Xba site at the 3′end; the other neuropeptide receptor non-coding sequence (neuropeptide receptor fragment 2) is amplified with a BamHI site at the 5′end and a HindIII site at the 3′end. The CMV promoter and neuropeptide receptor fragments are digested with the appropriate enzymes (CMV promoter—XbaI and BamHI; neuropeptide receptor fragment 1—XbaI; neuropeptide receptor fragment 2—BamHI) and ligated together. The resulting ligation product is digested with HindIII, and ligated with the HindIII-digested pUC 18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5.×10⁶ cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 degree C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 29

### Method of Treatment Using Gene Therapy—in vivo

Another aspect of the present invention is using in vivo gene therapy methods to treat disorders, diseases and conditions. The gene therapy method relates to the introduction of naked nucleic acid (DNA, RNA, and antisense DNA or RNA) neuropeptide receptor sequences into an animal to increase or decrease the expression of the neuropeptide receptor polypeptide. The neuropeptide receptor polynucleotide may be operatively linked to a promoter or any other genetic elements necessary for the expression of the neuropeptide receptor polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The neuropeptide receptor polynucleotide constructs may be delivered by any method that delivers injectable materials to the cells of an animal, such as, injection into the interstitial space of tissues (heart, muscle, skin, lung, liver, intestine and the like). The neuropeptide receptor polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the neuropeptide receptor polynucleotides may also be delivered in liposome formulations (such as those taught in Felgner P. L. et al. (1995) Ann. NY Acad. Sci. 772:126-139 and Abdallah B. et al. (1995) Biol. Cell 85(1): 1-7) which can be prepared by methods well known to those skilled in the art.

The neuropeptide receptor polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The neuropeptide receptor polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked neuropeptide receptor polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked neuropeptide receptor polynucleotide constructs can be delivered to arteries during angioplasty by the catheter used in the procedure.

The dose response effects of injected neuropeptide receptor polynucleotide in muscle in vivo is determined as follows. Suitable neuropeptide receptor template DNA for production of mRNA coding for neuropeptide receptor polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The neuropeptide receptor template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for neuropeptide receptor protein expression. A time course for neuropeptide receptor protein expression may be done in a similar fashion except that quadriceps from different mice are harvested at different times. Persistence of neuropeptide receptor DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using neuropeptide receptor naked DNA.

### Example 30

### Neuropeptide Receptor Transgenic Animals

The neuropeptide receptor polypeptides can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270(1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810⁻⁸¹³ (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred.

Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of neuropeptide receptor polypeptides, studying conditions and/or disorders associated with aberrant neuropeptide receptor expression, and in screening for compounds effective in ameliorating such conditions and/or disorders.

### Example 31

### Neuropeptide Receptor Knock-Out Animals

Endogenous neuropeptide receptor gene expression can also be reduced by inactivating or “knocking out” the neuropeptide receptor gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the neuropeptide receptor polypeptides. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Knock-out animals of the invention have uses which include, but are not limited to, animal model systems useful in elaborating the biological function of neuropeptide receptor polypeptides, studying conditions and/or disorders associated with aberrant neuropeptide receptor expression, and in screening for compounds effective in ameliorating such conditions and/or disorders.

### Example 32

### Assays Detecting Stimulation or Inhibition of B cell Proliferation and Differentiation

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL10, IL-13, IL-14 and IL-15. Interestingly, these signals are by themselves weak effectors but can, in combination with various co-stimulatory proteins, induce activation, proliferation, differentiation, homing, tolerance and death among B cell populations.

One of the best studied classes of B-cell co-stimulatory proteins is the TNF-superfamily. Within this family CD40, CD27, and CD30 along with their respective ligands CD 154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In Vitro Assay—Purified neuropeptide receptor protein, or truncated forms thereof, is assessed for its ability to induce activation, proliferation, differentiation or inhibition and/or death in B-cell populations and their precursors. The activity of neuropeptide receptor protein on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 10⁵ B-cells suspended in culture medium (RPMI 1640 containing 10% FBS, 5×10⁻⁵M 2ME, 100 U/ml penicillin, 10 ug/ml streptomycin, and 10⁻⁵ dilution of SAC) in a total volume of 150 ul. Proliferation or inhibition is quantitated by a 20 h pulse (1uCi/well) with 3H-thymidine (6.7 Ci/mM) beginning 72 h post factor addition. The positive and negative controls are IL2 and medium respectively.

In Vivo Assay—BALB/c mice are injected (i.p.) twice per day with buffer only, or 2 mg/Kg of neuropeptide receptor protein, or truncated forms thereof. Mice receive this treatment for 4 consecutive days, at which time they are sacrificed and various tissues and serum collected for analyses. Comparison of H&E sections from normal and neuropeptide receptor protein-treated spleens identify the results of the activity of neuropeptide receptor protein on spleen cells, such as the diffusion of peri-arterial lymphatic sheaths, and/or significant increases in the nucleated cellularity of the red pulp regions, which may indicate the activation of the differentiation and proliferation of B-cell populations. Immunohistochemically studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from neuropeptide receptor protein-treated mice is used to indicate whether neuropeptide receptor protein specifically increases the proportion of ThB+, CD45R(B220)dull B cells over that which is observed in control nice.

Likewise, a predicted consequence of increased mature B-cell representation in vivo is a relative increase in serum Ig titers. Accordingly, serum IgM and IgA levels are compared between buffer and neuropeptide receptor protein-treated mice.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 33

### T Cell Proliferation Assay

A CD3-induced proliferation assay is performed on PBMCs and is measured by the uptake of ³H-thymidine. The assay is performed as follows. Ninety-six well plates are coated with 100 μl/well of mAb to CD3 (HIT3a, Pharmingen) or isotype-matched control mAb (B33.1 ) overnight at 4° C. (1 μg/ml in 0.05M bicarbonate buffer, pH 9.5), then washed three times with PBS. PBMC are isolated by F/H gradient centrifugation from human peripheral blood and added to quadruplicate wells (5×10⁴/well) of mAb coated plates in RPMI containing 10% FCS and P/S in the presence of varying concentrations of neuropeptide receptor protein (total volume 200 μl). Relevant protein buffer and medium alone are controls. After 48 hr. culture at 37° C., plates are spun for 2 min. at 1000 rpm and 100 μl of supernatant is removed and stored -20° C. for measurement of IL-2 (or other cytokines) if effect on proliferation is observed. Wells are supplemented with 100 μl of medium containing 0.5 μCi of ³H-thymidine and cultured at 37° C. for 18-24 hr. Wells are harvested and incorporation of ³H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative controls for the effects of neuropeptide receptor proteins.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 34

### Effect of Neuropeptide Receptor on the Expression of MHC Class II, Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and MHC class II antigens). Treatment with activating factors, such as TNF-α, causes a rapid change in surface phenotype (increased expression of MHC class I and II, costimulatory and adhesion molecules, downregulation of FCγII, upregulation of CD83). These changes correlate with increased antigen-presenting capacity and with functional maturation of the dendritic cells.

FACS analysis of surface antigens is performed as follows. Cells are treated 1-3 days with increasing concentrations of neuropeptide receptor or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Effect on the production of cytokines. Cytokines generated by dendritic cells, in particular IL-12, are important in the initiation of T-cell dependent immune responses. IL-12 strongly influences the development of Thl helper T-cell immune response, and induces cytotoxic T and NK cell function. An ELISA is used to measure the IL-12 release as follows. Dendritic cells (10⁶/ml) are treated with increasing concentrations of neuropeptide receptor for 24 hours. LPS (100 ng/ml) is added to the cell culture as positive control. Supernatants from the cell cultures are then collected and analyzed for IL-12 content using commercial ELISA kit (e.g, R & D Systems (Minneapolis, Minn.)). The standard protocols provided with the kits are used.

Effect on the expression of MHC Class II, costimulatory and adhesion molecules. Three major families of cell surface antigens can be identified on monocytes: adhesion molecules, molecules involved in antigen presentation, and Fc receptor. Modulation of the expression of MHC class II antigens and other costimulatory molecules, such as B7 and ICAM1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of neuropeptide receptor or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Monocyte activation and/or increased survival. Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. Neuropeptide receptor, agonists, or antagonists of neuropeptide receptor can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation. Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×10⁶/ml in PBS containing PI at a final concentration of 5 μg/ml, and then incubaed at room temperature for 5 minutes before FACScan analysis. PI uptake has been demonstrated to correlate with DNA fragmentation in this experimental paradigm.

Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×10⁵ cells/ml with increasing concentrations of neuropeptide receptor and under the same conditions, but in the absence of neuropeptide receptor. For IL-12 production, the cells are primed overnight with IFN (100 U/ml) in presence of neuropeptide receptor. LPS (10 ng/ml) is then added. Conditioned media are collected after 24 h and kept frozen until use. Measurement of TNF-alpha, IL-10, MCP-1 and IL-8 is then performed using a commercially available ELISA kit (e.g, R & D Systems (Minneapolis, Minn.)) and applying the standard protocols provided with the kit.

Oxidative burst. Purified monocytes are plated in 96-w plate at 2-1×10⁵ cell/well. Increasing concentrations of neuropeptide receptor are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 mM PMA). The plates are incubated at 37° C. for 2 hours and the reaction is stopped by adding 20 μl 1N NaOH per well. The absorbance is read at 610 nm. To calculate the amount of H₂O₂ produced by the macrophages, a standard curve of a H₂O₂ solution of known molarity is performed for each experiment.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 35

### Neuropeptide Receptor Biological Effects

Astrocyte and Neuronal Assays

Recombinant neuropeptide receptor, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate neuropeptide receptor's activity on these cells.

Moreover, previous reports describing the biological effects of FGF-2 (basic FGF) on cortical or hippocampal neurons in vitro have demonstrated increases in both neuron survival and neurite outgrowth (Walicke, P. et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of neuropeptide receptor to induce neurite outgrowth can be compared to the response achieved with FGF-2 using, for example, a thymidine incorporation assay.

Fibroblast and Endothelial Cell Assays

Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE₂ assays, the human lung fibroblasts are cultured at 5,000 cells/well in a 96-well plate for one day. After a medium change to 0.1% BSA basal medium, the cells are incubated with FGF-2 or neuropeptide receptor with or without IL-1α for 24 hours. The supernatants are collected and assayed for PGE₂ by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or neuropeptide receptor with or without IL-1α for 24 hours. The supernatants are collected and assayed for IL-6 by ELISA kit (Endogen, Cambridge, Mass.).

Human lung fibroblasts are cultured with FGF-2 or neuropeptide receptor for 3 days in basal medium before the addition of Alamar Blue to assess effects on growth of the fibroblasts. FGF-2 should show a stimulation at 10-2500 ng/ml which can be used to compare stimulation with neuropeptide receptor.

Parkinson Models

The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP⁺) and released. Subsequently, MPP⁺ is actively accumulated in dopaminergic neurons by the high-affinity reuptake transporter for dopamine. MPP⁺ is then concentrated in mitochondria by the electrochemical gradient and selectively inhibits nicotidamide adenine disphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, neuropeptide receptor can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of neuropeptide receptor is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm² on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if neuropeptide receptor acts to prolong the survival of dopaminergic neurons, it would suggest that neuropeptide receptor may be involved in Parkinson's Disease.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 36

### The Effect of Neuropeptide Receptor on the Growth of Vascular Endothelial Cells

On day 1, human umbilical vein endothelial cells (HUVEC) are seeded at 2-5×10⁴ cells/35 mm dish density in M199 medium containing 4% fetal bovine serum (FBS), 16 units/ml heparin, and 50 units/ml endothelial cell growth supplements (ECGS, Biotechnique, Inc.). On day 2, the medium is replaced with M199 containing 10% FBS, 8 units/ml heparin. Neuropeptide receptor protein of SEQ ID NO. 2, and positive controls, such as VEGF and basic FGF (bFGF) are added, at varying concentrations. On days 4 and 6, the medium is replaced. On day 8, cell number is determined with a Coulter Counter.

An increase in the number of HUVEC cells indicates that neuropeptide receptor may proliferate vascular endothelial cells.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 37

### Stimulatory Effect of Neuropeptide Receptor on the Proliferation of Vascular Endothelial Cells

For evaluation of mitogenic activity of growth factors, the colorimetric MTS (3-(4,5-dimethylthiazol-2-yl)-5-(3-carboxymethoxyphenyl)-2-(4-sulfophenyl)2H-tetrazolium) assay with the electron coupling reagent PMS (phenazine methosulfate) was performed (CellTiter 96 AQ, Promega). Cells are seeded in a 96-well plate (5,000 cells/well) in 0.1 mL serum-supplemented medium and are allowed to attach overnight. After serum-starvation for 12 hours in 0.5% FBS, conditions (bFGF, VEGF₁₆₅ or neuropeptide receptor in 0.5% FBS) with or without Heparin (8 U/ml) are added to wells for 48 hours. 20 mg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. In Vitro Cell. Dev. Biol. 30A:512-518 (1994).

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 38

### Inhibition of PDGF-induced Vascular Smooth Muscle Cell Proliferation Stimulatory Effect

HAoSMC proliferation can be measured, for example, by BrdUrd incorporation. Briefly, subconfluent, quiescent cells grown on the 4-chamber slides are transfected with CRP or FYFC-labeled AT2-3LP. Then, the cells are pulsed with 10% calf serum and 6 mg/ml BrdUrd. After 24 h, immunocytochemistry is performed by using BrdUrd Staining Kit (Zymed Laboratories). In brief, the cells are incubated with the biotinylated mouse anti-BrdUrd antibody at 4 ° C. for 2 h after being exposed to denaturing solution and then incubated with the streptavidin-peroxidase and diaminobenzidine. After counterstaining with hematoxylin, the cells are mounted for microscopic examination, and the BrdUrd-positive cells are counted. The BrdUrd index is calculated as a percent of the BrdUrd-positive cells to the total cell number. In addition, the simultaneous detection of the BrdUrd staining (nucleus) and the FITC uptake (cytoplasm) is performed for individual cells by the concomitant use of bright field illumination and dark field-UV fluorescent illumination. See, Hayashida et al., J. Biol. Chem. 6:271(36):21985-21992 (1996).

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 39

### Stimulation of Endothelial Migration

This example will be used to explore the possibility that neuropeptide receptor may stimulate lymphatic endothelial cell migration.

Endothelial cell migration assays are performed using a 48 well michrochemotaxis chamber (Neuroprobe Inc., Cabin John, M D; Falk, W., et al., J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells suspended in 50 ul M199 containing 1% FBS are seeded in the upper compartment. The apparatus is then incubated for 5 hours at 37° C. in a humidified chamber with 5% CO2 to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 40

### Stimulation of Nitric Oxide Production by Endothelial Cells

Nitric oxide released by the vascular endothelium is believed to be a mediator of vascular endothelium relaxation. Thus, neuropeptide receptor activity can be assayed by determining nitric oxide production by endothelial cells in response to neuropeptide receptor.

Nitric oxide is measured in 96-well plates of confluent microvascular endothelial cells after 24 hours starvation and a subsequent 4 hr exposure to various levels of a positive control (such as VEGF-1) and neuropeptide receptor. Nitric oxide in the medium is determined by use of the Griess reagent to measure total nitrite after reduction of nitric oxide-derived nitrate by nitrate reductase. The effect of neuropeptide receptor on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2KNO₂+2KI+2H₂SO₄62NO+I₂+2H₂O+2K₂SO₄

The standard calibration curve is obtained by adding graded concentrations of KNO₂ (0, 5, 10, 25, 50, 100, 250, and 500 nmol/L) into the calibration solution containing KI and H₂SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values reported are means of four to six measurements in each group (number of cell culture wells). See, Leak et al. Biochem. and Biophys. Res. Comm. 217:96-105 (1995).

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 41

### Effect of Neuropeptide Receptor on Cord Formation in Angiogenesis

Another step in angiogenesis is cord formation, marked by differentiation of endothelial cells. This bioassay measures the ability of microvascular endothelial cells to form capillary-like structures (hollow structures) when cultured in vitro.

CADMEC (microvascular endothelial cells) are purchased from Cell Applications, Inc. as proliferating (passage 2) cells and are cultured in Cell Applications' C.ADMEC Growth Medium and used at passage 5. For the in vitro angiogenesis assay, the wells of a 48-well cell culture plate are coated with Cell Applications' Attachment Factor Medium (200 ml/well) for 30 min. at 37° C. CADMEC are seeded onto the coated wells at 7,500 cells/well and cultured overnight in Growth Medium. The Growth Medium is then replaced with 300 mg Cell Applications' Chord Formation Medium containing control buffer or neuropeptide receptor (0.1 to 100 ng/ml) and the cells are cultured for an additional 48 hr. The numbers and lengths of the capillary-like chords are quantitated through use of the Boeckeler VIA-170 video image analyzer. All assays are done in triplicate.

Commercial (R&D) VEGF (50 ng/ml) is used as a positive control. b-esteradiol (1 ng/ml) is used as a negative control. The appropriate buffer (without protein) is also utilized as a control.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 42

### Angiogenic Effect on Chick Chorioallantoic Membrane

Chick chorioallantoic membrane (CAM) is a well-established system to examine angiogenesis. Blood vessel formation on CAM is easily visible and quantifiable. The ability of neuropeptide receptor to stimulate angiogenesis in CAM can be examined.

Fertilized eggs of the White Leghorn chick (Gallus gallus) and the Japanese qual (Cotumix cotumix) are incubated at 37.8° C. and 80% humidity. Differentiated CAM of 16-day-old chick and 13-day-old qual embryos is studied with the following methods.

On Day 4 of development, a window is made into the egg shell of chick eggs. The embryos are checked for normal development and the eggs sealed with cellotape. They are further incubated until Day 13. Thermanox coverslips (Nunc, Naperville, Ill.) are cut into disks of about 5 mm in diameter. Sterile and salt-free growth factors are dissolved in distilled water and about 3.3 mg/5 ml are pipetted on the disks. After air-drying, the inverted disks are applied on CAM. After 3 days, the specimens are fixed in 3% glutaraldehyde and 2% formaldehyde and rinsed in 0.12 M sodium cacodylate buffer. They are photographed with a stereo microscope [Wild M8] and embedded for semi- and ultrathin sectioning as described above. Controls are performed with carrier disks alone.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 43

### Angiogenesis Assay Using a Matrigel Implant in Mouse

In vivo angiogenesis assay of neuropeptide receptor measures the ability of an existing capillary network to form new vessels in an implanted capsule of murine extracellular matrix material (Matrigel). The protein is mixed with the liquid Matrigel at 4 degree C. and the mixture is then injected subcutaneously in mice where it solidifies. After 7 days, the solid “plug” of Matrigel is removed and examined for the presence of new blood vessels. Matrigel is purchased from Becton Dickinson Labware/Collaborative Biomedical Products.

When thawed at 4 degree C. the Matrigel material is a liquid. The Matrigel is mixed with neuropeptide receptor at 150 ng/ml at 4 degree C. and drawn into cold 3 ml syringes. Female C57B1/6 mice approximately 8 weeks old are injected with the mixture of Matrigel and experimental protein at 2 sites at the midventral aspect of the abdomen (0.5 ml/site). After 7 days, the mice are sacrificed by cervical dislocation, the Matrigel plugs are removed and cleaned (i.e., all clinging membranes and fibrous tissue is removed). Replicate whole plugs are fixed in neutral buffered 10% formaldehyde, embedded in paraffin and used to produce sections for histological examination after staining with Masson's Trichrome. Cross sections from 3 different regions of each plug are processed. Selected sections are stained for the presence of vWF. The positive control for this assay is bovine basic FGF (150 ng/ml). Matrigel alone is used to determine basal levels of angiogenesis.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 44

### Rescue of Ischemia in Rabbit Lower Limb Model

To study the in vivo effects of neuropeptide receptor on ischemia, a rabbit hindlimb ischemia model is created by surgical removal of one femoral arteries as described previously (Takeshita, S. et al., Am J. Pathol 147:1649-1660 (1995)). The excision of the femoral artery results in retrograde propagation of thrombus and occlusion of the external iliac artery. Consequently, blood flow to the ischemic limb is dependent upon collateral vessels originating from the internal iliac artery (Takeshita, S. et al. Am J. Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 mg naked neuropeptide receptor expression plasmid by arterial gene transfer technology using a hydrogel-coated balloon catheter as described (Riessen, R. et al. Hum Gene Ther. 4:749-758 (1993); Leclerc, G. et al. J. Clin. Inivest. 90: 936-944 (1992)). When neuropeptide receptor is used in the treatment, a single bolus of 500 mg neuropeptide receptor protein or control is delivered into the internal iliac artery of the ischemic limb over a period of 1 min. through an infusion catheter. On day 30, various parameters are measured in these rabbits: (a) BP ratio—The blood pressure ratio of systolic pressure of the ischemiclimb to that of normal limb; (b) Blood Flow and Flow Reserve—Resting FL: the blood flow during undilated condition and Max FL: the blood flow during fully dilated condition (also an indirect measure of the blood vessel amount) and Flow Reserve is reflected by the ratio of max FL: resting FL; (c) Angiographic Score—This is measured by the angiogram of collateral vessels. A score is determined by the percentage of circles in an overlaying grid that with crossing opacified arteries divided by the total number m the rabbit thigh; (d) Capillary density—The number of collateral capillaries determined in light microscopic sections taken from hindlimbs.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 45

### Effect of Neuropeptide Receptor on Vasodilation

Since dilation of vascular endothelium is important in reducing blood pressure, the ability of neuropeptide receptor to affect the blood pressure in spontaneously hypertensive rats (SHR) is examined. Increasing doses (0, 10, 30, 100, 300, and 900 mg/kg) of the neuropeptide receptor are administered to 13-14 week old spontaneously hypertensive rats (SHR). Data are expressed as the mean +/− SEM. Statistical analysis are performed with a paired t-test and statistical significance is defined as p<0.05 vs. the response to buffer alone.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 46

### Rat Ischemic Skin Flap Model

The evaluation parameters include skin blood flow, skin temperature, and factor VIII immunohistochemistry or endothelial alkaline phosphatase reaction. Neuropeptide receptor expression, during the skin ischemia, is studied using in situ hybridization.

The study in this model is divided into three parts as follows:

a) Ischemic skin

b) Ischemnic skin wounds

c) Normal wounds

The experimental protocol includes:

a) Raising a 3×4 cm, single pedicle full-thickness random skin flap (myocutaneous flap over the lower back of the animal).

b) An excisional wounding (4-6 mm in diameter) in the ischemic skin (skin-flap).

c) Topical treatment with neuropeptide receptor of the excisional wounds (day 0, 1, 2, 3, 4 post-wounding) at the following various dosage ranges: 1 mg to 100 mg.

d) Harvesting the wound tissues at day 3, 5, 7, 10, 14 and 21 post-wounding for histological, immunohistochemical, and in situ studies.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 47

### Peripheral Arterial Disease Model

Angiogenic therapy using neuropeptide receptor is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) neuropeptide receptor protein, in a dosage range of 20 mg -500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of neuropeptide receptor expression and histology. Biopsy is also performed on the other side of normal muscle of the contralateral hindlimb.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 48

### Ischemic Myocardial Disease Model

Neuropeptide receptor is evaluated as a potent mitogen capable of stimulating the development of collateral vessels, and restructuring new vessels after coronary artery occlusion. Alteration of neuropeptide receptor expression is investigated in situ. The experimental protocol includes:

a) The heart is exposed through a left-side thoracotomy in the rat. Immediately, the left coronary artery is occluded with a thin suture (6-0) and the thorax is closed.

b) Neuropeptide receptor protein, in a dosage range of 20 mg -500 mg, is delivered intravenously and/or intramuscularly 3 times (perhaps more) per week for 2-4 weeks.

c) Thirty days after the surgery, the heart is removed and cross-sectioned for morphometric and in situ analyzes.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 49

### Rat Corneal Wound Healing Model

This animal model shows the effect of neuropeptide receptor on neovascularization. The experimental protocol includes:

a) Making a 1-1.5 mm long incision from the center of cornea into the stromal layer.

b) Inserting a spatula below the lip of the incision facing the outer corner of the eye.

c) Making a pocket (its base is 1-1.5 mm form the edge of the eye).

d) Positioning a pellet, containing 50 ng-5ug of neuropeptide receptor, within the pocket.

e) Neuropeptide receptor treatment can also be applied topically to the corneal wounds in a dosage range of 20 mg -500 mg (daily treatment for five days).

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 50

### Diabetic Mouse and Glucocorticoid-Impaired Wound Healing Models

A. Diabetic db+/db+ Mouse Model

To demonstrate that neuropeptide receptor accelerates the healing process, the genetically diabetic mouse model of wound healing is used. The full thickness wound healing model in the db+/db+ mouse is a well characterized, clinically relevant and reproducible model of impaired wound healing. Healing of the diabetic wound is dependent on formation of granulation tissue and re-epithelialization rather than contraction (Gartner, M. H. et al., J. Surg. Res. 52:389 (1992); Greenhalgh, D. G. et al., Am. J. Pathol. 136:1235 (1990)).

The diabetic animals have many of the characteristic features observed in Type II diabetes mellitus. Homozygous (db+/db+) mice are obese in comparison to their normal heterozygous (db+/+m) littermates. Mutant diabetic (db+/db+) mice have a single autosomal recessive mutation on chromosome 4 (db+) (Coleman et al. Proc. Natl. Acad. Sci. USA 77:283-293 (1982)). Animals show polyphagia, polydipsia and polyuria. Mutant diabetic mice (db+/db+) have elevated blood glucose, increased or normal insulin levels, and suppressed cell-mediated immunity (Mandel et al., J. Immunol. 120:1375 (1978); Debray-Sachs, M. et al., Clin. Exp. Immunol. 51(1)-1-7 (1983); Leiter et al., Am. J. of Pathol. 114:46-55 (1985)). Peripheral neuropathy, myocardial complications, and microvascular lesions, basement membrane thickening and glomerular filtration abnormalities have been described in these animals (Norido, F. et al., Exp. Neurol. 83(2):221-232 (1984); Robertson et al., Diabetes 29(1):60-67 (1980); Giacomelli et al., Lab Invest. 40(4):460-473 (1979); Coleman, D. L., Diabetes 31 (Suppl):1-6 (1982)). These homozygous diabetic mice develop hyperglycemia that is resistant to insulin analogous to human type II diabetes (Mandel et al., J. Immunol. 120:1375-1377 (1978)).

The characteristics observed in these animals suggests that healing in this model may be similar to the healing observed in human diabetes (Greenhalgh, et al., Am. J. of Pathol. 136:1235-1246 (1990)).

Genetically diabetic female C57BL/KsJ (db+/db+) mice and their non-diabetic (db+/+m) heterozygous littermates are used in this study (Jackson Laboratories). The animals are purchased at 6 weeks of age and are 8 weeks old at the beginning of the study. Animals are individually housed and received food and water ad libitum. All manipulations are performed using aseptic techniques. The experiments are conducted according to the rules and guidelines of Human Genome Sciences, Inc. Institutional Animal Care and Use Committee and the Guidelines for the Care and Use of Laboratory Animals.

Wounding protocol is performed according to previously reported methods (Tsuboi, R. and Rifkin, D. B., J. Exp. Med. 172:245-251 (1990)). Briefly, on the day of wounding, animals are anesthetized with an intraperitoneal injection of Avertin (0.01 mg/mL), 2,2,2-tribromoethanol and 2-methyl-2-butanol dissolved in deionized water. The dorsal region of the animal is shaved and the skin washed with 70% ethanol solution and iodine. The surgical area is dried with sterile gauze prior to wounding. An 8 mm full-thickness wound is then created using a Keyes tissue punch. Immediately following wounding, the surrounding skin is gently stretched to eliminate wound expansion. The wounds are left open for the duration of the experiment. Application of the treatment is given topically for 5 consecutive days commencing on the day of wounding. Prior to treatment, wounds are gently cleansed with sterile saline and gauze sponges.

Wounds are visually examined and photographed at a fixed distance at the day of surgery and at two day intervals thereafter. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

Neuropeptide receptor is administered using at a range different doses of neuropeptide receptor, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology and immunohistochemistry. Tissue specimens are placed in 10% neutral buffered formalin in tissue cassettes between biopsy sponges for further processing.

Three groups of 10 animals each (5 diabetic and 5 non-diabetic controls) are evaluated: 1) Vehicle placebo control, 2) neuropeptide receptor.

Wound closure is analyzed by measuring the area in the vertical and horizontal axis and obtaining the total square area of the wound. Contraction is then estimated by establishing the differences between the initial wound area (day 0) and that of post treatment (day 8). The wound area on day 1 is 64 mm², the corresponding size of the dermal punch. Calculations are made using the following formula:

[Open area on day 8]−[Open area on day 1]/[Open area on day 1]

Specimens are fixed in 10% buffered formalin and paraffin embedded blocks are sectioned perpendicular to the wound surface (5 mm) and cut using a Reichert-Jung microtome. Routine hematoxylin-eosin (H&E) staining is performed on cross-sections of bisected wounds. Histologic examination of the wounds are used to assess whether the healing process and the morphologic appearance of the repaired skin is altered by treatment with neuropeptide receptor. This assessment included verification of the presence of cell accumulation, inflammatory cells, capillaries, fibroblasts, re-epithelialization and epidermal maturity (Greenhalgh, D. G. et al., Am. J. Pathol. 136:1235 (1990)). A calibrated lens micrometer is used by a blinded observer.

Tissue sections are also stained immunohistochemically with a polyclonal rabbit anti-human keratin antibody using ABC Elite detection system. Human skin is used as a positive tissue control while non-immune IgG is used as a negative control. Keratinocyte growth is determined by evaluating the extent of reepithelialization of the wound using a calibrated lens micrometer.

Proliferating cell nuclear antigen/cyclin (PCNA) in skin specimens is demonstrated by using anti-PCNA antibody (1:50) with an ABC Elite detection system. Human colon cancer served as a positive tissue control and human brain tissue is used as a negative tissue control. Each specimen included a section with omission of the primary antibody and substitution with non-immune mouse IgG. Ranking of these sections is based on the extent of proliferation on a scale of 0-8, the lower side of the scale reflecting slight proliferation to the higher side reflecting intense proliferation.

Experimental data are analyzed using an unpaired t test. A p value of <0.05 is considered significant.

B. Steroid Impaired Rat Model

The inhibition of wound healing by steroids has been well documented in various in vitro and in vivo systems (Wahl, S. M. Glucocorticoids and Wound healing. In: Anti-Inflammatory Steroid Action: Basic and Clinical Aspects. 280-302 (1989); Wahl, S. M. et al., J. Immunol. 115: 476-481 (1975); Werb, Z. et al., J. Exp. Med. 147:1684-1694 (1978)). Glucocorticoids retard wound healing by inhibiting angiogenesis, decreasing vascular permeability ( Ebert, R. H., et al., An. Intern. Med. 37:701-705 (1952)), fibroblast proliferation, and collagen synthesis (Beck, L. S. et al., Growth Factors. 5: 295-304 (1991); Haynes, B. F. et al., J. Clin. Invest. 61: 703-797 (1978)) and producing a transient reduction of circulating monocytes (Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989)). The systemic administration of steroids to impaired wound healing is a well establish phenomenon in rats (Beck, L. S. et al., Growth Factors. 5: 295-304 (1991); Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989); Pierce, G. F. et al., Proc. Natl. Acad. Sci. USA 86: 2229-2233 (1989)).

To demonstrate that neuropeptide receptor can accelerate the healing process, the effects of multiple topical applications of neuropeptide receptor on full thickness excisional skin wounds in rats in which healing has been impaired by the systemic administration of methylprednisolone is assessed.

Young adult male Sprague Dawley rats weighing 250-300 g (Charles River Laboratories) are used in this example. The animals are purchased at 8 weeks of age and are 9 weeks old at the beginning of the study. The healing response of rats is impaired by the systemic administration of methylprednisolone (17 mg/kg/rat intramuscularly) at the time of wounding. Animals are individually housed and received food and water ad libitum. All manipulations are performed using aseptic techniques. This study is conducted according to the rules and guidelines of Human Genome Sciences, Inc. Institutional Animal Care and Use Committee and the Guidelines for the Care and Use of Laboratory Animals.

The wounding protocol is followed according to section A, above. On the day of wounding, animals are anesthetized with an intramuscular injection of ketamine (50 mg/kg) and xylazine (5 mg/kg). The dorsal region of the animal is shaved and the skin washed with 70% ethanol and iodine solutions. The surgical area is dried with sterile gauze prior to wounding. An 8 mm full-thickness wound is created using a Keyes tissue punch. The wounds are left open for the duration of the experiment. Applications of the testing materials are given topically once a day for 7 consecutive days commencing on the day of wounding and subsequent to methylprednisolone administration. Prior to treatment, wounds are gently cleansed with sterile saline and gauze sponges.

Wounds are visually examined and photographed at a fixed distance at the day of wounding and at the end of treatment. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

Neuropeptide receptor is administered using at a range different doses of neuropeptide receptor, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology. Tissue specimens are placed in 10% neutral buffered formalin in tissue cassettes between biopsy sponges for further processing.

Four groups of 10 animals each (5 with methylprednisolone and 5 without glucocorticoid) are evaluated: 1) Untreated group 2) Vehicle placebo control 3) neuropeptide receptor treated groups.

Wound closure is analyzed by measuring the area in the vertical and horizontal axis and obtaining the total area of the wound. Closure is then estimated by establishing the differences between the initial wound area (day 0) and that of post treatment (day 8). The wound area on day 1 is 64 mm², the corresponding size of the dermal punch. Calculations are made using the following formula:

[Open area on day 8]−[Open area on day 1]/[Open area on day 1]

Specimens are fixed in 10% buffered formalin and paraffin embedded blocks are sectioned perpendicular to the wound surface (5 mm) and cut using an Olympus microtome. Routine hematoxylin-eosin (H&E) staining is performed on cross-sections of bisected wounds. Histologic examination of the wounds allows assessment of whether the healing process and the morphologic appearance of the repaired skin is improved by treatment with neuropeptide receptor. A calibrated lens micrometer is used by a blinded observer to determine the distance of the wound gap.

Experimental data are analyzed using an unpaired t test. A p value of <0.05 is considered significant.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 51

### Lymphadema Animal Model

The purpose of this experimental approach is to create an appropriate and consistent lymphedema model for testing the therapeutic effects of neuropeptide receptor in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 3-4 weeks.

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated or suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues.

Care is taken to control any mild bleeding resulting from this procedure. After lymphatics are occluded, the skin flaps are sealed by using liquid skin (Vetbond) (AJ Buck). The separated skin edges are sealed to the underlying muscle tissue while leaving a gap of ˜0.5 cm around the leg. Skin also may be anchored by suturing to underlying muscle when necessary.

To avoid infection, animals are housed individually with mesh (no bedding). Recovering animals are checked daily through the optimal edematous peak, which typically occurred by day 5-7. The plateau edematous peak are then observed. To evaluate the intensity of the lymphedema, the circumference and volumes of 2 designated places on each paw before operation and daily for 7 days are measured. The effect plasma proteins on lymphedema is determined and whether protein analysis is a useful testing perimeter is also investigated. The weights of both control and edematous limbs are evaluated at 2 μlaces. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: On the day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software(Chen/Victor). Data is recorded by one person, while the other is dipping the limb to marked area.

Blood-plasma protein measurements: Blood is drawn, spun, and serum separated prior to surgery and then at conclusion for total protein and Ca2+ comparison.

Limb Weight Comparison: After drawing blood, the animal is prepared for tissue collection. The limbs are amputated using a quillitine, then both experimental and control legs are cut at the ligature and weighed. A second weighing is done as the tibio-cacaneal joint is disarticulated and the foot is weighed.

Histological Preparations: The transverse muscle located behind the knee (popliteal) area is dissected and arranged in a metal mold, filled with freezeGel, dipped into cold methylbutane, placed into labeled sample bags at −80EC until sectioning. Upon sectioning, the muscle is observed under fluorescent microscopy for lymphatics.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

### Example 52

### Suppression of TNF Alpha-induced Adhesion Molecule Expression by Neuropeptide Receptor

The recruitment of lymphocytes to areas of inflammation and angiogenesis involves specific receptor-ligand interactions between cell surface adhesion molecules (CAMs) on lymphocytes and the vascular endothelium. The adhesion process, in both normal and pathological settings, follows a multi-step cascade that involves intercellular adhesion molecule-1 (ICAM-1), vascular cell adhesion molecule-1 (VCAM-1), and endothelial leukocyte adhesion molecule-1 (E-selectin) expression on endothelial cells (EC). The expression of these molecules and others on the vascular endothelium determines the efficiency with which leukocytes may adhere to the local vasculature and extravasate into the local tissue during the development of an inflammatory response. The local concentration of cytokines and growth factor participate in the modulation of the expression of these CAMs.

Tumor necrosis factor alpha (TNF-a), a potent proinflammatory cytokine, is a stimulator of all three CAMs on endothelial cells and may be involved in a wide variety of inflammatory responses, often resulting in a pathological outcome.

The potential of neuropeptide receptor to mediate a suppression of TNF-a induced CAM expression can be examined. A modified ELISA assay which uses ECs as a solid phase absorbent is employed to measure the amount of CAM expression on TNF-a treated ECs when co-stimulated with a member of the FGF family of proteins.

To perform the experiment, human umbilical vein endothelial cell (HUVEC) cultures are obtained from pooled cord harvests and maintained in growth medium (EGM-2; Clonetics, San Diego, Calif.) supplemented with 10% FCS and 1% penicillin/streptomycin in a 37 degree C. humidified incubator containing 5% CO₂. HUVECs are seeded in 96-well plates at concentrations of 1×10⁴ cells/well in EGM medium at 37 degree C. for 18-24 hrs or until confluent. The monolayers are subsequently washed 3 times with a serum-free solution of RPMI-1640 supplemented with 100 U/ml penicillin and 100 mg/ml streptomycin, and treated with a given cytokine and/or growth factor(s) for 24 h at 37 degree C. Following incubation, the cells are then evaluated for CAM expression.

Human Umbilical Vein Endothelial cells (HUVECs) are grown in a standard 96 well plate to confluence. Growth medium is removed from the cells and replaced with 90 ul of 199 Medium (10% FBS). Samples for testing and positive or negative controls are added to the plate in triplicate (in 10 ul volumes). Plates are incubated at 37 degree C. for either 5 h (selectin and integrin expression) or 24 h (integrin expression only). Plates are aspirated to remove medium and 100 μl of 0.1% paraformaldehyde-PBS(with Ca++ and Mg++) is added to each well. Plates are held at 4° C. for 30 min.

Fixative is then removed from the wells and wells are washed 1×with PBS(+Ca, Mg)+0.5% BSA and drained. Do not allow the wells to dry. Add 10 μl of diluted primary antibody to the test and control wells. Anti-ICAM-1-Biotin, Anti-VCAM-1-Biotin and Anti-E-selectin-Biotin are used at a concentration of 10 μg/ml (1:10 dilution of 0.1 mg/ml stock antibody). Cells are incubated at 37° C. for 30 min. in a humidified environment. Wells are washed×3 with PBS(+Ca, Mg)+0.5% BSA.

Then add 20 μl of diluted ExtrAvidin-Alkaline Phosphotase (1:5,000 dilution) to each well and incubated at 37° C. for 30 min. Wells are washed×3 with PBS(+Ca, Mg)+0.5% BSA. 1 tablet of p-Nitrophenol Phosphate pNPP is dissolved in 5 ml of glycine buffer (pH 10.4). 100 μl of pNPP substrate in glycine buffer is added to each test well. Standard wells in triplicate are prepared from the working dilution of the ExtrAvidin-Alkaline Phosphotase in glycine buffer: 1:5,000 (10⁰)>10^{-0.5}>10⁻¹>10^{-1.5}0.5 μl of each dilution is added to triplicate wells and the resulting AP content in each well is 5.50 ng, 1.74 ng, 0.55 ng, 0.18 ng. 100 μl of pNNP reagent must then be added to each of the standard wells. The plate must be incubated at 37° C. for 4h. A volume of 50 μl of 3M NaOH is added to all wells. The results are quantified on a plate reader at 405 nm. The background subtraction option is used on blank wells filled with glycine buffer only. The template is set up to indicate the concentration of AP-conjugate in each standard well [5.50 ng; 1.74 ng; 0.55 ng; 0.18 ng]. Results are indicated as amount of bound AP-conjugate in each sample.

The studies described in this example tested activity in neuropeptide receptor protein. However, one skilled in the art could easily modify the exemplified studies to test the activity of neuropeptide receptor polynucleotides (e.g., gene therapy), agonists, and/or antagonists of neuropeptide receptor.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Moreover, the sequence listing from U.S. application Ser. No. US 08/462,509 and PCT application WO 96/34877 are hereby incorporated by reference.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:1, 3, or 5, or a polynucleotide fragment of the cDNA sequence included in ATCC Deposit No: 97128;
(b) a polynucleotide encoding a polypeptide fragment of SEQ ID NO:2, 4, or 6, or the cDNA sequence included in ATCC Deposit No: 97128;
(c) a polynucleotide encoding a polypeptide domain of SEQ ID NO:2, 4, or 6 or the cDNA sequence included in ATCC Deposit No: 97128;
(d) a polynucleotide encoding a polypeptide epitope of SEQ ID NO:2, 4, or 6, or the cDNA sequence included in ATCC Deposit No: 97128;
(e) a polynucleotide encoding a polypeptide of SEQ ID NO:2, 4, or 6, or the cDNA sequence included in ATCC Deposit No: 97128 having biological activity;
(f) a polynucleotide which is a variant of SEQ ID NO:1, 3, or 5;
(g) a polynucleotide which is an allelic variant of SEQ ID NO:1, 3, or 5;
(h) a polynucleotide which encodes a species homologue of the SEQ ID NO:2, 4, or 6;
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a mature form or a secreted protein.

3. The isolated nucleic acid molecule of claim 1, wherein the polynucleotide fragment comprises a nucleotide sequence encoding the sequence identified as SEQ ID NO:2, 4, or 6, or the coding sequence included in ATCC Deposit No: 97128.

4. The isolated nucleic acid molecule of claim 1, wherein the polynucleotide fragment comprises the entire nucleotide sequence of SEQ ID NO:1, 3, or 5, or the cDNA sequence included in ATCC Deposit No: 97128.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 9.

10. The recombinant host cell of claim 9 comprising vector sequences.

11. An isolated polypeptide comprising an amino acid sequence at least 95% identical to a sequence selected from the group consisting of:
(a) a polypeptide fragment of SEQ ID NO:2, 4, or 6 or the encoded sequence included in ATCC Deposit No: 97128;
(b) a polypeptide fragment of SEQ ID NO:2, 4, or 6, or the encoded sequence included in ATCC Deposit No: 97128 having biological activity;
(c) a polypeptide domain of SEQ ID NO:2, 4, or 6 or the encoded sequence included in ATCC Deposit No: 97128;
(d) a polypeptide epitope of SEQ ID NO:2, 4, or 6 or the encoded sequence included in ATCC Deposit No: 97128;
(e) a mature form of a secreted protein;
(f) a full length secreted protein;
(g) a variant of SEQ ID NO:2, 4, or 6;
(h) an allelic variant of SEQ ID NO:2, 4, or 6; or
(i) a species homologue of the SEQ ID NO:2, 4, or 6.

12. The isolated polypeptide of claim 11, wherein the mature form or the full length secreted protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition which comprises administering to a mammalian subject a therapeutically effective amount of the polypeptide of claim 11.

18. A method of diagnosing a pathological condition or a susceptibility to a pathological condition in a subject related to expression or activity of a secreted protein comprising:
(a) determining the presence or absence of a mutation in the polynucleotide of claim 1;
(b) diagnosing a pathological condition or a susceptibility to a pathological condition based on the presence or absence of said mutation.

19. A method of diagnosing a pathological condition or a susceptibility to a pathological condition in a subject related to expression or activity of a secreted protein comprising:
(a) determining the presence or amount of expression of the polypeptide of claim 11 in a biological sample;
(b) diagnosing a pathological condition or a susceptibility to a pathological condition based on the presence or amount of expression of the polypeptide.

20. A method for identifying binding partner to the polypeptide of claim 11 comprising:
(a) contacting the polypeptide of claim 11 with a binding partner; and
(b) determining whether the binding partner effects an activity of the polypeptide.

21. The gene corresponding to the cDNA sequence of SEQ ID NO:2, 4, or 6.

22. A method of identifying an activity in a biological assay, wherein the method comprises:
(a) expressing SEQ ID NO:1, 3, or 5 in a cell;
(b) isolating the supernatant;
(c) detecting an activity in a biological assay; and
(d) identifying the protein in the supernatant having the activity.

23. The product produced by the method of claim 22.

24. A method for preventing, treating, or ameliorating a medical condition which comprises administering to a mammalian subject a therapeutically effective amount of the polynucleotide of claim 1.


SEQUENCE LISTING (23 sequences)

SEQ ID NO: 1 - DNA, 1209 bp, Homo sapiens
  CDS (1)..(1209)
atggagccctcagccaccccaggggcccagatgggggtcccccctggcagcagagagccgtcccctgtgcctccagactatgaagatgagtttctccgctatctgtggcgtgattatctgtacccaaaacagtatgagtgggtcctcatcgcagcctatgtggctgtgttcgtcgtggccctggtgggcaacacgctggtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcagatgacctctatcccaagatctaccacagttgcttctttattgtcacctacctggccccactgggcctcatggccatggcctatttccagatattccgcaagctctggggccgccagatccccggcaccacctcagcactggtgcggaactggaagcgcccctcagaccagctgggggacctggagcagggcctgagtggagagccccagccccggggccgcgccttcctggctgaagtgaagcagatgcgtgcaaggaggaagacagccaagatgctgatggtggtgctgctggtcttcgccctctgctacctgcccatcagcgtcctcaatgtccttaagagggtgttcgggatgttccgccaagccagtgaccgcgaagctgtctacgcctgcttcaccttctcccactggctggtgtacgccaacagcgctgccaaccccatcatctacaacttcctcagtggcaaattccgggagcagtttaaggctgccttctcctgctgcctgcctggcctgggtccctgcggctctctgaaggcccctagtccccgctcctctgccagccacaagtccttgtccttgtag

SEQ ID NO: 2 - protein, 402 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSL

SEQ ID NO: 3 - DNA, 1110 bp, Homo sapiens
  CDS (1)..(1110)
atggagccctcagccaccccaggggcccagatgggggtcccccctggcagcagagagccgtcccctgtgcctccagactatgaagatgagtttctccgctatctgtggcgtgattatctgtacccaaaacagtatgagtgggtcctcatcgcagcctatgtggctgtgttcgtcgtggccctggtgggcaacacgctggtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcagatgacctctatcccaagatctaccacagttgcttctttattgtcacctacctggccccactgggcctcatggccatggcctatttccagatattccgcaagctctggggccgccagatccccggcaccacctcagcactggtgcggaactggaagcgcccctcagaccagctgggggacctggagcagggcctgagtggagagccccagccccggggccgcgccttcctggctgaagtgaagcagatgcgtgcaaggaggaagacagccaagatgctgatggtggtgctgctggtcttcgccctctgctacctccccatcagcgtcctcaatgtccttaagagggtgttcgggatgttccgccaagccagtgaccgcgaagctgtctacgcctgcttcaccttctcccactggctggtgtacgccaacagcgctgccaaccccatcatctacaacttcctcagtggccttccctggagtctgctctaa

SEQ ID NO: 4 - protein, 369 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGLPWSLL

SEQ ID NO: 5 - DNA, 1133 bp, Homo sapiens
  CDS (1)..(1116)
atggagccctcagccaccccaggggcccagatgggggtcccccctggcagcagagagccctcccctgtgcctccagactatgaagatgagtttctccgctatctgtggcgtgattatctgtacccaaaacagtatgagtgggtcctcatcgcagcctatgtggctgtgttcgtcgtggccctggtgggcaacacgctggtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcagatgacctctatcccaagatctaccacagttgcttctttattgtcacctacctggccccactgggcctcatggccatggcctatttccagatattccgcaagctctggggccgccagatccccggcaccacctcagcactggtgcggaactggaagcgcccctcagaccagctgggggacctggagcagggcctgagtggagagccccagccccggggccgcgccttcctggctgaagtgaagcagatgcgtgcaaggaggaagacagccaagatgctgatggtggtgctgctggtcttcgccctctgctacctgcccatcagcgtcctcaatgtccttaagagggtgttcgggatgttccgccaagccagtgaccgcgaagctgtctacgcctgcttcaccttctcccactggctggtgtacgccaacagcgctgccaaccccatcatctacaacttcctcagtggatgtaaagagaagagtctagttctgtcctgaccatcgtgccccgg

SEQ ID NO: 6 - protein, 372 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGCKEKSLVLS

SEQ ID NO: 7 - DNA, 30 bp, Homo sapiens
cactaaagcttaatggagccctcagccacc

SEQ ID NO: 8 - DNA, 25 bp, Homo sapiens
acaagtccttgtccttctagagggc

SEQ ID NO: 9 - DNA, 26 bp, Homo sapiens
cctaggatgcccctctgctgcagcgg

SEQ ID NO: 10 - DNA, 25 bp, Homo sapiens
acaagtccttgtccttctagagggc

SEQ ID NO: 11 - DNA, 32 bp, Homo sapiens
cgggatccgccatcatggagccctcagccacc

SEQ ID NO: 12 - DNA, 25 bp, Homo sapiens
acaagtccttgtccttctagagggc

SEQ ID NO: 13 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 14 - protein, 5 aa, Homo sapiens
  site (3): Xaa equals any amino acid
WSXWS

SEQ ID NO: 15 - DNA, 86 bp, Homo sapiens
gcgcctcgagatttccccgaaatctagatttccccgaaatgatttccccgaaatgatttccccgaaatatctgccatctcaattag

SEQ ID NO: 16 - DNA, 27 bp, Homo sapiens
gcggcaagctttttgcaaagcctaggc

SEQ ID NO: 17 - DNA, 271 bp, Homo sapiens
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 18 - DNA, 32 bp, Homo sapiens
gcgctcgagggatgacagcgatagaaccccgg

SEQ ID NO: 19 - DNA, 31 bp, Homo sapiens
gcgaagcttcgcgactccccggatccgcctc

SEQ ID NO: 20 - DNA, 12 bp, Homo sapiens
ggggactttccc

SEQ ID NO: 21 - DNA, 73 bp, Homo sapiens
gcggcctcgaggggactttcccggggactttccggggactttccgggactttccatcctgccatctcaattag

SEQ ID NO: 22 - DNA, 256 bp, Homo sapiens
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 23 - protein, 384 aa, Homo sapiens
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMDKMRDNKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI